(19) United States
(12) Patent Application Publication
(10) Pub. No.: US 2026/0345792 A1
(43) Pub. Date: Nov. 5, 2026

(54) PROTACs AND HyT-PD MOLECULES FOR TARGETED PROTEIN DEGRADATION OF DCAF15 AND THEIR USE IN THE TREATMENT OF AMYLOIDOSIS

(71) Applicants: KRÆFTENS BEKÆMPELSE, Copenhagen Ø (DK); DANMARKS TEKNISKE UNIVERSITET, Kongens Lyngby (DK)

(72) Inventors: Sólveig Hlín BRYNJÓLFSDÓTTIR, Copenhagen Ø (DK); Lisa Beth FRANKEL, Copenhagen Ø (DK); Luca LARAIA, Kongens Lyngby (DK); Joseph Franz Georg HOOCK, Kongens Lyngby (DK); Maria Brandt SCHJØNNING, Kongens Lyngby (DK); Matilde Lind Hartvig NIELSEN, Kongens Lyngby (DK)

(73) Assignees: KRÆFTENS BEKÆMPELSE, Copenhagen Ø (DK); DANMARKS TEKNISKE UNIVERSITET, Kongens Lyngby (DK)

(21) Appl. No.: 19/485,836

(22) PCT Filed: Jun. 6, 2024
(86) PCT No.: PCT/EP2024/065601

§ 371 Date: Nov. 19, 2025

(30) Foreign Application Priority Data
Jun. 7, 2023 (EP) 23177872.1
Jan. 19, 2024 (EP) 24152991.6

(51) Int. Cl. A61K 47/55; A61P 25/28

(52) U.S. Cl. CPC A61K 47/55; A61P 25/28

(57) ABSTRACT

The present invention relates to the finding of the role of DCAF15 in protein aggregation. The present invention further relates to PROTACs and HyT-PD molecules for targeted protein degradation (TPD) of DCAF15 as well as their use as a medicament for the treatment of amyloidosis, in particular their use in the treatment of synucleinopathies.

### FIELD OF THE INVENTION

The present invention relates to the finding of the role of DDB1 and CUL4 associated factor 15 (DCAF15) in protein aggregation in cells. The present invention further relates to proteolysis-targeting chimera (PROTACs) and hydrophobic tag-based protein degradation (HyT-PD) molecules for targeted protein degradation (TPD) of DCAF15 and their use in the treatment of amyloidosis, in particular in the treatment of synucleinopathies.

### BACKGROUND OF THE INVENTION

Proteins are essential for living organisms as they perform a vast array of functions, such as DNA replication and transcription, acting as enzymes for catalysing reactions, forming receptors for the cell to respond to stimuli and transporting molecules from one location to another. Once proteins have been synthesized in the cells, they fold into specific three-dimensional conformations (i.e. secondary and tertiary structures) that are thermodynamically favourable (i.e. their native state). This folding process is essential for the protein to acquire its proper structure and function and is driven by a tendency for hydrophobic portions of the protein to shield themselves from the hydrophilic environment of the cell by burying the hydrophobic portions into the interior of the protein. The native state of the protein is stabilized by non-covalent interactions (e.g. hydrogen bonds, salt bridges, and van der Waals forces), as well as disulfide bonds between cysteine residues.

Cellular stress may lead to damaged or misfolded proteins. In healthy cells, damaged or misfolded proteins are degraded by two main routes: the ubiquitin-proteasome system (UPS) and autophagy/lysosomal proteolysis. If the cell fails to degrade misfolded protein, it may eventually lead to protein aggregation. Amyloids are aggregates of proteins characterised by a fibrillar morphology, which have been associated with a group of diseases referred to as amyloidosis. About 60 amyloid proteins have been identified so far wherein at least 36 have been associated with human disease. Among the amyloidosis, there is a group of neurodegenerative diseases referred to as synucleinopathies (also called α-synucleinopathies). The common hallmark of this heterogeneous group of neurodegenerative diseases is amyloid deposits of the protein α-synuclein in neurons, nerve fibres and/or glial cells. These alpha-synuclein deposits (also referred to as Lewy bodies) can be seen in neurons under a microscope.

Parkinson's disease (PD), dementia with Lewy bodies (DLB), and multiple system atrophy (MSA) are examples of synucleinopathies, wherein the cells are unable to effectively remove the highly cytotoxic alpha-synuclein deposits, damaging the cell and eventually leading to a loss of neurons (i.e. neurodegeneration). For PD, the main pathological characteristics are cell death of dopamine secreting neurons (dopaminergic neurons) in the brain's basal ganglia leading to low dopamine levels. The loss of dopaminergic neurons is accompanied by the death of astrocytes (star-shaped glial cells) and a significant increase in the number of microglia in the substantia nigra. To date, no cure for synucleinopathies exists, and the treatments are aimed at managing symptoms. For example, the symptomatic treatment in PD aims at restoring dopamine levels with e.g. the dopamine precursor levodopa (L-DOPA) or dopamine agonists and/or preventing the metabolism of dopamine with ocatechol-O-methyltransferase (COMT) inhibitors or monoamine oxidase-B (MAO-B) inhibitors. However, as the disease progresses, the medications become less effective due to increasing loss of neurons. Thus, there is need in the art for identifying new treatment strategies for amyloidosis, in particular synucleinopathies that target the underlying pathology (i.e. that prevent the deposits of α-synuclein in neurons) leading to neurodegeneration, rather than merely providing symptomatic relief.

In the cells, the ubiquitin-proteasome system (UPS) plays a critical role in maintaining intracellular protein homeostasis by eliminating misfolded, damaged, and worn-out proteins to prevent aggregation. This process consists of a cascade of distinct steps, starting with ubiquitin activation by enzyme E1. Ubiquitin is then passed to the E2 or ubiquitin-conjugating enzyme by transthioesterification. Subsequently, E3 ubiquitin ligases promote the transfer of ubiquitin onto a lysine of the substrate protein, after which it is targeted for proteasomal degradation. The human genome includes two members of the E1 enzyme family, roughly 40 E2s, and more than 600 E3 ubiquitin ligases (Kleiger and Mayor, 2014). The E3 ligases are categorized into three classes based on their mechanism of ubiquitin transfer, namely RING (really interesting new gene), HECT (homologous to E6AP C-terminus), and RBR (RING between RING). The most abundant class RING is characterized by the direct transfer of ubiquitin from E2 to a substrate and includes approximately 600 RING E3 ligases.

Hijacking of the UPS for TPD has attracted substantial interest in the last decade owing to its potential to therapeutically modulate proteins that have previously been considered undruggable or proved difficult to target with conventional small molecules. A major class of molecules that may enable such proteins to be modulated through TPD are known as proteolysis-targeting chimera (PROTAC) protein degraders. These are heterobifunctional small molecules consisting of two ligands joined by a linker: one ligand recruits and binds a protein of interest (POI) while the other recruits and binds an E3 ubiquitin ligase. Simultaneous binding of the POI and ligase by the PROTAC highjacks the UPS and induces ubiquitylation of the POI and its subsequent degradation, after which the PROTAC is recycled to target another copy of the POI.

As opposed to small molecule inhibitors which need to continuously occupy the active site of the POI (i.e. “occupancy driven”) to exert a lasting inhibitory effect, PROTACs do not require extremely high affinity nor precise action sites to achieve the degradation effects due to its ability to degrade proteins in an “event-driven” manner. Compared to traditional small molecule inhibitors, PROTACs possess several advantages, including reduced drug resistance and the ability to mediate the degradation of what was previously considered undruggable proteins. However, the pharmacokinetic properties of PROTACs are often flawed due to their violation of Lipinski's rule of five.

Hydrophobic tag-based protein degradation (HyT-PD) also referred to as hydrophobic tagging (HyT) is another strategy for TPD. As previously mentioned, hydrophobic domains of native proteins are buried within the interior of the protein structure and only become exposed on the surface if the protein is misfolded. If hydrophobic domains are exposed on the protein surface, cells interpret them as unstable or misfolded proteins, leading to the degradation of these proteins through protein quality control (PQC) mechanisms. HyT-PD molecules exploit this principle. HyT-PD molecules (or hydrophobic tag tethering degraders (HyTTDs)) comprise a ligand for the POI linked to a highly hydrophobic group, such as adamantane, that serves as the hydrophobic tag (HyT). Upon binding of the HyT-PD molecule to the POI, the hydrophobic tag is exposed on the surface of the POI, leading to the degradation of the POI. The precise mechanism of action of HyT-PD molecules remains elusive and HyT-PD molecules have received relatively limited attention. Studies have suggested that HyT-PD molecules may facilitate POI degradation via multiple pathways, such as the UPS pathway, the autophagy pathway, the unfolded protein response (UPR) pathway, and the ubiquitin-independent proteasome system (UIPS) pathway. Leveraging their lower molecular weight and reduced numbers of hydrogen bond donors/acceptors (HBDs/HBAs) in comparison with PROTACs, HyT-PD molecules may present a compelling approach for enhancing druglike properties. Furthermore, HyT-PD molecules are only affected by POI mutations, and the inactivation of a single protein quality control (PQC) pathway does not completely block the degradation of the POI. On the contrary, PROTACs require the simultaneous binding of E3 ubiquitin ligase and the POI, and thus any mutation in either related protein or failure to form the PROTAC-E3-POI complex may result in the inactivation of PROTACs and the development of drug resistance.

The present invention relates to the finding that knock-out cells lacking DCAF15 are effectively protected against protein aggregation compared to wild-type cells. This finding may provide basis for a paradigm shift in the treatment of diseases caused by or associated with amyloid aggregates by targeted protein degradation (TPD) of DCAF15 using PROTACs or HyT-PD molecules based on ligands for DCAF15 known in the art. For example, indisulam (an aryl sulfonamide drug) that inhibits the proliferation of certain human cancer cell lines as well as tasisulam and chloroquinoxaline sulfonamide (CQS) (commonly known as SPLAMs—SPLicing inhibitor sulfonAMides) have shown to act as ligands for DCAF15 (see e.g. Han et. al., 2017 and WO 2022/169755). Furthermore, a vast number of E3 ubiquitin ligase ligands (see e.g. Bricelj et al., 2021) and hydrophobic tags (HyTs) are known in the art (see e.g. He et al., 2023). The present inventors have previously developed a number of PROTACs for degradation of DCAF15 (see EP23177872.1). Albeit these PROTACs were shown to prevent aggregation in cells, they are less optimal in terms of drug-like properties due to violation of the Lipinski rule of 5, which may hamper their use as orally active drugs. The present inventors have now surprisingly identified that HyT-PD molecules may also be effectively designed for targeted protein degradation of DCAF15. In particular, the HyT-PD molecules have more druglike properties, such as lower molecular weight (MW) and less hydrogen bond donors/acceptors, which provides more drug-like properties making them suitable as orally active drugs.

### SUMMARY OF THE INVENTION

The present invention relates to the finding of the pivotal role of DCAF15 in protein aggregation. The present inventors found that DCAF15 knock-out cells (i.e. DCAF15−/− cells) were protected against the formation of stress-induced protein aggregates compared to wild-type cells (Examples 1, 2 and 3), that DCAF15−/− cells showed improved cellular fitness in response to proteotoxic stress compared to wild-type cells (Example 4), and that DCAF15−/− neuroblastoma cells were protected against α-synuclein aggregation compared to wild-type cells (Example 5). These important findings provide a completely new basis for providing effective treatments of the underlying pathology of amyloidosis, in particular synucleinopathies, by proteolysis-targeting chimeras (PROTACs) or HyT-PD molecules. In the first aspect described herein, the present invention relates to PROTACs for targeted protein degradation of DCAF15 capable of preventing protein aggregation in cells. In the second aspect described herein, the present invention relates to the medical use of said PROTACs. In a third aspect described herein, the present invention relates to HyT-PD molecules for targeted protein degradation of DCAF15 capable of preventing aggregation in cells. In a fourth aspect described herein, the present invention relates to the medical use of said HyT-PD molecules. In particular, the present inventors surprisingly found that rather small HyT-PD molecules having lower MW, fewer hydrogen bond donors and/or acceptors, compared to the PROTACs, were effective in targeted protein degradation of DCAF15 making them suitable as orally active drugs. The invention is set out in the detailed description and in the appended set of claims.

### BRIEF DESCRIPTION OF THE FIGURES

Embodiments of the invention will be described in more detail in the following with regard to the accompanying figures.

FIG. 1A shows HCT116 cells with the indicated genotypes treated with 5 μg/ml puromycin for 4 h or left untreated (NT) before fixation. Cells were stained for ubiquitinated proteins and p62. Separate and merged channels from a representative experiment are shown (n>3).

FIG. 1B shows the quantification of number of ubiquitin and p62 co-stained aggregates per cell from FIG. 1A.

FIG. 1C shows BE2C cells with the indicated genotypes treated with 5 μg/ml puromycin for 4 h or left untreated (NT) before fixation. Cells were stained for ubiquitinated proteins and p62. Separate and merged channels from a representative experiment are shown (n>3).

FIG. 1D shows the quantification of the number of ubiquitin and p62 co-stained aggregates per cell from FIG. 1C.

FIG. 2A shows HCT116 cells treated with 10 μM MG132, 10 μg/ml Thapsigargin or 400 μM H₂O₂ for 4 h as indicated and analysed for ubiquitin and p62 positive protein aggregates. Representative experiments are shown, MG132 (n=4), Thapsigargin (n=3), H₂O₂ (n=1).

FIG. 2B shows quantifications of aggregates per cell from MG132 treatment in FIG. 2A for indicated three cell lines.

FIG. 3A shows HCT116 cells treated with 5 μg/ml puromycin for 4 h alone or in combination with 200 nM Bafilomycin-1 or 5 μM MG132. Cells were fixed and stained for ubiquitin and p62. A representative experiment is shown (n=3).

FIG. 3B shows quantification of double positive puncta per cell from FIG. 3A.

FIG. 4A shows HCT116 cells treated with 5 μg/ml puromycin for 4 h (bottom) or left untreated (top) before being released into normal growth media for 72 h. Confluency was recorded with imaging every 4 h. Representative experiments with technical triplicates are shown (n=3).

FIG. 4B shows HCT116 cells treated with 5 μg/ml puromycin for 4 h before release into full media or left untreated for 7 days before fixation and crystal violet staining.

FIG. 4C shows quantification of FIG. 4B as integrated density per well.

FIG. 5A shows time series from live-cell imaging of α-synuclein aggregation in BE2C cells (WT or DCAF15−/−) stably expressing GFP-α-synuclein A53T. Aggregation was induced by transfection of 600 ng/ml pre-formed fibrils (PFF) and imaging performed once per hour for 24 h to monitor aggregation (n=3). Representative images from every 4th hour (left) and a representative single cell after 24 h PFF treatment (right) are shown.

FIG. 5B shows quantification of α-synuclein inclusion number (>2 μm diameter) from FIG. 5A.

FIG. 5C shows quantification of total GFP levels from indicated cell lines/treatments (time point 1 in FIG. 5A).

FIG. 6A shows HCT116 DCAF15−/− cells stably expressing doxycycline-inducible GFP-DCAF15-3xF. Cells were treated with doxycycline for 24 h to induce GFP-DCAF15-3xF expression and treated with indicated PROTACS (0.5-2 uM) for 24 h. Cells were lysed and analyzed for GFP-DCAF15 levels by western blot using anti-GFP antibody.

FIG. 6B shows BE2C WT cells treated with 0.5-2 uM of indicated PROTACs (or DMSO control) for 4 h. 5 ug/ml puromycin was added for 2 h to induce aggregation and cells fixed and stained for ubiquitin and p62. “Aggregates per cell” represents the mean no. of double positive puncta (for ubiquitin and p62) per cell. Experiment was performed with technical duplicates analyzing >110 cells per sample.

FIG. 6C shows BE2C WT cells treated with 1, 2 or 5 uM of indicated HyT-PD molecule (or DMSO control) for 24 h. Compound 24 was tested in HCT116 WT cells where cells were treated for 6 h with the compound. 5 ug/ml puromycin was added for the last 4 h to induce aggregation and cells fixed and stained for ubiquitin and p62. “Aggregates per cell” represents the mean no. of double positive puncta (for ubiquitin and p62) per cell. Experiment was performed with technical triplicates analyzing >120 cells per sample (see FIG. 6C).

FIG. 6D shows HEK293T cells expressing HiBit-DCAF15 at endogenous level were treated with DMSO, Indisulam, E7820 and the indicated PROTACs and HyT-PD molecules at the indicated concentrations for 24 h (n=3). HiBit levels were measured using the HiBit lytic detection system developed by Promega, following manufactures instructions. The obtained values were compared and plotted as the fold change vs the mean of the DMSO control sample (see FIG. 6D).

FIG. 6E shows HEK293T cells expressing HiBit-DCAF15 at endogenous level were treated with DMSO, Indisulam, E7820 and the indicated HyT-PD molecules at the indicated concentrations for 24 h (n=2). HiBit levels were measured using the HiBit lytic detection system developed by Promega, following manufactures instructions. The obtained values were compared and plotted as the fold change vs the mean of the DMSO control sample.

### DEFINITIONS

In the present context, a linker comprising a PEG (polyethylene glycol) chain should be understood to include the motif —(O—CH₂—CH₂)ₙ—O— in the linker, wherein n is an integer from 1-6. In the present context, a linker comprising an alkyl chain should be understood to include the motif —(CH₂)ₙ— (i.e. alkanediyl or alkylene) in the linker, wherein n is an integer from 1-16. In the present context, a linker comprising an extended glycol chain should be understood to include the motif, —(O—CH₂—(CH₂)ₙ—CH₂—O)—, in the linker, wherein n is an integer from 1-2. In the present context, a linker comprising a cyclic motif should be understood to include a(n) (un)saturated carbocycle or heterocycle as part of the linker to rigify the linker. Preferably, the cyclic motif of the linker comprises a cyclohexane ring system, a benzene ring system, a piperidine ring system, and/or a piperazine ring system of Formula (L1-L4) as shown below:

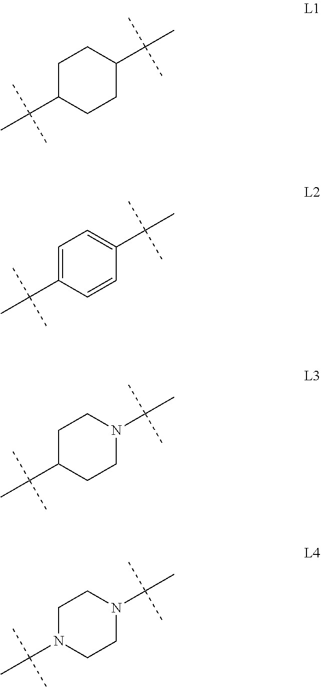

In the present context, a linker may comprise mixtures of PEG chains, extended glycol chains, or alkyl chain as exemplified herein. It should be understood that the linkers may be linear, branched or comprise one or more cyclic motifs. Preferably, the linkers are linear or rigidified with a cyclic motif. For the PROTACs, the linkers may have a total chain length of 6-24 atoms, preferably 6-22 atoms, more preferably 6-20 atoms, even more preferably 6-18 atoms, most preferably 8-16 atoms. For the HyT-PD molecules, the linkers may have a chain length of 3-18 atoms, preferably 3-14 atoms, such as 3-10 atoms, more preferably 3-8 atoms, most preferably 3-6 atoms. In calculation of the linker chain length, any functional groups used to connect the linker to the E3 ligase ligand or the hydrophobic tag (HyT) in one end and the linker to the DCAF15 ligand in the opposite end are included. As a non-limiting example, the linker of PROTAC 1 has a chain length of 9 atoms. As another non-limiting example, HyT-PD molecule 31 has a chain length of 4 atoms (see Table 1). In the present context, it should be appreciated that the linkers comprising a PEG chain, an alkyl chain, and/or an extended glycol chain may be covalently attached to the DCAF15 ligand in one end and to the E3 ligase/HyT in the opposite via a variety of functional groups commonly used in PROTACs or HyT-PD molecules for linker attachment. Such functional groups may include e.g. amides, sulfonamides, sulfonates, carbamates, esters, thioesters, ketones, ethers, thiol ethers, amines, alkynes, triazoles or tetrazoles due to their prior use in PROTACs/HyT-PD molecules design and their synthetic accessibility from commercial building blocks. Most preferably, the linker is covalently connected to the DCAF15 ligand with an amide or sulfonamide.

In the present context, the PROTACs or HyT-PD molecules according to the invention may be in the form of a pharmaceutically acceptable salt and/or solvate thereof. The salt may be acid addition salts or basic salts depending on whether acid or basic moieties are present in the DCAF15 ligand, the E3 ligase ligand, or the linker. Examples of pharmaceutically acceptable salts can be found in e.g. Handbook of Pharmaceutical Salts.

In the present context, amyloidosis refers to a group of diseases in which abnormal accumulation of amyloid fibrils (also referred to as amyloids) occurs. Preferably, the amyloidosis is caused by amyloid fibrils of Tau, ß amyloid (Aβ), huntingtin (HTT) or α-synuclein (αSyn), more preferably α-synuclein (αSyn). In the present context, synucleinopathies (also called α-synucleinopathies) refers to a group of neurodegenerative diseases characterised by the abnormal accumulation of amyloid fibrils of α-synuclein. Examples of synucleinopathies are Parkinson's disease (PD), dementia with Lewy bodies (DLB), and multiple system atrophy (MSA).

### DETAILED DESCRIPTION OF THE INVENTION

The present inventors found that DCAF15 knock-out cells were protected against protein aggregation compared to wild-type cells. This finding allows for the treatment of diseases caused by or associated with amyloid aggregates by targeted protein degradation (TPD) of DCAF15. Such diseases include amyloidosis characterized by build-up of amyloid fibrils in tissue(s) or synucleinopathies characterised by the accumulation of alpha-synuclein aggregates in neurons, nerve fibres, or glial cells. The skilled person will appreciate that PROTACs for degradation of DCAF15 may be designed based on various known ligands for DCAF15 (POI) covalently linked to various known ligands for an E3 ubiquitin ligase other than the ones exemplified herein. Likewise, the skilled person will appreciate that HyT-PD molecules for degradation of DCAF15 may be designed based on various known ligands for DCAF15 (POI) covalently linked to various known hydrophobic tags (HyT) other than the ones exemplified herein.

### Aspect 1: PROTACs

Ligands for DCAF15 (i.e. Protein of Interest)

A number of ligands for DCAF15 are known in the art, such as the SPLicing inhibitor sulfonAMides indisulam, tasisulam, and chloroquinoxaline sulfonamide (CQS). Furthermore, various analogues of the indisulam scaffold have been synthesized and shown to bind to DCAF15, such as those described in e.g. WO 2022/169755. It should be appreciated that any of the known ligands for DCAF15 may be used as ligand in the PROTACs according to the invention. In a preferred embodiment of the invention, the ligand for DCAF15 comprises a structure of Formula (DCAF15-I), (DCAF15-II), (DCAF15-III),

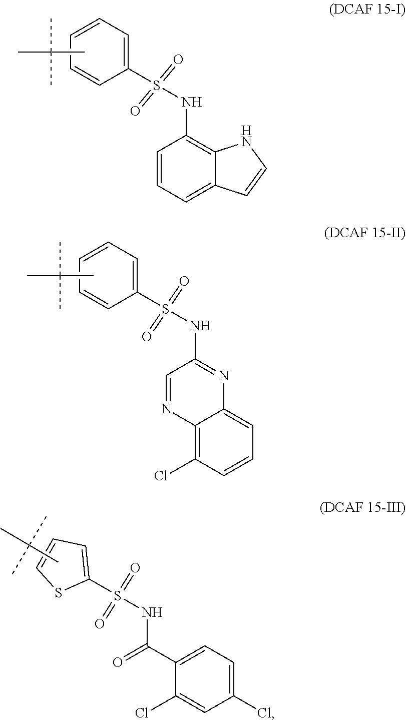

wherein the dotted lines denote a covalent connection to an E3 ligase ligand via a linker.

In a more preferred embodiment of the invention, the DCAF15 ligand is selected from a compound of Formula (DCAF15-I),

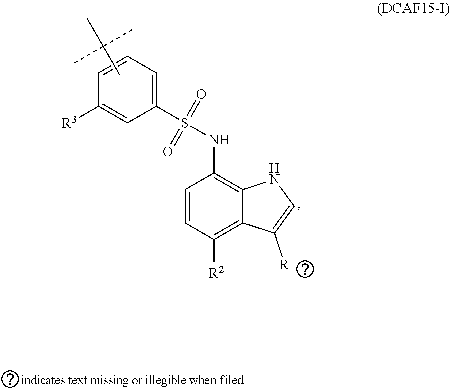

wherein R¹ is selected as F, Cl, Br, I, CN, Me, or CF₃; R² is selected as H, CN, CF₃ or Me; R³ is selected as CN or H; and wherein the dotted line denotes a covalent connection to an E3 ligase ligand via a linker. Preferably, R¹ is selected as Cl or CN, most preferably Cl; R² is selected as H or Me, most preferably H; and R³ is selected as H.

In an even more preferred embodiment, the DCAF15 ligand is selected from a compound of Formula (DCAF15-Ia) or (DCAF15-Ib),

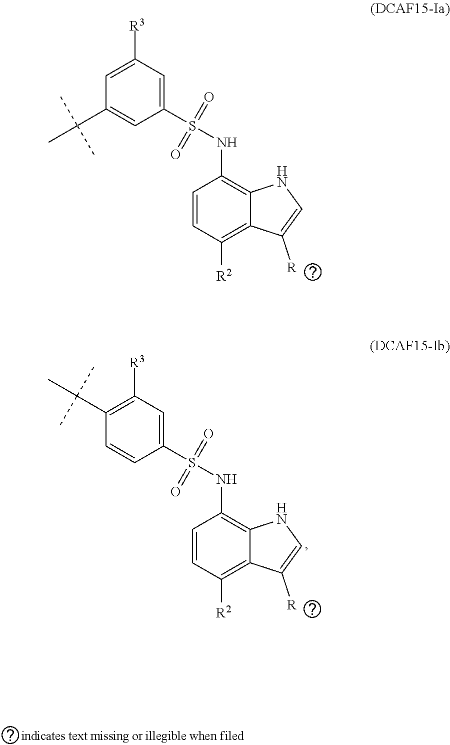

wherein, R¹ is selected as F, Cl, Br, I, CN, Me, or CF₃; R² is selected as H, CN, CF₃ or Me; R³ is selected as CN or H; and wherein the dotted lines denote a covalent connection to an E3 ubiquitin ligase ligand via a linker. Preferably, R¹ is selected as Cl or CN, most preferably Cl; R² is selected as H or Me, most preferably H; and R³ is selected as H.

In a most preferred embodiment, the DCAF15 ligand is selected from a compound of Formula (DCAF15-Ia)

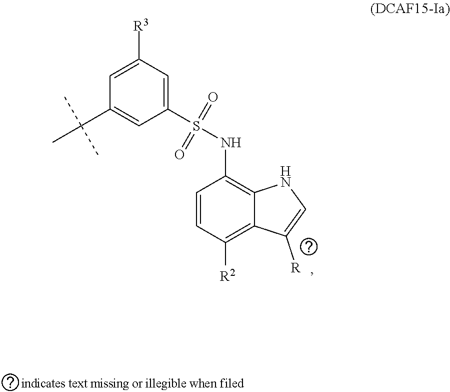

wherein R¹ is selected as F, Cl, Br, I, CN, Me, or CF₃; R² is selected as H, CN, CF₃ or Me; R³ is selected as CN or H; and wherein the dotted line denotes a covalent connection to an E3 ubiquitin ligase ligand via a linker. Preferably, R¹ is selected as Cl or CN, most preferably Cl; R² is selected as H or Me, most preferably H; and R³ is selected as H.

### E3 Ubiquitin Ligase Ligands for PROTACs

A number of ligands for E3 ubiquitin ligases are known in the art, which are either commercially available or may be synthesized using known procedures. Such ligands include ligands for the cereblon (CRBN), von Hippel-Lindau (VHL), ring finger protein 114 (RNF114), DDB1- and CUL4-associated factor 11 (DCAF11), DDB1 and CUL4 associated factor 15 (DCAF15), or Mouse double minute 2 homolog (MDM2) E3 ubiquitin ligases as described in e.g. Bricelj et al., 2021. Development of potent E3 ubiquitin ligase ligands has primarily targeted the von Hippel-Lindau (VHL) and cereblon (CRBN) E3 ubiquitin ligases, and the majority of recently reported PROTACs still utilize either VHL or CRBN as E3 ligases. In the present context, it should be appreciated that any known E3 ubiquitin ligase ligand may be used in the PROTACs according to the invention. In an embodiment of the invention, the E3 ubiquitin ligase ligand is selected from a ligand for CRBN, VHL, RNF114, DCAF11, DCAF15, or MDM2 E3 ubiquitin ligases. In a highly preferred embodiment of the invention, the E3 ubiquitin ligase ligand is selected from a CRBN or a VHL E3 ubiquitin ligase ligand.

### Examples of Known CRBN E3 Ubiquitin Ligase Ligands

It will be apparent to those skilled in the art that any of the known CRBN ligands may be used in the PROTACs according to the invention. These ligands are based on thalidomide or analogues thereof and are commercially available or may be synthesized via known synthetic routes.

In a highly preferred embodiment of the invention, the E3 ubiquitin ligase ligand is a CRBN ligand selected from a compound of Formula (CRBN-I), (CRBN-II)

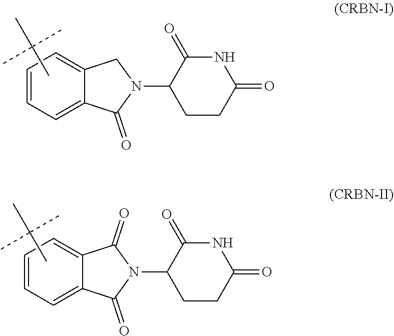

wherein the dotted lines denote a covalent connection to an DCAF15 ligand via a linker. In a more preferred embodiment of the invention, the CRBN ligase ligand is a compound of Formula (CRBN-II). In a most preferred embodiment of the invention, the CRBN ligase ligand is a compound of Formula (CRBN-II)

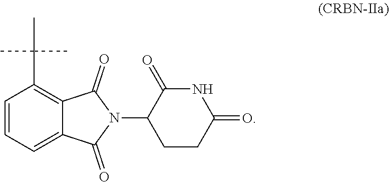

### Examples of Known VHL E3 Ubiquitin Ligase Ligands

It will be apparent to those skilled in the art that any of the known VHL ligands may be used in the PROTACs according to the invention. A number of different VHL ligands are commercially available or may be synthesized via known synthetic routes. In a highly preferred embodiment of the invention, the E3 ligase ligand is a VHL ligand selected from a compound of Formula (VHL-I), (VHL-II), (VHL-III), (VHL-IV), (VHL-V), (VHL-VI), or (VHL-VII)

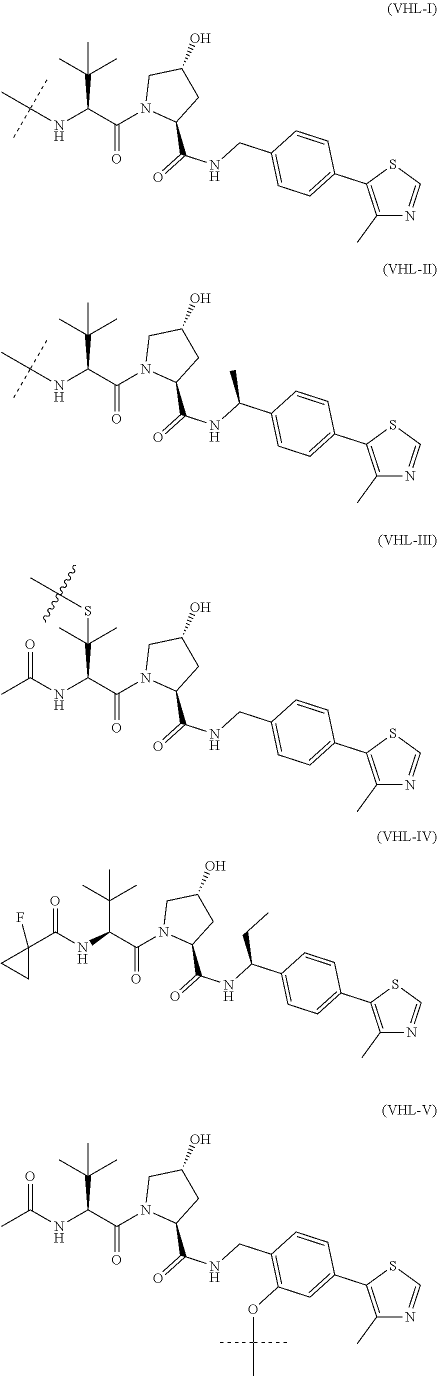

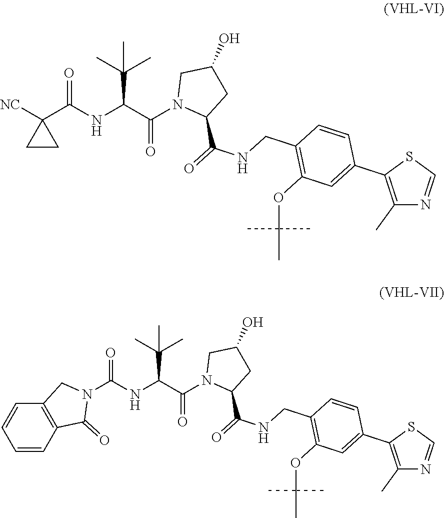

wherein the dotted lines denote a covalent connection to an DCAF15 ligand via a linker. In a most preferred embodiment of the invention, the VHL ligand consists of a compound of Formula (VHL-I).

### Examples of Known RNF114 E3 Ubiquitin Ligase Ligands

It will be apparent to those skilled in the art that any known RNF114 ligands may be used in the PROTACs according to the invention. A number of different RNF114 ligands are commercially available or may be synthesized via known synthetic routes. In a highly preferred embodiment of the invention, the E3 ubiquitin ligase ligand is a RNF114 ligand of Formula (RNF114-I) or (RNF114-II), preferably (RNF114-I),

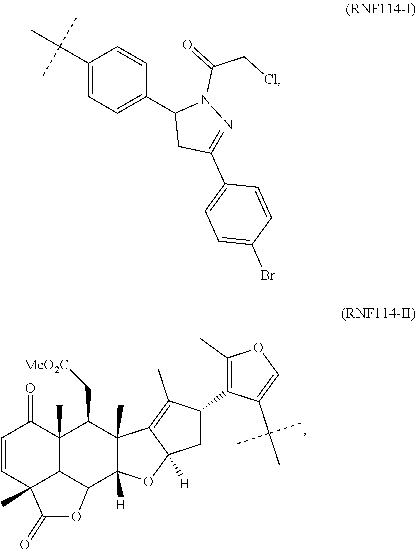

wherein the dotted lines denote a covalent connection to an DCAF15 ligand via a linker.

In a more preferred embodiment of the invention, the E3 ubiquitin ligase ligand is a RNF114 ligand of Formula (RNF114-Ia),

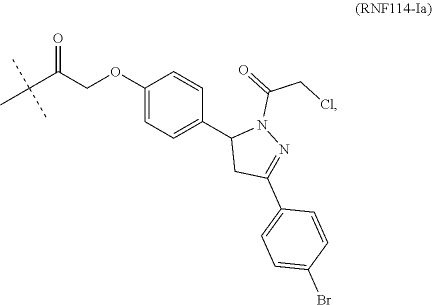

wherein the dotted line denotes a covalent connection to an DCAF15 ligand via a linker.

### Examples of Known DCAF11 E3 Ubiquitin Ligase Ligands

It will be apparent to those skilled in the art that any of the known DCAF11 ligands may be used in the PROTACs according to the invention. A number of different DCAF11 ligands are commercially available or may be synthesized via known synthetic routes. In a highly preferred embodiment of the invention, the E3 ligase ligand is a DCAF11 ligand having the structure of Formula (DCAF11-I-XXI)

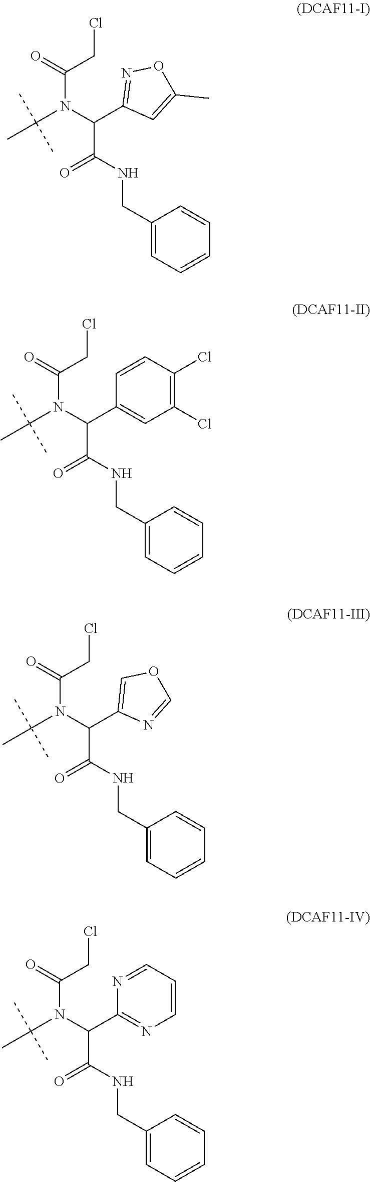

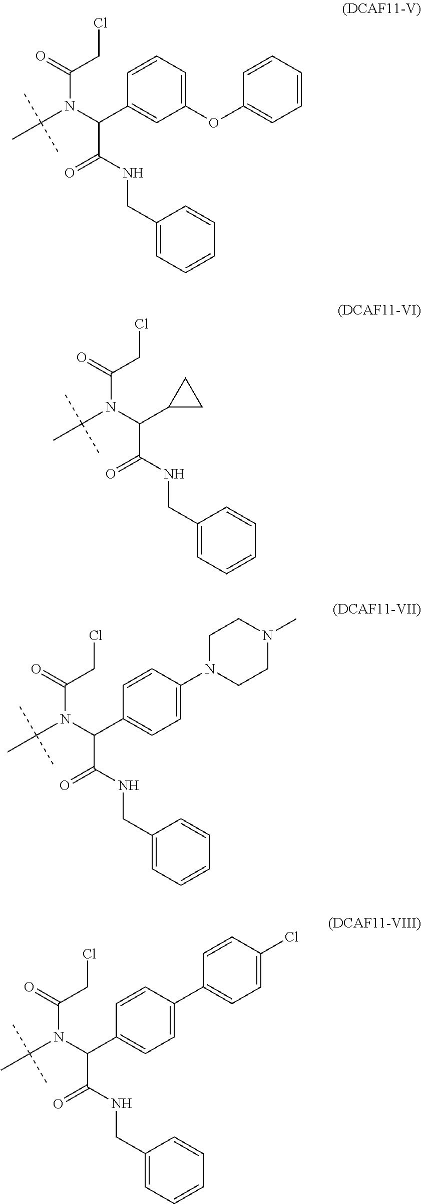

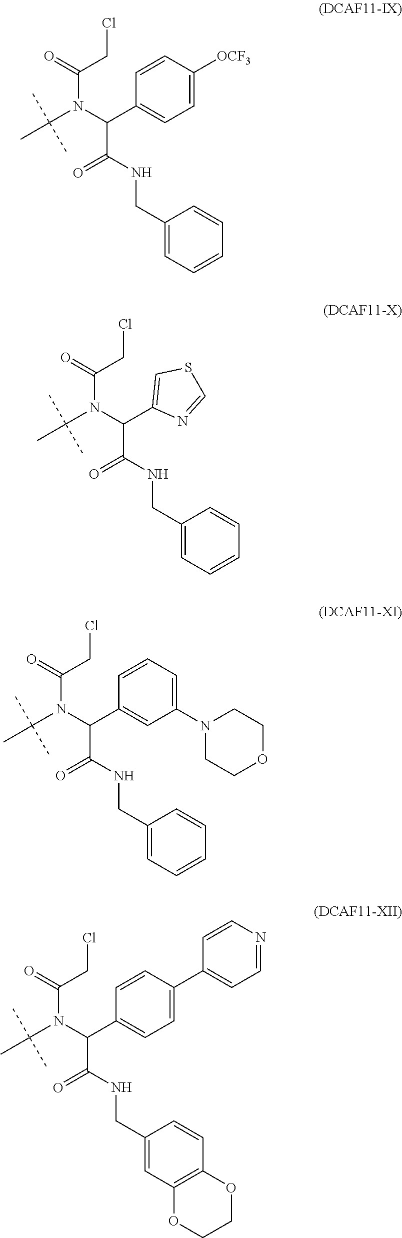

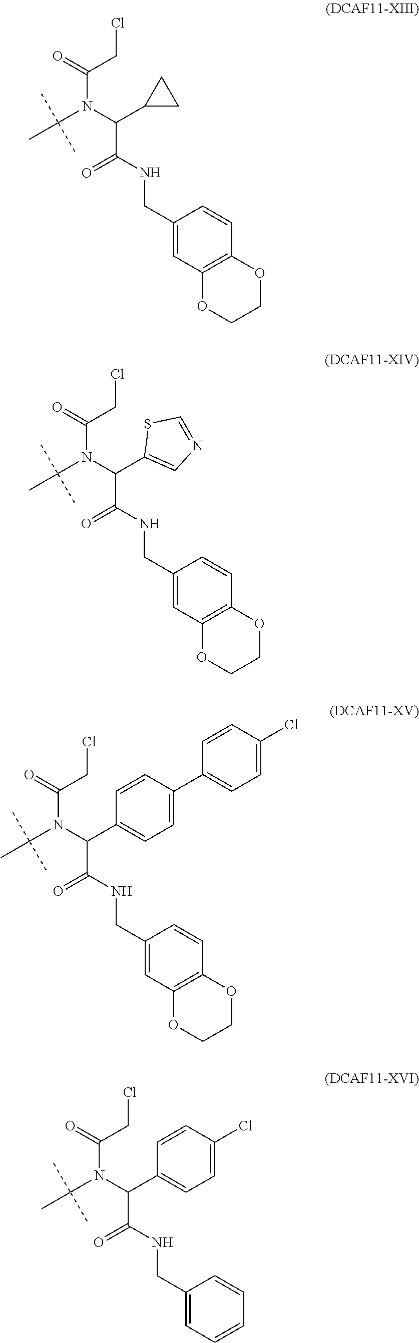

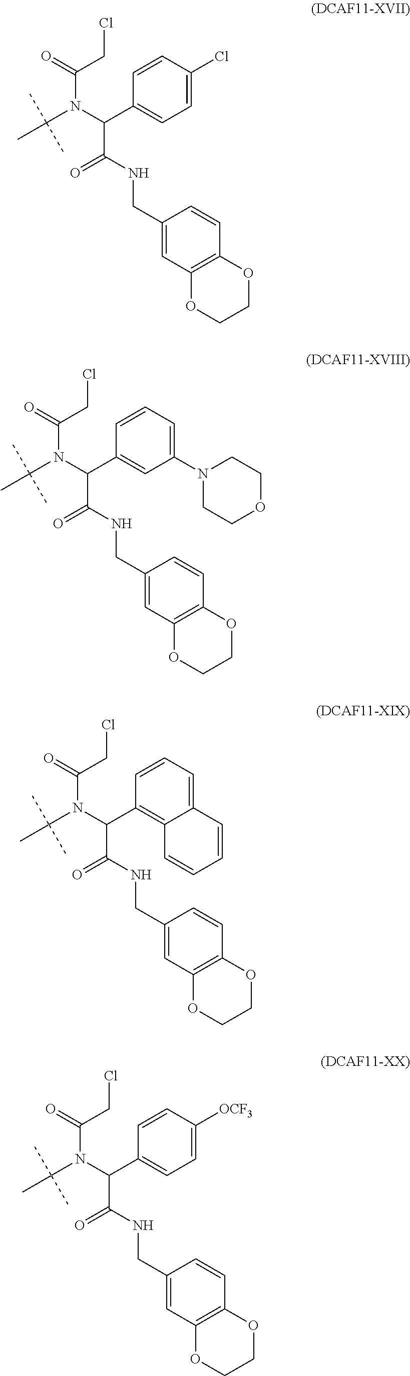

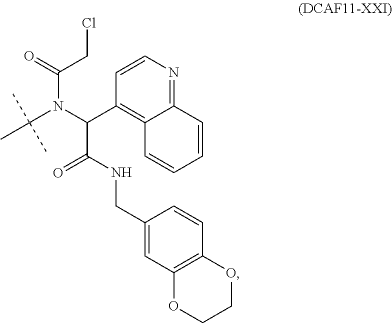

wherein the dotted lines denote a covalent connection to an DCAF15 ligand via a linker.

In a more preferred embodiment of the invention, the E3 ligase ligand is a DCAF11 ligand having the structure of Formula (DCAF11-I).

### Examples of Known DCAF15 E3 Ubiquitin Ligase Ligands

The protein DCAF15 forms in itself part of an E3 ligase complex (i.e. CUL4-DDB1-DDA1-DCAF15 E3 ubiquitin ligase complex). This allows for degradation of DCAF15 (POI) by recruiting the CUL4-DDB1-DDA1-DCAF15 E3 ubiquitin ligase complex. Thus, in some embodiments the ligand for recruiting the E3 ubiquitin ligase may also be a ligand for DCAF15. It will be apparent to those skilled in the art that any of the known DCAF15 ligands may also be used as a ligand for recruiting the E3 ubiquitin ligase in the PROTACs according to the invention. Hence, in some embodiments of the invention, the PROTAC may comprise two ligands for DCAF15 covalently connected via a linker (one acting to bind the POI and the other acting to recruit the E3 ubiquitin ligase). A number of different DCAF15 ligands are commercially available or may be synthesized via known synthetic routes. Thus, in an embodiment, the ligand for recruiting the E3 ubiquitin ligase comprises a compound selected from Formula (DCAF15-I), (DCAF15-II), or (DCAF15-III)

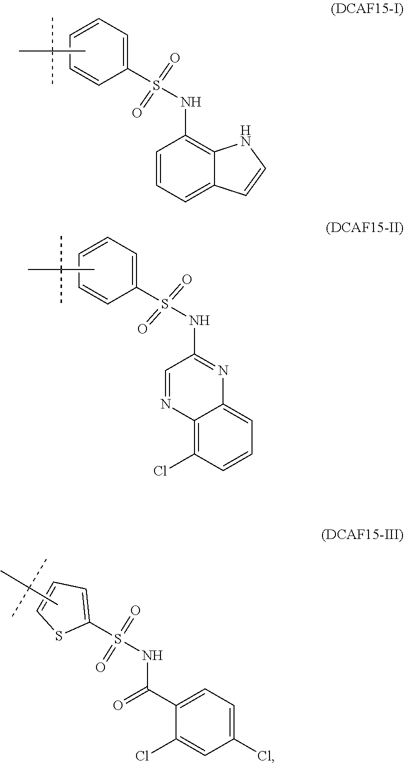

wherein the dotted lines denote a covalent connection to another DCAF15 ligand via a linker.

In a preferred embodiment of the invention, the E3 ligase ligand is a DCAF15 ligand (acting to recruit the E3 ligase) of Formula (DCAF15-I),

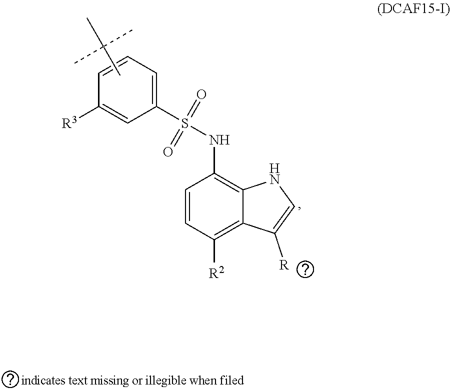

wherein R¹ is selected as F, Cl, Br, I, CN, Me, or CF₃; R² is selected as H, CN, CF₃ or Me; R³ is selected as CN or H; and wherein the dotted line denotes a covalent connection to another DCAF15 ligand via a linker. Preferably, R¹ is selected as Cl or CN, most preferably Cl; R² is selected as H or Me, most preferably H; and R³ is selected as H.

In an even more preferred embodiment of the invention, the E3 ligase ligand is a DCAF15 ligand (acting to recruit the E3 ligase) consisting of a compound of Formula (DCAF15-Ia),

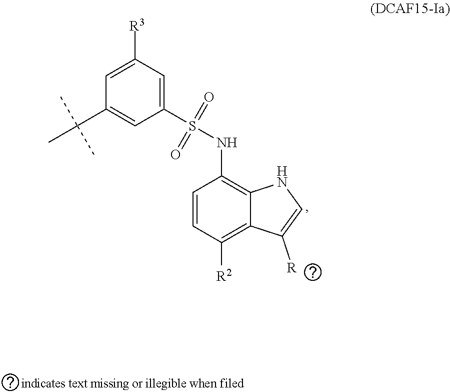

wherein R¹ is selected as F, Cl, Br, I, CN, Me, or CF₃; R² is selected as H, CN, CF₃ or Me; R³ is selected as CN or H; and wherein the dotted line denotes a covalent connection to another DCAF15 ligand via a linker. Preferably, R¹ is selected as Cl or CN, most preferably Cl; and R² is selected as H or Me, most preferably H; and R³ is selected as H.

It follows that in the embodiments, wherein the PROTAC comprises two ligands for DCAF15 covalently connected via a linker (one acting to bind the POI and the other acting to recruit the E3 ubiquitin ligase), the PROTACs may be either a heterobifunctional small molecule (e.g. a ligand of Formula (DCAF15-I) covalently linked to a ligand of Formula (DCAF15-II)) or a homobifunctional small molecule (e.g. a ligand of Formula (DCAF15-I) covalently linked to another ligand of Formula (DCAF15-I)). Most preferably, the PROTAC is homobifunctional, and most preferably, the DCAF15 ligand comprises the structure of Formula (DCAF15-I), most preferably (DCAF15-Ia).

### Examples of Known MDM2 E3 Ubiquitin Ligase Ligands

It will be apparent to those skilled in the art that any of the known MDM2 ligands in the prior art may be used in the PROTACs according to the invention. Several different MDM2 ligands are commercially available or may be synthesized via known synthetic routes. In a highly preferred embodiment of the invention, the E3 ubiquitin ligase ligand is a MDM2 ligand selected from a compound of Formula (MDM2-I), (MDM2-II), (MDM2-III), or Formula (MDM2-IV),

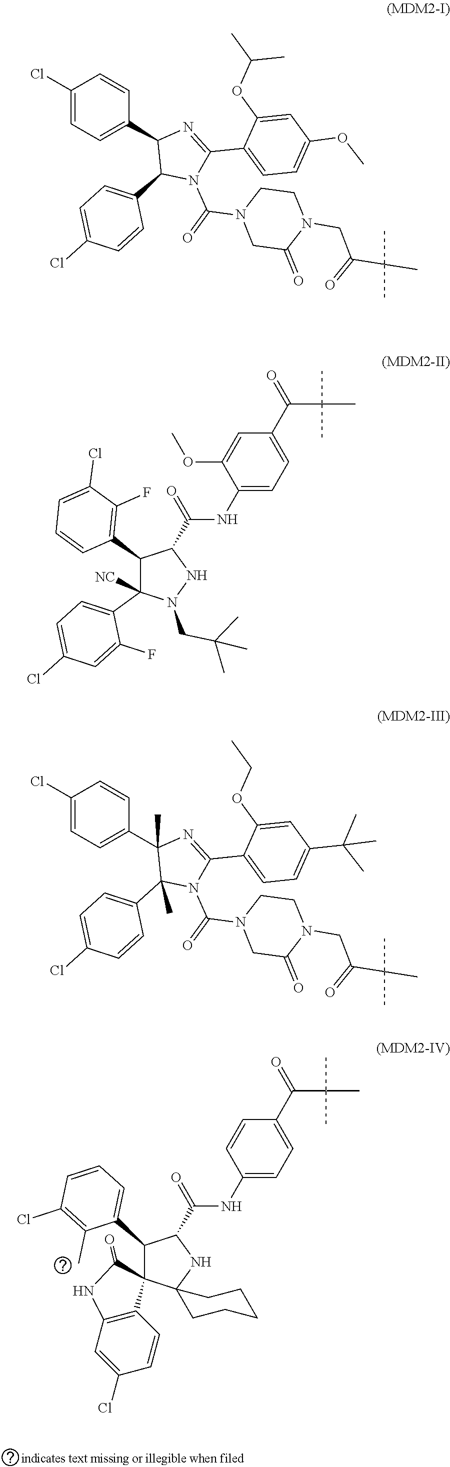

wherein the dotted lines denote a covalent connection to an DCAF15 ligand via a linker.

### Examples of Linkers in PROTACs

A variety of linkers have been successfully employed in PROTACs in the prior art. Alkyl chains, PEG chains, and extended glycol chains are the most common linker motifs found in PROTACs. These linkers offer some key advantages, including their commercial availability, their synthetic accessibility, their flexibility, and their ability to easily tune their length and composition via a wide array of robust chemical methods. Furthermore, the development of PROTACs have shown that the binding affinity may be improved by lowering the number of rotational bonds in the linker by introducing ring systems, such as (un)saturated carbocycles or heterocycles to rigidify the linker, e.g. a cyclohexane ring system, a benzene ring system, a piperidine ring system and/or a piperazine ring system, hence decreasing the degree of rotational freedom of the linker and “locking” the molecule in an active binding conformation. Thus, in some embodiments of the present invention, the linker comprises a PEG chain, an extended glycol chain, an alkyl chain, and/or a chain comprising a cyclic motif selected from a disubstituted cyclohexane ring system, a disubstituted benzene ring system, a disubstituted piperidine ring system and/or a disubstituted piperazine ring system to rigidify the linker. The linkers may be linear or branched or comprise one or more cyclic motif(s). Preferably, the linkers are linear or comprise one or more cyclic motifs.

For the PROTACs according to the present invention, the linkers comprising a PEG chain, an extended glycol chain, an alkyl chain, and/or a chain comprising a cyclic motif, the chain length is from 6-24 atoms, preferably 6-22 atoms, more preferably 6-20 atoms, even more preferably 6-18 atoms, most preferably 8-16 atoms.

The linkers comprising a PEG chain, an extended glycol chain, an alkyl chain, and/or a chain comprising a cyclic motif is covalently attached to the DCAF15 ligand in one end and to the E3 ubiquitin ligase ligand in the opposite end via common functional groups. Such functional groups may include e.g. amides, sulfonamides, sulfinamides, sulfonates, sulfinates, carbamates, thiocarbamates, esters, ketones, ethers, thiol ethers, amines, alkynes, or tetrazoles depending on the E3 ligase ligand in the PROTAC. For example, if the E3 ligase ligand is selected from a scaffold of Formula (VHL-I) or (VHL-II), the linker is covalently attached to the amine (denoted with the dotted line) in Formula (VHL-I) or (VHL-II). Thus, the functional group for attachment of the linker to the ligand may be e.g. an amide, sulfonamide, sulfinamide, carbamate, or an amine, preferably an amide or amine commonly employed in PROTACs with VHL based ligands. Likewise, if e.g. the E3 ubiquitin ligase ligand is selected from a scaffold of Formula (VHL-V), (VHL-VI) or (VHL-VII), the linker is attached to the hydroxy group (denoted with the dotted line) in Formula (VHL-V), (VHL-VI) or (VHL-VII). Thus, the functional group for attachment of the linker to the E3 ligase ligand may be an e.g. a sulfonate, sulfinate, carbamate, ester, or ether, preferably an ether commonly employed in PROTACs with such VHL based ligands. Likewise, if e.g. the E3 ligase ligand is selected from a scaffold of Formula (MDM2-I), (MDM2-II), (MDM2-III) or (MDM2-IV), the linker is covalently attached to the acyl group (denoted with the dotted line) in Formula (MDM2-I), (MDM2-II), (MDM2-III) or (MDM2-IV). Thus, the functional group attachment of the linker to the ligand may be e.g. a ketone, amide, or an ester, preferably an amide. Likewise, if e.g. the E3 ligase ligand is selected from a scaffold of Formula (CRBN-I) or (CRBN-II), the linker is covalently attached to the phenyl group (denoted with the dotted line) in Formula (CRBN-I) or (CRBN-II). Thus, the functional group for attachment of the linker to the ligand may be e.g. an amide, sulfonamide, sulfinamide, sulfonate, sulfinate, carbamate, thiocarbamate, ester, ketone, ether, thiol ether, amine, alkyne, alkene, alkane, a triazole or a tetrazole. Preferably, the functional group for attachment of the linker to the CRBN ligands is an amine, ether, alkane, alkyne, or amide commonly employed in PROTACs with CRBN based ligands.

Preferably the linker comprises a structure selected from Formula L1-L8:

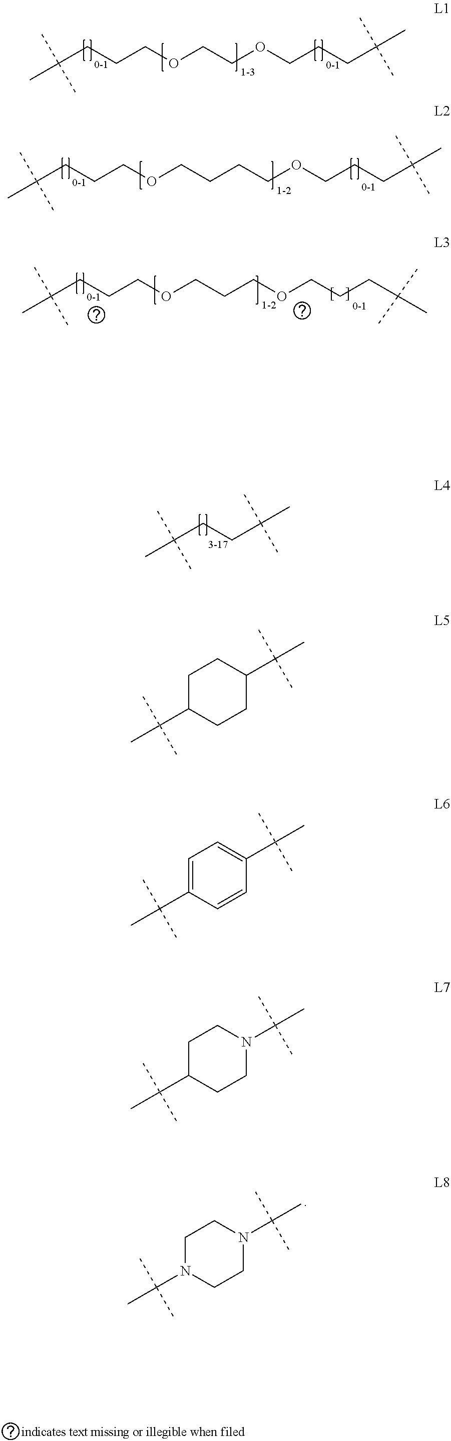

Most preferably the linker is covalently connected to the DCAF15 ligand via a functional group selected from an amide, sulfonamide, or ether, more preferably an amide or sulfonamide, most preferably an amide as illustrated herein. However, it will be apparent to the skilled person that the linker may be connected to the DCAF15 ligand and the E3 ligase ligand via different functional groups well known in the art. Thus, in a highly preferred embodiment the linker consists of a structure selected from the list consisting of:

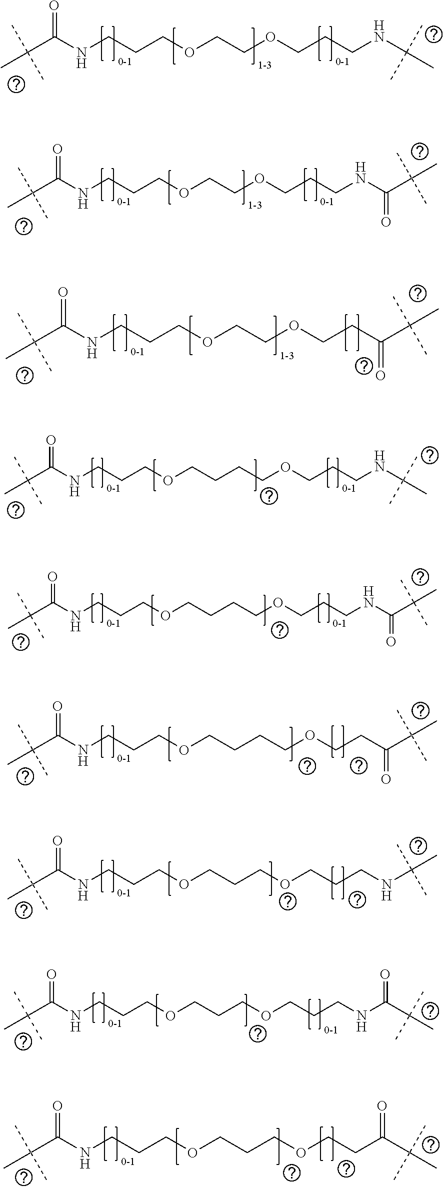

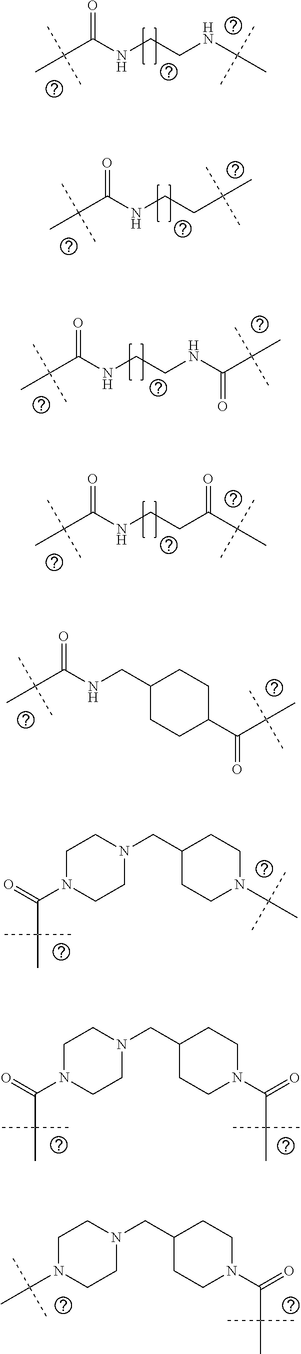

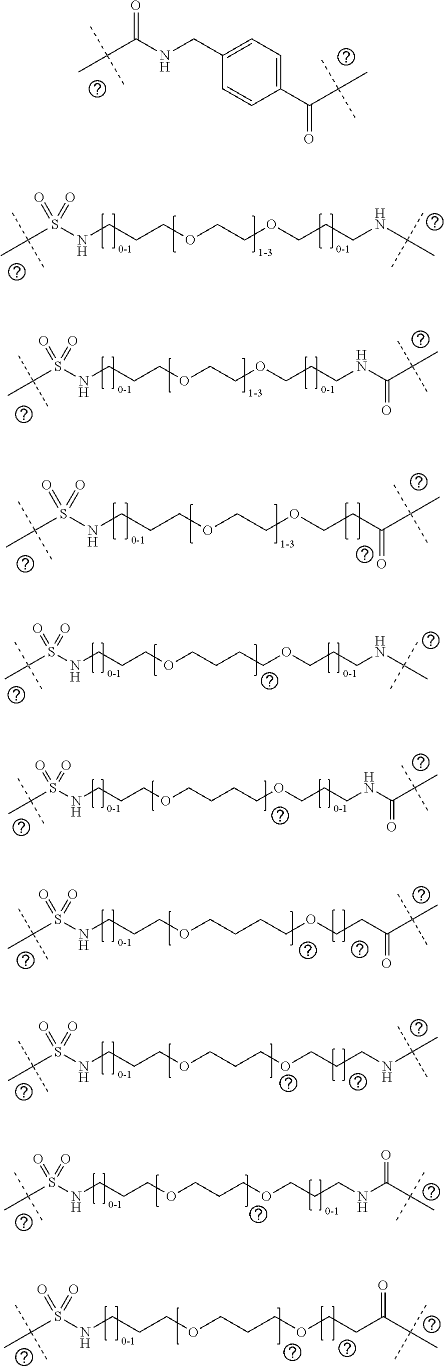

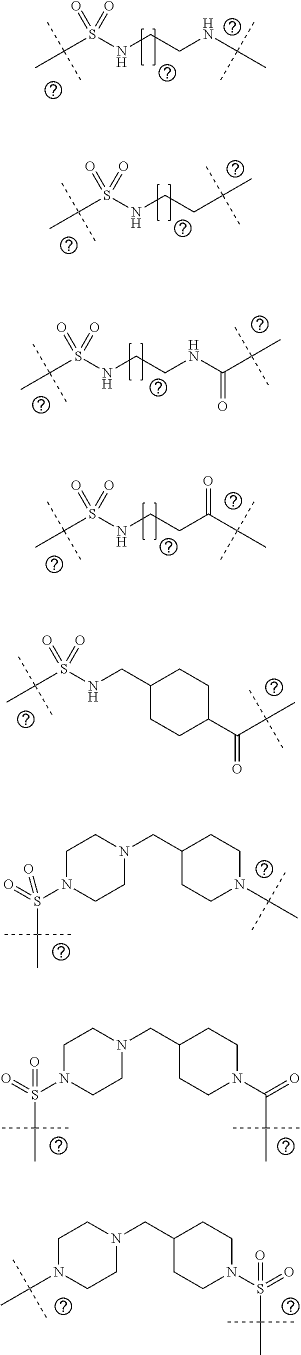

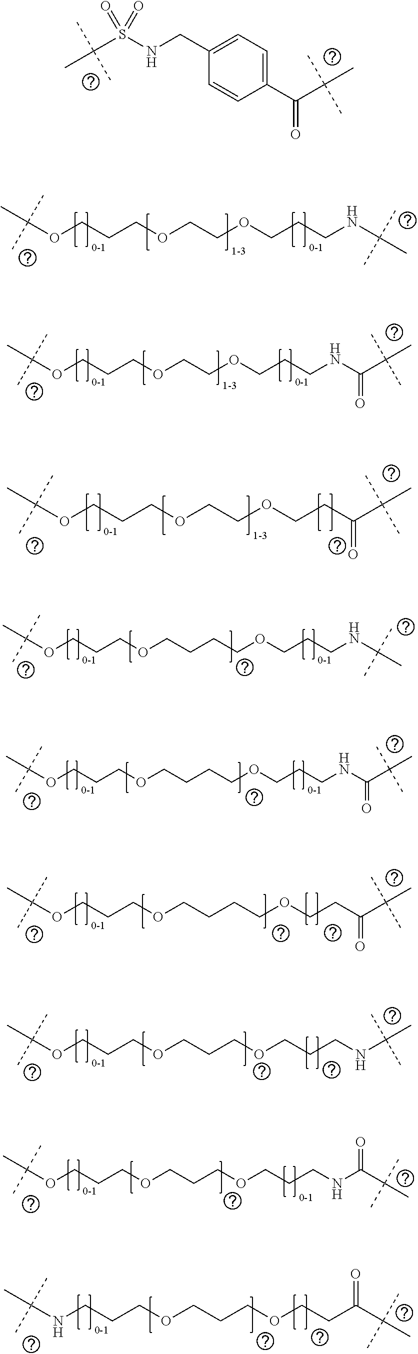

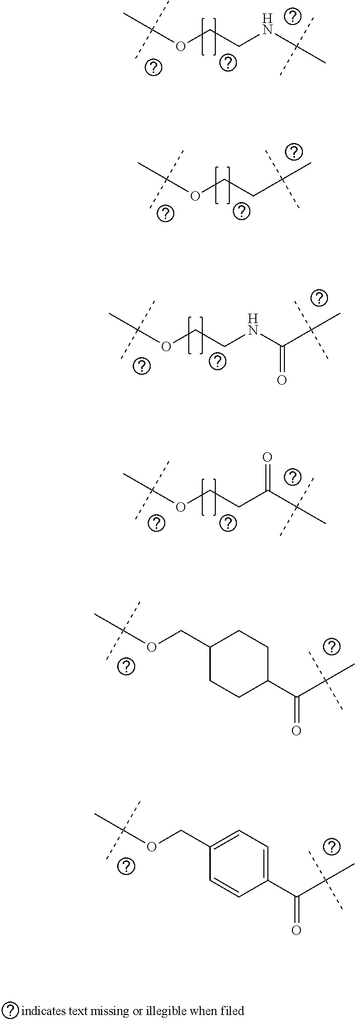

wherein the dotted lines marked * denote the end of the linker covalently connected to the DCAF15 ligand and the dotted lines marked ** denote the end of the linker covalently connected to the E3 ligase ligand.

In a first aspect, the present invention relates to a proteolysis targeting chimera (PROTAC) or a pharmaceutically acceptable salt thereof, said PROTAC comprising a ligand for DCAF15 (i.e. protein of interest) covalently connected to an E3 ubiquitin ligase ligand via a linker, wherein
the ligand for DCAF15 is selected from a compound of Formula (DCAF15-I), (DCAF15-II), or (DCAF15-III)

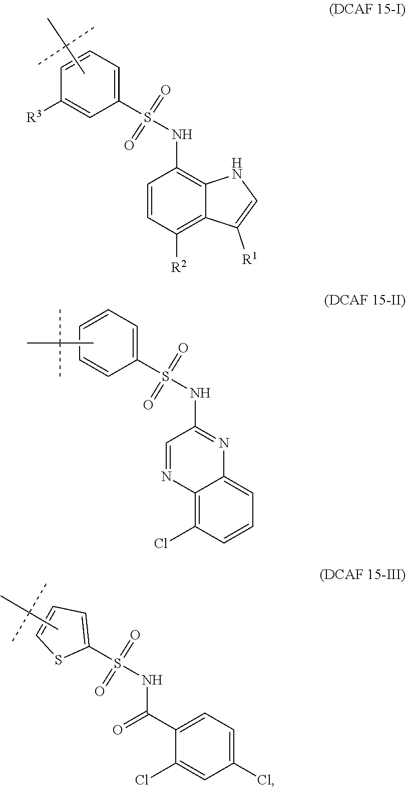

wherein R¹ is selected as F, Cl, Br, I, CN, Me, or CF₃; R² is selected as H, CN, CF₃, or Me; R³ is selected as CN or H; and wherein the dotted lines denote a covalent connection to the E3 ubiquitin ligase ligand via the linker;
the E3 ubiquitin ligase ligand is selected from a CRBN-ligand, a VHL-ligand, a RNF114-ligand, a DCAF11-ligand, or a MDM2-ligand.

In an embodiment, the present invention relates to a proteolysis targeting chimera (PROTAC) or a pharmaceutically acceptable salt thereof, said PROTAC comprising a ligand for DCAF15 (i.e. protein of interest) covalently connected to an E3 ubiquitin ligase ligand via a linker, wherein
the linker comprises a PEG chain, an extended glycol chain, an alkyl chain, or a chain having a cyclic motif selected from a structure of Formula L1-L4,

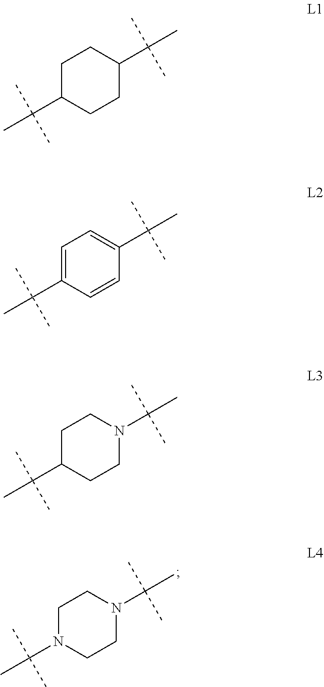

the ligand for DCAF15 comprises a compound of Formula (DCAF15-I), (DCAF15-II), or (DCAF15-III),

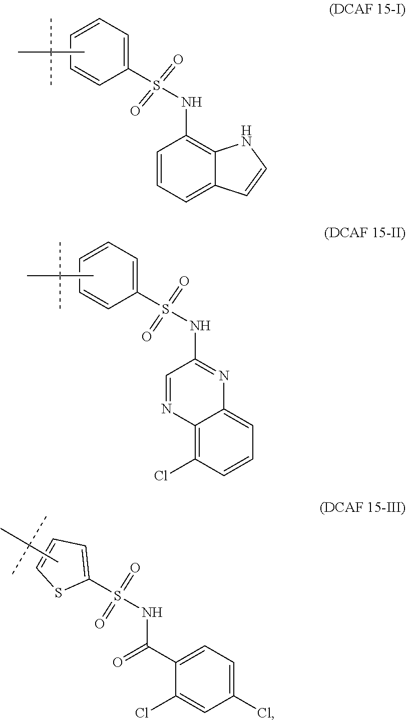

wherein the dotted lines denote a covalent connection to the E3 ligase ligand via the linker;
the E3 ubiquitin ligase ligand is selected from a CRBN-ligand, a VHL-ligand, a RNF114-ligand, a DCAF11-ligand, a DCAF15-ligand, or a MDM2-ligand.

Most preferably, the ligand for DCAF15 is selected from a compound of Formula (DCAF15-I).

In an embodiment, the present invention relates to a proteolysis targeting chimera (PROTAC) or a pharmaceutically acceptable salt thereof, said PROTAC comprising a ligand for DCAF15 (i.e. protein of interest) covalently connected to an E3 ubiquitin ligase ligand via a linker, wherein
the linker comprises a PEG chain, an extended glycol chain, an alkyl chain, or a chain having a cyclic motif selected from a structure of Formula L1-L4,

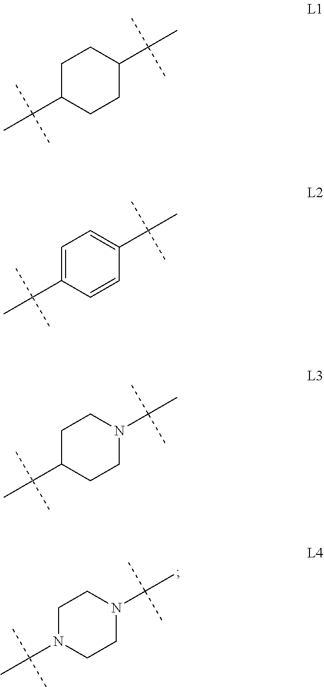

the ligand for DCAF15 comprises a compound of Formula (DCAF15-I),

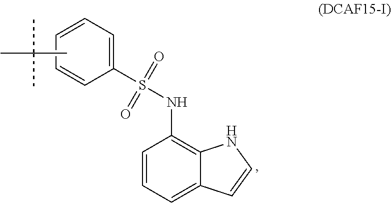

wherein the dotted line denotes a covalent connection to the E3 ubiquitin ligase ligand via the linker;
the E3 ubiquitin ligase ligand is selected from a CRBN-ligand, a VHL-ligand, a RNF114-ligand, a DCAF11-ligand, a DCAF15-ligand, or a MDM2-ligand.

In a preferred embodiment, the present invention relates to a proteolysis targeting chimera (PROTAC) or a pharmaceutically acceptable salt thereof, said PROTAC comprising a ligand for DCAF15 (i.e. protein of interest) covalently connected to an E3 ubiquitin ligase ligand via a linker, wherein
the linker comprises a PEG chain, an extended glycol chain, an alkyl chain, or a chain having a cyclic motif selected from a structure of Formula L1-L4,

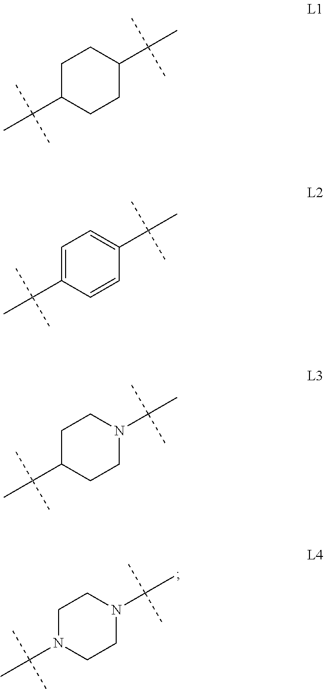

the ligand for DCAF15 is selected from a compound of Formula (DCAF15-I),

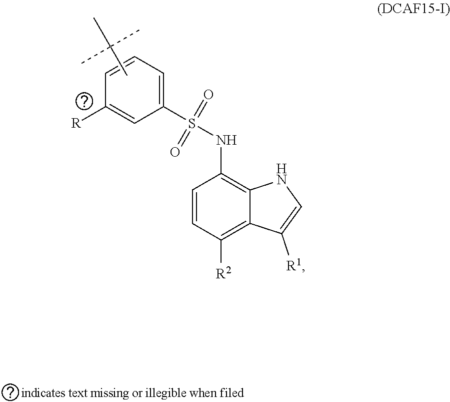

wherein R¹ is selected as F, Cl, Br, I, CN, Me, or CF₃; R² is selected as H, CN, CF₃ or Me; R³ is selected as CN or H; wherein the dotted line denotes a covalent connection to an E3 ubiquitin ligase ligand via the linker;
the E3 ligase ligand is selected from a CRBN-ligand, a VHL-ligand, a RNF114-ligand, a DCAF11-ligand, or a MDM2-ligand; preferably, the E3 ligase ligand is selected from a CRBN-ligand, a VHL-ligand, a RNF114-ligand, a DCAF11-ligand, or a MDM2-ligand; most preferably the E3 ligase ligand is selected from a CRBN-ligand, a VHL-ligand.

In aspect 1 and its embodiments, the CRBN-ligand, the VHL-ligand, the RNF114-ligand, the DCAF11-ligand, or the MDM2-ligand is preferably selected from a ligand illustrated herein under the examples of known CRBN E3 ubiquitin ligase ligands, the examples of known VHL E3 ubiquitin ligase ligands, the examples of known RNF114 E3 ubiquitin ligase ligands, the examples of known DCAF11 E3 ubiquitin ligase ligands, or the examples of known MDM2 E3 ubiquitin ligase ligands. In any of the above aspects and embodiments, the CRBN-ligand, the VHL-ligand, the RNF114-ligand, the DCAF11-ligand, or the MDM2-ligand is preferably selected from the following structures:

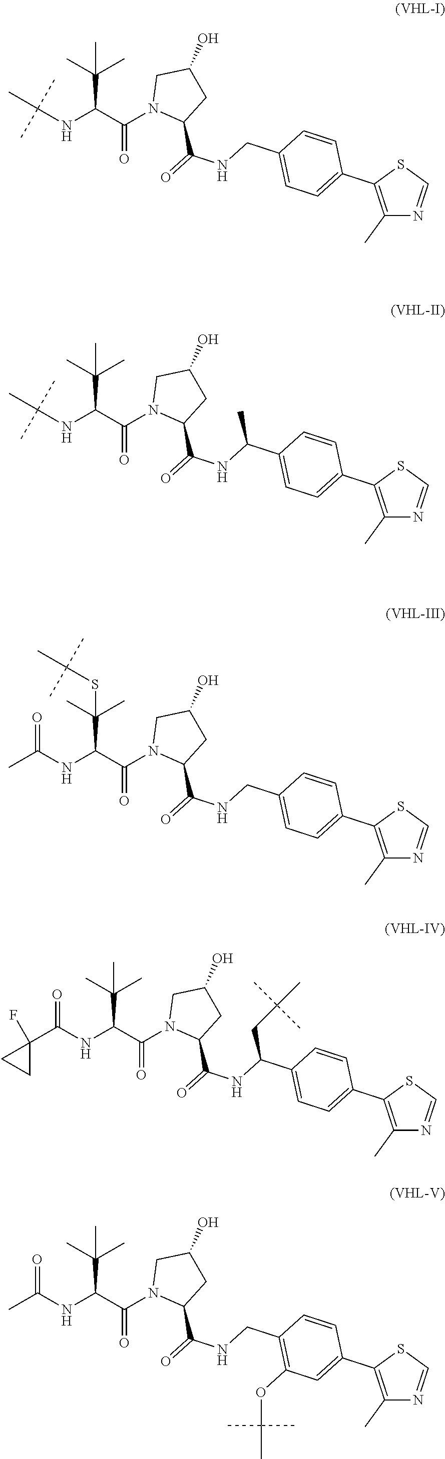

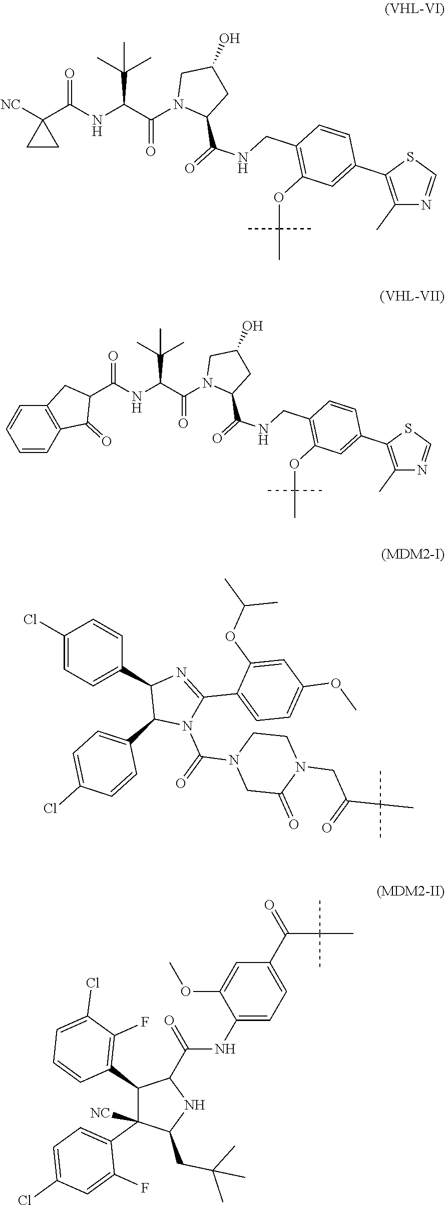

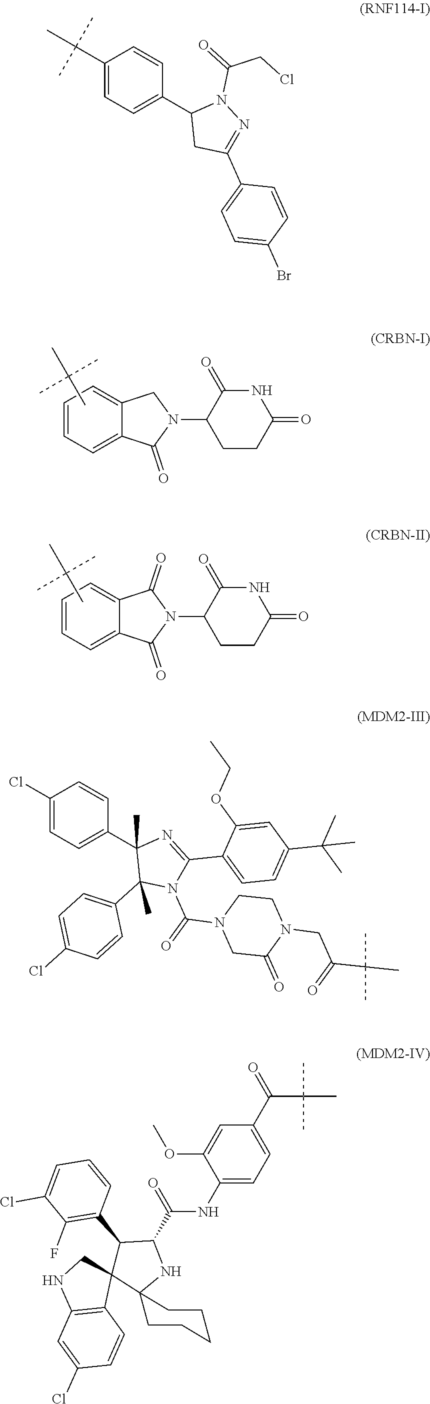

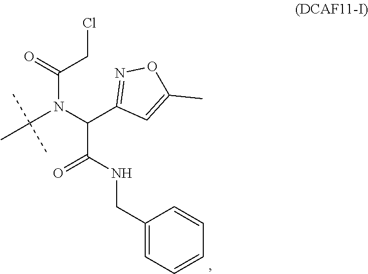

wherein the dotted lines denote a covalent connection to an DCAF15 ligand via the linker.

Thus, in a more preferred embodiment, the present invention relates to a proteolysis targeting chimera (PROTAC) or a pharmaceutically acceptable salt thereof, said PROTAC comprising a ligand for DCAF15 (i.e. protein of interest) covalently connected to an E3 ubiquitin ligase ligand via a linker, wherein
the linker comprises a PEG chain, an extended glycol chain, an alkyl chain, or a chain having a cyclic motif selected from a structure of Formula L1-L4,

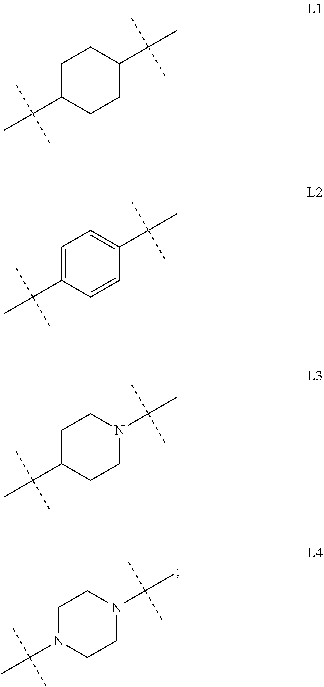

the ligand for DCAF15 is selected from a compound of Formula (DCAF15-I),

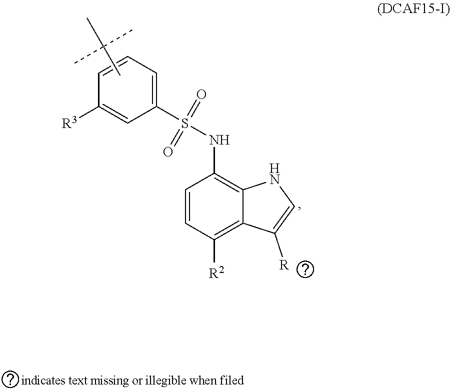

wherein, R¹ is selected as F, Cl, Br, I, CN, Me, or CF₃; R² is selected as H, CN, CF₃ or Me; R³ is selected as CN or H; wherein the dotted line denotes a covalent connection to an E3 ubiquitin ligase ligand via a linker;
the E3 ligase ligand is selected from a compound of Formula (VHL-I), (VHL-II), (VHL-III), (VHL-IV), (VHL-V), (VHL-VI), (VHL-VII), (DCAF15-I), (MDM2-I), (MDM2-II), (MDM2-III), (MDM2-IV), (DCAF11-I), (RNF114-I), (CRBN-I), or (CRBN-II); preferably, the E3 ligase ligand is selected from a compound of Formula (VHL-I), (VHL-II), (VHL-III), (VHL-IV), (VHL-V), (VHL-VI), (VHL-VII), (MDM2-I), (MDM2-II), (MDM2-III), (MDM2-IV), (DCAF11-I), (RNF114-I), (CRBN-I), or (CRBN-II); more preferably the E3 ligase ligand is selected from a compound of Formula (VHL-I), (VHL-II), (VHL-III), (VHL-IV), (VHL-V), (VHL-VI), (VHL-VII), (DCAF11-I), (RNF114-I), (CRBN-I), or (CRBN-II); most preferably the E3 ligase ligand is selected from a compound of Formula (VHL-I) or (CRBN-II).

In aspect 1 and its embodiments described above, it is highly preferred that the ligand for DCAF15 is selected from a compound of Formula (DCAF15a)

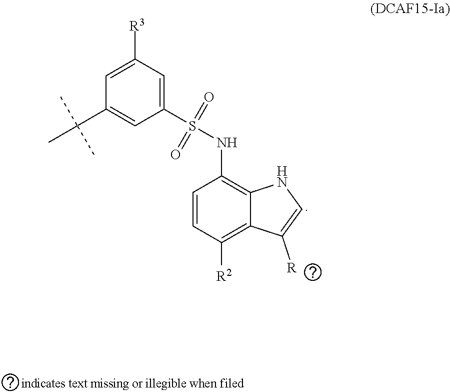

It is also highly preferred that R¹ is selected as Cl or CN, most preferably Cl; that R² is selected as H or Me, most preferably H; and that R³ is selected as H. It is further preferred, that the linker comprises a structure selected from Formula L1-L8:

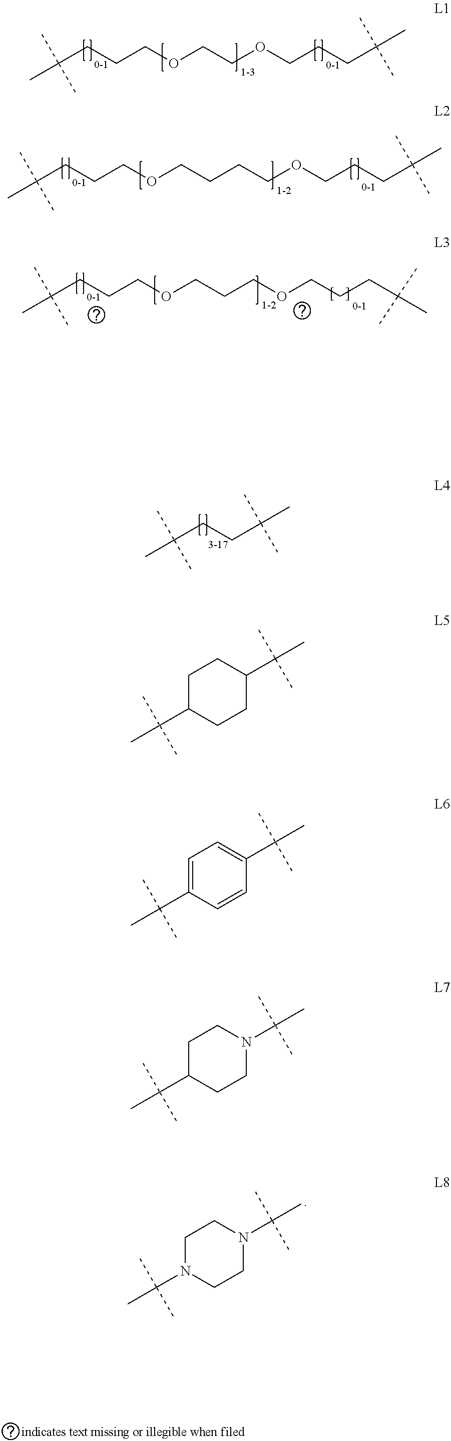

More preferably, the linker comprises a PEG chain, an extended glycol chain, or an alkyl chain. It is further highly preferred that the linker has a chain length of 6-24 atoms, preferably 6-22 atoms, more preferably 6-20 atoms, even more preferably 6-18 atoms, most preferably 8-16 atoms.

In the above aspects and embodiments, the linker preferably consists of a structure selected from list consisting of:

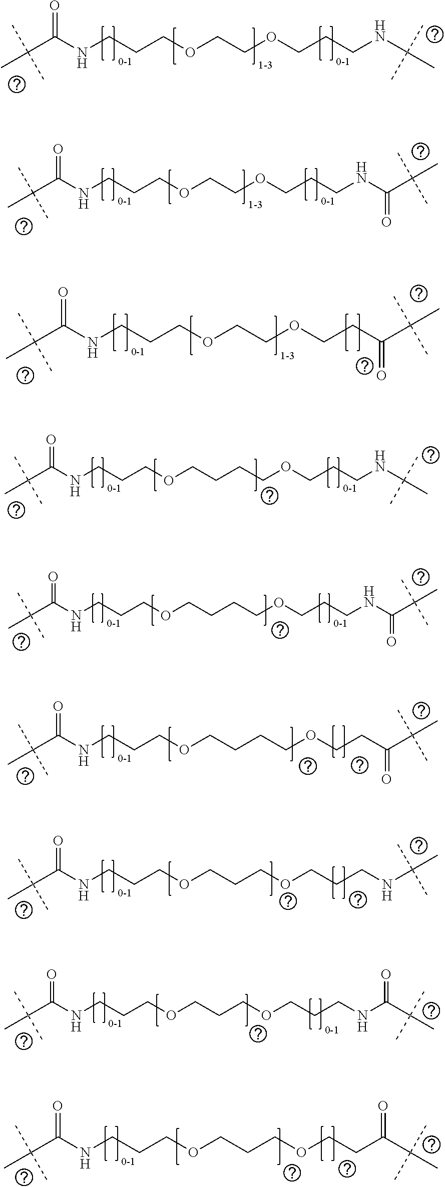

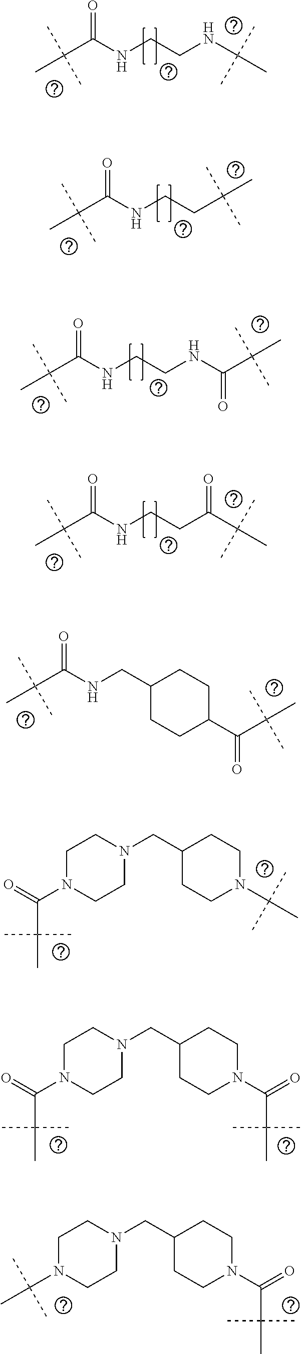

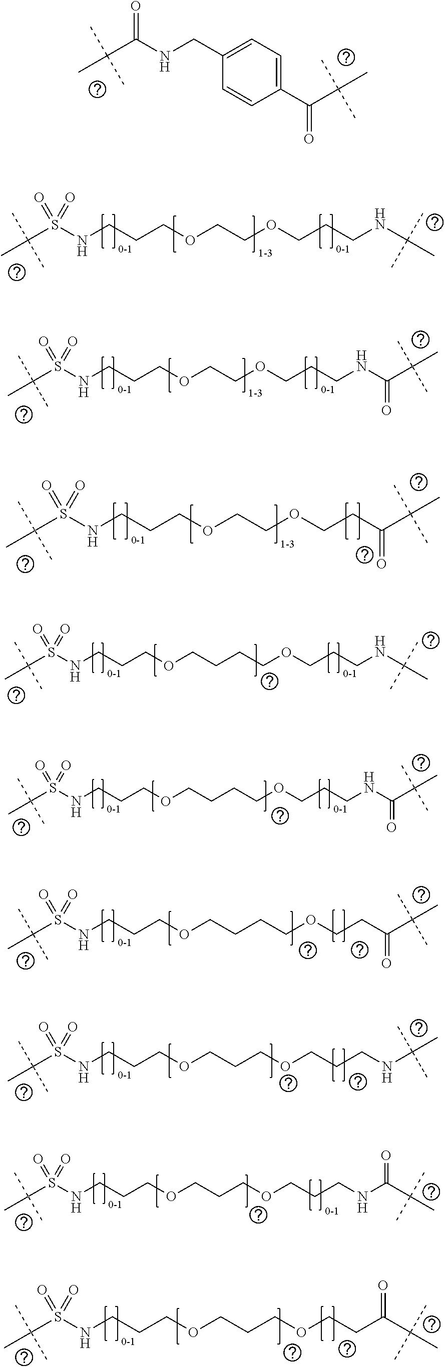

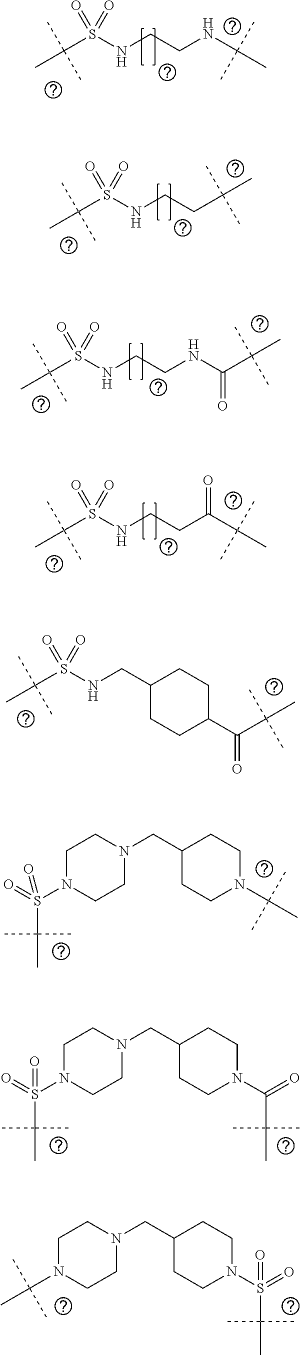

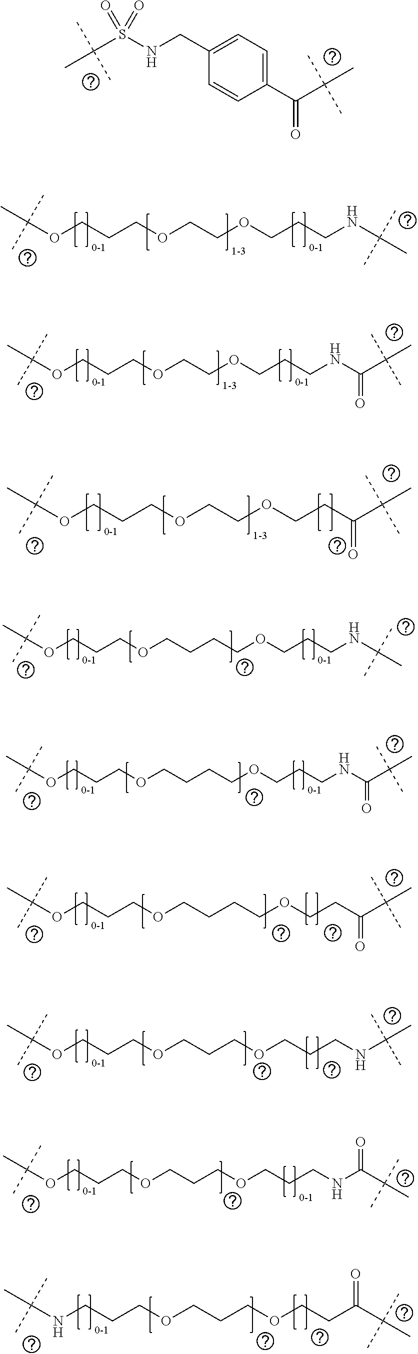

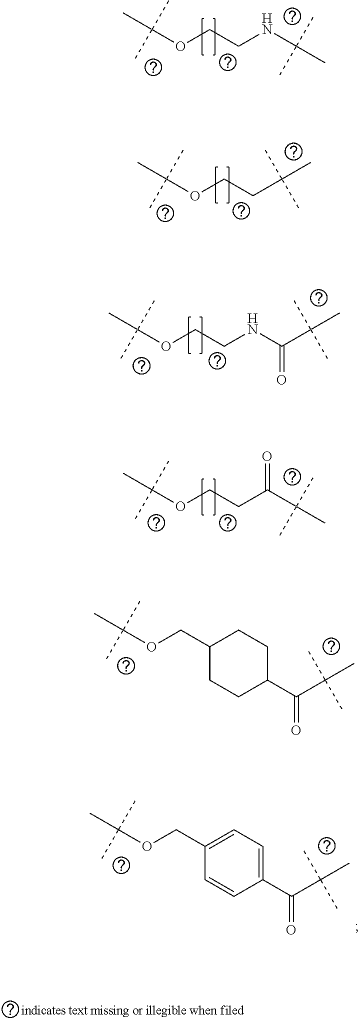

more preferably, the linker consists of a structure selected from list consisting of:

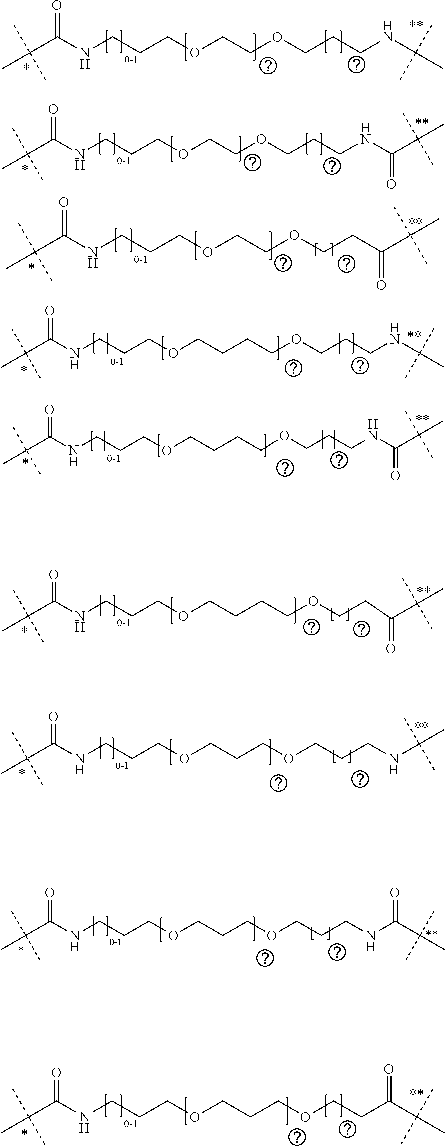

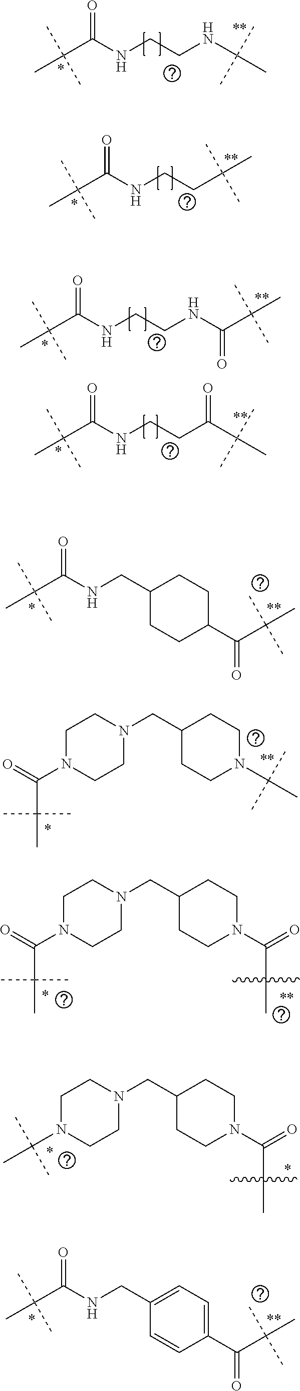

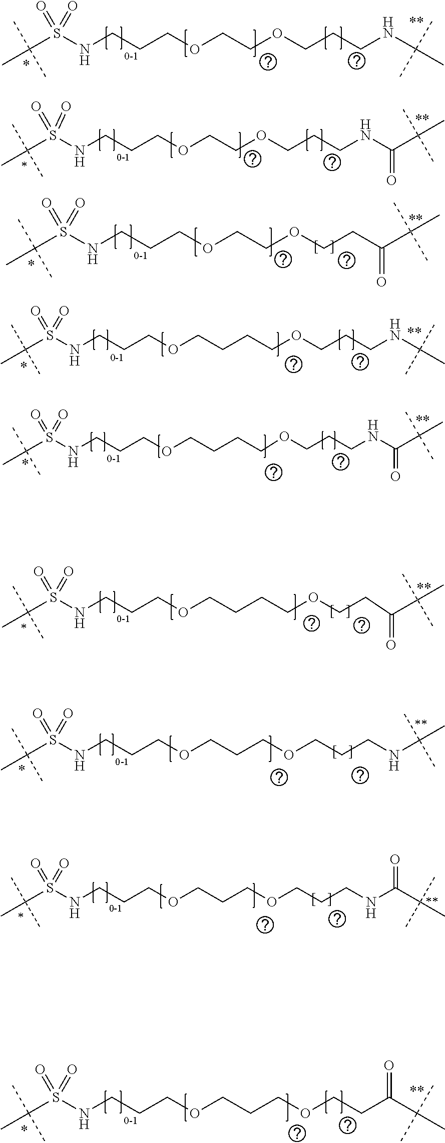

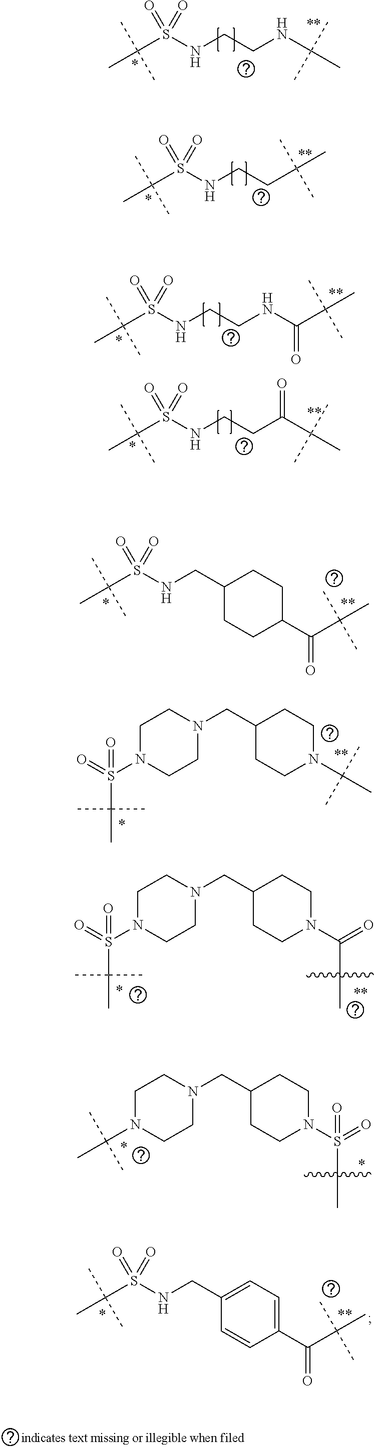

even more preferably, the linker consists of a structure selected from list consisting of:

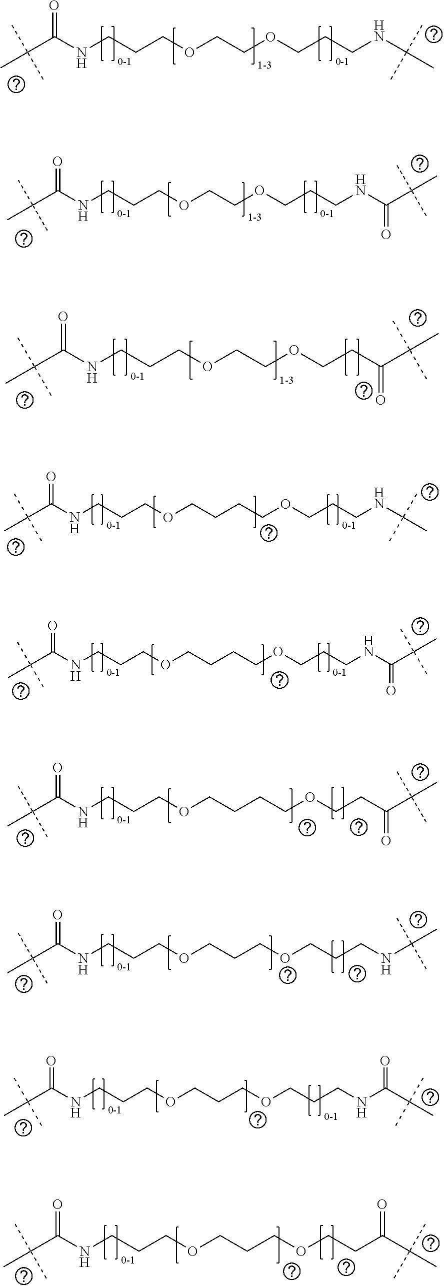

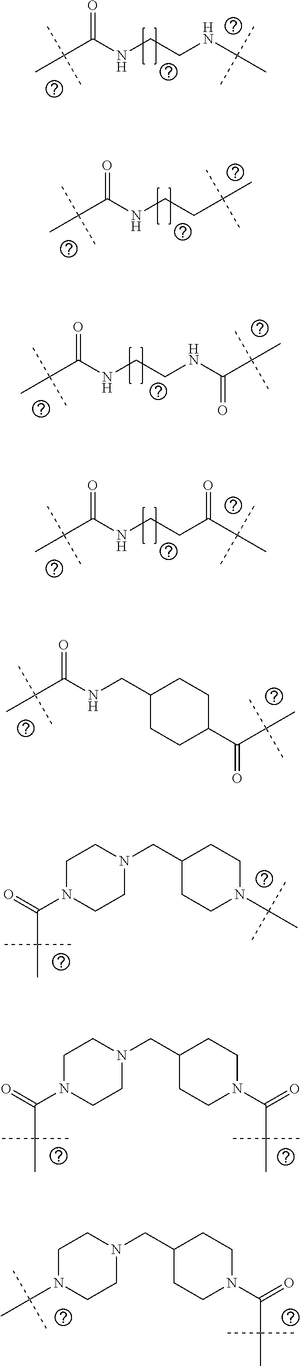

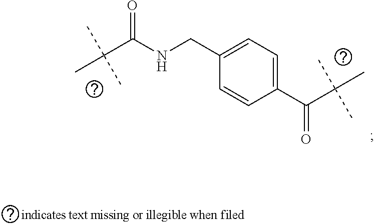

most preferably, the linker consists of a structure selected from list consisting of:

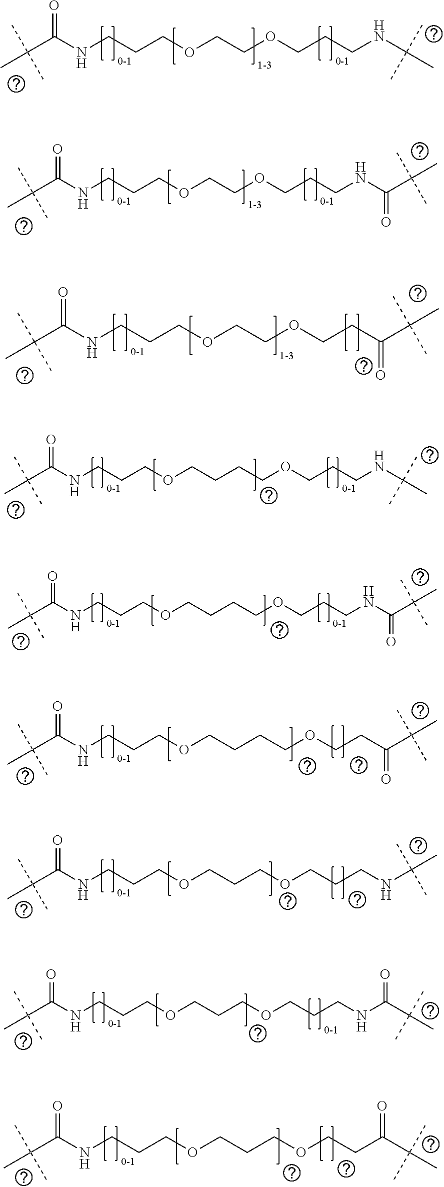

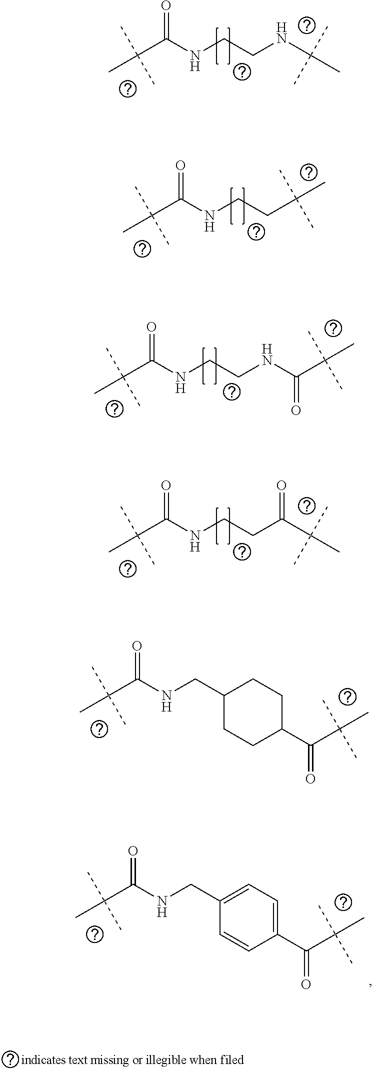

wherein the dotted lines marked * denote the end of the linker covalently connected to the DCAF15 ligand and the dotted lines marked ** denote the end of the linker covalently connected to the E3 ligase ligand. Most preferably, the linkers drawn above have a chain length of 6-24 atoms, preferably 6-22 atoms, more preferably 6-20 atoms, even more preferably 6-18 atoms, most preferably 8-16 atoms.

### Items of Aspect 1:

1. A proteolysis targeting chimera (PROTAC) or a pharmaceutically acceptable salt thereof, said PROTAC comprising a ligand for DCAF15 (i.e. protein of interest) covalently connected to an E3 ubiquitin ligase ligand via a linker,
   wherein
      the ligand for DCAF15 is selected from a compound of Formula (DCAF15-I), (DCAF15-II), or (DCAF15-III)

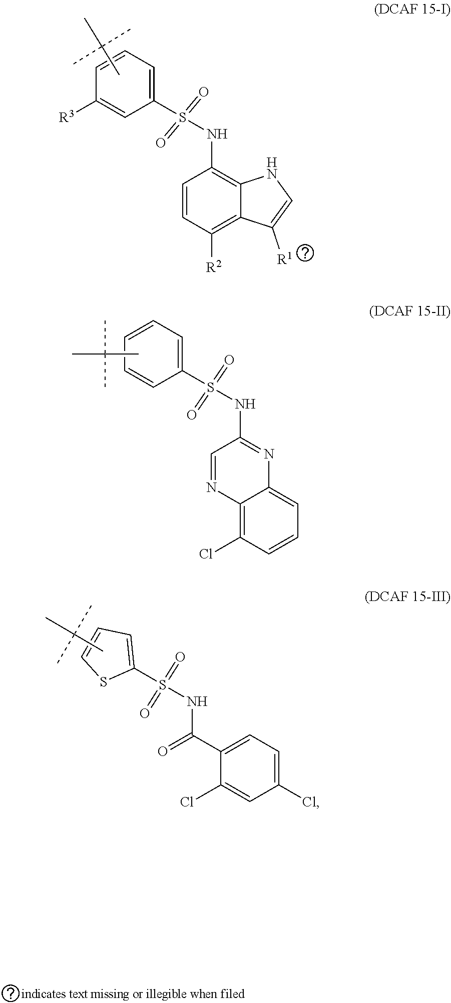

wherein R¹ is selected as F, Cl, Br, I, CN, Me, or CF₃; R² is selected as H, CN, CF₃, or Me; R³ is selected as CN or H; and wherein the dotted lines denote a covalent connection to the E3 ubiquitin ligase ligand via the linker;
the E3 ubiquitin ligase ligand is selected from a compound of Formula (VHL-I), (VHL-II), (VHL-III), (VHL-IV), (VHL-V), (VHL-VI), (VHL-VII), (MDM2-I), (MDM2-II), (MDM2-III), (MDM2-IV), (DCAF11-I), (RNF114-I), (CRBN-I), or (CRBN-II) having the structure:

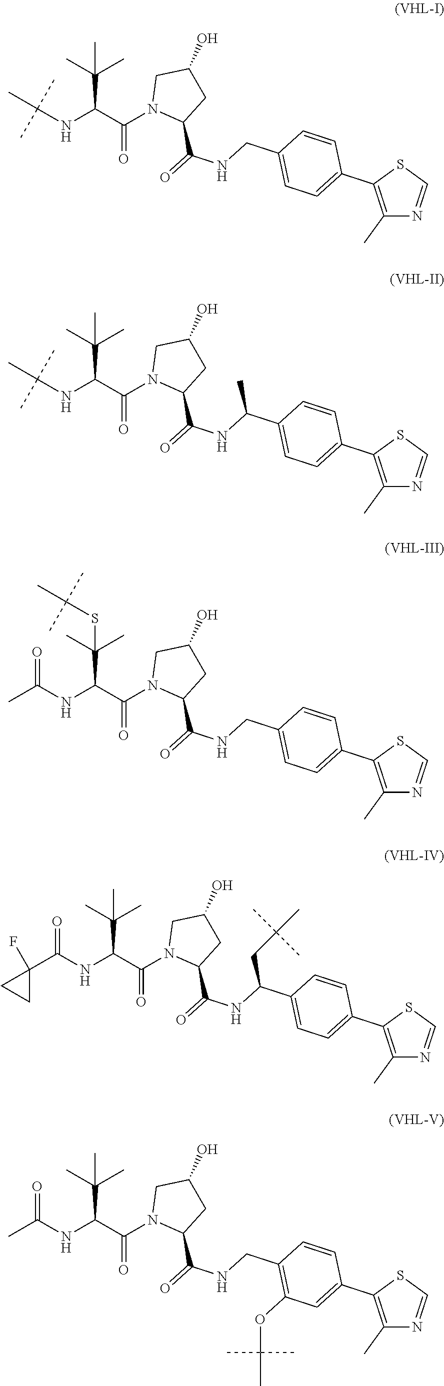

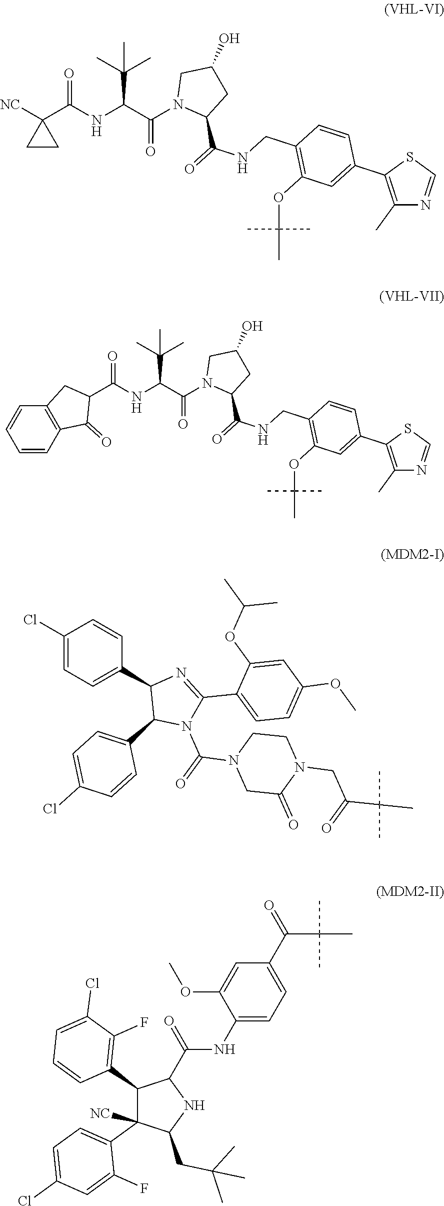

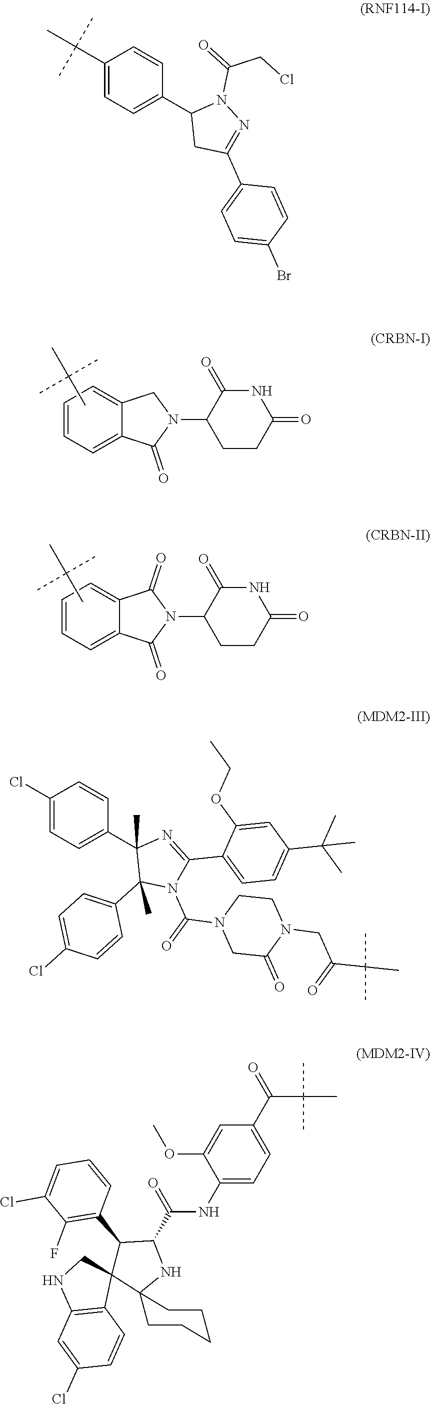

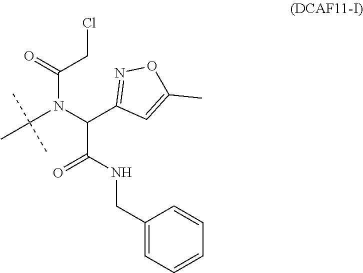

wherein the dotted lines denote a covalent connection to the DCAF15 ligand via the linker.
2. A PROTAC according to item 1, wherein the ligand for DCAF15 is selected from a compound of Formula (DCAF-I).
3. A PROTAC according to any one of the preceding items, wherein the ligand for DCAF15 is selected from a compound of Formula (DCAF15-Ia) or (DCAF15-Ib):

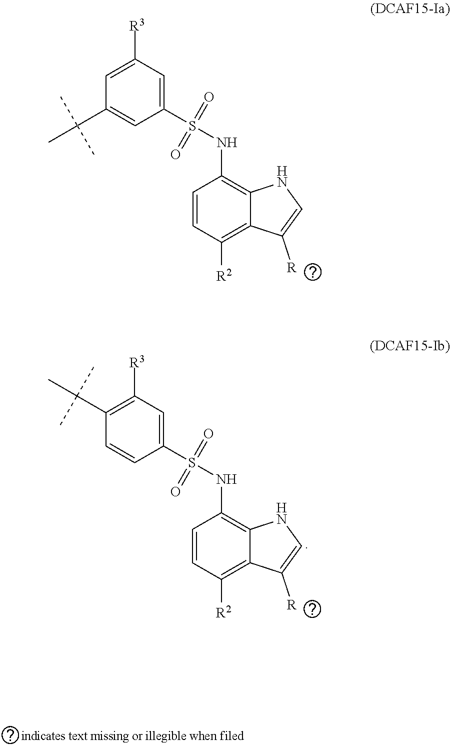

4. A PROTAC according to any one of the preceding items, wherein the ligand for DCAF15 is selected from a compound of Formula (DCAF15-Ia)

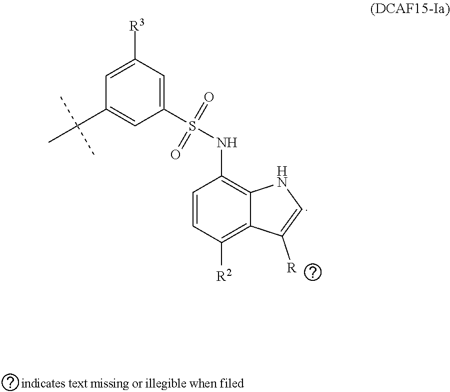

5. A PROTAC according to any one of the preceding items, wherein the E3 ubiquitin ligase ligand is selected from a compound of Formula (VHL-I), (VHL-II), (VHL-III), (VHL-IV), (VHL-V), (VHL-VI), (VHL-VII), (CRBN-I), or (CRBN-II).
6. A PROTAC according to any one of the preceding items, wherein the E3 ubiquitin ligase ligand is selected from a compound of Formula (VHL-I) or (CRBN-II).
7. A PROTAC according to any one of the preceding items, wherein the linker has a chain length of 6-24 atoms, preferably 6-22 atoms, more preferably 6-20 atoms, even more preferably 6-18 atoms, most preferably 8-16 atoms.
8. A PROTAC according to any one of the preceding items, wherein R³ is selected as H.
9. A PROTAC according to any one of the preceding items, wherein R² is selected as H.
10. A PROTAC according to any one of the preceding items, wherein R¹ is selected as Cl.
11. A PROTAC according to any one of the preceding items, wherein the linker comprises a PEG chain, an extended glycol chain, an alkyl chain, or a chain having a cyclic motif selected from a structure of Formula L1-L4,

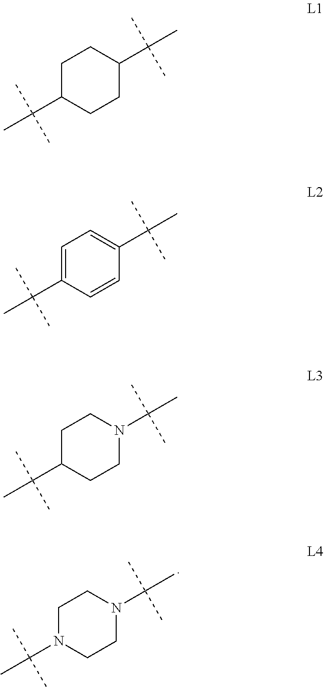

12. A PROTAC according to any one of the preceding items, wherein the linker comprises a PEG chain, an extended glycol chain, or an alkyl chain.
13. A PROTAC according to any one of the preceding items, wherein the linker comprises a structure selected from Formula L1-L8:

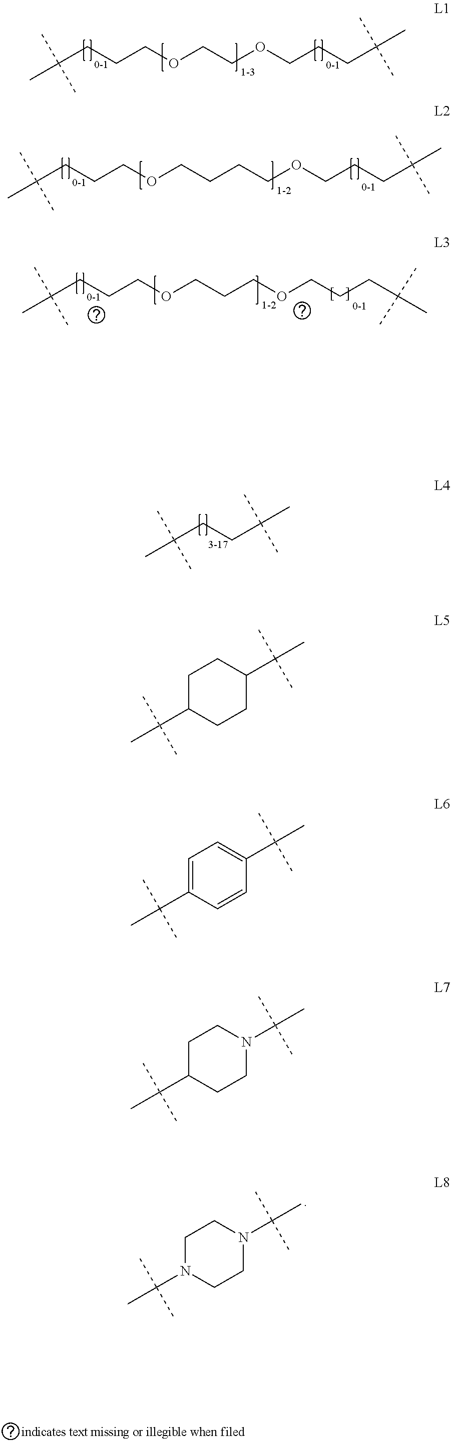

14. A PROTAC according to any one of the preceding items, wherein the linker consists of a structure selected from the list consisting of:

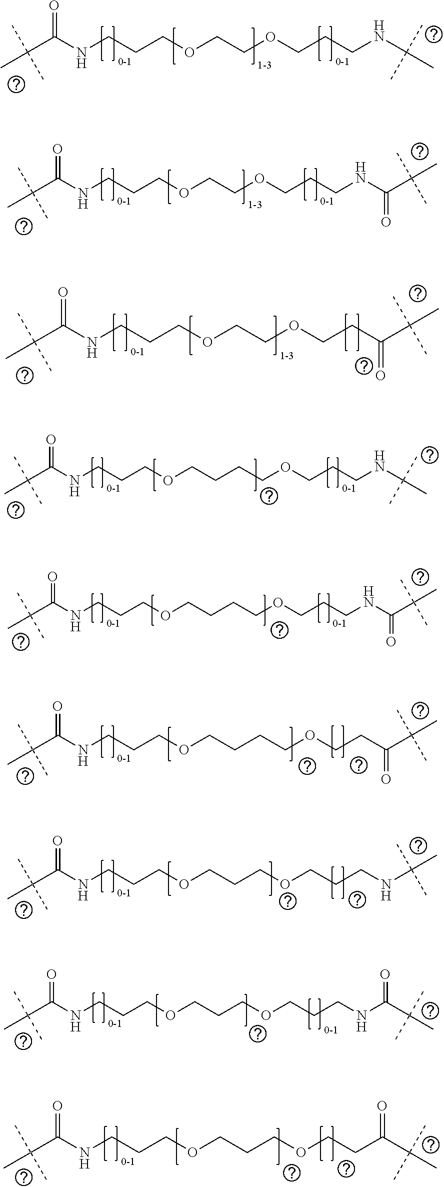

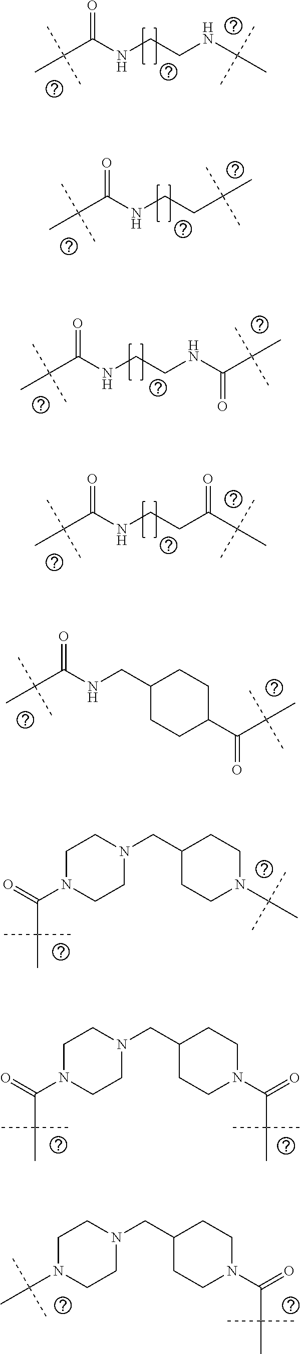

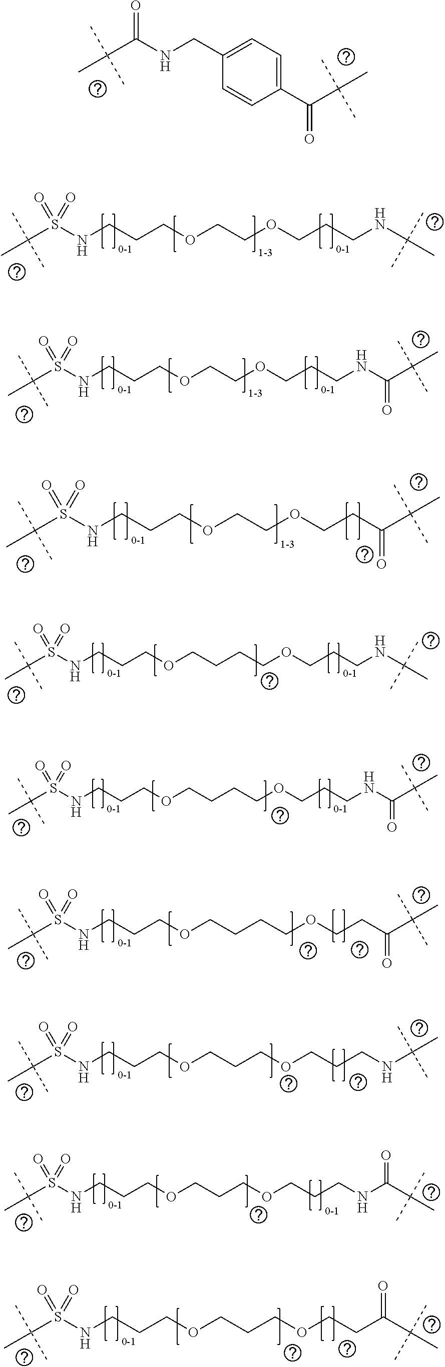

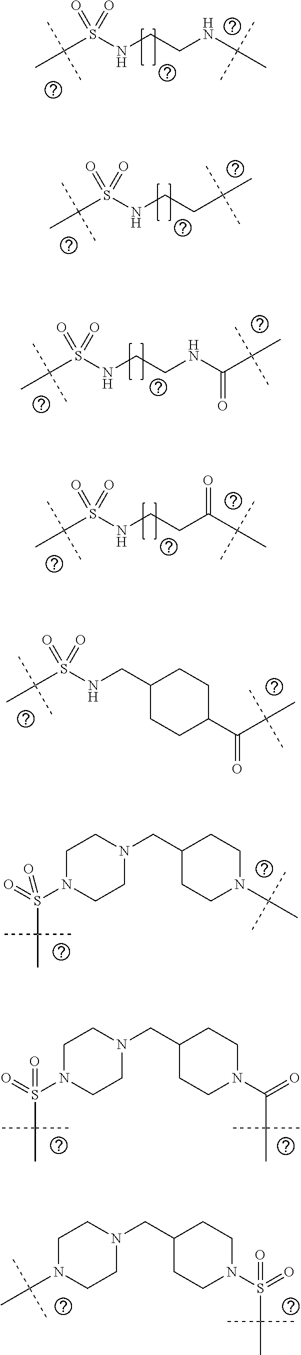

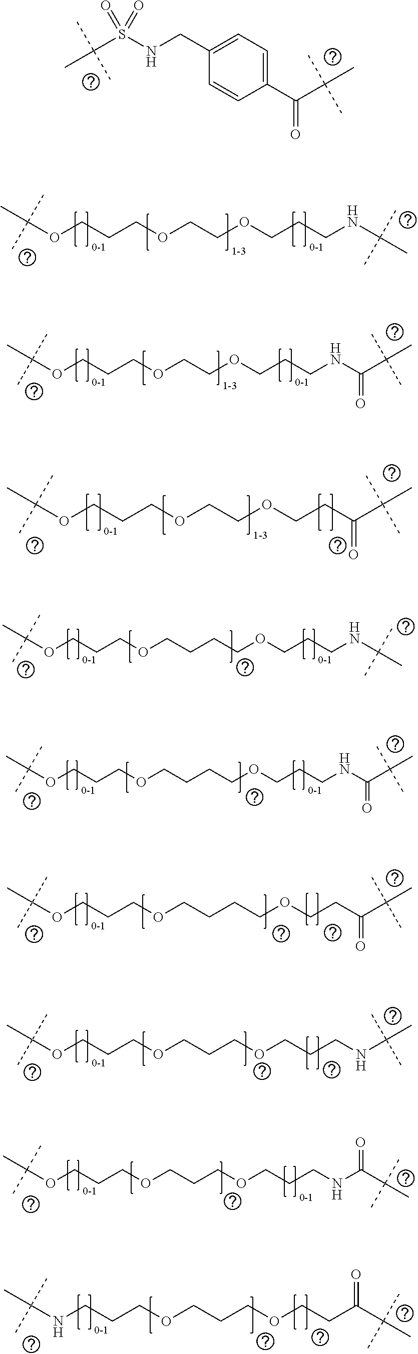

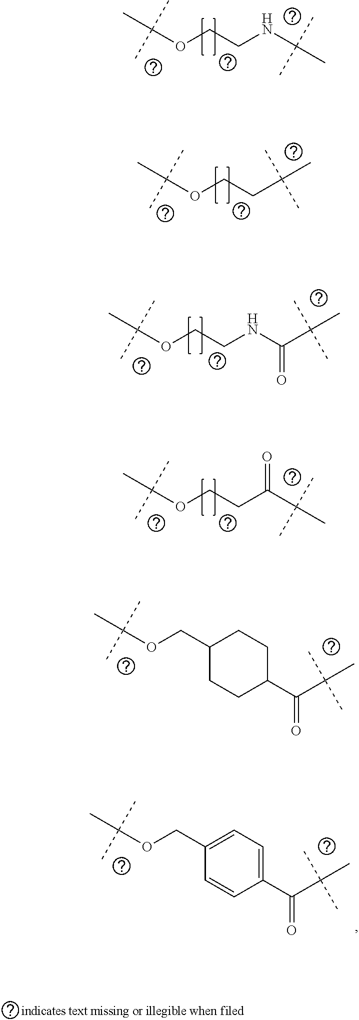

wherein the dotted lines marked * denote the end of the linker covalently connected to the DCAF15 ligand and the dotted lines marked ** denote the end of the linker covalently connected to the E3 ligase ligand.
15. A PROTAC according to item 1, wherein the PROTAC has the structure of compound nos. 1-23.

### Aspect 2: Medical Use of Protacs

In a second aspect, the present invention relates to PROTACs, according to the first aspect and any one of its embodiments and items, for use as a medicament. Preferably, the PROTACs is for use in the treatment of amyloidosis; more preferably for use in the treatment of synucleinopathies; even more preferably for use in the treatment of Parkinson's disease (PD), dementia with Lewy bodies (DLB), and multiple system atrophy (MSA); most preferably, for use in the treatment of Parkinson's disease (PD).

### Items of Aspect 2

1. A proteolysis targeting chimera (PROTAC) or a pharmaceutically acceptable salt thereof for use in the treatment of amyloidosis, said PROTAC comprising a ligand for DCAF15 (i.e. protein of interest) covalently connected to an E3 ubiquitin ligase ligand via a linker,
   wherein
      the ligand for DCAF15 is selected from a compound of Formula (DCAF15-I), (DCAF15-II), or (DCAF15-III)

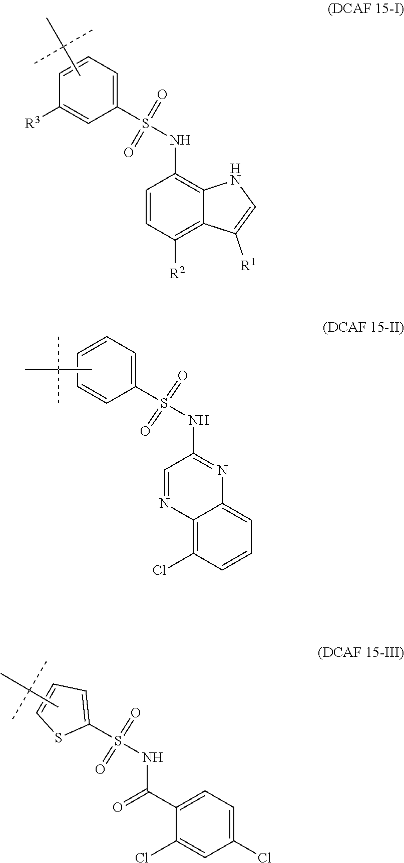

wherein, R¹ is selected as F, Cl, Br, I, CN, Me, or CF₃; R² is selected as H, CN, CF₃ or Me; R³ is selected as CN or H; and wherein the dotted lines denote a covalent connection to the E3 ubiquitin ligase ligand via the linker;
the E3 ligase ligand is selected from a CRBN-ligand, a VHL-ligand, a RNF114-ligand, a DCAF11-ligand, a DCAF15-ligand, or a MDM2-ligand.
2. A PROTAC for use according to item 1, wherein the ligand for DCAF15 consists of a compound of Formula (DCAF15-I).
3. A PROTAC for use according to any one of the preceding items, wherein the E3 ligase ligand is selected from a CRBN-ligand or a VHL-ligand.
4. A PROTAC for use according to any one of items 1-2, wherein the E3 ubiquitin ligase ligand is selected from a compound of Formula (VHL-I), (VHL-II), (VHL-III), (VHL-IV), (VHL-V), (VHL-VI), (VHL-VII), (MDM2-I), (MDM2-II), (MDM2-III), (MDM2-IV), (DCAF11-I), (DCAF15-I), (RNF114-I), (CRBN-I), or (CRBN-II) having the structure:

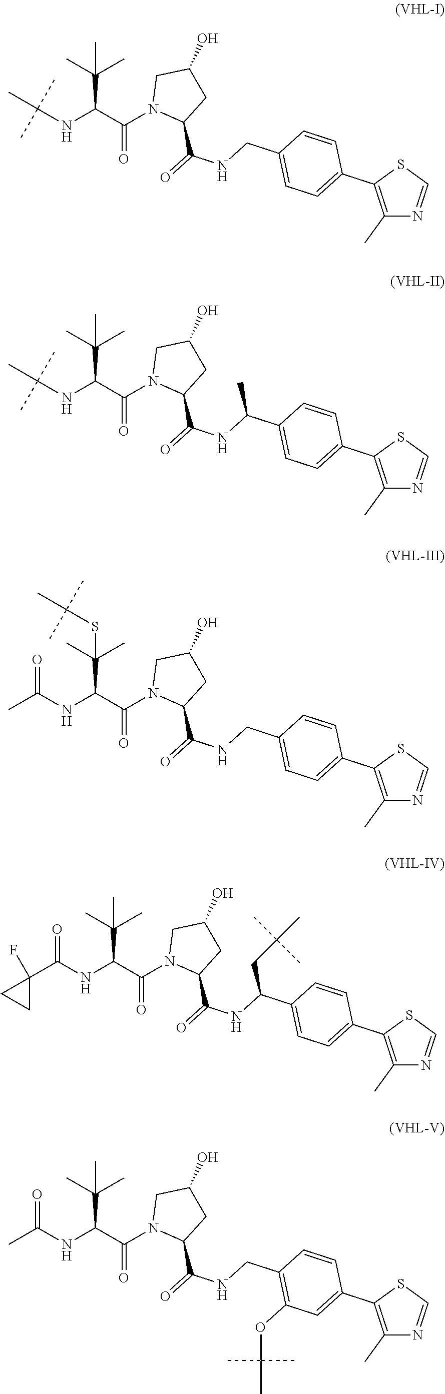

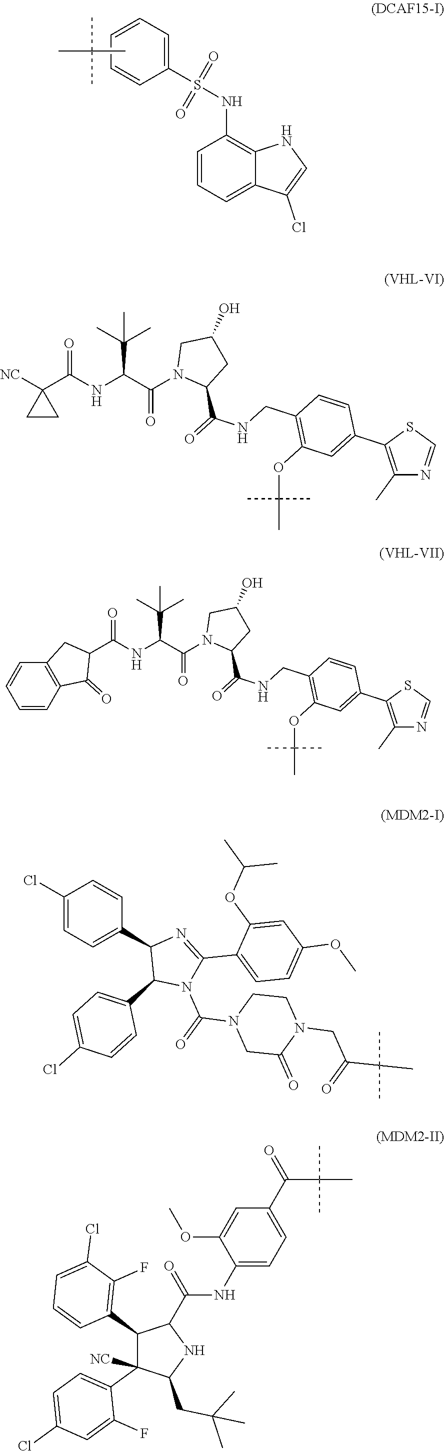

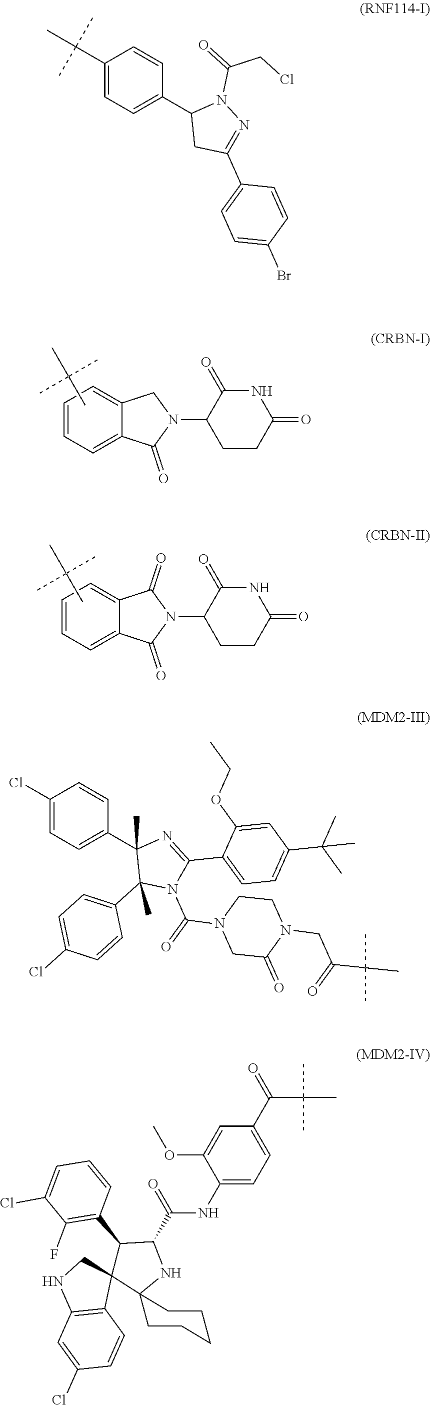

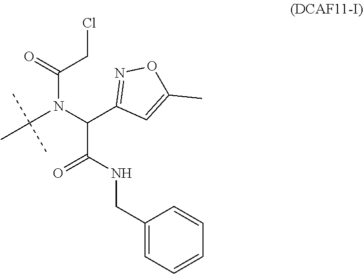

5. A PROTAC for use according to any one of the preceding items, wherein the E3 ubiquitin ligase ligand is selected from a compound of Formula (VHL-I), (VHL-II), (VHL-III), (VHL-IV), (VHL-V), (VHL-VI), (VHL-VII), (CRBN-I), or (CRBN-II).
6. A PROTAC for use according to any one of the preceding items, wherein the ligand for DCAF15 is selected from a compound of Formula (DCAF15-Ia) or (DCAF15-Ib)

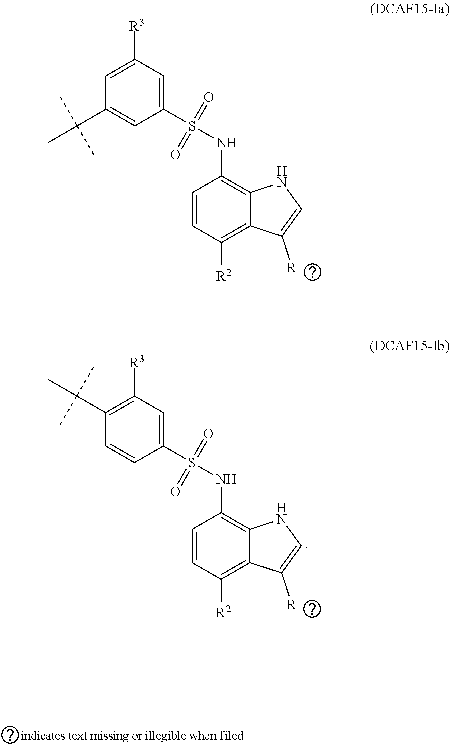

7. A PROTAC for use according to any one of the preceding items, wherein the ligand for DCAF15 is selected from a compound of Formula (DCAF15-Ia)

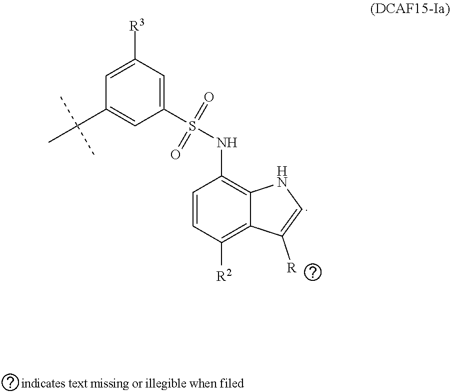

8. A PROTAC for use according to any one of the preceding items, wherein the linker has a chain length of 6-24 atoms, preferably 6-22 atoms, more preferably 6-20 atoms, even more preferably 6-18 atoms, most preferably 8-16 atoms.
9. A PROTAC for use according to any one of the preceding items, wherein R³ is selected as H.
10. A PROTAC for use according to any one of the preceding items, wherein R² is selected as H.
11. A PROTAC for use according to any one of the preceding items, wherein R¹ is selected as Cl.
12. A PROTAC for use according to any one of the preceding items, wherein the linker comprises a PEG chain, an extended glycol chain, an alkyl chain or a chain having a cyclic motif selected from a structure of Formula L1-L4

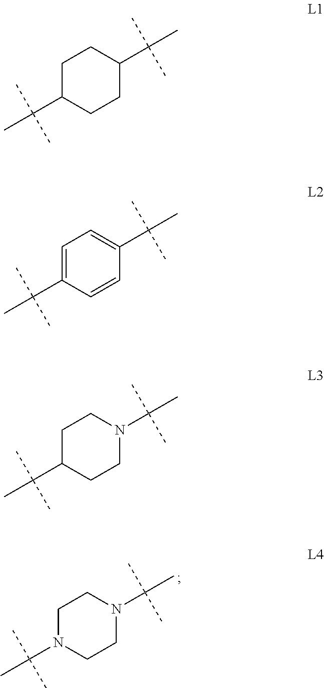

13. A PROTAC for use according to any one of the preceding items, wherein the linker comprises a PEG chain, an extended glycol chain, or an alkyl chain.
14. A PROTAC for use according to item 1, wherein the PROTAC has the structure of compound nos. 1-23.
15. A PROTAC for use according to any one of the preceding items, wherein the PROTACs is for use in the treatment of synucleinopathies.

### Aspect 3: HyT-PD Molecules

Ligands for DCAF15 (i.e. Protein of Interest)

A number of ligands for DCAF15 are known in the art, such as the SPLicing inhibitor sulfonAMides indisulam, tasisulam, and chloroquinoxaline sulfonamide (CQS). Furthermore, various analogues of the indisulam scaffold have been synthesized and shown to bind to DCAF15, such as those described in e.g. WO 2022/169755. It should be appreciated that any of the known ligands for DCAF15 may be used as ligand in the HyT-PD molecules according to the invention. In a preferred embodiment, the ligand for DCAF15 comprises the structure of Formula (DCAF15-I), (DCAF15-II), (DCAF15-III),

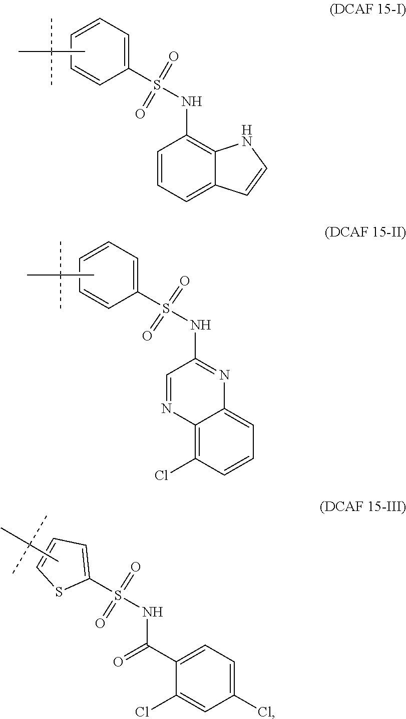

wherein the dotted lines denote a covalent connection to a hydrophobic tag (HyT) via a linker.

In a more preferred embodiment, the DCAF15 ligand is selected from a compound of Formula (DCAF15-I),

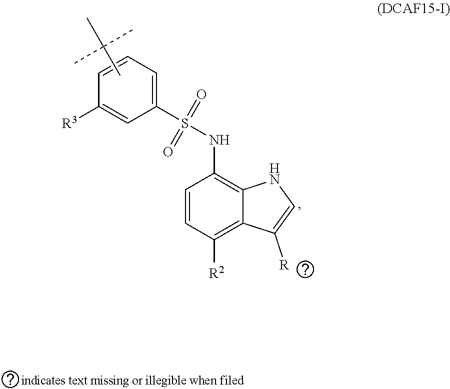

wherein R¹ is selected as F, Cl, Br, I, CN, Me, or CF₃; R² is selected as H, CN, CF₃ or Me; R³ is selected as CN or H; and wherein the dotted lines denote a covalent connection to a hydrophobic tag (HyT) via a linker. Preferably, R¹ is selected as Cl or CN, most preferably Cl; R² is selected as H or Me, most preferably H; and R³ is selected as H.

In an even more preferred embodiment, the DCAF15 ligand is selected from a compound of Formula (DCAF15-Ia) or (DCAF15-Ib)

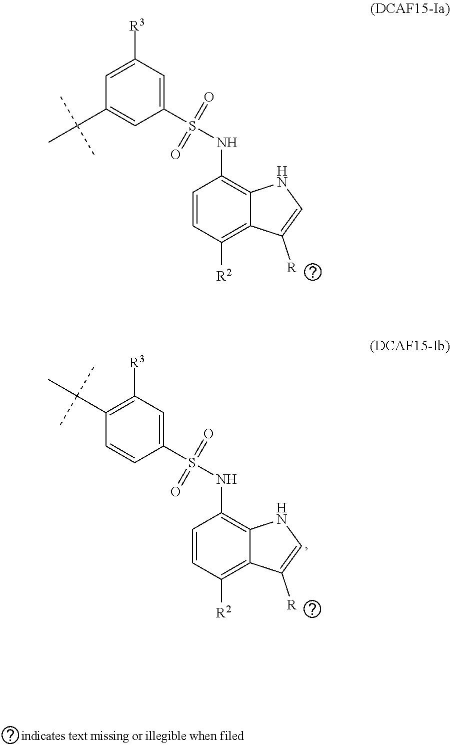

wherein R¹ is selected as F, Cl, Br, I, CN, Me, or CF₃; R² is selected as H, CN, CF₃ or Me; R³ is selected as CN or H; and wherein the dotted lines denote a covalent connection to a hydrophobic tag (HyT) via a linker. Preferably, R¹ is selected as Cl or CN, most preferably Cl; R² is selected as H or Me, most preferably H; and R³ is selected as H.

In a most preferred embodiment, the DCAF15 ligand consists of a compound of Formula (DCAF15-Ia)

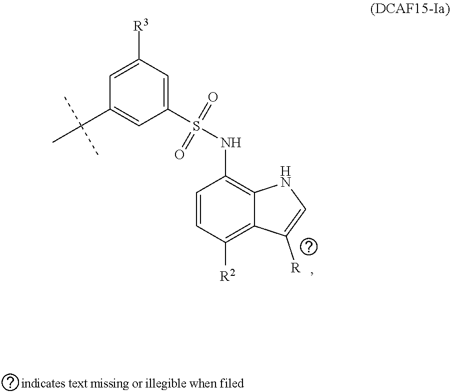

wherein R¹ is selected as F, Cl, Br, I, CN, Me, or CF₃; R² is selected as H, CN, CF₃ or Me; R³ is selected as CN or H; and wherein the dotted lines denote a covalent connection to a hydrophobic tag (HyT) via a linker. Preferably, R¹ is selected as Cl or CN, most preferably Cl; R² is selected as H or Me, most preferably H; and R³ is selected as H.

### Hydrophobic Tags for HyT-PD Molecules

A variety of hydrophobic tags have been successfully employed in HyT-PD molecules in the art. Among the hydrophobic tags are e.g. polycyclic aromatic hydrocarbons (PAH), a class of aromatic hydrocarbons that contain two or more fused benzene rings, such as naphthalene (Formula II), pyrene or β-naphthoflavone. Another example of a hydrophobic tag is fluorene (Formula III) a rigid planar biphenyl compound. In particularly, adamantane (Formula IV) has been widely employed as a hydrophobic tag in many HyT-PD molecules. Notably, HyT-PD molecules with an adamantane tag have demonstrated superior protein degradation efficacy and some improved ADME properties. Further examples of hydrophobic tags include norbornene (Formula V), tri-Boc protected arginine (Formula VI), or menthoxyacetyl (Formula VII). It should be appreciated that any hydrophobic tag can be used in the synthesis of HyT-PD molecules according to the invention. In a highly preferred embodiment, the hydrophobic tag (HyT) is selected from a compound of Formula (I)-(VII),

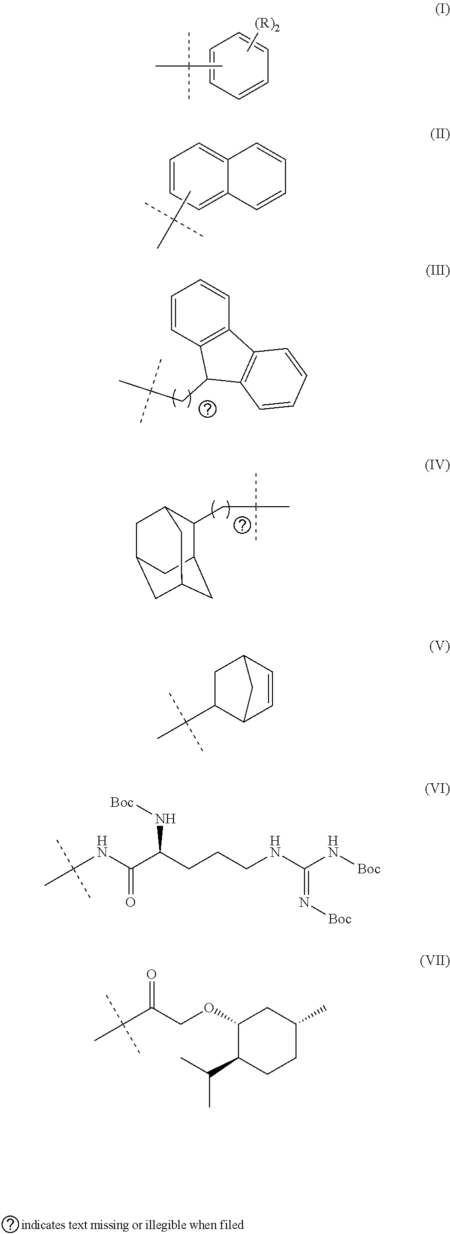

wherein the dotted lines denote a covalent connection to the DCAF15 ligand via the linker; z is an integer of 0, 1, or 2; and R is/are independently selected from the list consisting of halogens, —O—(C₁-C₅-alkyl), —(C₁-C₅-alkyl), —(C₁-C₅-fluoroalkyl), —O-phenyl, sulfonamide (—SO₂NH₂), and alkyl sulfonamide (—SO₂NH(C₁-C₅-alkyl) or —SO₂N(C₁-C₅-alkyl)₂).

It is highly preferred that the hydrophobic tag (HyT) is selected from a compound for Formula (I)-(IV),

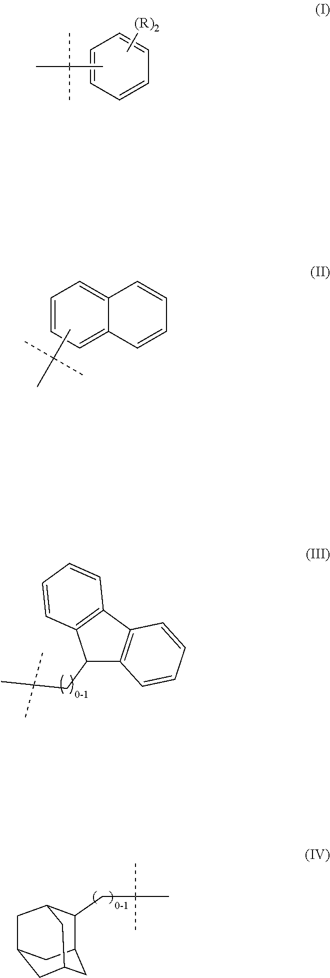

wherein the dotted lines denote a covalent connection to the DCAF15 ligand via the linker; z is an integer of 0, 1, or 2; R is/are independently selected from the list consisting of halogens, —O—(C₁-C₅-alkyl), —(C₁-C₅-alkyl), —O—(C₁-C₅-fluoroalkyl), —(C₁-C₅-fluoroalkyl), —O-phenyl, sulfonamide (—SO₂NH₂), and alkyl sulfonamide (—SO₂NH (C₁-C₅-alkyl) or —SO₂N (C₁-C₅-alkyl)₂).

More preferably, the hydrophobic tag (HyT) is selected from a compound for Formula (I)-(IV),

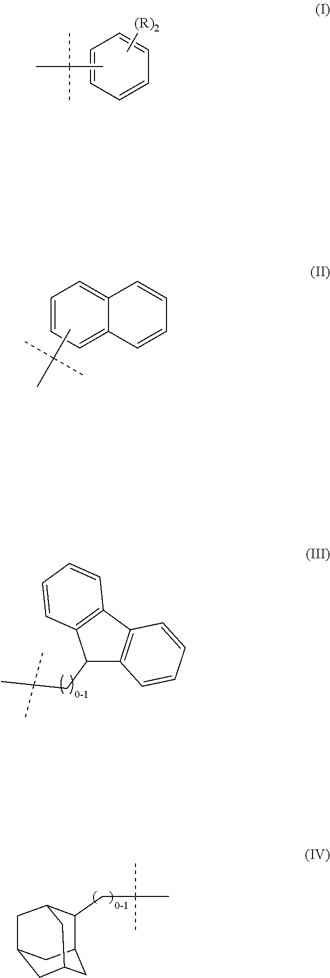

wherein the dotted lines denote a covalent connection to the DCAF15 ligand via the linker; z is an integer of 0, 1, or 2; R is/are independently selected from the list consisting of halogens, —O—(C₁-C₅-alkyl), —(C₁-C₅-alkyl), —O-phenyl, sulfonamide (—SO₂NH₂).

### Examples of Linkers in HyT-PD Molecules

A variety of linkers have been successfully employed in HyT-PD molecules in the art. Alkyl chains, PEG chains, and extended glycol chains are common linker motifs found in HyT-PD molecules. These linkers offer some key advantages, including their commercial availability, their synthetic accessibility, their flexibility, and their ability to easily tune their length and composition via a wide array of robust chemical methods. Thus, in some embodiments of the present invention, the linker comprises a PEG chain, an extended glycol chain, an alkyl chain, and/or a chain comprising a cyclic motif selected from a disubstituted cyclohexane ring system, a disubstituted benzene ring system, a disubstituted piperidine ring system and/or a disubstituted piperazine ring system to rigidify the linker. The linkers may be linear or branched or comprise one or more cyclic motif(s). Preferably, the linkers are linear or comprise one or more cyclic motifs. For the HyT-PD molecules, the linkers may have a chain length of 3-18 atoms, preferably 3-14 atoms, more preferably 3-10 atoms, even more preferably 3-8 atoms, most preferably 3-6 atoms. In calculation of the linker chain length, any functional groups used to connect the linker to the hydrophobic tag (HyT) in one end and the linker to the DCAF15 ligand in the opposite end are included. Thus, the chain length is calculated as the number of atoms in a linear chain between the DCAF15 ligand and the hydrophobic tag (see Table 1 for examples). As another non-limiting example, HyT-PD molecule compound no. 30 has a chain length of 3 atoms, and HyT-PD molecule compound no. 25 has a chain length of 16 atoms. For the HyT-PD molecules, a short linker length of e.g. 3 atoms was found to be sufficient for degradation of DCAF15. Shorter linkers have the benefit of lower molecular weight and improved drug-like properties.

In the present context, it should be appreciated that the linkers comprising a PEG chain, an alkyl chain, and/or an extended glycol chain may be covalently attached to the DCAF15 ligand in one end and to the HyT in the opposite end via a variety of functional groups commonly used in HyT-PD molecules for linker attachment. Such functional groups may include e.g. amides, sulfonamides, sulfinamides, sulfonates, sulfinates, carbamates, thiocarbamates, esters, thioesters, ketones, ethers, thiol ethers, amines, alkynes, triazoles or tetrazoles depending on the hydrophobic tag in the HyT-PD molecule used and their synthetic accessibility from commercial building blocks. Thus, it will be appreciated that the linkers can be designed in variety of different ways. Most preferably, the linker is covalently connected to the DCAF15 ligand with an amide or sulfonamide, most preferably an amide as illustrated herein. In the HyT-PD molecules, the linker preferably comprises a PEG chain, an extended glycol chain, an alkyl chain or a chain having a cyclic motif selected from a structure of Formula L1-L4,

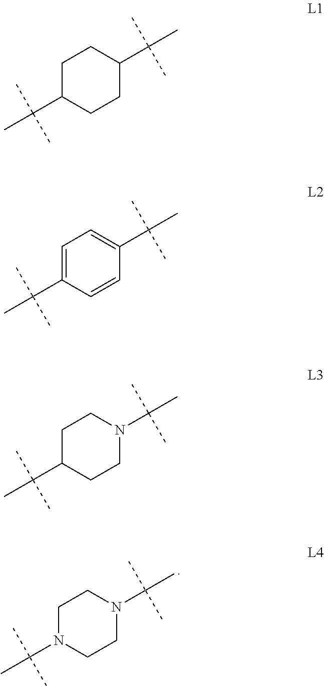

In a preferred embodiment, the linker comprises a PEG chain, an extended glycol chain, an alkyl chain, most preferably, the linker comprises an alkyl chain. Preferably, the linker comprises a structural motif of Formula L1-L8.

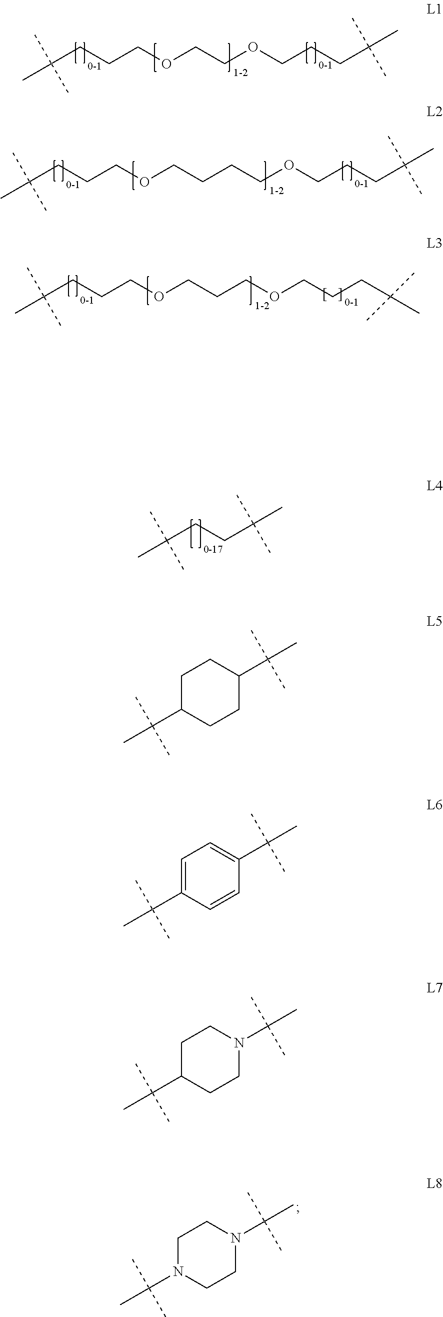

more preferably the linker comprises a structural motif of Formula L1-L4 or L8:

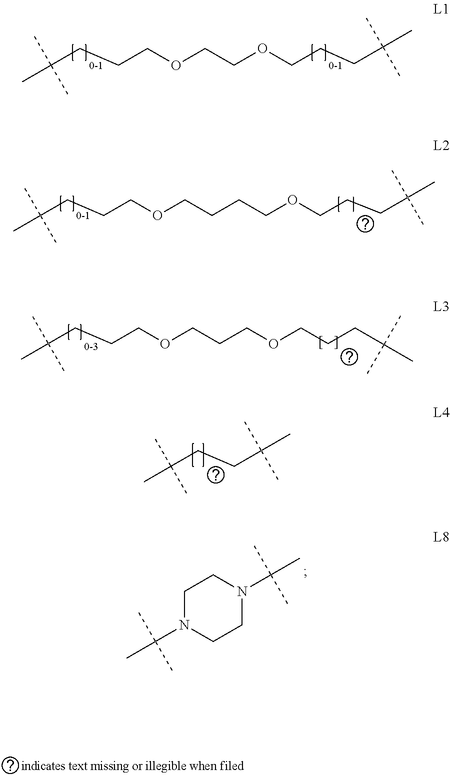

even more preferably the linker comprises a structural motif of Formula L1, L4 or L8:

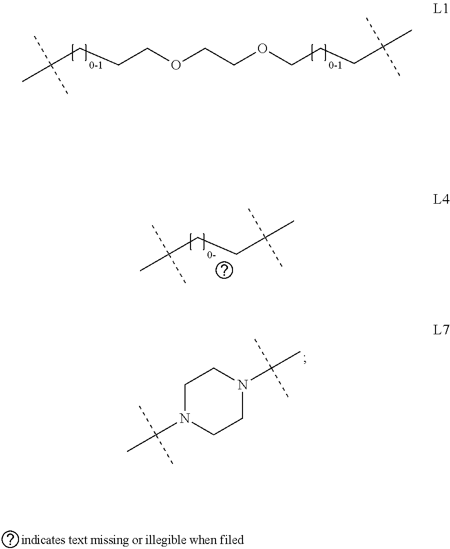

yet even more preferably the linker comprises a structural motif of Formula L4:

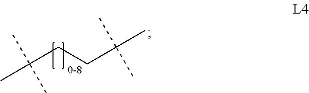

even more preferably the linker comprises a structural motif of Formula L4:

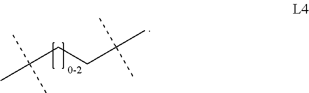

Most preferably the linker is covalently connected to the DCAF15 ligand via a functional group selected from an amide, sulfonamide, or ether, more preferably an amide or sulfonamide, most preferably an amide as illustrated herein. Most preferably the linker is covalently connected to the hydrophobic tag via an amine, amide, ketone or directly via a methylene (—CH₂—) as illustrated herein. However, it will be apparent to the skilled person that the linker may be connected to the DCAF15 ligand and the hydrophobic tag via different functional groups well known in the art.

Thus, in a highly preferred embodiment, the linker consists of a structure selected from the list consisting of:

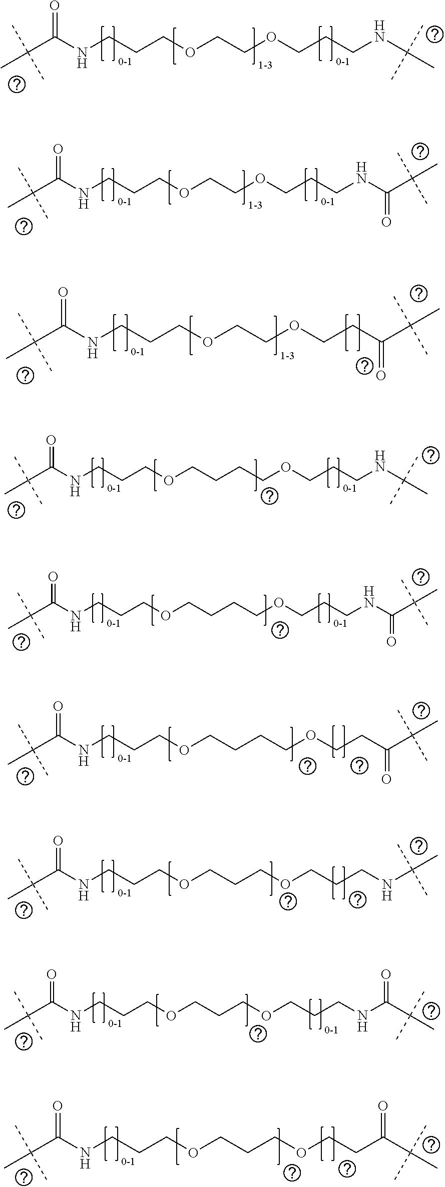

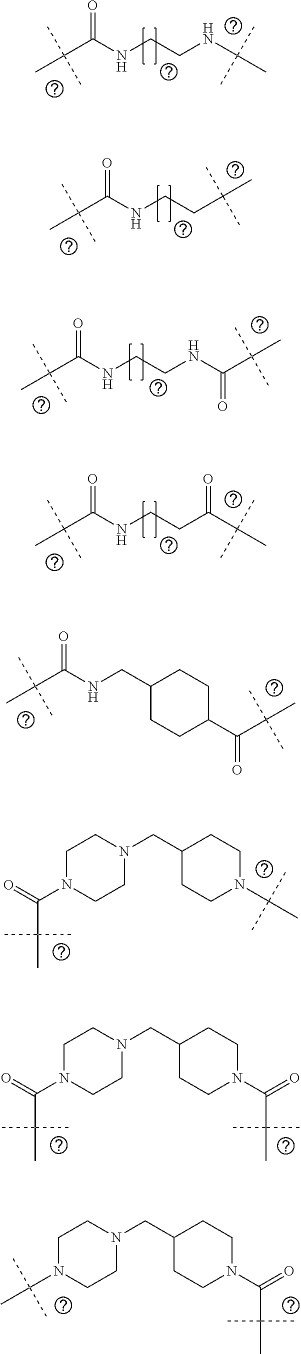

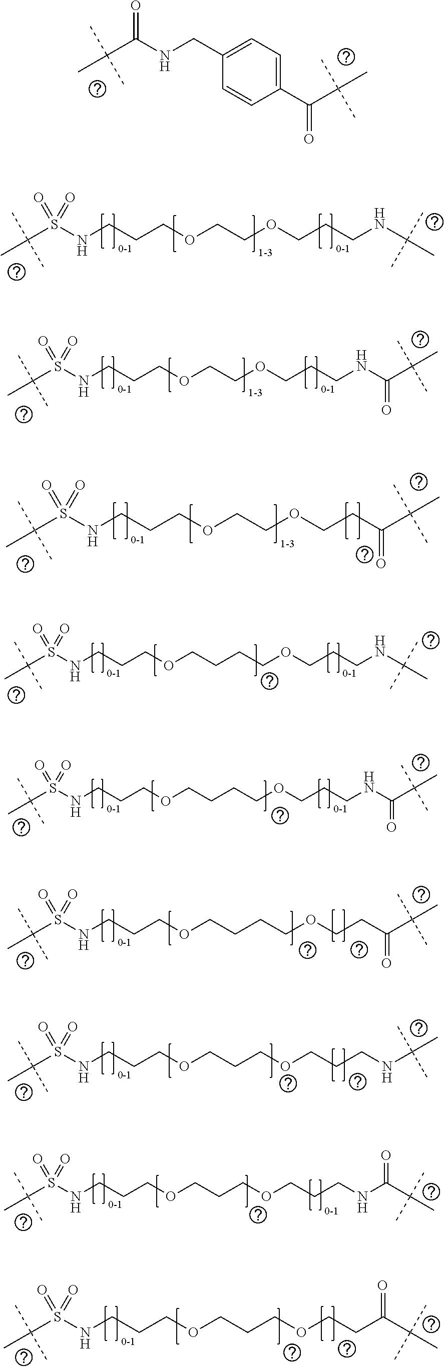

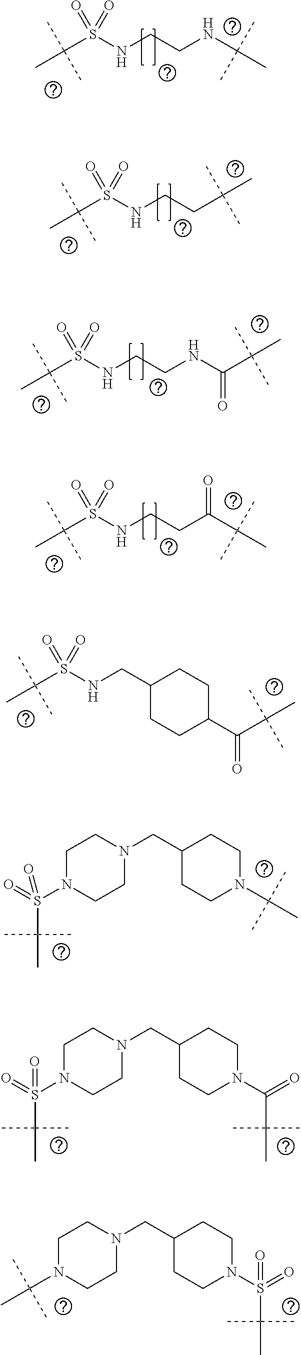

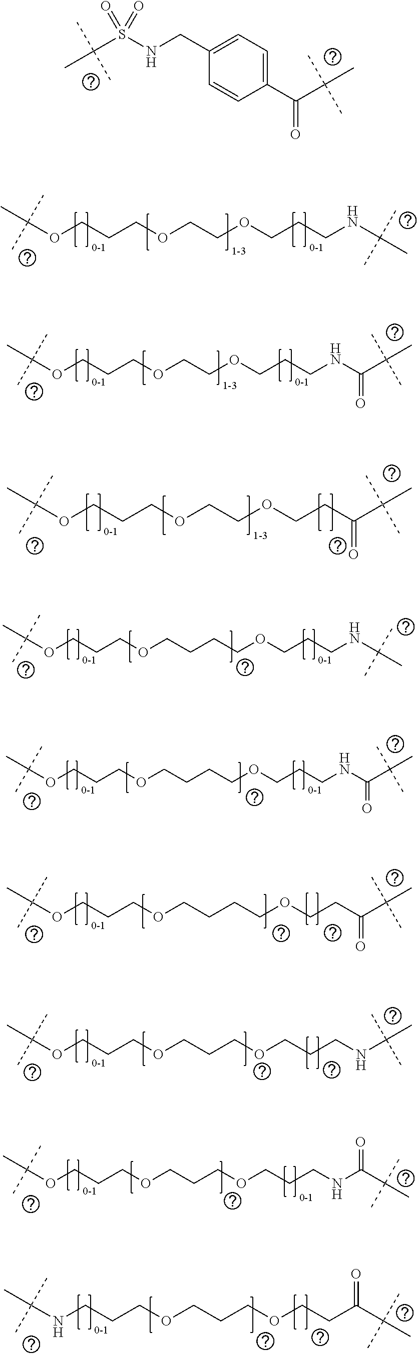

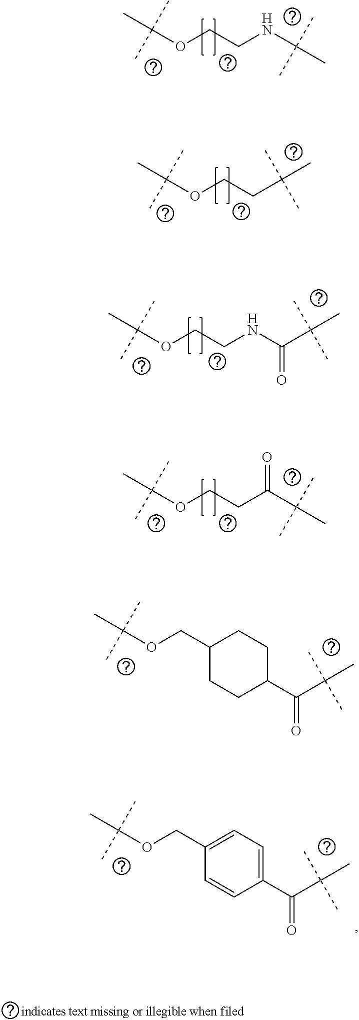

wherein the dotted lines marked * denote the end of the linker covalently connected to the DCAF15 ligand and the dotted lines marked ** denote the end of the linker covalently connected to the hydrophobic tag (HyT); more preferably the linker consists of a structure selected from the list consisting of:

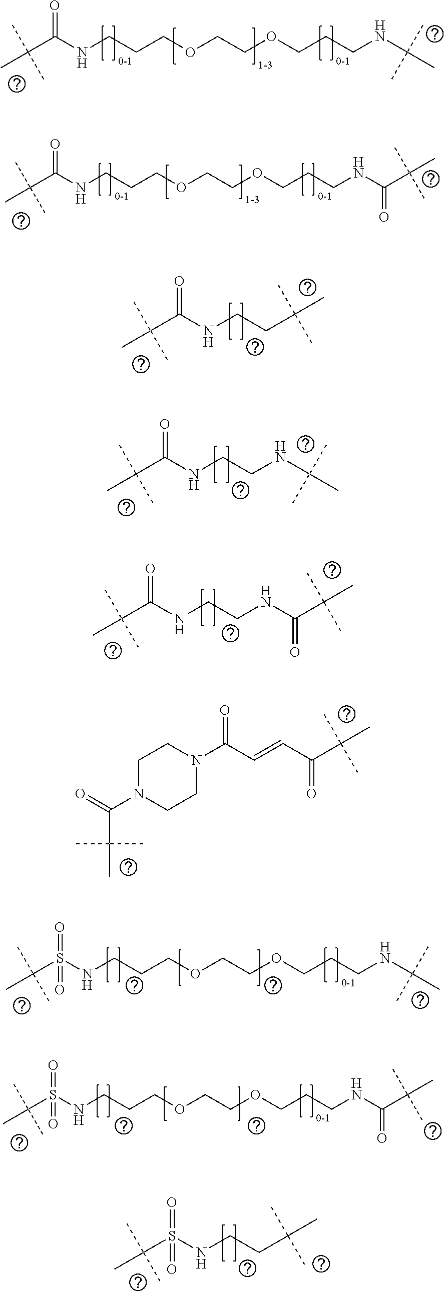

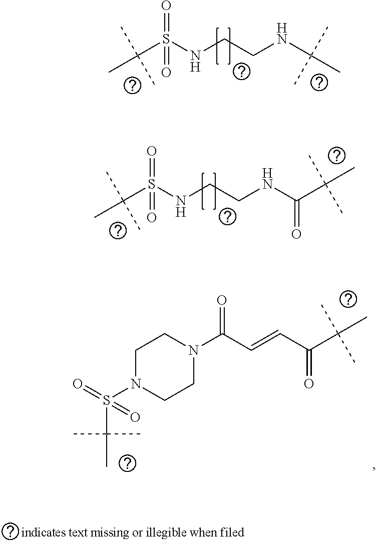

wherein the dotted lines marked * denote the end of the linker covalently connected to the DCAF15 ligand and the dotted lines marked ** denote the end of the linker covalently connected to the hydrophobic tag (HyT); even preferably the linker consists of a structure selected from the list consisting of:

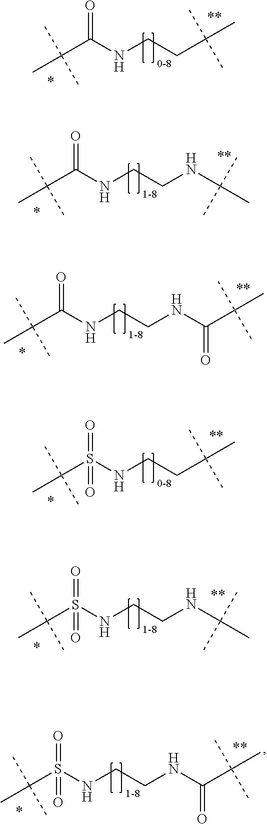

wherein the dotted lines marked * denote the end of the linker covalently connected to the DCAF15 ligand and the dotted lines marked ** denote the end of the linker covalently connected to the hydrophobic tag (HyT); most preferably the linker consists of a structure selected from the list consisting of:

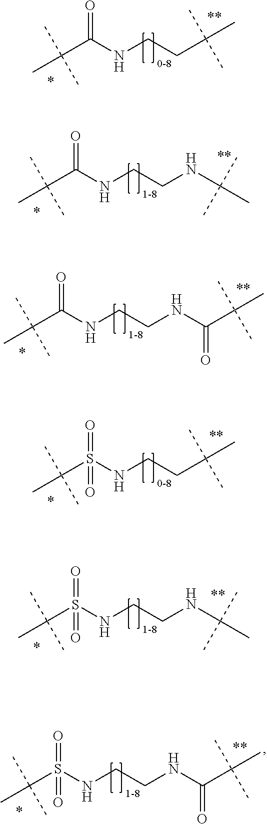

wherein the dotted lines marked * denote the end of the linker covalently connected to the DCAF15 ligand and the dotted lines marked ** denote the end of the linker covalently connected to the hydrophobic tag (HyT).

Most preferably, the linkers drawn above in aspect 3 have a chain length of 3-18 atoms, preferably 3-14 atoms, such as 3-10 atoms, more preferably 3-8 atoms, most preferably 3-6 atoms.

In a third aspect, the present invention relates to a hydrophobic tag-based protein degradation (HyT-PD) molecule or a pharmaceutically acceptable salt thereof, said HyT-PD molecule comprising a ligand for DCAF15 (i.e. protein of interest) covalently connected to a hydrophobic tag (HyT) via a linker,
wherein
   the ligand for DCAF15 is selected from a compound of Formula (DCAF15-I), (DCAF15-II), or (DCAF15-III)

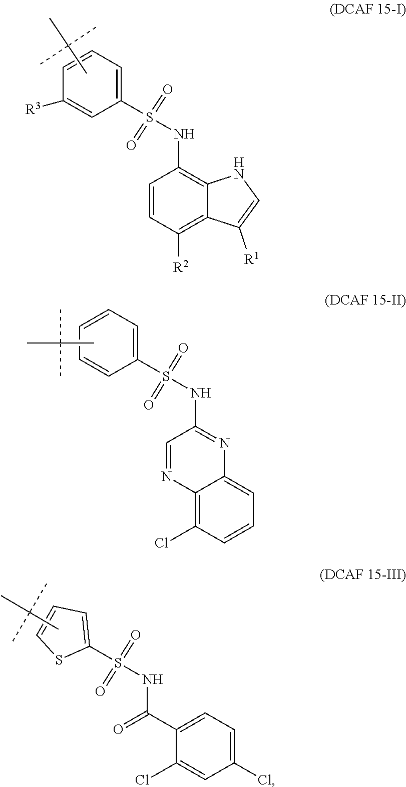

wherein R¹ is selected as F, Cl, Br, I, CN, Me, or CF₃; R² is selected as H, CN, CF₃, or Me; R³ is selected as CN or H; and wherein the dotted lines denote a covalent connection to the hydrophobic tag (HyT) via the linker;
the hydrophobic tag (HyT) is selected from a compound of Formula (I)-(VII),

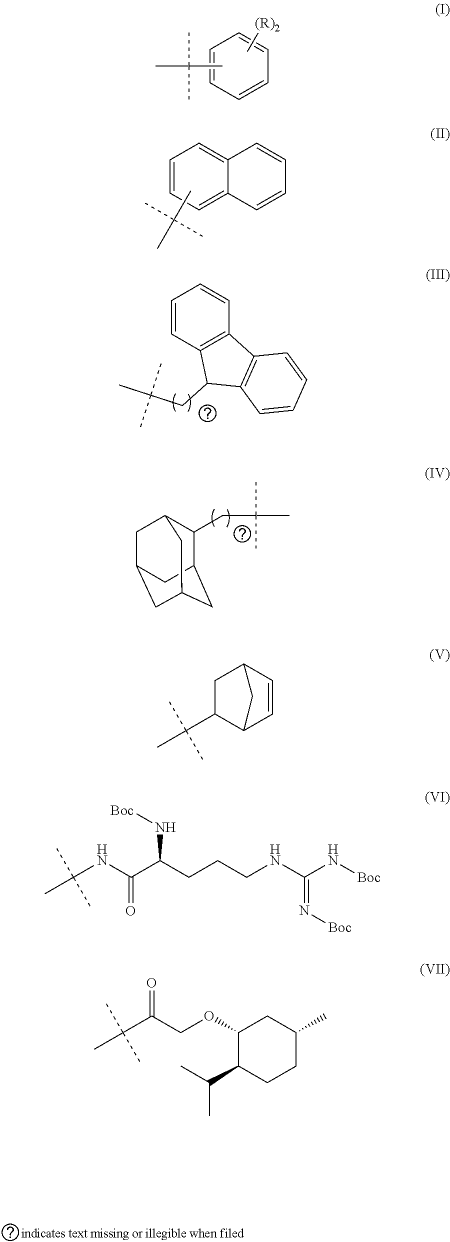

wherein the dotted lines denote a covalent connection to the DCAF15 ligand via the linker;
z is an integer of 0, 1, or 2;
R is/are independently selected from the list consisting of halogens, —O—(C₁-C₅-alkyl), —(C₁-C₅-alkyl), —O—(C₁-C₅-fluoroalkyl), —(C₁-C₅-fluoroalkyl), —O-phenyl, sulfonamide (—SO₂NH₂), and alkyl sulfonamide (—SO₂NH(C₁-C₆-alkyl) or —SO₂N (C₁-C₅-alkyl)₂).

Preferably, the ligand for DCAF15 is selected from a compound of Formula (DCAF15-I). Thus, in a preferred embodiment, the present invention relates to a hydrophobic tag-based protein degradation (HyT-PD) molecule or a pharmaceutically acceptable salt thereof, said HyT-PD molecule comprising a ligand for DCAF15 (i.e. protein of interest) covalently connected to a hydrophobic tag (HyT) via a linker,
wherein
   the ligand for DCAF15 is selected from a compound of Formula (DCAF15-I),

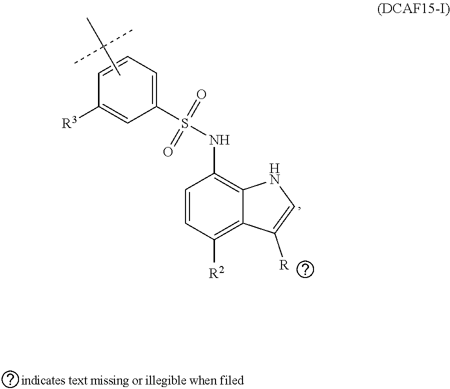

wherein R¹ is selected as F, Cl, Br, I, CN, Me, or CF₃; R² is selected as H, CN, CF₃, or Me; R³ is selected as CN or H; and wherein the dotted line denotes a covalent connection to the hydrophobic tag (HyT) via a linker;
the hydrophobic tag (HyT) is selected from a compound of Formula (I)-(VII),

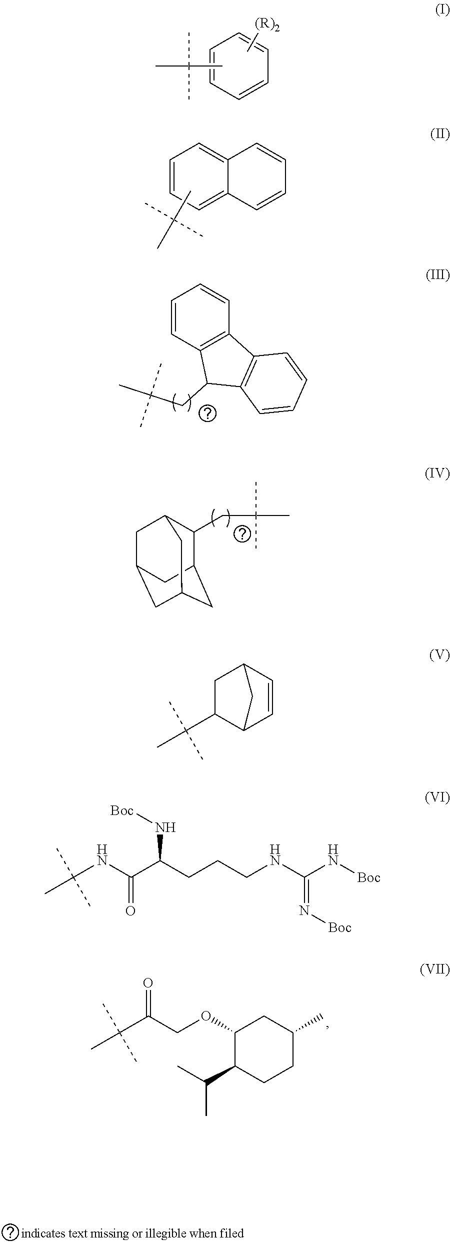

wherein the dotted lines denote a covalent connection to the DCAF15 ligand via the linker;
z is an integer of 0, 1, or 2;
R is/are independently selected from the list consisting of halogens, —O—(C₁-C₅-alkyl), —(C₁-C₅-alkyl), —O—(C₁-C₅-fluoroalkyl), —(C₁-C₅-fluoroalkyl), —O-phenyl, sulfonamide (—SO₂NH₂), and alkyl sulfonamide (—SO₂NH (C₁-C₅-alkyl) or —SO₂N(C₁-C₆-alkyl)₂).

In a more preferred embodiment, the present invention relates to a hydrophobic tag-based protein degradation (HyT-PD) molecule or a pharmaceutically acceptable salt thereof, said HyT-PD molecule comprising a ligand for DCAF15 (i.e. protein of interest) covalently connected to a hydrophobic tag (HyT) via a linker,
wherein
   the ligand for DCAF15 is selected from a compound of Formula (DCAF15-I),

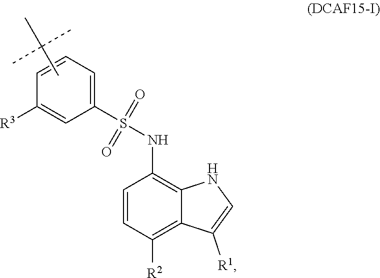

wherein R¹ is selected as F, Cl, Br, I, CN, Me, or CF₃; R² is selected as H, CN, CF₃, or Me; R³ is selected as CN or H; and wherein the dotted line denotes a covalent connection to the hydrophobic tag (HyT) via a linker;
the hydrophobic tag (HyT) is selected from a compound of Formula (I-IV),

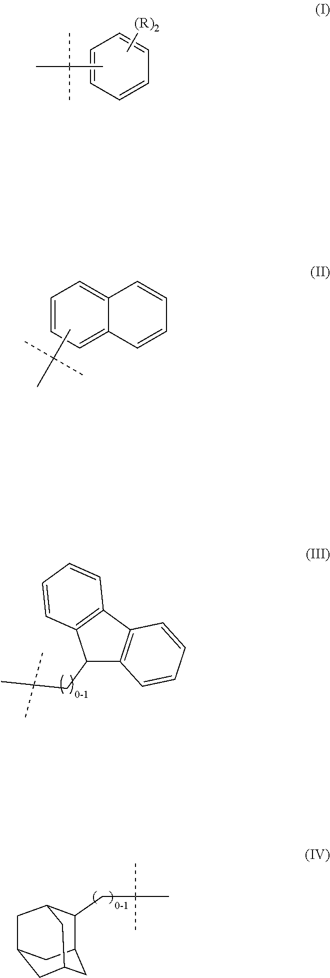

wherein the dotted lines denote a covalent connection to the DCAF15 ligand via the linker; z is an integer of 0, 1, or 2; R is/are independently selected from the list consisting of halogens, —O—(C₁-C₅-alkyl), —(C₁-C₅-alkyl), —O—(C₁-C₅-fluoroalkyl), —(C₁-C₅-fluoroalkyl), —O-phenyl, sulfonamide (—SO₂NH₂), and alkyl sulfonamide (—SO₂NH (C₁-C₅-alkyl) or —SO₂N (C₁-C₅-alkyl)₂).

In aspect 3 and its embodiments described above, it is highly preferred that the ligand for DCAF15 is selected from a compound of Formula (DCAF15a)

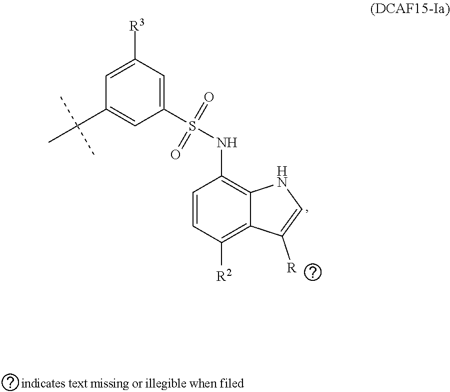

It is also highly preferred that R¹ is selected as Cl or CN, most preferably Cl; that R² is selected as H or Me, most preferably H; and that R³ is selected as H. It is preferred, that the linker comprises a structure selected from Formula L1-L8:

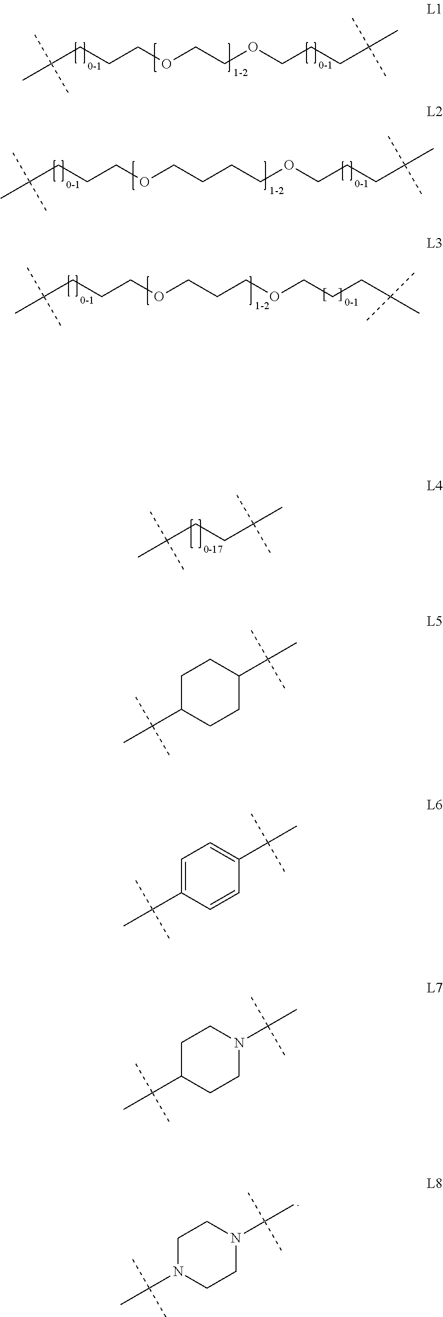

More preferably, the linker comprises a PEG chain, an extended glycol chain, or an alkyl chain. It is further highly preferred that the linker has a chain length of 3-18 atoms, preferably 3-14 atoms, such as 3-10 atoms, more preferably 3-8 atoms, most preferably 3-6 atoms.

### Items of Aspect 3

1. A hydrophobic tag-based protein degradation (HyT-PD) molecule or a pharmaceutically acceptable salt thereof, said HyT-PD molecule comprising a ligand for DCAF15 (i.e. protein of interest) covalently connected to a hydrophobic tag (HyT) via a linker,
   wherein
      the ligand for DCAF15 is selected from a compound of Formula (DCAF15-I), (DCAF15-II), or (DCAF15-III)

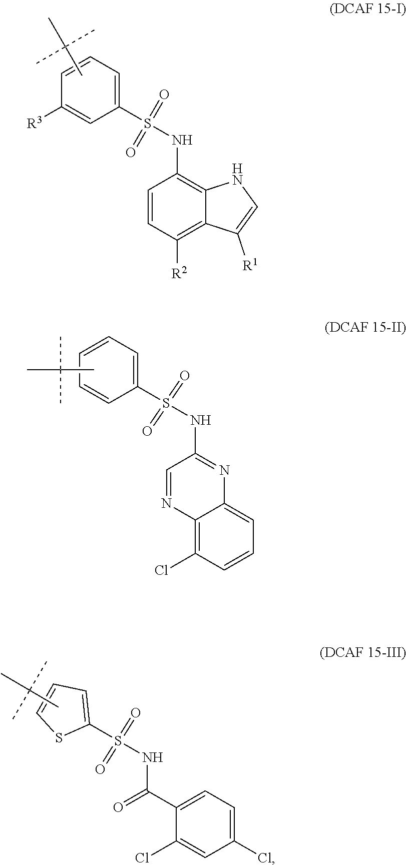

wherein R¹ is selected as F, Cl, Br, I, CN, Me, or CF₃; R² is selected as H, CN, CF₃, or Me; R³ is selected as CN or H; and wherein the dotted lines denote a covalent connection to the hydrophobic tag (HyT) via the linker;
the hydrophobic tag (HyT) is selected from a compound of Formula (I)-(VII),

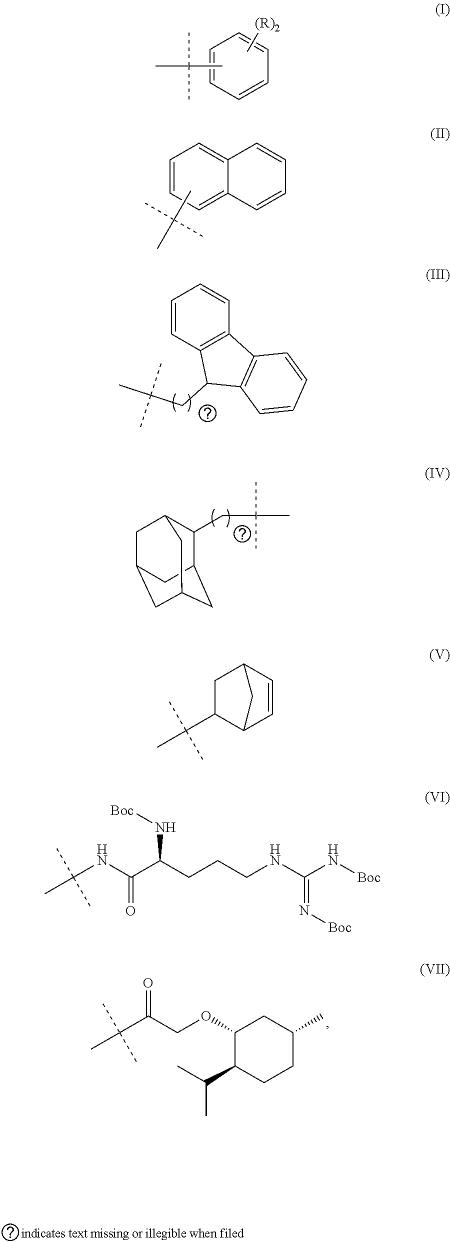

wherein the dotted lines denote a covalent connection to the DCAF15 ligand via the linker;
z is an integer of 0, 1, or 2;
R is/are independently selected from the list consisting of halogens, —O—(C₁-C₅-alkyl), —(C₁-C₅-alkyl), —O—(C₁-C₅-fluoroalkyl), —(C₁-C₅-fluoroalkyl), —O-phenyl, sulfonamide (—SO₂NH₂), and alkyl sulfonamide (—SO₂NH (C₁-C₅-alkyl) or —SO₂N (C₁-C₅-alkyl)₂).
2. The HyT-PD molecule according to item 1, wherein the ligand for DCAF15 is selected from a compound of Formula (DCAF15-I).
3. The HyT-PD molecule according to any one of the preceding items, wherein the ligand for DCAF15 is selected from a compound of Formula (DCAF15-Ia) or (DCAF15-Ib):

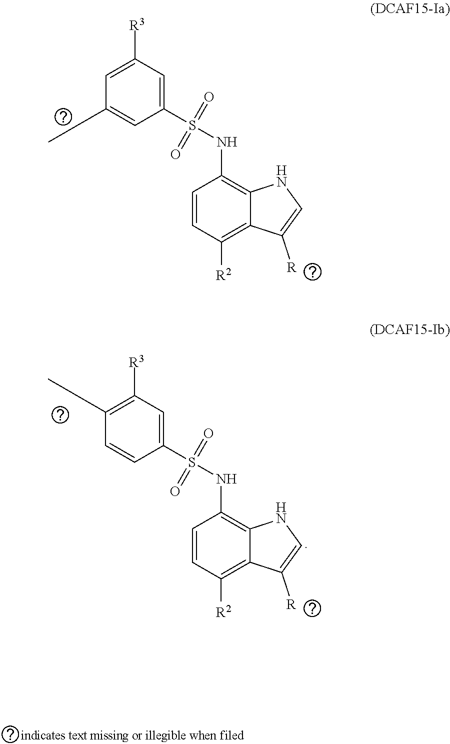

4. The HyT-PD molecule according to any one of the preceding items, wherein ligand for DCAF15 is selected from a compound of Formula (DCAF15-Ia)

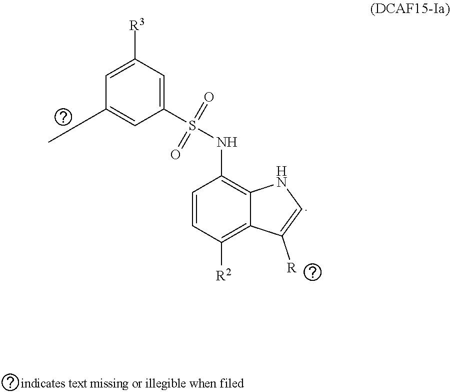

5. The HyT-PD molecule according to any one of the preceding items, wherein R¹ is selected as CI, and R² is selected as H.
6. The HyT-PD molecule according to any one of the preceding items, wherein R³ is selected as H.
7. The HyT-PD molecule according to any one of the preceding items, wherein the hydrophobic tag (HyT) is selected from a compound of Formula (I)-(IV),

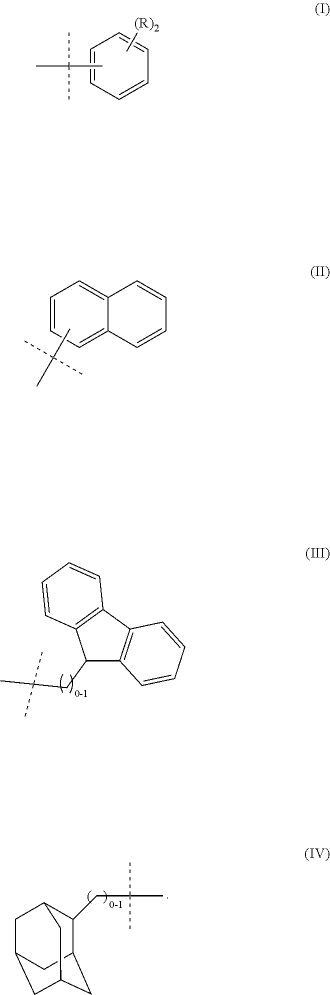

8. The HyT-PD molecule according to any one of the preceding items, wherein linker has a chain length of 3-18 atoms, preferably 3-14 atoms, more preferably 3-10 atoms, even more preferably 3-8 atoms, most preferably 3-6 atoms.
9. The HyT-PD molecule according to any one of the preceding items, wherein the linker comprises a PEG chain, an extended glycol chain, an alkyl chain or a chain having a cyclic motif selected from a structure of Formula L1-L4,

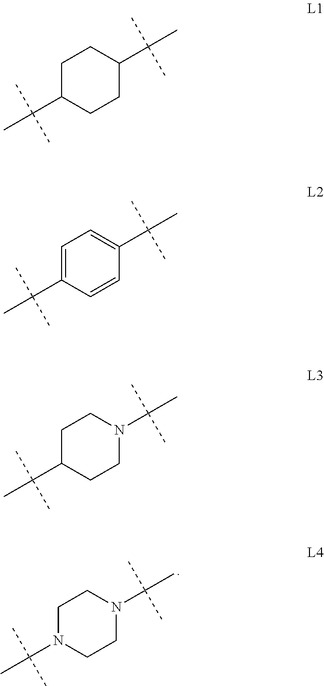

10. The HyT-PD molecule according to any one of the preceding items, wherein the linker comprises a PEG chain, an extended glycol chain, or an alkyl chain; preferably a PEG chain or an alkyl chain.
11. The HyT-PD molecule according to any one of the preceding items, wherein the linker comprises a structural motif of Formula L1-L8:

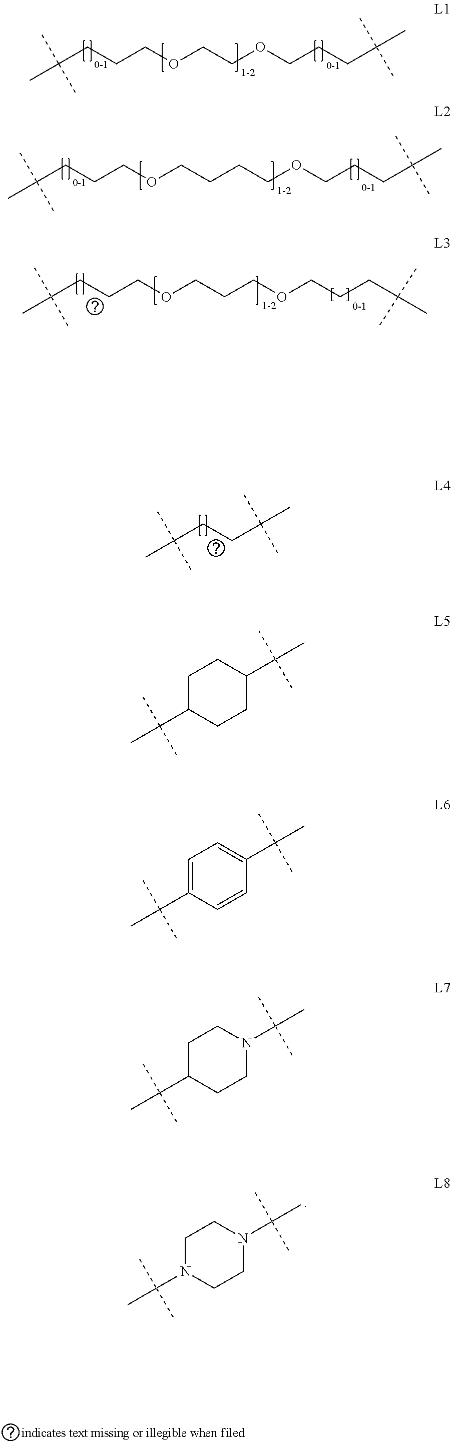

12. The HyT-PD molecule according to any one of the preceding items, wherein the linker consists of a structural motif selected from the list consisting of:

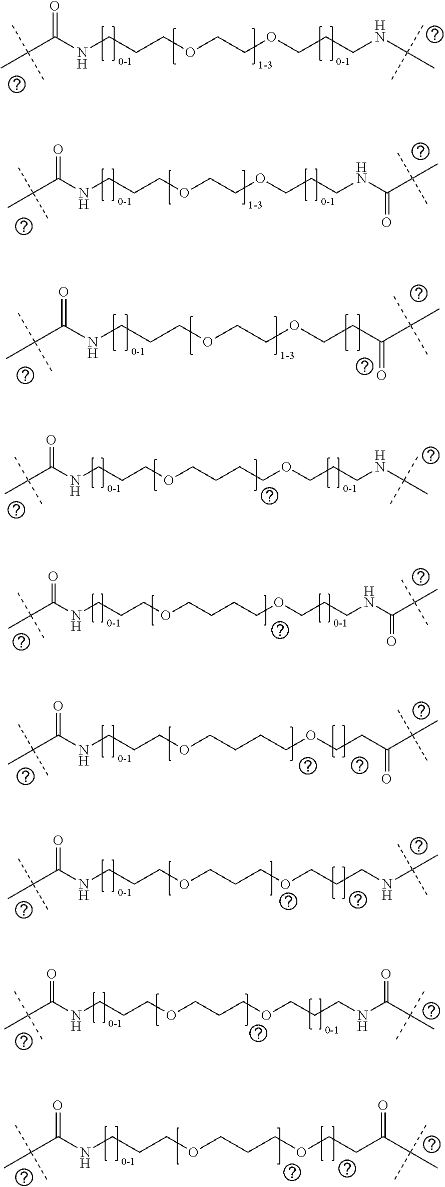

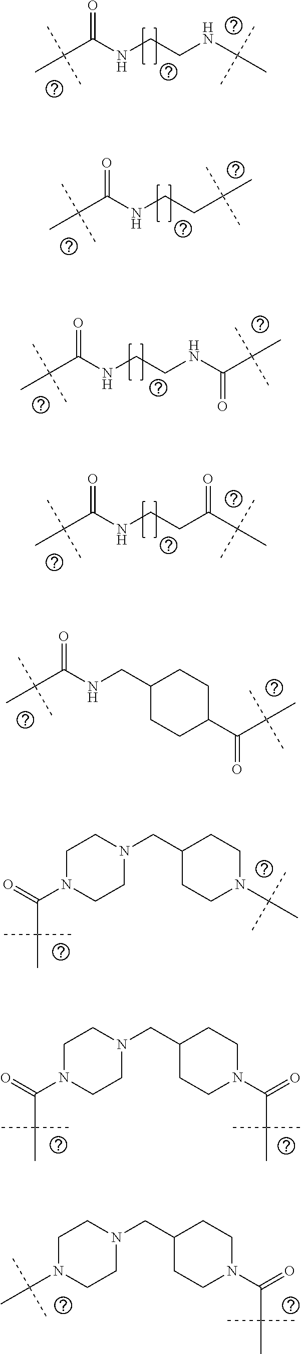

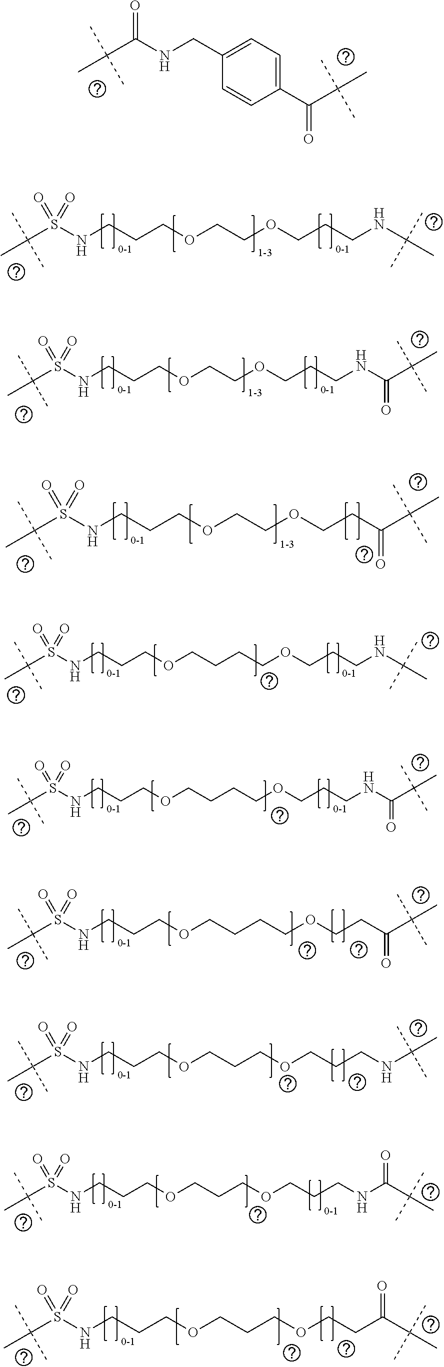

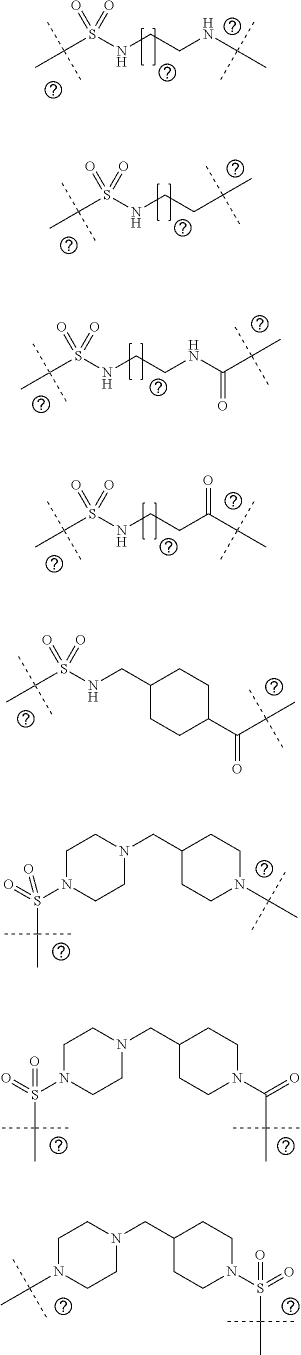

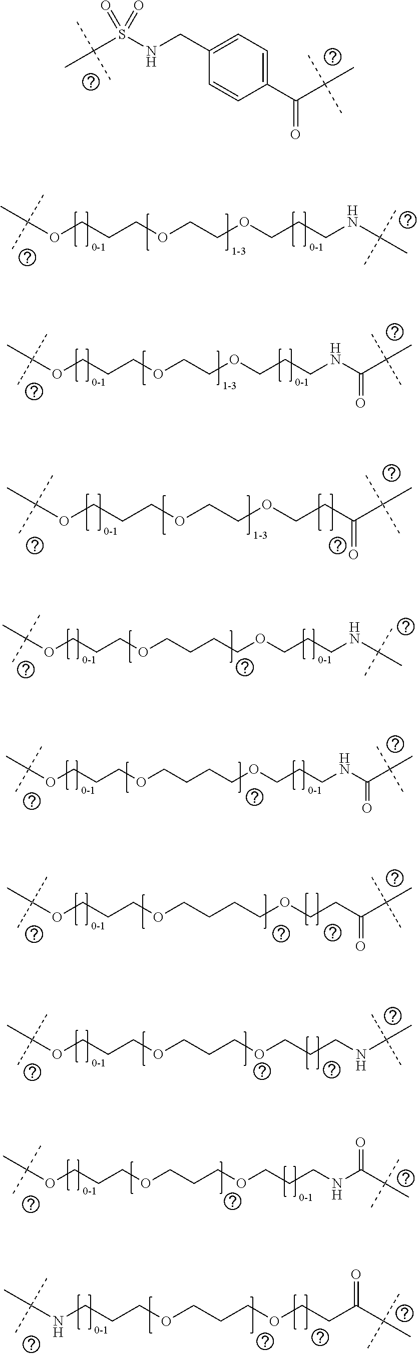

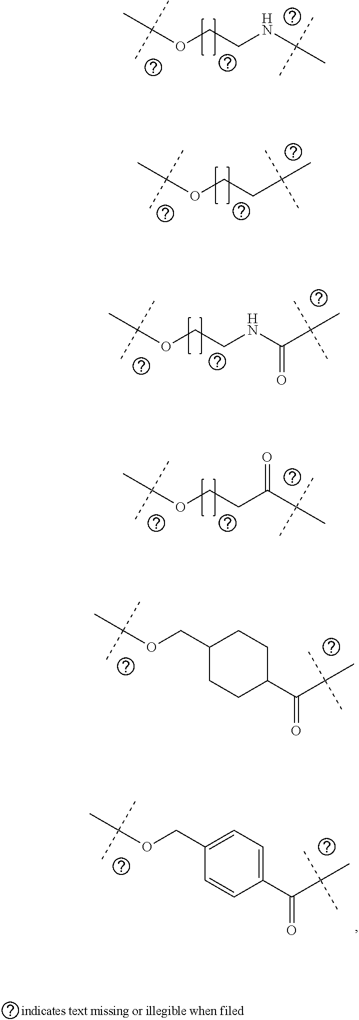

wherein the dotted lines marked * denote the end of the linker covalently connected to the DCAF15 ligand and the dotted lines marked ** denote the end of the linker covalently connected to the hydrophobic tag (HyT).
13. The HyT-PD molecule according to any one of the preceding items, wherein the linker consists of a structural motif selected from the list consisting of:

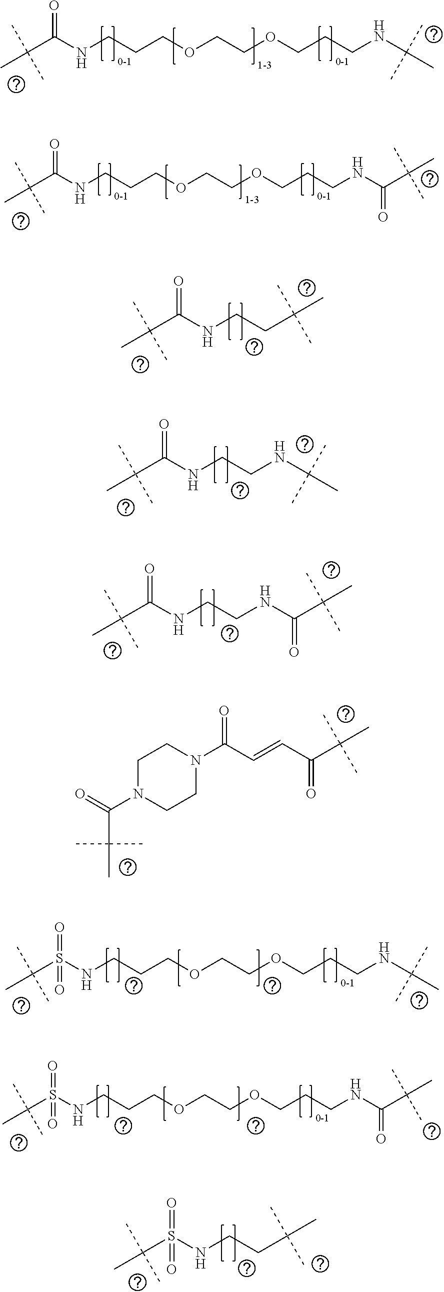

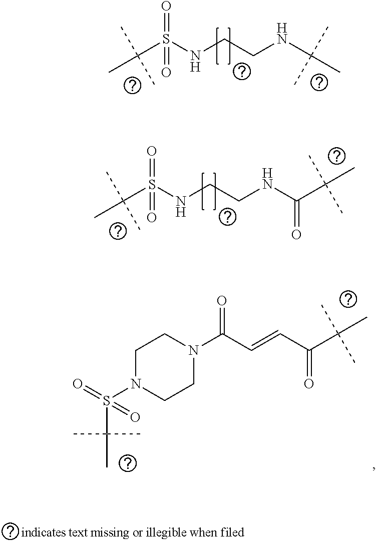

wherein the dotted lines marked * denote the end of the linker covalently connected to the DCAF15 ligand and the dotted lines marked ** denote the end of the linker covalently connected to the hydrophobic tag (HyT).
The HyT-PD molecule according to any one of the preceding items, wherein the linker consists of a structural motif selected from the list consisting of:

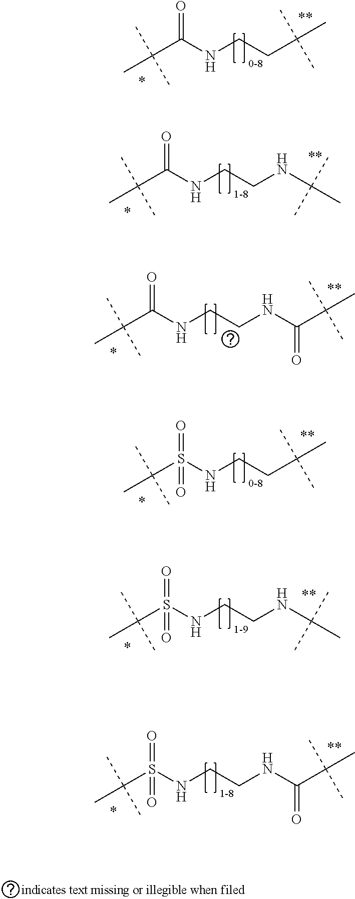

14. The HyT-PD molecule according to any one of the preceding items, wherein the linker consists of a structural motif selected from the list consisting of:

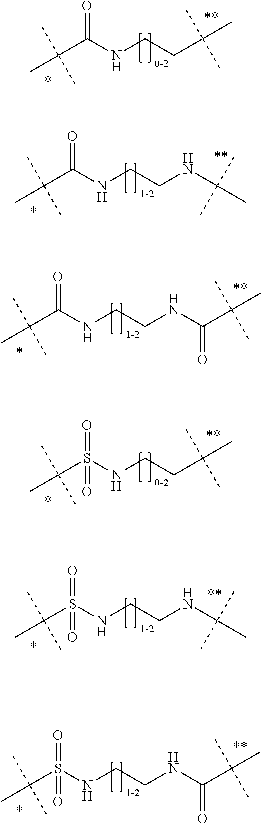

15. The HyT-PD molecule according to any one of the preceding items, wherein the HyT-PD molecule has the structure of compound nos. 24-41.

### Aspect 4: Medical Use of HyT-PD Molecules

In a fourth aspect, the present invention relates to a HyT-PD molecule, according to the third aspect and any one of its embodiments and items, for use as a medicament. Preferably, the HyT-PD molecules are for use in the treatment of amyloidosis; more preferably for use in the treatment of synucleinopathies; even more preferably for use in the treatment of Parkinson's disease (PD), dementia with Lewy bodies (DLB), and multiple system atrophy (MSA); most preferably, for use in the treatment of Parkinson's disease (PD). In the fourth aspect, the hydrophobic tag may also be selected as a Boc group. Hence, in the fourth aspect, the hydrophobic tag (HyT) is selected from a compound of Formula (I)-(VIII),

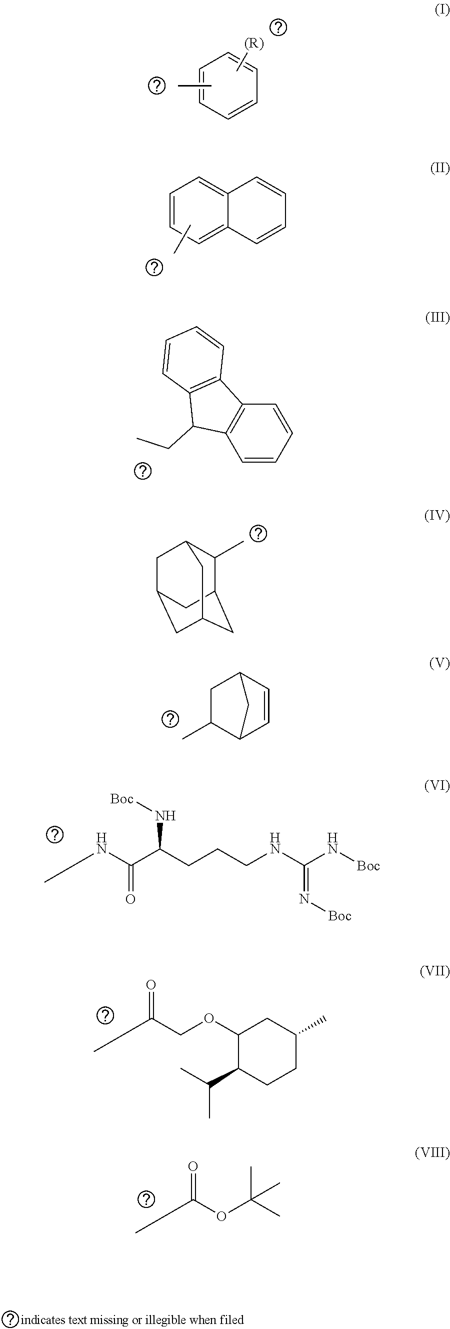

### Exemplified Protacs and HyT-PD Molecules According to the Invention

Table 1 exemplifies PROTACs and HyT-PD molecules according to the invention, using a ligand for DCAF15 covalently connected to different E3 ubiquitin ligase ligands or different hydrophobic tags (HyT) with a linker comprising a PEG chain, an extended glycol chain, an alkyl chain, or a chain comprising cyclic structures.

TABLE 1
Exemplified PROTACs and HyT-PD molecules according to the invention

| PRO- | | | |
|---|---|---|---|
| TAC | | | |
| comp. | | | |
| no. | DCAF15 ligand | Linker | E3 ligase ligand |
| 1 | 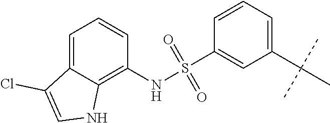 | 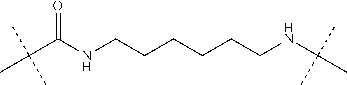 alkyl linker/length 9 atoms | 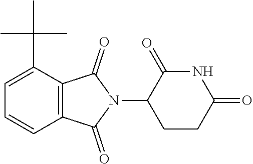 [SMILES: CC(C)(C)c1cccc2c1C(=O)N(C1CCC(=O)NC1=O)C2=O] CRBN ligand |
| 2 | 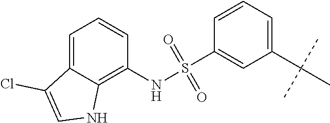 | 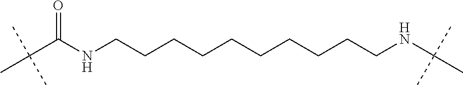 alkyl linker/length 13 atoms | 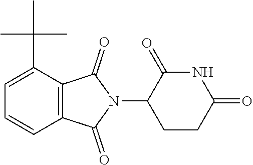 [SMILES: CC(C)(C)c1cccc2c1C(=O)N(C1CCC(=O)NC1=O)C2=O] CRBN ligand |
| 3 | 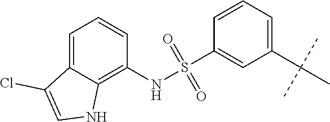 | 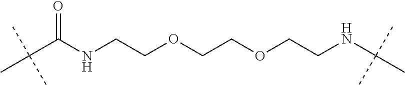 PEG linker/length 11 atoms | 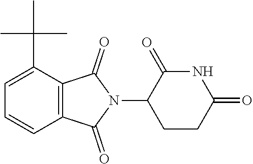 [SMILES: CC(C)(C)c1cccc2c1C(=O)N(C1CCC(=O)NC1=O)C2=O] CRBN ligand |
| 4 | 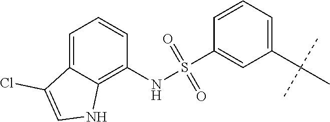 | 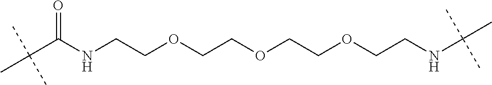 PEG linker/length 14 atoms | 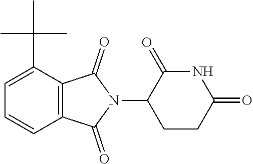 [SMILES: CC(C)(C)c1cccc2c1C(=O)N(C1CCC(=O)NC1=O)C2=O] CRBN ligand |
| 5 | 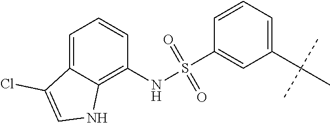 | 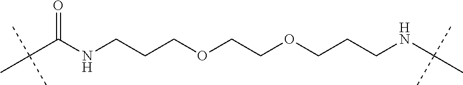 PEG linker/length 13 atoms | 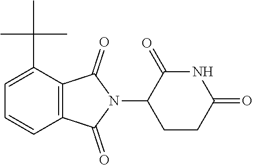 [SMILES: CC(C)(C)c1cccc2c1C(=O)N(C1CCC(=O)NC1=O)C2=O] CRBN ligand |
| 6 | 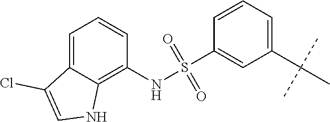 | 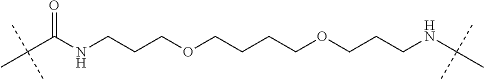 Extended glycol/length 15 atoms | 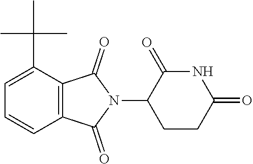 [SMILES: CC(C)(C)c1cccc2c1C(=O)N(C1CCC(=O)NC1=O)C2=O] CRBN ligand |
| 7 | 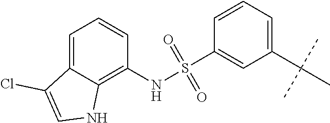 | 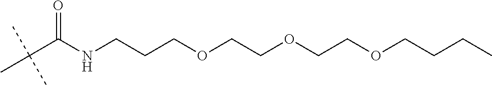 PEG linker/length 16 atoms | 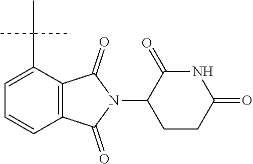 CRBN ligand |
| 8 | 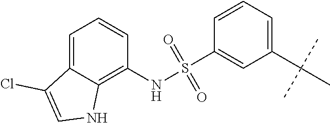 | 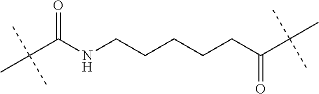 alkyl linker/length 8 atoms | 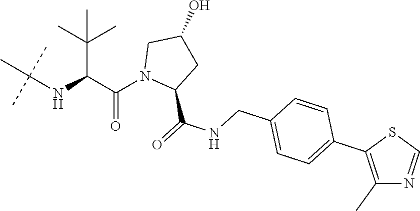 VHL ligand |
| 9 | 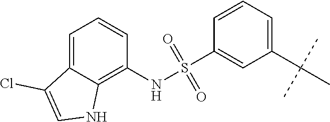 | 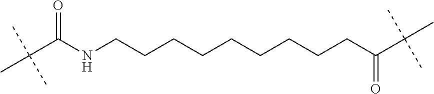 alkyl linker/length 12 atoms | 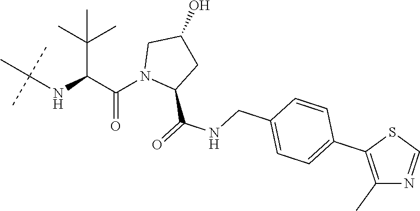 VHL ligand |
| 10 | 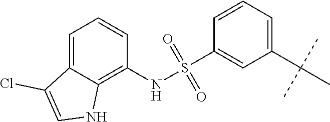 | 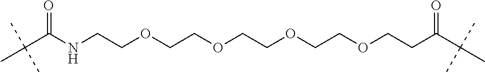 PEG linker/length 17 atoms | 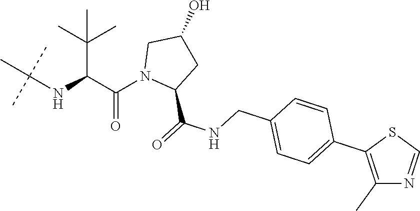 VHL ligand |
| 11 | 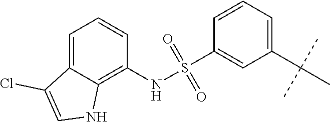 | 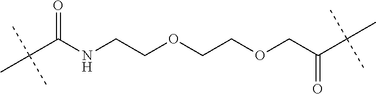 PEG linker/length 10 atoms | 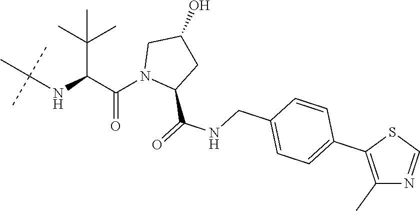 VHL ligand |
| 12 | 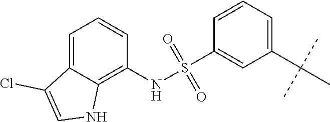 | 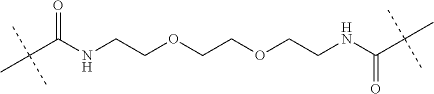 PEG linker/length 12 atoms | 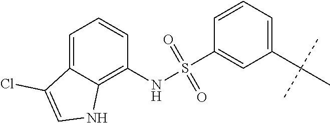 DCAF15 ligand |
| 13 | 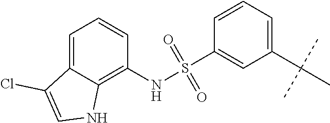 | 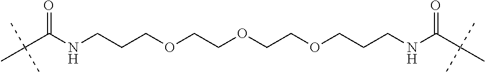 PEG linker/length 17 atoms | 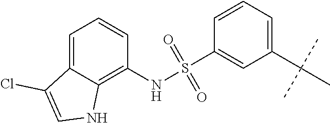 DCAF15 ligand |
| 14 | 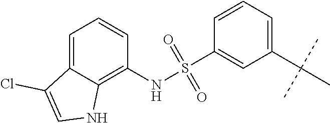 | 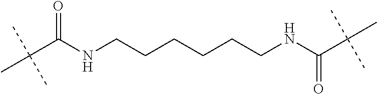 alkyl linker/length 10 atoms | 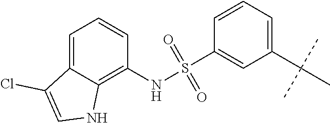 DCAF15 ligand |
| 15 | 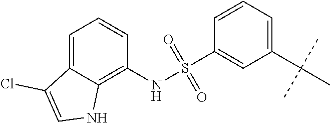 | 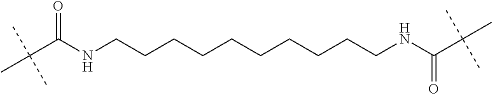 alkyl linker/length 14 atoms | 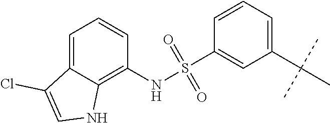 DCAF15 ligand |
| 16 | 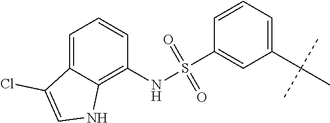 | 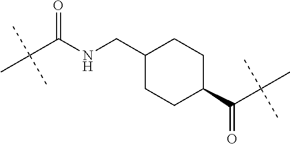 cyclic linker/length 8 atoms | 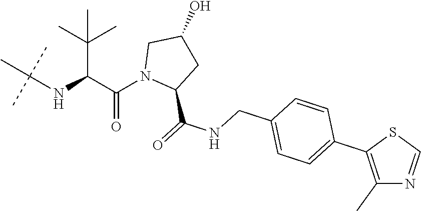 VHL ligand |
| 17 | 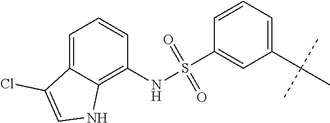 | 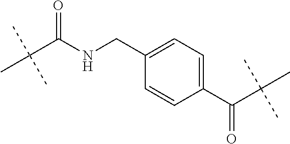 cyclic linker/length 8 atoms | 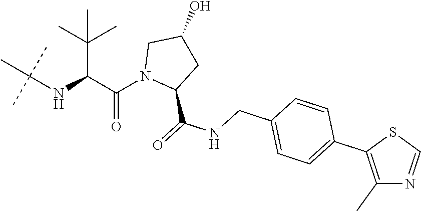 VHL ligand |
| 18 | 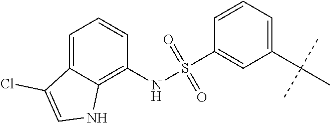 | 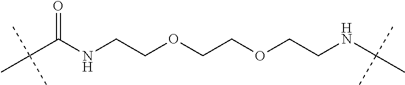 PEG linker/length 11 atoms | 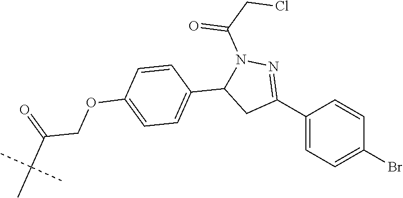 RNF114 ligand |
| 19 | 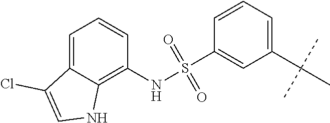 | 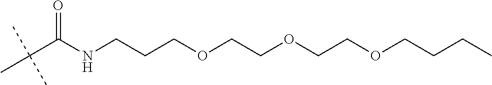 PEG linker/length 16 atoms | 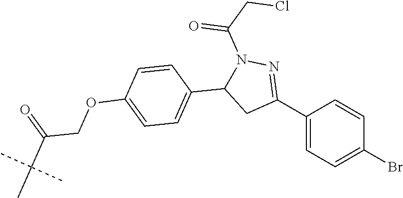 RNF114 ligand |
| 20 | 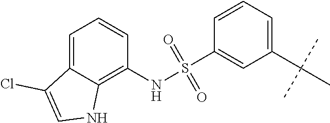 | 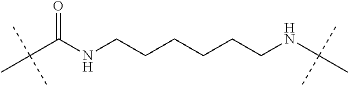 alkyl linker/length 9 atoms | 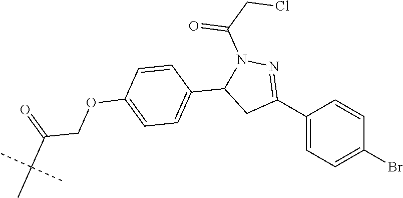 RNF114 ligand |
| 21 | 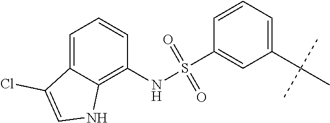 | 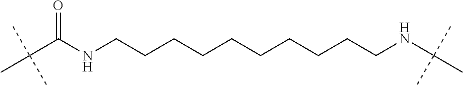 alkyl linker/length 13 atoms | 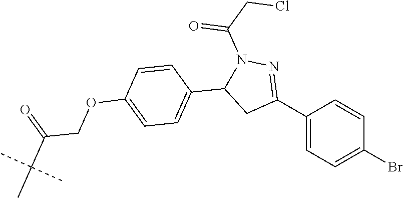 RNF114 ligand |
| 22 | 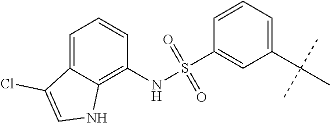 | 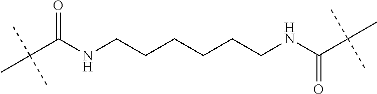 alkyl linker/length 8 atoms | 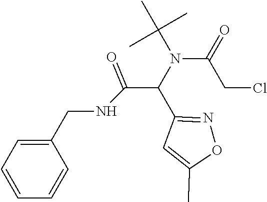 [SMILES: Cc1cc(C(C(=O)NCc2ccccc2)N(C(=O)CCl)C(C)(C)C)no1] DCAF11 ligand |
| 23 | 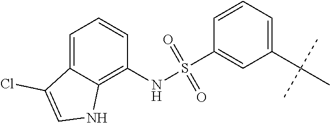 | 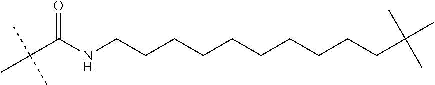 alkyl linker/length 12 atoms | 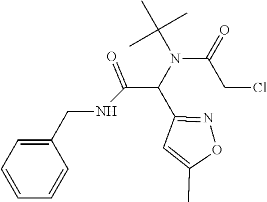 [SMILES: Cc1cc(C(C(=O)NCc2ccccc2)N(C(=O)CCl)C(C)(C)C)no1] DCAF11 ligand |
| 24 | 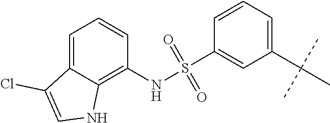 | 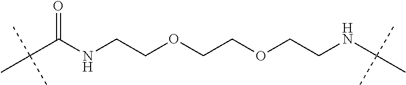 PEG linker/length 11 atoms | 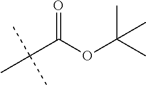 Boc group |
| HyT | | | |
| mole- | | | |
| cule | | | |
| comp | | | |
| no. | | | |
| TPD | | | |
| mole- | | | |
| cule | DCAF15 ligand | Linker | HyT |
| 25 | 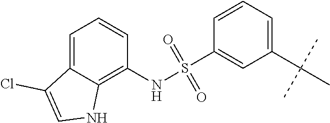 | 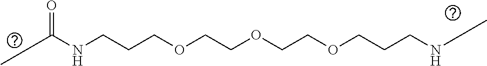 [SMILES: CCNCCCOCCOCCOCCCNC(=O)CC] PEG linker/length 16 atoms | 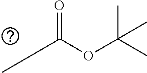 Boc group |
| 26 | 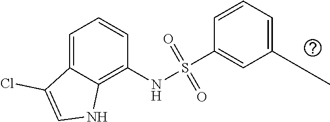 [SMILES: CCc1cccc(S(=O)(=O)Nc2cccc3c(Cl)c[nH]c23)c1] | 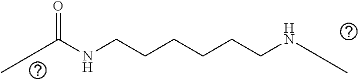 alkyl linker/length 9 atoms | 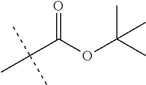 Boc group |
| 27 | 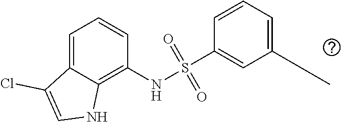 [SMILES: CCc1cccc(S(=O)(=O)Nc2cccc3c(Cl)c[nH]c23)c1] | 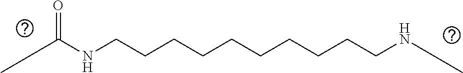 [SMILES: CCNCCCCCCCCCCNC(=O)CC] alkyl linker/length 13 atoms | 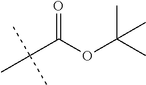 Boc group |
| 28 | 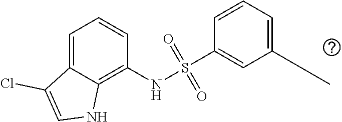 [SMILES: CCc1cccc(S(=O)(=O)Nc2cccc3c(Cl)c[nH]c23)c1] | 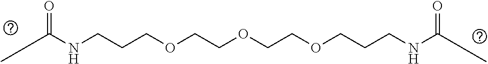 [SMILES: CCC(=O)NCCCOCCOCCOCCCNC(=O)CC] PEG linker/length 17 atoms | 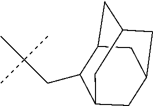 Adamantyl group |
| 29 | 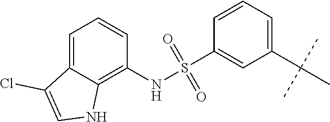 | 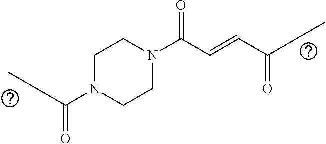 [SMILES: CCC(=O)/C=C/C(=O)N1CCN(C(=O)CC)CC1] cyclic linker/length 9 atoms | 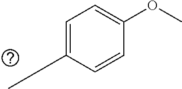 Aryl group |
| 30 | 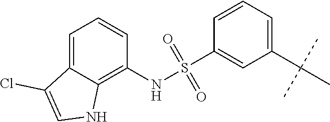 | 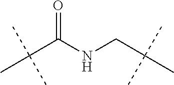 alkyl linker/length 3 atoms | 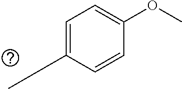 Aryl group |
| 31 | 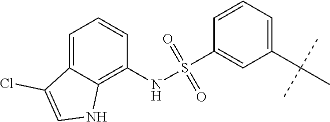 | 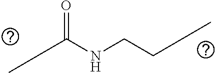 alkyl linker/length 4 atoms | 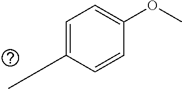 Aryl group |
| 32 | 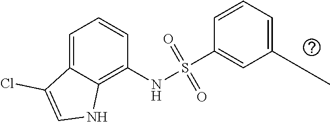 [SMILES: CCc1cccc(S(=O)(=O)Nc2cccc3c(Cl)c[nH]c23)c1] | 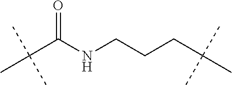 alkyl linker/length 5 atoms | 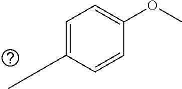 Aryl group |
| 33 | 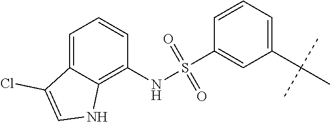 | 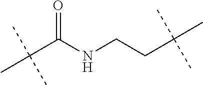 alkyl linker/length 4 atoms | 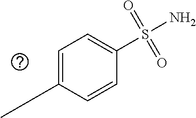 Aryl group |
| 34 | 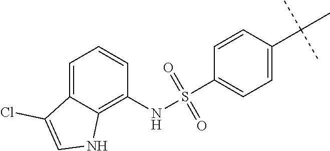 | 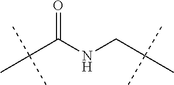 alkyl linker/length 3 atoms | 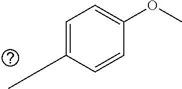 Aryl group |
| 35 | 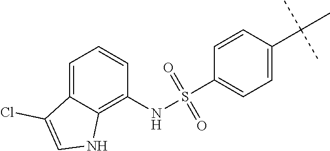 | 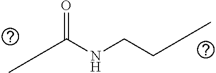 [symbol] alkyl linker/length 4 atoms | 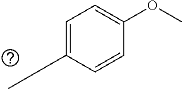 Aryl group |
| 36 | 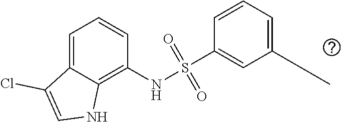 [SMILES: CCc1cccc(S(=O)(=O)Nc2cccc3c(Cl)c[nH]c23)c1] | 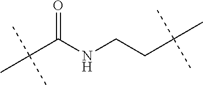 alkyl linker/length 4 atoms | 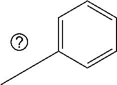 Aryl group |
| 37 | 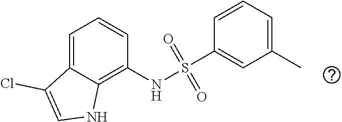 [SMILES: Cc1cccc(S(=O)(=O)Nc2cccc3c(Cl)c[nH]c23)c1] | 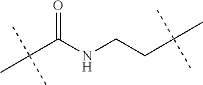 alkyl linker/length 4 atoms | 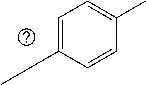 Aryl group |
| 38 | 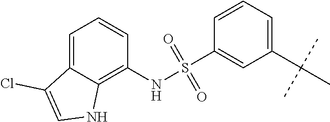 | 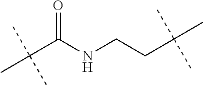 alkyl linker/length 4 atoms | 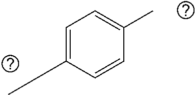 Aryl group |
| 39 | 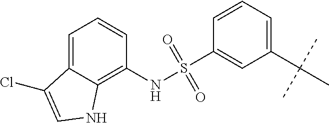 | 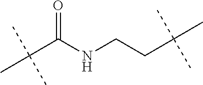 alkyl linker/length 4 atoms | 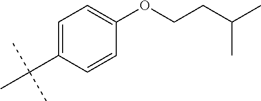 Aryl group |
| 40 | 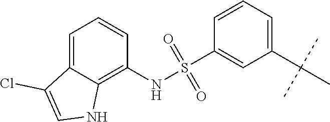 | 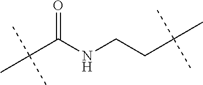 alkyl linker/length 4 atoms | 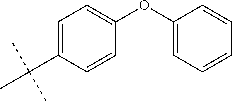 Aryl group |
| 41 | 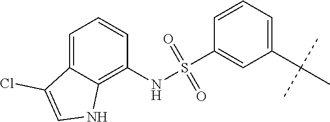 | 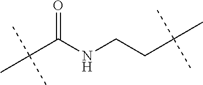 alkyl linker/length 4 atoms | 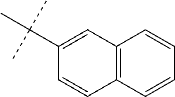 Naphthyl group |
| 42 | 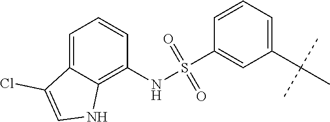 | 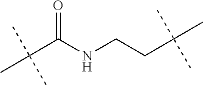 alkyl linker/length 4 atoms | 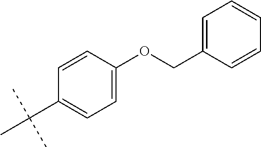 Aryl group |
| 43 | 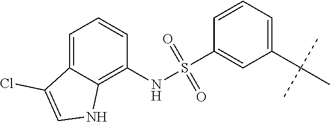 | 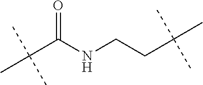 alkyl linker/length 4 atoms | 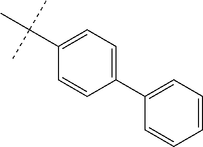 Aryl group |
| 44 | 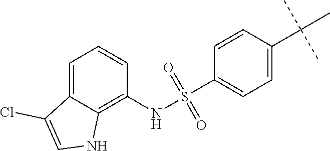 | 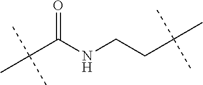 alkyl linker/length 4 atoms | 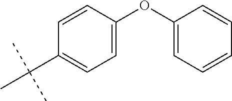 Aryl group |
| 45 | 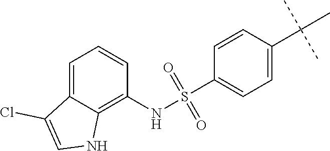 | 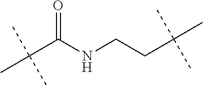 alkyl linker/length 4 atoms | 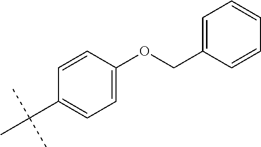 Aryl group |
| 46 | 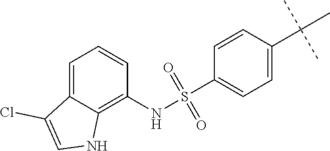 | 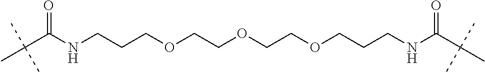 PEG linker/length 17 atoms | 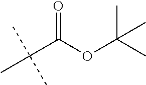 Boc group |
| 47 | 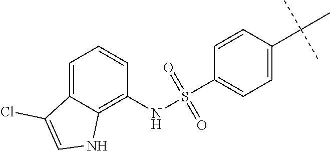 | 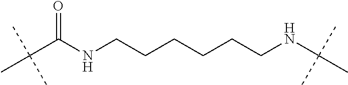 alkyl linker/length 9 atoms | 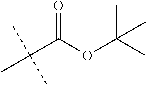 Boc group |
| 48 | 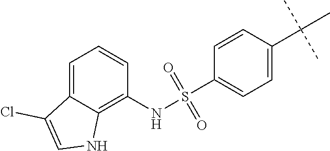 | 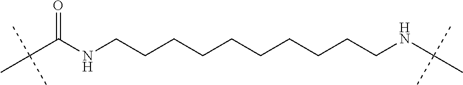 alkyl linker/length 13 atoms | 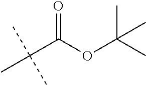 Boc group |
| 49 | 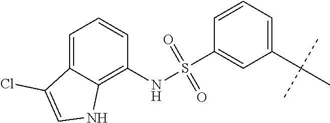 | 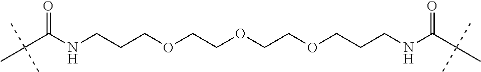 PEG linker/length 17 atoms | 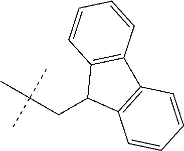 Fluorene group |
| 50 | 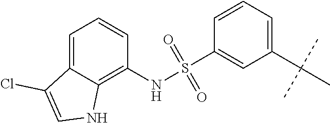 | 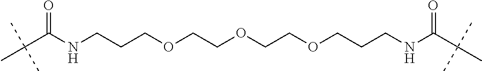 PEG linker/length 17 atoms | 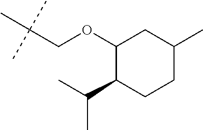 Menthone group |
| 51 | 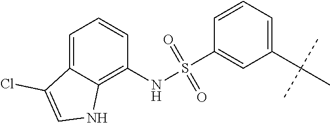 | 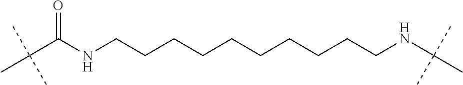 alkyl linker/length 13 atoms | 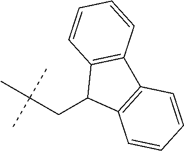 Fluorene group |
| 52 | 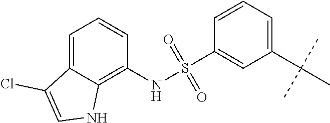 | 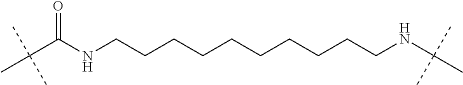 alkyl linker/length 13 atoms | 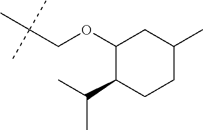 Menthone group |
| 53 | 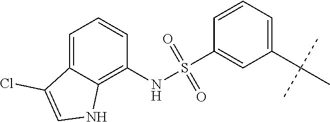 | 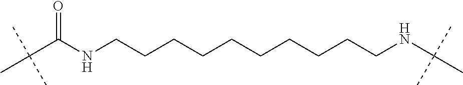 alkyl linker/length 13 atoms | 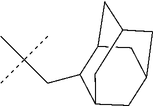 Adamantyl group |
| 54 | 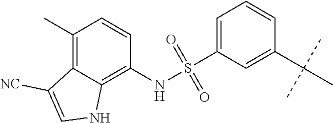 | 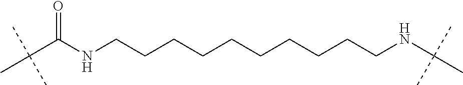 alkyl linker/length 13 atoms | 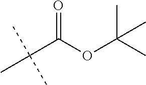 Boc group |
| 55 | 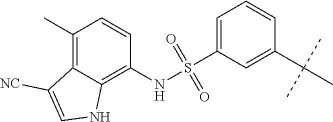 | 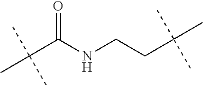 alkyl linker/length 4 atoms | 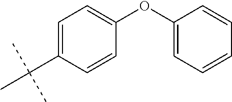 Aryl group |
| 56 | 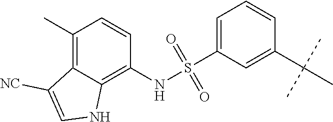 | 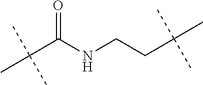 alkyl linker/length 4 atoms | 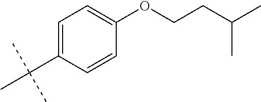 Aryl group |

[symbol]

indicates data missing or illegible when filed

### EXPERIMENTAL SECTION

### Example 1: DCAF15 is Required for Protein Aggregation Induced by Translational Stress

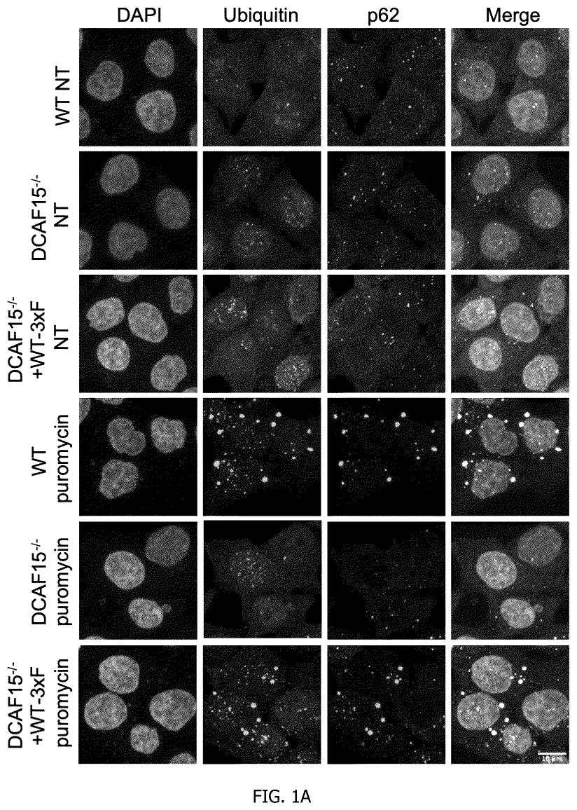

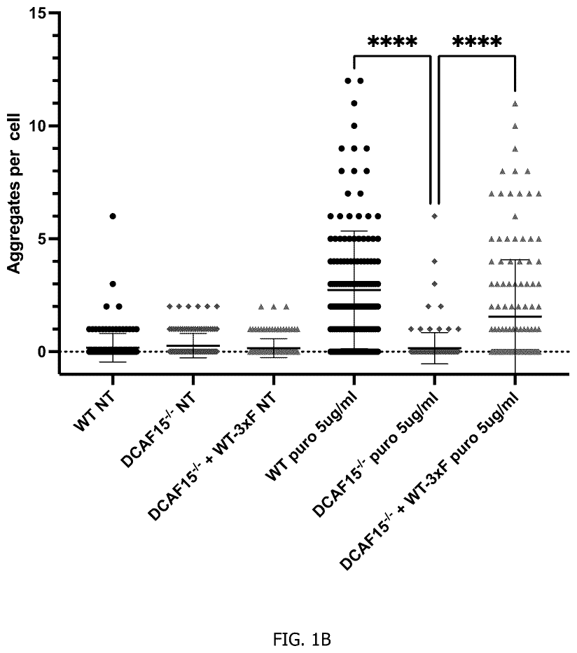

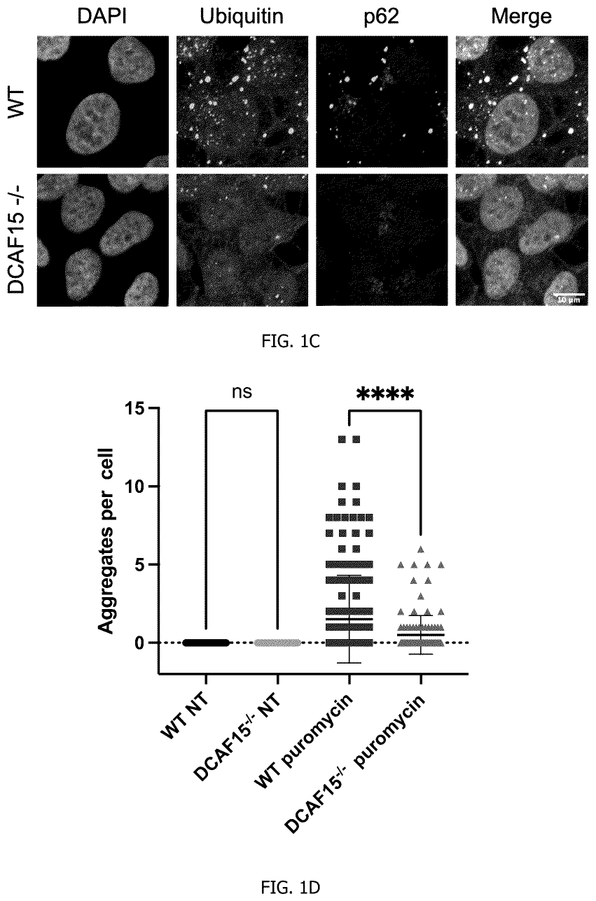

HCT116 cells with indicated genotypes were treated with 5 μg/ml puromycin for 4 h or left untreated (NT) before fixation. Cells were stained for ubiquitinated proteins and p62. Separate and merged channels from a representative experiment are shown (n>3) (see FIG. 1A). The number of ubiquitin and p62 co-stained aggregates per cell were quantified (see FIG. 1B.).

BE2C cells with indicated genotypes were treated and analysed as above (n=3). Separate and merged channels from a representative experiment are shown (n>3) (see FIG. 1C). The number of ubiquitin and p62 co-stained aggregates per cell were quantified (see FIG. 1D.).

Cell lines: WT (wild-type), DCAF15−/− (CRISPR-mediated stable DCAF15−/− knock-out cells), DCAF15−/−+WT 3xF (DCAF15−/− cells reconstituted with WT DCAF15 with 3×flag-tag).

Conclusion: Puromycin-induced misfolding is well-known to cause protein aggregation, quantified by co-localization of ubiquitin and p62 (visualized by puncta in merged images). The data confirm a significant reduction of aggregates in DCAF15−/− cells relative to WT and reconstituted cell lines.

### Example 2: DCAF15 is Required for Protein Aggregation Following Different Types of Proteotoxic Stress

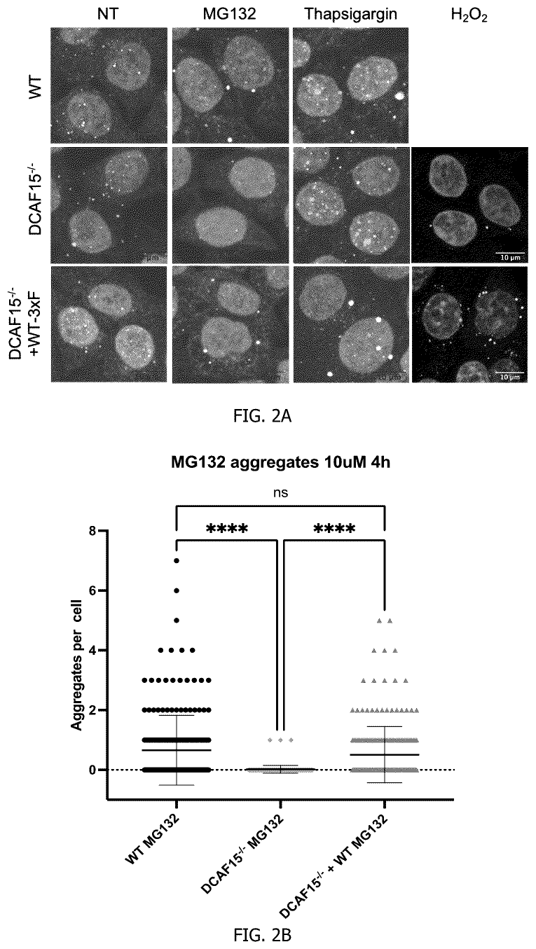

HCT116 cells were treated with 10 μM MG132, 10 μg/ml Thapsigargin or 400 μM H₂O₂ for 4 h as indicated and analysed for ubiquitin and p62 positive protein aggregates (see FIG. 2A). Representative experiments are shown, MG132 (n=4), Thapsigargin (n=3), H₂O₂ (n=1). Aggregates per cell from MG132 treatment in the three indicated cell lines were quantified (see FIG. 2B).

Conclusion: Using a similar setup and readout as in Example 1, protein aggregation was induced by different types of proteotoxic stress (beyond translational stress by puromycin). The data show a similar phenotype as in FIGS. 1A, 1B, 1C, and 1D for all stress types, and hence suggest a broader role for DCAF15 in facilitating protein aggregation in response to various types of proteotoxic stress.

### Example 3. DCAF15 does not Affect Clearance of Protein Aggregates Via Autophagy or the Proteasome

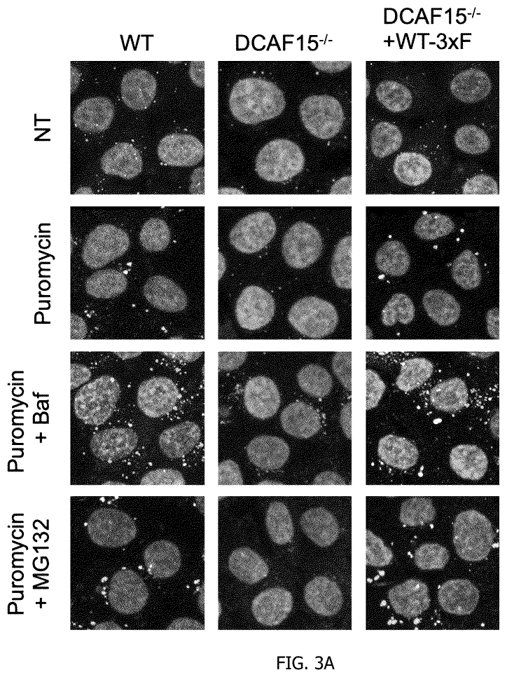

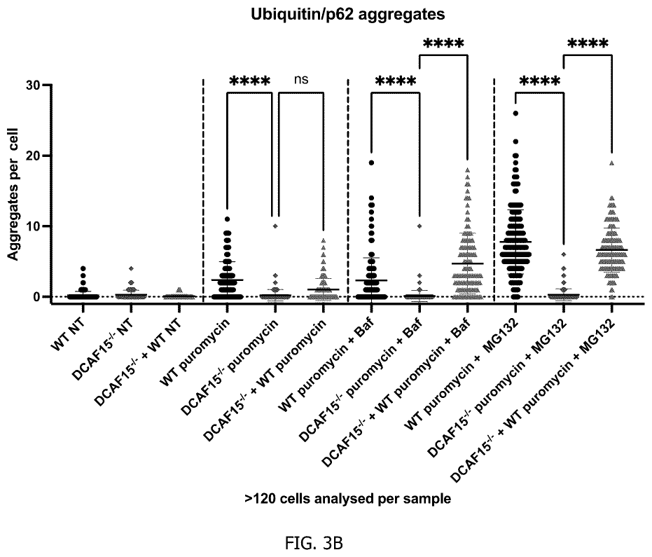

HCT116 cells were treated with 5 μg/ml puromycin for 4 h alone or in combination with 200 nM Bafilomycin-1 or 5 μM MG132. Cells were fixed and stained for ubiquitin and p62. A representative experiment is shown (n=3) (see FIG. 3A). The double positive puncta per cell was quantified (see FIG. 3B).

Conclusion: The data from Examples 1 and 2 could theoretically be explained by an altered clearance of protein aggregates in DCAF15−/− cells. To address this possibility, we repeated the assay while inhibiting the two known intracellular clearance routes for aggregates: autophagy (inhibited by Bafilomycin A1) and the proteasome (inhibited by MG132). While these treatments enhance aggregate numbers in WT and reconstituted cell lines (as expected), we do not detect any re-appearance of aggregates in DCAF15−/− cells after treatments. Examples 1-3 therefore collectively support that rather than affecting aggregate clearance, DCAF15 facilitates protein aggregate formation. Therefore, examples 1-3 collectively demonstrate that targeted protein degradation (TPD) of DCAF15 is a viable approach to treat or prevent diseases caused by or associated with protein aggregation.

### Example 4: Loss of DCAF15 is Beneficial for Cellular Fitness Following Proteotoxic Stress

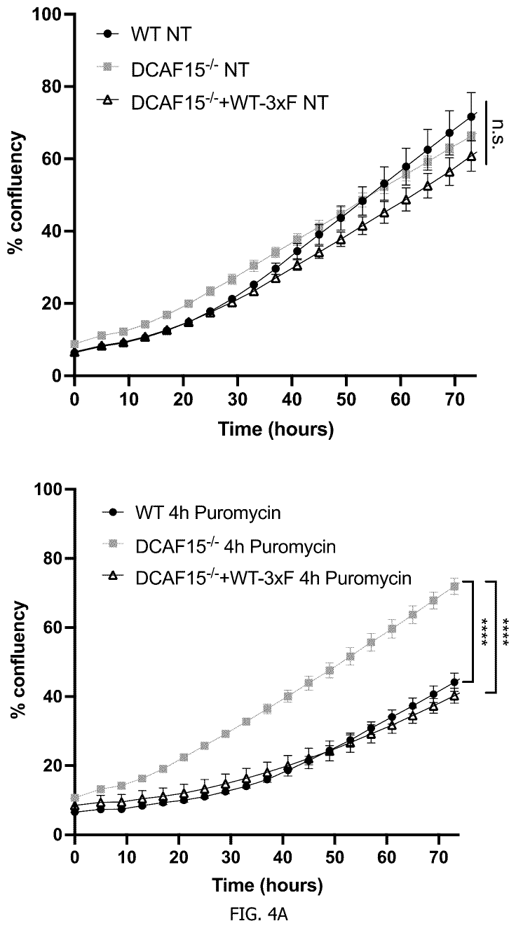

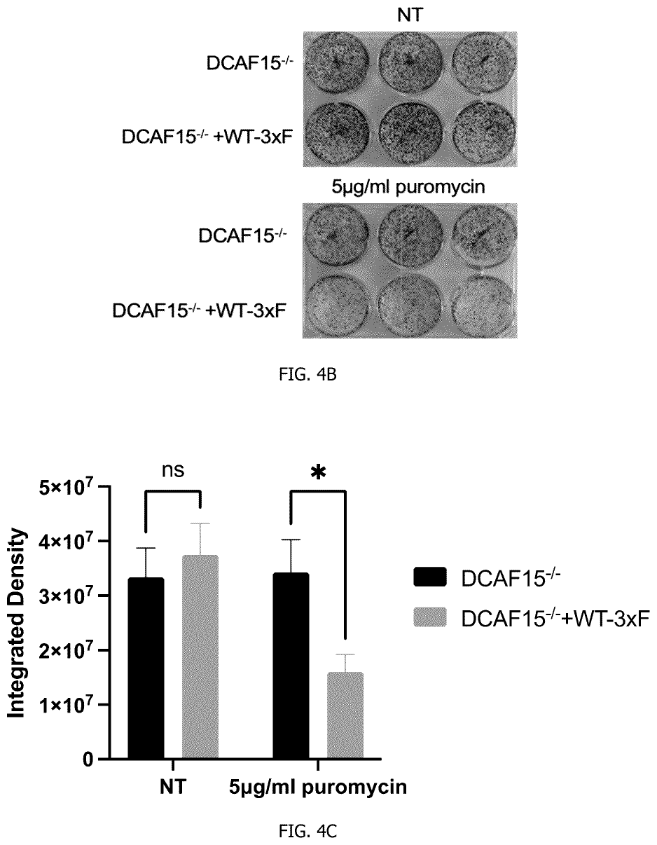

HCT116 cells were treated with 5 μg/ml puromycin for 4 h or left untreated before being released into normal growth media for 72 h. Confluency was recorded with imaging every 4 h, representative experiments with technical triplicates (n=3) are shown (see FIG. 4A.). HCT116 cells were treated with 5 μg/ml puromycin for 4 h before release into full media or left untreated for 7 days before fixation and crystal violet staining (see FIG. 4B.). Quantification of FIG. 4B as integrated density per well (see FIG. 4C).

Conclusion: By monitoring proliferation after proteotoxic insult, the ability for cellular recovery can be estimated, which gives an indication of cellular fitness. While the three HCT116 cell lines do not differ in their basal growth rates (FIG. 4A, top), DCAF15−/− cells remain undeterred by puromycin treatment, while both WT and reconstituted cell lines display significantly reduced proliferation rates (FIG. 4A, bottom). These results are similarly confirmed by a clonogenic survival assay, which suggests insensitivity of DCAF15−/− cells to puromycin-induced toxicity (FIGS. 4B and 4C). Collectively, these results suggest that lack of DCAF15 confers enhanced cellular fitness after proteotoxic stress.

### Example 5: DCAF15 Promotes Aggregation of α-Synuclein in Neuroblastoma Cells

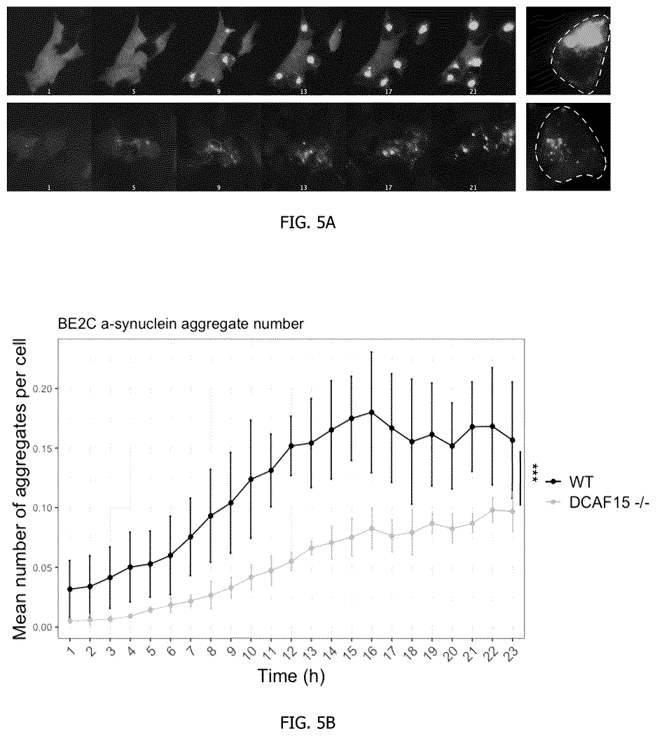

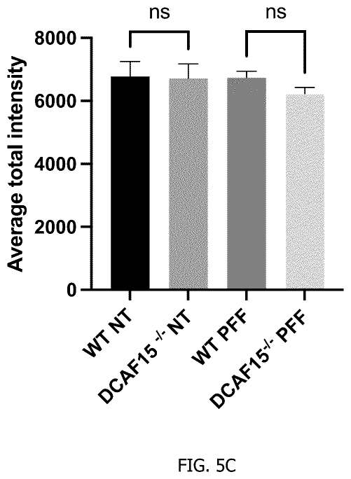

Time series from live-cell imaging of α-synuclein aggregation in BE2C cells (WT or DCAF15−/−) stably expressing GFP-μ-synuclein A53T were made (see FIG. 5A). Aggregation was induced by transfection of 600 ng/ml pre-formed fibrils (PFF) and imaging performed once per hour for 24 h to monitor aggregation (n=3). Representative images from every 4 h hour (left) and a representative single cell after 24 h PFF treatment (right). Quantification of α-synuclein inclusion number (>2 μm diameter) from A (see FIG. 5B). Quantification of total GFP levels from indicated cell lines/treatments (time point 1 in A) (see FIG. 5C).

Conclusion: A cellular system to study α-synuclein aggregation in BE2C neuroblastoma cells was developed by stably expressing a GFP-tagged α-synuclein, containing a well-known A53T mutation that is especially prone to aggregation. To induce aggregation, purified pre-formed α-synuclein fibrils (PFF) were transfected and formation of α-synuclein inclusions monitored by live imaging. α-synuclein inclusions were distinctly fewer (and smaller in size) in DCAF15−/− cells relative to WT, while overall GFP intensity did not differ between the two cell lines. The data suggest that DCAF15 facilitates PFF-induced α-synuclein aggregation/inclusion formation.

### Example 6: Targeted Protein Degradation of DCAF15 Via PROTACs or HyT-PD Molecules Inhibits Aggregation

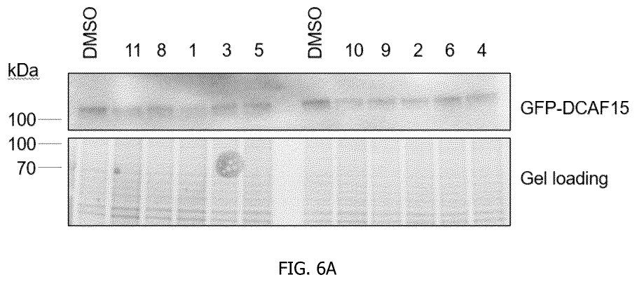

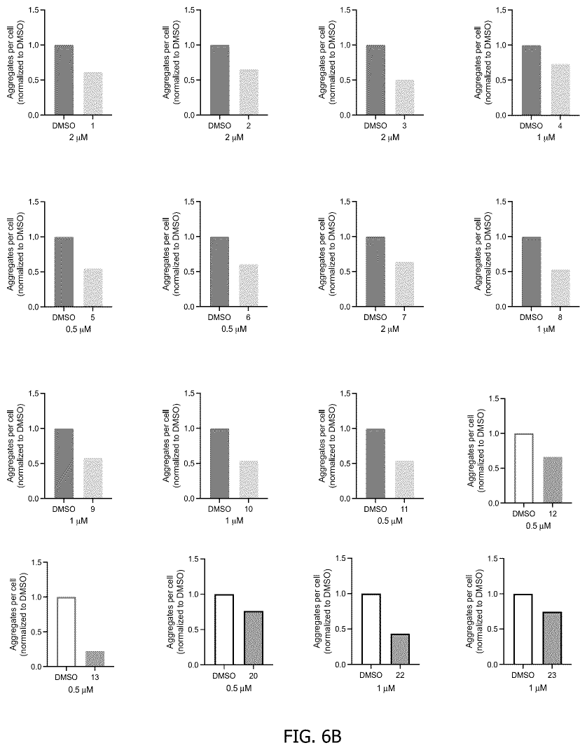

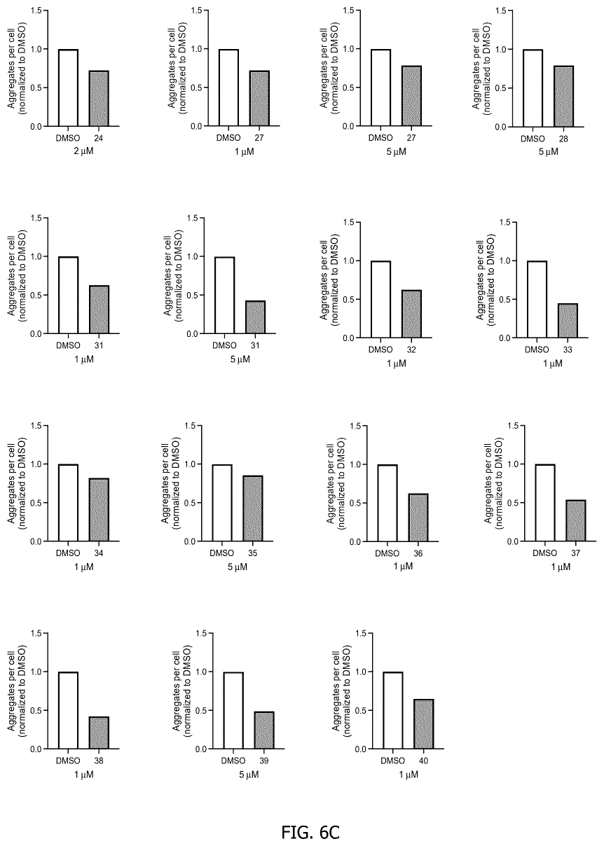

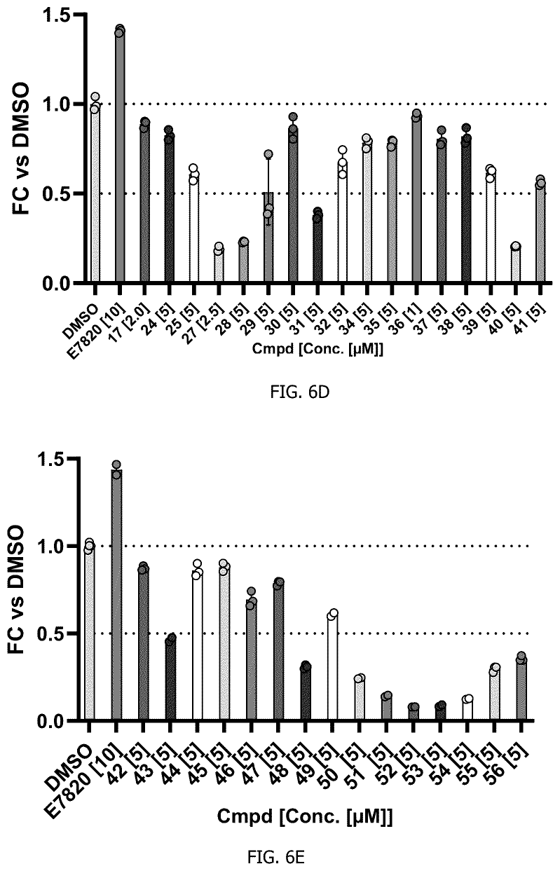

HCT116 DCAF15−/− cells stably expressing doxycycline-inducible GFP-DCAF15-3xF. Cells were treated with doxycycline for 24 h to induce GFP-DCAF15-3xF expression and treated with indicated PROTACS (2 uM) for 24 h. Cells were lysed and analyzed for GFP-DCAF15 levels by western blot using anti-GFP antibody (see FIG. 6A).

FIG. 6B shows BE2C WT cells treated with 0.5-2 uM of indicated PROTACs (or DMSO control) for 4 h. 5 ug/ml puromycin was added for 2 h to induce aggregation and cells fixed and stained for ubiquitin and p62. “Aggregates per cell” represents the mean no. of double positive puncta (for ubiquitin and p62) per cell. Experiment was performed with technical duplicates analyzing >110 cells per sample.

FIG. 6C shows BE2C WT cells treated with 1, 2 or 5 uM of indicated HyT-PD molecule (or DMSO control) for 24 h. Compound 24 was tested in HCT116 WT cells where cells were treated for 6 h with the compound. 5 ug/ml puromycin was added for the last 4 h to induce aggregation and cells fixed and stained for ubiquitin and p62. “Aggregates per cell” represents the mean no. of double positive puncta (for ubiquitin and p62) per cell. Experiment was performed with technical triplicates analyzing >120 cells per sample (see FIG. 6C).

Conclusion: The data in FIG. 6A-C collectively supports that the PROTACs or HyT-PD molecules mediated decay of DCAF15 and prevented stress-induced aggregate formation.

HEK293T cells expressing HiBit-DCAF15 at endogenous level were treated with the indicated compounds (i.e. PROTACs or HyT-PD molecule) and concentrations for 24 h (n=3 technical replicates). HiBit levels were measured using the HiBit lytic detection system developed by Promega, following the manufactures instructions. The obtained values were compared and plotted as the fold change vs the mean of the DMSO control sample (see FIGS. 6D and 6E).

Conclusion: The data of FIGS. 6D and 6E supports that PROTAC or HyT-PD molecule mediated decay of DCAF15 and prevented stress-induced aggregate formation.

### Example 7: Synthesis of Exemplified PROTACs and HyT-PD Molecules

### General

All reactions were run under an air atmosphere unless specified and monitored by thin-layer chromatography (TLC) and/or reversed-phase ultra-performance liquid chromatography-mass spectrometry (RP-UPLC-MS). Commercially available reagents were purified according to standard procedures or were used as received from Sigma Aldrich, Alfa Aesar, Acros Organics, Combi-Blocks, Fisher Scientific, Strem, and Merck. All solvents used were of HPLC quality and dry solvents (DCM) were obtained from a PureSolv system (Innovative Technology, Tronyx). Analytical TLC was conducted on Merck aluminium sheets covered with silica (C60). The plates were either visualized under ultraviolet (UV)-light or stained by dipping in a developing agent followed by heating. Ninhydrin was used as developing agent. Flash column chromatography was performed using Merck Geduran® Si 60 (40-63 μm) silica gel. All purified compounds were characterized by Nuclear Magnetic Resonance Spectroscopy (NMR), and LRMS electrospray ionization (ESI) (by-products were not fully characterized). Melting point and optical rotation were measured when appropriate. Structural assignments were made, when possible, for new compounds using COSY, HSQC, HMBC spectra where appropriate. For the recording of 1H NMR and 13C NMR spectra, a Bruker Avance III 400 spectrometer with a Bruker Ascend 400 magnet and a Prodigy CryoProbe (operating at 400 MHz for proton and 101 MHz for carbon) was used. For the recording of some 1H, and some 13C spectra, a Bruker Avance III 800 spectrometer with a Bruker Ascend 800 magnet and a 5 mm TCI CryoProbe (operating at 800 MHz for proton, 201 MHz for carbon) was used. The chemical shifts (0) are reported in parts per million (ppm) and the coupling constants (J) in Hz. Spectra were referenced using the residual solvent peaks of the respective solvent; DMSO-d6 (δ=2.50 ppm for 1H NMR and δ=39.52 ppm for 13C NMR), CDCl3 (δ=7.26 ppm for 1H NMR and δ=77.16 ppm for 13C NMR). The following abbreviations were used to report peak multiplicities: s=singlet, d=doublet, t=triplet, q=quartet, dd=doublet of doublets, dt=doublet of triblets, dtd=doublet of triblets of doublets, tt=triplet of triplets, m=multiplet, bs=broad singlet. Undetected 13C peaks in 1D 13C NMR spectra were assigned on the basis of detectable cross peaks in 2D 1H-13C HSQC and HMBC spectra. If so, it will be noted within the characterization, in addition to the mentioning of exchanged protons in 1D 1H NMR.

Analytical RP-UPLC-MS (ESI) analysis was performed on a S2 Waters AQUITY RP-UPLC system equipped with a photo diode array (PDA) detector using an Thermo Accucore C18 column (d=2.6 μm, 2.1×50 mm; column temp.=50° C.; flow=0.50 mL/min). Eluent A (0.1% HCOOH in H₂O) and B (0.1% HCOOH in MeCN) used in a linear gradient (5% B to 100% B) in 2.4 or 4.8 min, hold 0.1 min, total run time 2.6 or 5.0 min. The LC system was coupled to a SQD electrospray mass spectrometer.

Melting points were obtained using a Stuart SMP30 melting point apparatus. Optical rotation was carried out using a PerkinElmer Polarimeter 241 using the D-line from sodium-vapor lamp, and the temperature for all recordings was approximately 20° C.

### Synthesis of DCAF15 Ligand—Methyl 3-(N-(3-chloro-1H-indol-7-yl)sulfamoyl)benzoate

3-chloro-1H-indol-7-amine (2.05 g, 12.3 mmol, 1.0 equiv.) was dissolved in EtOAc (50 ml), to which 3-methylacetatebenzenesolfonyl chloride (3.09 g, 13.17 mmol, 1.1 equiv.) and pyridine (1.95 ml, 24.11 mmol, 2 equiv.) were added. The reaction mixture was stirred at rt for 3-4 h, diluted with EtOAc (50 ml) and washed with 1 M aqueous HCl (25 ml), H₂O (100 ml), sat. aqueous solution of NaHCO₃ (100 ml) and brine (100 ml). The organic layer was dried using MgSO₄, filtered and put on the rotary evaporator. The crude product was purified by flash column chromatography using a gradient from 10-60% EtOAc/heptane and compound (3) was afforded as a white powder (2.19 g, 49%). Rf: 0.11 (3:7 EtOAc/Heptane). ¹H NMR (400 MHz, DMSO-d6) δ ppm: 11.07 (s, 1H), 10.11 (s, 1H), 8.30 (t, J=1.8 Hz, 1H), 8.15 (dt, J=7.8, 1.4 Hz, 1H), 7.92 (dt, J=7.9, 1.7 Hz, 1H), 7.67 (t, J=7.8 Hz, 1H), 7.48 (d, J=2.7 Hz, 1H), 7.29 (d, J=8.0 Hz, 1H), 6.95 (t, J=7.8 Hz, 1H), 6.72 (dd, J=7.6, 1.0 Hz, 1H), 3.87 (s, 3H). ¹³C NMR (101 MHz, DMSO-d6) δ ppm: 165.3, 140.5, 133.7, 131.7, 130.9, 130.4, 130.2, 127.8, 126.7, 123.5, 121.9, 120.4, 117.3, 115.7, 104.0, 53.1. LC-MS (ESI+) m/z: 362.8 found for [M−H]−. 363.0 calcd, for C₁₆H₁₂ClN₂O₄S—, ret. time=1.78 min, purity (UV)=97%.

### Synthesis of DCAF15 Ligand—3-(N-(3-chloro-1H-indol-7-yl)sulfamoyl)benzoic Acid

Methyl 3-(N-(3-chloro-1H-indol-7-yl)sulfamoyl)benzoate (1) (2.19 g, 6.0 mmol, 1. equiv.) was dissolved in THF/H₂O (1:1, 50 ml). LiOH (1.44 g, 60.03 mmol, 10. equiv.) was added and the reaction mixture was stirred for 1-2 h until complete conversion of starting material. The mixture was acidified by addition of conc. HCl (until pH 3) and the product was extracted using DCM (3×50 ml). The combined organic phases were dried (Na₂SO₄) and put on rotary evaporator. A purple solid was afforded (1.77 g, 84%). MP: 242-248° C. (decomposed). ¹H NMR (400 MHz, DMSO-d6) δ ppm: 13.47 (s, 1H,), 11.07 (d, J=2.8 Hz, 1H), 10.09 (s, 1H), 8.29 (t, J=1.8 Hz), 8.13 (dt, J=7.8, 1.4 Hz, 1H), 7.90 (dt, J=7.9, 1.6 Hz, 1H), 7.65 (t, J=7.8 Hz, 1H), 7.48 (d, J=2.7 Hz, 1H), 7.28 (d, J=7.9 Hz, 1H), 6.95 (t, J=7.8 Hz, 1H), 6.72 (dd, J=7.6, 1.0 Hz, 1H). ¹³C NMR (101 MHz, DMSO-d6) δ ppm: 165.9, 139.9, 133.4, 131.7, 130.9, 129.8, 129.6, 127.5, 126.2, 123.1, 121.6, 120.0, 116.7, 115.2, 103.6. LC-MS (ESI+) m/z: 351.0 found for [M+H]+. 351.0 calcd, for C₁₅H₁₂ClN₂O₄S⁺, ret. time=1.52 min, purity (UV)=89%.

### General Procedure for Preparation of CRBN Ligand-Linker Conjugate (Intermediates for PROTAC 1-7)

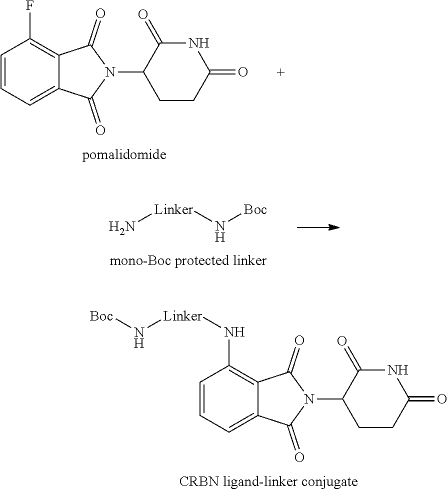

The mono-Boc protected linker (1.0 equiv.) was dissolved in dry DCM (10 ml) and kept under nitrogen atmosphere. DIPEA (4 equiv.) and pomalidomide (1.0 equiv.) was added and the reaction mixture was heated to 90° C. for 6 h. The reaction mixture was cooled to room temperature and portioned between half sat. brine and EtOAc (3×50 mL). The combined organic phases were further washed with sat. NH₄Cl (50 ml), H₂O (6×50 mL) and sat. brine (50 mL). The organic phase was dried (Na₂SO₄), filtered and put on rotary evaporator. The crude product was purified by flash column chromatography to afford the CRBN ligand-linker conjugates.

### General Procedure for Preparation of VHL Ligand-Linker Conjugate (Intermediates for PROTAC 8-11, 16-17)

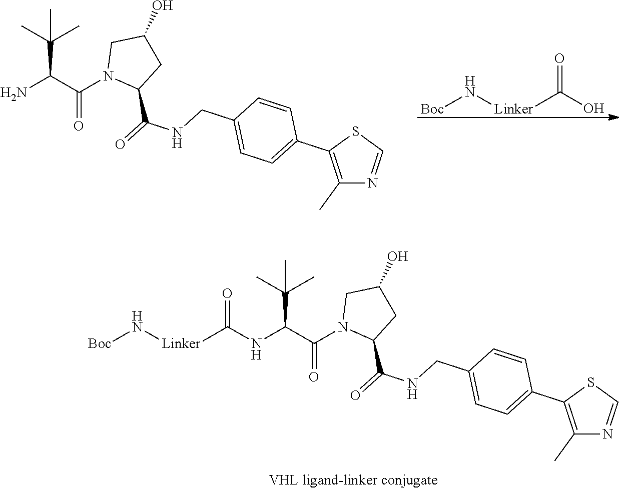

Boc-protected VH032 (1.0 equiv.) was dissolved in DCM (2 mL), followed by the addition of 4M HCl in dioxane (0.2 ml, ca. 1 mL/mmol). The reaction mixture was stirred at rt for 1 h. After complete deprotection was observed by TLC, the solvent was removed under reduced pressure and co-evaporated with Et₂O (3×10 mL). The residue was further dried in high vacuum for 2 h. Simultaneously, Boc-protected carboxylic acid linker (1.0 equiv.) was dissolved in DCM (2 mL) followed by the addition of 3-(((ethylimino)methylene)amino)-N,N-dimethylpropan-1-amine hydrogen chloride (EDC×HCl, 2.0 equiv.) and 1H-benzo[d][1,2,3]triazol-1-ol (HOBt×H₂O, 1.2 equiv.) and activation of the carboxylic acid for 30 min. Then, the reaction mixture was cooled to 0° C. and the VH032-amine (1.0 equiv.) dissolved in DCM (2 mL) and then N, N-Diisopropylethylamine (DIPEA, 2.0 equiv.) were sequentially charged into the reaction flask. The reaction was warmed to rt and monitored by TLC analysis. After full conversion (typically 1 h), the solution was diluted with (DCM, 10 mL), washed with water and extracted with DCM (3×20 ml). The combined organic layers were washed with brine (2×50 mL), dried over Na₂SO₄, filtered, and concentrated in vacuo. The crude product was purified by column chromatography (DCM/MeOH 1 to 5%) to give a white solid.

### General Procedure for Preparation of DCAF15-Linker Conjugate (Intermediates for PROTAC 18-21)

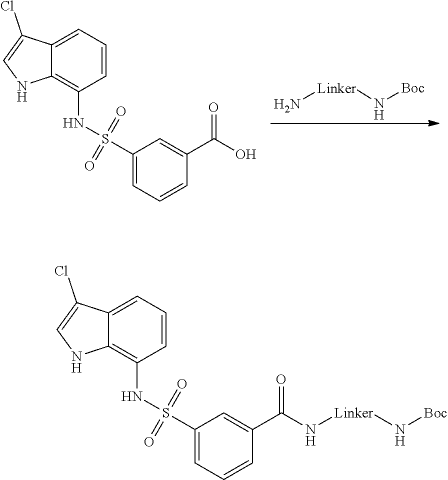

The indisulam carboxylic acid derivative (1.0 equiv.) was dissolved in DMF (2 mL) followed by the addition of 1-[Bis(dimethylamino)methylene]-1H-1,2,3-triazolo[4,5-b]pyridinium3-oxidhexafluoro-phosphate (HATU, 2.0 equiv.) and activation of the carboxylic acid for 30 min. Then, the reaction mixture was cooled to 0° C. and the respective mono-Boc protected diamine linker (0.5 equiv.) and then N,N-Diisopropylethylamine (DIPEA, 2.0 equiv.) were sequentially charged into the reaction flask. The reaction was continued until full conversion was observed via TLC analysis (typically 1 h). Then, the solution was diluted with Dichloromethane (DCM, 10 mL), washed with water and extracted with DCM (3×20 ml). The combined organic layers were washed with brine (2×50 mL), dried over Na₂SO₄, filtered, and concentrated in vacuo. The crude product was purified by column chromatography (DCM/MeOH 1 to 5%) to give an off-white solid.

### General Procedure for Synthesis of PROTAC 1-7

The Boc-protected linker-pomalidomide conjugate (1.1 equiv.) was dissolved in DCM (2 mL) and treated with TFA (0.75 mL). The reaction mixture was stirred at rt for 30 min to 1 h until complete deprotection and quenched by addition of sat. NaHCO₃ (15 mL). The aqueous solution was extracted with DCM (6×20 mL) until the organic phase is no longer fluorescent. The combined organic layers were dried using Na₂SO₄ and put on rotary evaporator. The residue was afterwards put on high vacuum. In a separate flask, 3-(N-(3-chloro-1H-indol-7-yl)sulfamoyl)benzoic acid (1.0 equiv.) is dissolved in DCM (2 mL), to which EDC×HCl (3.0 equiv.), HOBt hydrate (1.2 equiv.), and DIPEA (3.0 equiv.) were added. The deprotected amine and DIPEA (3.0 equiv.) were dissolved in DCM (4 mL) and added to the acid mixture, followed by stirring at rt for 1-18 h. The reaction is monitored by TLC and when reflecting zero change in spot intensities the reaction is quenched by diluting with water (10 mL) and extracting with DCM (3×10 mL). The combined organic phase was washed with brine (50 mL) dried using Na₂SO₄ and put on rotary evaporator. The crude product was purified by flash column chromatography yielding a yellow neon-like solid/oil.

### General Procedure for Synthesis of PROTAC 8-11 and 16-17

The Boc-protected VHL-linker conjugate (1.0 equiv.) was dissolved in DCM (2 mL), followed by the addition of 4M HCl in dioxane (0.2 ml, ca. 1 mL/mmol). The reaction mixture was stirred at rt for 1 h. After complete deprotection was observed by TLC, the solvent was removed under reduced pressure and co-evaporated with Et₂O (3×10 mL). The residue was further dried in high vacuum for 2 h. Simultaneously, the indisulam carboxylic acid derivative (1.0 equiv.) was dissolved in DMF (2 mL) followed by the addition of 1-[Bis (dimethylamino)methylene]-1H-1,2,3-triazolo[4,5-b]pyridinium3-oxidhexafluorophosphate (HATU, 2.0 equiv.) and activation of the carboxylic acid for 30 min. Then, the reaction mixture was cooled to 0° C. and the respective VH032-linker conjugate (1.0 equiv.) dissolved in DCM (2 mL) and then N,N-Diisopropylethylamine (DIPEA, 2.0 equiv.) were sequentially charged into the reaction flask. The reaction was warmed to rt and monitored by TLC analysis. After full conversion (typically 1 h) the solution was diluted with (DCM, 10 mL), washed with water and extracted with DCM (3×20 ml). The combined organic layers were washed with brine (2×50 mL), dried over Na₂SO₄, filtered, and concentrated in vacuo. The crude product was purified by column chromatography (DCM/MeOH 1 to 5%) to give a white solid.

### General Procedure for Synthesis of PROTAC 12-15

The indisulam carboxylic acid derivative (1.0 equiv.) was dissolved in DMF (2 mL) followed by the addition of 1-[Bis(dimethylamino)methylene]-1H-1,2,3-triazolo[4,5-b]pyridinium3-oxidhexafluoro-phosphate (HATU, 2.0 equiv.) and activation of the carboxylic acid for 30 min. Then, the reaction mixture was cooled to 0° C. and the respective diamine linker (0.5 equiv.) and then N,N-Diisopropylethylamine (DIPEA, 2.0 equiv.) were sequentially charged into the reaction flask. The reaction was continued until full conversion was observed via TLC analysis (typically 1 h). Then, the solution was diluted with Dichloromethane (DCM, 10 mL), washed with water and extracted with DCM (3×20 ml). The combined organic layers were washed with brine (2×50 mL), dried over Na₂SO₄, filtered, and concentrated in vacuo. The crude product was purified by column chromatography (DCM/MeOH 1 to 5%) to give a white solid.

### General Procedure for Synthesis of PROTAC 18-21

The RNF114 ligand as well as the DCAF15-RNF114 ligands were synthesized by closely following a literature procedure (Luo et al.). Shortly, the Boc-protected RNF114 ligand (1.0 equiv.) was dissolved in DCM (2 mL) and trifluoroacetic acid (TFA, 5 mL) added slowly over the course of 5 minutes. After stirring for an additional 20 minutes, deprotection was complete and the solvent was removed in vacuo. To remove residual TFA, the crude material was co-evaporated with toluene (3×10 mL) and then dried in high vacuum for 2 h. Meanwhile, the DCAF15-linker conjugate (1.0 equiv.) was Boc-deprotected by the addition of 4M HCl in dioxane (1 mL). After stirring for 30 min, the solvent was evaporated, and residues were co-evaporated with Et₂O (3×10 mL). The crude was dried in high vacuum for 2 h and used directly for the next step. The crude RNF114 acid was dissolved in DMF (1 mL) and HATU (2.0 equiv) was added, and the mixture was stirred for 1 h. Then, the reaction mixture was cooled to 0° C. and the respective DCAF15-linker amine (1.0 equiv.), dissolved in DCM (2 mL) and then N,N-Diisopropylethylamine (DIPEA, 2.0 equiv.) were sequentially charged into the reaction flask. The reaction was warmed to rt and monitored by TLC analysis. After full conversion (typically 1 h) the solution was diluted with (DCM, 10 mL), washed with water and extracted with DCM (3×20 ml). The combined organic layers were washed with brine (2×50 mL), dried over Na₂SO₄, filtered, and concentrated in vacuo. The crude product was purified by column chromatography (DCM/MeOH 1 to 3%) to give an off-white solid.

### General Procedure for Synthesis of PROTAC 22-23

The DCAF11-based PROTACs were synthesized by closely following a literature procedure (Zhang et. al.). Shortly, the DCAF15-Linker conjugate was dissolved in DCM (1 mL) and TFA (2 mL) was added slowly over the course of 5 minutes. After stirring for an additional 20 minutes, toluene was added, and the solvent was removed in vacuo. To generate the free amine, the resulting residue was dissolved in EtOAc (10 mL) and washed with sat. NaHCO₃ (2×5 mL). The organic layer was dried with Na₂SO₄, filtered, and concentrated in vacuo for 2 h. In the second step, 5-methylisoxazole-3-carboxaldehyde (1 eq) was added to a solution of the free amine (1.0 equiv.) in MeOH (0.3 mL, 0.5 M) and the reaction was stirred at room temperature for 4 h. At this point, benzylisocyanide (1.2 eq) and chloroacetic acid (1.2 eq) were added and the reaction was stirred for 3 days. At this point, additional aldehyde (1.2 equiv.) was added, and the reaction mixture was heated to 40° C. for 4 h. Then, MeOH was evaporated, and the crude residue was purified by column chromatography (DCM/MeOH 1 to 3%) to give an off-white solid.

3-(N-(3-chloro-1H-indol-7-yl)sulfamoyl)-N-(6-((2-(2,6-dioxopiperidin-3-yl)-1,3-dioxoisoindolin-4-yl)amino)hexyl)benzamide (PROTAC 1): In the column chromatography an eluent of 98:2 DCM/MeOH was used. PROTAC (1) was afforded as a yellow solid (27.3 mg, 27%). Rf: 0.32 (95:5 DCM/MeOH). ¹H NMR (400 MHz, DMSO-d6) δ ppm: 11.09 (s, 1H), 11.06 (d, J=2.8 Hz, 1H), 10.05 (s, 1H), 8.65 (t, J=5.6 Hz, 1H), 8.24 (t, J=1.8 Hz, 1H), 8.03 (dt, J=7.8, 1.4 Hz), 7.82 (ddd, J=7.9, 1.9, 1.1 Hz, 1H), 7.63-7.51 (m, 2H), 7.47 (d, J=2.7 Hz, 1H), 7.25 (d, J=8.0 Hz, 1H), 7.08 (d, J=8.6 Hz, 1H), 7.01 (d, J=7.0 Hz, 1H), 6.92 (t, J=7.8 Hz, 1H), 6.74 (dd, J=7.6, 1.0 Hz, 1H), 6.53 (t, J=5.9 Hz, 1H), 5.05 (dd, J=12.9, 5.4 Hz, 1H), 3.30 (d, J=3.7 Hz, 2H), 3.24 (q, J=6.7 Hz, 2H), 2.88 (ddd, J=17.5, 14.1, 5.5 Hz, 1H), 2.64-2.52 (m, 2H), 2.02 (m, 1H), 1.58 (m, 2H), 1.51 (m, 2H), 1.41-1.29 (m, 4H). ¹³C NMR (101 MHz, DMSO-d6) δ ppm: 172.8, 170.1, 169.0, 167.3, 164.5, 146.4, 139.7, 136.3, 135.4, 132.2, 131.2, 129.4, 129.3, 129.1, 126.2, 125.9, 123.0, 121.7, 120.0, 117.2, 116.3, 114.9, 110.4, 109.0, 103.6, 48.5, 41.8, 39.3, 31.0, 28.9, 28.6, 26.2, 26.1, 22.2. LC-MS (ESI+) m/z: 705.2 found for [M+H]+. 705.2 calcd. for C₃₄H₃₄ClN₆O₇S+, ret. time=1.87 min, purity (UV)=93%.

3-(N-(3-chloro-1H-indol-7-yl)sulfamoyl)-N-(10-((2-(2,6-dioxopiperidin-3-yl)-1,3-dioxoisoindolin-4-yl)amino) decyl)benzamide (PROTAC 2): In the column chromatography an eluent of DCM/MeOH 98:2 was used and later increased to DCM/MeOH 95:5. PROTAC 2 was afforded as a yellow solid (34.1 mg, 32%). Rf: 0.4 (DCM/MeOH, 95:5) ¹H NMR (400 MHz, DMSO-d6) δ 11.09 (s, 1H), 11.06 (d, J=2.8 Hz, 1H), 10.05 (s, 1H), 8.64 (t, J=5.6 Hz, 1H), 8.24 (t, J=1.8 Hz), 8.03 (dt, J=7.9, 1.4 Hz, 1H), 7.82 (dt, J=7.8, 1.5 Hz, 1H), 7.63-7.53 (m, 2H), 7.47 (d, J=2.7 Hz, 1H), 7.25 (d, J=8.0 Hz, 1H), 7.08 (d, J=8.6 Hz, 1H), 7.01 (d, J=7.0 Hz, 1H), 6.93 (t, J=7.8 Hz, 1H), 6.74 (d, J=7.6 Hz, 1H), 6.51 (t, J=5.9 Hz, 1H), 5.05 (dd, J=12.9, 5.4 Hz, 1H), 3.29-3.18 (m, 4H), 2.94-2.81 (m, 1H), 2.63-2.58 (m, 1H), 2.58-2.52 (m, 1H), 2.07-1.97 (m, 1H), 1.60-1.44 (m, 4H), 1.26 (s, 12H). ¹³C NMR (101 MHz, DMSO-d6) δ 173.3, 170.5, 169.4, 167.8, 164.9, 146.9, 140.1, 136.7, 135.8, 132.6, 131.6, 129.8, 129.7, 129.5, 126.6, 126.4, 123.4, 122.2, 120.4, 117.6, 116.7, 115.4, 110.8, 109.4, 104.1, 49.0, 42.3, 31.4, 29.4, 29.4, 29.2, 29.2, 29.1, 26.9, 26.8, 22.6. LCMS (ESI): m/z [M+H]+ calcd for C₃₈H₄₁ClN₆O₇S⁺ 760.24. found 761.2.

3-(N-(3-chloro-1H-indol-7-yl)sulfamoyl)-N-(2-(2-(2-((2-(2,6-dioxopiperidin-3-yl)-1,3-dioxoisoindolin-4-yl)amino)ethoxy)ethoxy)ethyl)benzamide (PROTAC 3): In the column chromatography an eluent of 98:2 DCM/MeOH. PROTAC 3 was afforded as a yellow solid (37.9 mg, 36%). Rf: 0.61 (9:1 DCM/MeOH). ¹H NMR (400 MHz, DMSO-d6) δ ppm: 11.09 (s, 1H), 11.06 (d, J=2.8 Hz, 1H,), 10.06 (s, 1H), 8.73 (t, J=5.5 Hz, 1H), 8.26 (t, J=1.8 Hz, 1H), 8.04 (dt, J=7.8, 1.4 Hz, 1H), 7.82 (ddd, J=7.9, 1.9, 1.0 Hz, 1H), 7.62-7.51 (m, 2H), 7.47 (d, J=2.7 Hz, 1H), 7.24 (d, J=8.0 Hz, 1H), 7.09 (d, J=8.6 Hz, 1H), 7.03 (d, J=7.0 Hz, 1H), 6.92 (t, J=7.8 Hz, 1H), 6.73 (dd, J=7.6, 1.0 Hz, 1H), 6.58 (t, J=5.8 Hz, 1H), 5.05 (dd, J=12.9, 5.4 Hz, 1H), 3.60 (t, J=5.5 Hz, 2H), 3.57-3.54 (m, 4H), 3.53 (t, J=6.0 Hz, 2H), 3.44-3.37 (m, 4H), 2.87 (ddd, J=17.4, 14.0, 5.4 Hz, 1H), 2.69-2.52 (m, 2H), 2.01 (ddt, J=12.8, 5.5, 2.3 Hz, 1H). ¹³C NMR (101 MHz, DMSO-d6) δ ppm: 172.8, 170.1, 168.9, 167.3, 164.7, 146.4, 139.7, 136.2, 135.1, 132.1, 131.2, 129.4, 129.3, 129.2, 126.2, 126.0, 123.0, 121.7, 120.0, 117.4, 116.3, 114.9, 110.7, 109.2, 103.6, 69.7, 69.6, 68.9, 68.8, 48.5, 41.7, 39.3, 31.0, 22.1. LC-MS (ESI+) m/z: 737.2 found for [M+H]⁺. 737.2 calcd, for C₃₄H₃₄ClN₆O₉S⁺, ret. time=1.70 min, purity (UV)=97%.

3-(N-(3-chloro-1H-indol-7-yl)sulfamoyl)-N-(2-(2-(2-(2-((2-(2,6-dioxopiperidin-3-yl)-1,3-dioxoisoindolin-4-yl)amino)ethoxy)ethoxy)ethoxy)ethyl)benzamide (PROTAC 4): In the column chromatography an eluent of DCM/MeOH 98:2 was used. PROTAC 4 was afforded as a yellow solid (22.2 mg, 25%). Rf: 0.39 (DCM/MeOH, 95:5) ¹H NMR (400 MHz, DMSO-d6) δ 11.10 (s, 1H), 11.07 (s, 1H), 10.07 (s, 1H), 8.74 (t, J=5.5 Hz, 1H), 8.27 (t, J=1.8 Hz, 1H), 8.05 (d, J=7.9 Hz, 1H), 7.83 (d, J=8.7 Hz, 1H), 7.63-7.54 (m, 2H), 7.48 (d, J=2.7 Hz, 1H), 7.25 (d, J=7.9 Hz, 1H), 7.12 (d, J=8.6 Hz, 1H), 7.04 (d, J=7.0 Hz, 1H), 6.93 (t, J=7.8 Hz, 1H), 6.74 (d, J=7.6 Hz, 1H), 6.59 (t, J=5.8 Hz, 1H), 5.06 (dd, J=12.9, 5.4 Hz, 1H), 3.59 (t, J=5.4 Hz, 2H), 3.52 (s, 10H), 3.47-3.37 (m, 4H), 2.94-2.82 (m, 1H), 2.64-2.59 (m, 1H), 2.58-2.53 (m, 1H), 2.07-1.98 (m, 1H). ¹³C NMR (101 MHz, DMSO-d6) δ 172.7, 170.0, 168.8, 167.2, 164.6, 146.3, 139.6, 136.1, 135.0, 132.0, 131.1, 129.3, 129.2, 129.1, 126.1, 125.8, 122.9, 121.6, 119.8, 117.3, 116.2, 114.8, 110.6, 109.1, 103.5, 69.6, 69.6, 69.5, 68.7, 68.6, 48.4, 41.5, 30.9, 22.0. LCMS (ESI): m/z [M+H]⁺ calcd for C₃₆H₃₇ClN₆O₁₀S⁺ 780.20. found 781.3.

3-(N-(3-chloro-1H-indol-7-yl)sulfamoyl)-N-(3-(2-(3-((2-(2,6-dioxopiperidin-3-yl)-1,3-dioxoisoindolin-4-yl)amino)propoxy)ethoxy)propyl)benzamide Preparation of DCAF15-linker-CRBN PROTAC (PROTAC 5): In the column chromatography an eluent of 98:2 DCM/MeOH was used. PROTAC 5 was afforded as a yellow solid (103 mg, 56%). Rf: 0.27 (95:5 DCM/MeOH). ¹H NMR (400 MHz, DMSO-d6) δ ppm: 11.09 (s, 1H), 11.06 (d, J=2.8 Hz, 1H), 10.06 (s, 1H), 8.64 (t, J=5.5 Hz, 1H), 8.24 (t, J=1.8 Hz, 1H), 8.02 (dt, J=8.0, 1.3 Hz, 1H), 7.82 (ddd, J=7.8, 1.9, 1.0 Hz, 1H), 7.62-7.51 (m, 2H), 7.47 (d, J=2.7 Hz, 1H), 7.25 (d, J=7.9 Hz, 1H), 7.07 (d, J=8.6 Hz, 1H), 7.00 (d, J=7.0 Hz, 1H), 6.92 (t, J=7.8 Hz, 1H), 6.74 (dd, J=7.6, 1.0 Hz, 1H), 6.65 (t, J=5.9 Hz, 1H), 5.04 (dd, J=12.9, 5.4 Hz, 1H), 3.54-3.41 (m, 8H), 3.37-3.32 (m, 2H), 3.31-3.26 (m, 2H), 2.88 (ddd, J=17.4, 14.0, 5.4 Hz, 1H), 2.62-2.52 (m, 2H), 2.06-1.97 (m, 1H), 1.83-1.69 (m, 4H). ¹³C NMR (101 MHz, DMSO-d6) δ ppm: 172.7, 170.0, 168.8, 167.2, 164.5, 146.3, 139.6, 136.2, 135.2, 132.1, 131.1, 129.3, 129.2, 129.1, 126.1, 125.8, 122.9, 121.7, 119.9, 117.0, 116.2, 114.9, 110.3, 109.0, 103.5, 69.6, 69.4, 68.1, 48.4, 39.5, 36.7, 30.9, 29.1, 28.8, 22.1. LC-MS (ESI+) m/z: 765.3 found for [M+H]⁺. 765.2 calcd, for C₃₆H₃₈ClN₆O₉S⁺, ret. time=1.78 min, purity (UV)=93%.

3-(N-(3-chloro-1H-indol-7-yl)sulfamoyl)-N-(3-(4-(3-((2-(2,6-dioxopiperidin-3-yl)-1,3-dioxoisoindolin-4-yl)amino)propoxy) butoxy)propyl)benzamide (PROTAC 6): In the column chromatography an eluent of DCM/MeOH 98:2 was used. PROTAC 6 was afforded as a yellow solid (33.5 mg, 30%). Rf: 0.43 (DCM/MeOH, 9:1) ¹H NMR (400 MHz, DMSO-d6) δ 11.09 (s, 1H), 11.06 (s, 1H), 10.06 (s, 1H), 8.64 (t, J=5.6 Hz, 1H), 8.23 (t, J=1.8 Hz, 1H), 8.03 (d, J=7.9 Hz, 1H), 7.82 (d, J=7.8 Hz, 1H), 7.62-7.53 (m, 2H), 7.46 (d, J=2.7 Hz, 1H), 7.25 (d, J=8.0 Hz, 1H), 7.07 (d, J=8.6 Hz, 1H), 7.01 (d, J=7.0 Hz, 1H), 6.93 (t, J=7.8 Hz, 1H), 6.74 (d, J=7.7 Hz, 1H), 6.65 (t, J=5.9 Hz, 1H), 5.04 (dd, J=12.9, 5.4 Hz, 1H), 3.44 (t, J=5.9 Hz, 2H), 3.29 (q, J=6.7 Hz, 2H), 2.93-2.81 (m, 1H), 2.63-2.58 (m, 1H), 2.58-2.52 (m, 1H), 2.06-1.97 (m, 1H), 1.79 (q, J=6.4 Hz, 2H), 1.72 (p, J=6.5 Hz, 2H), 1.53 (s, 4H). ¹³C NMR (101 MHz, DMSO-d6) δ 173.3, 170.5, 169.3, 167.8, 165.0, 146.9, 140.2, 136.7, 135.8, 132.6, 131.6, 129.8, 129.7, 129.6, 126.6, 126.4, 123.4, 122.2, 120.4, 117.5, 116.7, 115.4, 110.8, 109.5, 104.1, 70.5, 70.3, 68.3, 68.2, 49.0, 37.3, 31.4, 29.7, 29.3, 26.5, 26.4, 22.6. LCMS (ESI): m/z [M+H]⁺ calcd for C₃₈H₄₁ClN₆O9S⁺ 792.23. found 793.0.

### 3-(N-(3-chloro-1H-indol-7-yl)sulfamoyl)-N-(3-(2-(2-(3-((2-(2,6-dioxopiperidin-3-yl)-1,3-dioxoisoindolin-4-yl)amino)propoxy)ethoxy)ethoxy)propyl)benzamide (PROTAC 7)

Yield: 51.7 mg (74%); ¹H NMR (400 MHz, DMSO): δ 11.12-11.04 (m, 2H), 10.06 (s, 1H), 8.65 (t, J=5.5 Hz, 1H), 8.25 (t, J=1.8 Hz, 1H), 8.06-7.99 (m, 1H), 7.86-7.79 (m, 1H), 7.64-7.52 (m, 2H), 7.47 (d, J=2.7 Hz, 1H), 7.25 (d, J=7.9 Hz, 1H), 7.01 (d, J=7.0 Hz, 1H), 6.93 (t, J=7.8 Hz, 1H), 6.75 (dd, J=7.6, 1.0 Hz, 1H), 6.66 (t, J=5.9 Hz, 1H), 5.05 (dd, J=12.9, 5.4 Hz, 1H), 3.58-3.49 (m, 4H), 3.52-3.44 (m, 6H), 3.43 (t, J=6.3 Hz, 2H), 3.40-3.33 (m, 2H), 3.34-3.25 (m, 2H), 2.95-2.81 (m, 2H), 2.67-2.55 (m, 2H), 2.08-1.95 (m, 1H), 1.85-1.68 (m, 4H) ppm. ¹³C NMR (101 MHz, DMSO): δ 172.8, 170.1, 168.8, 167.3, 164.6, 146.4, 139.8, 136.3, 135.3, 132.2, 131.2, 129.4, 129.2, 129.1, 126.2, 125.9, 123.0, 120.0, 117.1, 116.2, 114.9, 110.4, 109.1, 103.6, 69.8, 69.7, 69.7, 69.6, 68.2, 68.2, 48.5, 36.8, 31.0, 29.2, 28.9, 22.2 ppm. LCMS (ES): calcd. C₃₈H₄₁ClN₆O₁₀S ([M+H]+): m/z 809.2. found 809.2.

(2S,4R)-1-((S)-2-(6-(3-(N-(3-chloro-1H-indol-7-yl)sulfamoyl)benzamido)hexanamido)-3,3-dimethylbutanoyl)-4-hydroxy-N-(4-(4-methylthiazol-5-yl)benzyl)pyrrolidine-2-carboxamide (PROTAC 8): In the column chromatography an eluent of 95:5 to 93:7 DCM/MeOH was used. PROTAC 7 was afforded as a white solid (68.7 mg, 53%). Rf: 0.45 (9:1 DCM/MeOH). [α] 589.3 nm 2: −17.88. ¹H NMR (400 MHz, DMSO-d6) δ ppm: 11.06 (d, J=2.8 Hz, 1H), 10.06 (s, 1H), 8.98 (s, 1H), 8.65 (t, J=5.6 Hz, 1H), 8.56 (t, J=6.0 Hz, 1H), 8.25 (t, J=1.8 Hz, 1H), 8.03 (dt, J=7.8, 1.4 Hz, 1H), 7.89-7.76 (m, 2H), 7.59 (t, J=7.8 Hz, 1H), 7.47 (d, J=2.7 Hz, 1H), 7.40 (m, 4H), 7.25 (d, J=8.0 Hz, 1H), 6.93 (t, J=7.8 Hz, 1H), 6.74 (dt, J=7.7, 1.5 Hz, 1H), 5.12 (d, J=3.5 Hz, 1H), 4.54 (d, J=9.4 Hz, 1H), 4.45 (d, J=6.7 Hz, 1H), 4.41 (d, J=7.5 Hz, 1H), 4.35 (q, J=3.6 Hz, 1H), 4.26-4.16 (m, 1H), 3.72-3.57 (m, 2H), 3.27-3.17 (m, 2H), 2.44 (s, 3H), 2.26 (dt, J=14.8, 7.8 Hz, 1H), 2.17-2.10 (m, 1H), 2.05-2.00 (m, 1H), 1.90 (ddd, J=12.9, 8.7, 4.6 Hz, 1H), 1.50 (m, 4H), 1.31-1.22 (m, 2H), 0.92 (s, 9H). ¹³C NMR (101 MHz, DMSO-d6) δ ppm: 172.0, 171.9, 169.7, 164.5, 151.5, 147.7, 139.8, 139.5, 135.4, 131.2, 129.6, 129.4, 129.2, 129.1, 128.6, 127.4, 126.2, 125.9, 123.0, 120.0, 116.2, 114.9, 103.6, 68.9, 58.7, 56.4, 56.3, 41.6, 39.4, 37.9, 35.2, 34.8, 28.7, 26.4, 26.2, 25.2, 15.9. From HMBC data, following 13C δ were found: 121.8, 130.1. LC-MS (ESI+) m/z: 876.5 found for [M+H]⁺. 876.3 calcd, for C₄₃H₅₁ClN₇O₇S₂⁺, ret. time=1.75 min, purity (UV)=98%.

(2S,4R)-1-((S)-2-(10-(3-(N-(3-chloro-1H-indol-7-yl)sulfamoyl)benzamido)decanamido)-3,3-dimethylbutanoyl)-4-hydroxy-N-(4-(4-methylthiazol-5-yl)benzyl)pyrrolidine-2-carboxamide (PROTAC 9): In the column chromatography DCM/MeOH 97:3 was used as eluent. PROTAC 9 as afforded as white needle crystals (399 mg, 61%). Rf: 0.65 (DCM/MeOH, 9:1) ¹H NMR (400 MHz, DMSO-d6) δ 8.99 (s, 1H), 8.56 (t, J=6.1 Hz, 1H), 7.84 (d, J=9.3 Hz, 1H), 7.41 (q, J=8.4 Hz, 4H), 6.75 (t, J=5.9 Hz, 1H), 5.12 (d, J=3.6 Hz, 1H), 4.55 (d, J=9.4 Hz, 1H), 4.48-4.44 (m, 1H), 4.43-4.40 (m, 1H), 4.35 (s, 1H), 4.22 (dd, J=15.9, 5.5 Hz, 1H), 3.70-3.62 (m, 2H), 2.88 (q, J=6.6 Hz, 2H), 2.45 (s, 3H), 2.31-2.21 (m, 1H), 2.15-2.05 (m, 1H), 2.05-1.99 (m, 1H), 1.95-1.86 (m, 1H), 1.55-1.42 (m, 2H), 1.36 (s, 9H), 1.36-1.30 (m, 2H), 1.23 (s, 10H), 0.94 (s, 9H). ¹³C NMR (101 MHz, DMSO-d6) δ 172.5, 172.4, 170.2, 156.0, 151.9, 148.2, 140.0, 131.6, 130.1, 129.1, 127.9, 77.7, 69.3, 59.1, 56.8, 56.7, 42.1, 38.4, 35.7, 35.3, 29.9, 29.4, 29.1, 29.1, 28.7, 26.8, 26.7, 25.9, 16.4. LCMS (ESI): m/z [M+H]⁺ calcd for C₃₇H₅₇N₅O₆S⁺ 699.40. found 700.3. Retention time: 1.91.

(2S,4R)-1-((S)-19-(tert-butyl)-1-(3-(N-(3-chloro-1H-indol-7-yl)sulfamoyl)phenyl)-1,17-dioxo-5,8,11,14-tetraoxa-2,18-diazaicosan-20-oyl)-4-hydroxy-N-(4-(4-methylthiazol-5-yl)benzyl)pyrrolidine-2-carboxamide (PROTAC 10): In the column chromatography an eluent of DCM/MeOH 95:5 was used and later increased to DCM/MeOH 93:7. PROTAC 10 was afforded as a colourless solid (25.6 mg, 37%). Rf: 0.3 (DCM/MeOH, 9:1) ¹H NMR (400 MHz, DMSO-d6) δ 11.06 (d, J=2.9 Hz, 1H), 10.08 (s, 1H), 8.98 (s, 1H), 8.75 (t, J=5.6 Hz, 1H), 8.57 (t, J=6.1 Hz, 1H), 8.27 (t, J=1.8 Hz, 1H), 8.04 (dt, J=7.8, 1.4 Hz, 1H), 7.92 (d, J=9.4 Hz, 1H), 7.82 (dt, J=8.0, 1.3 Hz, 1H), 7.59 (t, J=7.8 Hz, 1H), 7.46 (d, J=2.6 Hz, 1H), 7.44-7.36 (m, 4H), 7.23 (d, J=7.9 Hz, 1H), 6.92 (t, J=7.8 Hz, 1H), 6.74 (d, J=7.6 Hz, 1H), 5.13 (d, J=3.5 Hz, 1H), 4.55 (d, J=9.4 Hz, 1H), 4.45 (t, J=3.3 Hz, 1H), 4.42 (dd, J=7.4, 2.7 Hz, 1H), 4.35 (s, 1H), 4.22 (dd, J=15.9, 5.3 Hz, 1H), 3.71-3.56 (m, 4H), 3.55-3.44 (m, 14H), 3.40 (d, J=5.7 Hz, 2H), 2.60-2.52 (m, 1H), 2.44 (s, 3H), 2.40-2.30 (m, 1H), 2.09-1.99 (m, 1H), 1.95-186 (m, 1H), 0.93 (s, 9H). ¹³C NMR (101 MHz, DMSO-d6) δ 172.4, 170.4, 170.0, 165.2, 151.9, 148.2, 140.0, 135.5, 131.6, 131.6, 130.1, 129.9, 129.7, 129.1, 127.9, 126.6, 126.4, 123.3, 120.4, 116.5, 155.2, 104.0, 70.2, 70.1, 70.1, 70.0, 69.9, 69.3, 69.2, 67.4, 59.2, 56.8, 56.7, 42.1, 38.4, 36.1, 35.8, 26.8, 16.4. LCMS (ES): m/z [M+H]+ calcd for C₄₈H₆₀ClN₇O₁₁S₂⁺ 1009.35. found 1010.6.

(2S,4R)-1-((S)-12-(tert-butyl)-1-(3-(N-(3-chloro-1H-indol-7-yl)sulfamoyl)phenyl)-1,10-dioxo-5,8-dioxa-2,11-diazatridecan-13-oyl)-4-hydroxy-N-(4-(4-methylthiazol-5-yl)benzyl)pyrrolidine-2-carboxamide (PROTAC 11): In the column chromatography an eluent of 95:5 DCM/MeOH was used. PROTAC 11 was afforded as a white solid (17.3 mg, 34%). Rf: 0.34 (9:1 DCM/MeOH). [α] 589.3 nm20: −26.71 ¹H NMR (400 MHz, CDCl₃) δ ppm: 9.89 (d, J=2.7 Hz, 1H), 8.67 (s, 1H), 8.20 (t, J=1.8 Hz, 1H), 8.09 (dt, J=7.9, 1.4 Hz, 1H), 7.76 (d, J=9.3 Hz, 1H), 7.64 (t, J=5.3 Hz, 1H), 7.51 (dt, J=7.9, 1.5 Hz, 1H), 7.41 (dd, J=7.9, 1.0 Hz, 1H), 7.36-7.27 (m, 5H), 7.08 (d, J=2.4 Hz, 1H), 7.02-6.95 (m, 1H), 6.93-6.88 (m, 1H), 4.82-4.71 (m, 2H), 4.47 (m, 2H), 4.29 (dd, J=15.0, 5.4 Hz, 1H), 4.02 (m, 3H), 3.79-3.55 (m, 8H), 3.42 (dt, J=14.4, 5.2 Hz, 1H), 2.50 (s, 3H), 2.36 (td, J=8.9, 4.4 Hz, 1H), 2.21 (ddd, J=12.8, 8.5, 2.4 Hz, 1H), 0.99 (s, 9H). ¹³C NMR (101 MHz, DMSO-d6) δ ppm: 171.2, 171.1, 171.0, 165.8, 150.4, 148.5, 138.8, 137.8, 135.4, 133.0, 131.5, 131.1, 130.3, 129.8, 129.5, 129.1, 128.0, 127.1, 124.1, 121.7, 120.8, 120.4, 117.7, 116.6, 106.3, 71.1, 70.8, 70.4, 70.3, 69.6, 59.1, 57.4, 57.3, 43.2, 40.1, 36.6, 36.4, 26.5, 16.1. LC-MS (ESI+) m/z: 908.5 found for [M+H]⁺. 908.3 calcd, for C₄₃H₅₀ClN₇O₉S₂⁺. ret. time=1.71 min, purity (UV)=76%. The three most acidic protons are not observed in the 1D 1H NMR due to rapid exchange.

3-(N-(3-chloro-1H-indol-7-yl)sulfamoyl)-N-(2-(2-(2-(3-(N-(3-chloro-1H-indol-7-yl)sulfamoyl)benzamido)ethoxy)ethoxy)ethyl)benzamide (PROTAC 12): Yield: 18.8 mg (16%); ¹H NMR (400 MHz, CDCl₃): δ 9.76 (d, J=2.6 Hz, 2H), 8.22 (d, J=1.8 Hz, 2H), 7.90 (dt, J=6.6, 2.0 Hz, 2H), 7.53 (dd, J=7.6, 1.4 Hz, 2H), 7.32-7.25 (m, 6H), 7.22 (t, J=5.2 Hz, 2H), 7.09-6.99 (m, 4H), 6.92 (d, J=2.5 Hz, 2H), 3.89-3.80 (m, 4H), 3.79-3.75 (m, 8H) ppm. ¹³C NMR (101 MHz, CDCl₃): δ 167.4, 139.4, 134.8, 131.8, 130.5, 130.5, 129.5, 127.1, 124.3, 121.7, 120.5, 120.5, 119.7, 117.3, 106.4, 70.2, 69.2, 40.3 ppm. LCMS (ES): calcd. C₃₆H₃₄Cl₂N₆O₈S₂ ([M+H]+): m/z 813.1. found 813.0.

3-(N-(3-chloro-1H-indol-7-yl)sulfamoyl)-N-(1-(3-(N-(3-chloro-1H-indol-7-yl)sulfamoyl)phenyl)-1-oxo-6,9,12-trioxa-2-azapentadecan-15-yl)benzamide (PROTAC 13): Yield: 43.0 mg (34%); ¹H NMR (400 MHz, CDCl₃) δ 9.78 (s, 2H), 8.60 (s, 2H), 7.94 (d, J=7.8 Hz, 2H), 7.74 (t, J=5.4 Hz, 2H), 7.50-7.44 (m, 4H), 7.35-7.27 (m, 2H), 7.11-7.00 (m, 6H), 3.59-3.49 (m, 8H), 3.48-3.37 (m, 8H), 1.84-1.74 (m, 4H) ppm. ¹³C NMR (101 MHz, CDCl₃): δ 166.8, 139.3, 135.6, 132.0, 130.5, 130.2, 129.2, 127.2, 125.6, 121.9, 120.7, 120.6, 118.8, 117.0, 106.4, 70.1, 69.8, 69.7, 39.2, 28.5 ppm. LCMS (ES): calcd. C₄₀H₄₂Cl₂N₆O₈S₂ ([M+H]+): m/z 885.2. found 884.7.

3-(N-(3-chloro-1H-indol-7-yl)sulfamoyl)-N-(6-(3-(N-(3-chloro-1H-indol-7-yl)sulfamoyl)benzamido) hexyl)benzamide (PROTAC 14): Yield: 39.6 mg (16%); ¹H NMR (400 MHz, DMSO): δ 11.57 (s, 2H), 10.40 (s, 2H), 8.74 (t, J=5.6 Hz, 2H), 8.33 (t, J=1.8 Hz, 2H), 8.07-8.00 (m, 2H), 7.93-7.86 (m, 2H), 7.55 (t, J=7.8 Hz, 2H), 7.44 (d, J=2.7 Hz, 2H), 7.24-7.17 (m, 2H), 6.98-6.88 (m, 4H), 3.23 (q, J=6.6 Hz, 4H), 1.56-1.47 (s, 4H), 1.35-1.26 (m, 4H) ppm. ¹³C NMR (101 MHz, DMSO): δ 165.0, 140.3, 135.7, 131.7, 129.6, 129.5, 129.2, 126.5, 126.4, 123.2, 122.6, 120.4, 116.0, 114.9, 104.0, 79.7, 40.6, 40.4, 40.4, 40.2, 40.2, 39.9, 39.7, 39.5, 39.3, 29.4, 26.6 ppm. One CH2 signal missing due to superimposition with the DMSO-d6 peak. LCMS (ES): calcd. C₃₆H₃₄ClN₆O₆S₂ ([M+H]+): m/z 781.1. found 780.6.

3-(N-(3-chloro-1H-indol-7-yl)sulfamoyl)-N-(10-(3-(N-(3-chloro-1H-indol-7-yl)sulfamoyl)benzamido) decyl)benzamide (PROTAC 15): Yield: 22.0 mg (18%); ¹H NMR (400 MHz, DMSO): δ 11.07 (s, 2H), 10.05 (s, 2H), 8.64 (t, J=5.6 Hz, 2H), 8.24 (d, J=1.8 Hz, 2H), 8.07-8.00 (m, 2H), 7.85-7.78 (m, 2H), 7.59 (t, J=7.8 Hz, 2H), 7.47 (d, J=2.7 Hz, 2H), 7.25 (d, J=8.0 Hz, 2H), 6.93 (t, J=7.8 Hz, 2H), 6.74 (dd, J=7.6, 1.0 Hz, 2H), 3.22 (q, J=6.6 Hz, 4H), 1.48 (d, J=7.0 Hz, 4H), 1.32-1.21 (m, 12H) ppm. ¹³C NMR (101 MHz, DMSO): δ 164.5, 139.7, 135.4, 131.2, 129.4, 129.2, 129.1, 126.2, 125.9, 123.0, 121.8, 119.9, 116.3, 114.9, 103.6, 40.2, 40.1, 40.0, 39.9, 39.8, 39.7, 39.6, 39.5, 39.4, 39.3, 39.1, 29.0, 28.9, 28.8, 26.5 ppm. One CH2 signal missing due to superimposition with the DMSO-d6 peak. LCMS (ES): calcd. C₄₀H₄₂Cl₂N₆O₆S₂ ([M+H]+): m/z 837.2. found 837.1.

(2S,4R)-1-((S)-2-((1r,4S)-4-((3-(N-(3-chloro-1H-indol-7-yl)sulfamoyl)benzamido) methyl)cyclohexane-1-carboxamido)-3,3-dimethylbutanoyl)-4-hydroxy-N-(4-(4-methylthiazol-5-yl)benzyl)pyrrolidine-2-carboxamide (PROTAC 16): Yield: 81.8 mg (64%); ¹H NMR (400 MHz, DMSO): ð 10.95 (s, 1H), 9.98 (s, 1H), 8.90 (s, 1H), 8.59 (t, J=5.6 Hz, 1H), 8.52 (t, J=6.1 Hz, 1H), 8.26 (t, J=1.8 Hz, 1H), 8.02 (dt, J=7.8, 1.4 Hz, 1H), 7.79 (ddd, J=7.9, 1.9, 1.1 Hz, 1H), 7.59 (d, J=9.3 Hz, 1H), 7.53 (t, J=7.8 Hz, 1H), 7.44-7.31 (m, 6H), 7.22 (d, J=7.9 Hz, 1H), 6.91 (t, J=7.8 Hz, 1H), 6.80 (d, J=7.6 Hz, 1H), 5.08 (s, 1H), 4.52 (d, J=9.3 Hz, 1H), 4.48-4.40 (m, 2H), 4.39-4.32 (m, 1H), 4.21 (dd, J=15.8, 5.4 Hz, 1H), 3.70-3.63 (m, 2H), 3.11 (td, J=8.0, 7.4, 4.3 Hz, 2H), 2.44 (s, 3H), 2.37-2.25 (m, 1H), 2.09-1.99 (m, 1H), 1.96-1.88 (m, 1H), 1.83-1.63 (m, 4H), 1.57-1.43 (m, 1H), 1.39-1.25 (m, 2H), 1.00-8.43 (m, 11H) ppm. ¹³C NMR (101 MHz, DMSO): δ 174.9, 171.9, 169.6, 164.6, 151.0, 147.6, 139.6, 139.3, 135.3, 131.2, 131.1, 129.6, 129.0, 129.0, 128.9, 128.8, 128.5, 127.3, 126.1, 125.9, 122.6, 121.7, 119.7, 115.9, 114.7, 103.8, 68.8, 58.7, 56.2, 56.0, 45.5, 43.3, 41.7, 40.2, 40.1, 39.9, 39.7, 39.5, 39.3, 39.1, 38.9, 37.8, 36.9, 35.3, 29.8, 29.6, 29.5, 28.2, 26.3, 15.8 ppm. LCMS (ES): calcd. C₄₅H₅₂ClN₇O₇S₂ ([M+H]+): m/z 902.3. found 902.1.

(2S,4R)-1-((S)-2-(4-((3-(N-(3-chloro-1H-indol-7-yl)sulfamoyl)benzamido) methyl)benzamido)-3,3-dimethylbutanoyl)-4-hydroxy-N-(4-(4-methylthiazol-5-yl)benzyl)pyrrolidine-2-carboxamide (PROTAC 17): Yield: 88.6 mg (69%); ¹H NMR (400 MHz, DMSO): δ 11.07 (d, J=2.8 Hz, 1H), 10.06 (s, 1H), 9.32 (t, J=6.0 Hz, 1H), 8.98 (s, 1H), 8.59 (t, J=6.1 Hz, 1H), 8.29 (t, J=1.8 Hz, 1H), 8.14-8.06 (m, 1H), 7.92 (d, J=9.1 Hz, 1H), 7.87-7.81 (m, 3H), 7.63 (t, J=7.8 Hz, 1H), 7.47 (d, J=2.7 Hz, 1H), 7.43-7.34 (m, 6H), 7.27 (d, J=8.0 Hz, 1H), 6.94 (t, J=7.8 Hz, 1H), 6.75 (dd, J=7.6, 1.0 Hz, 1H), 5.15 (d, J=3.6 Hz, 1H), 4.77 (d, J=9.1 Hz, 1H), 4.51 (d, J=5.8 Hz, 2H), 4.49-4.33 (m, 3H), 4.24 (dd, J=15.8, 5.5 Hz, 1H), 3.73 (d, J=3.0 Hz, 2H), 2.44 (s, 3H), 2.11-2.01 (m, 1H), 1.97-1.86 (m, 1H), 1.03 (s, 9H) ppm. ¹³C NMR (101 MHz, DMSO): δ 171.9, 169.5, 166.2, 164.7, 151.5, 151.5, 147.7, 142.8, 139.8, 139.5, 134.9, 132.7, 131.4, 131.2, 129.7, 129.5, 129.4, 128.7, 127.7, 127.5, 127.0, 126.2, 126.0, 123.0, 121.7, 120.0, 116.4, 115.0, 103.6, 68.9, 58.8, 57.2, 56.4, 54.9, 42.5, 41.7, 40.2, 40.1, 40.0, 39.9, 39.8, 39.7, 39.5, 39.3, 39.1, 38.9, 37.9, 35.6, 26.5, 16.0 ppm. LCMS (ES): calcd. C₄₅H₄₆ClN₇O₇S₂ ([M+H]+): m/z 896.3. found 896.0.

N-(2-(2-(2-(2-(4-(3-(4-bromophenyl)-1-(2-chloroacetyl)-4,5-dihydro-1H-pyrazol-5-yl) phenoxy) acetamido)ethoxy)ethoxy)ethyl)-3-(N-(3-chloro-1H-indol-7-yl)sulfamoyl)benzamide (PROTAC 18): Yield: 37.0 mg (46%); ¹H NMR (400 MHz, CDCl₃): δ 9.77 (s, 1H), 8.52 (s, 1H), 8.22 (t, J=1.8 Hz, 1H), 7.98 (dt, J=7.8, 1.4 Hz, 1H), 7.63-7.54 (m, 5H), 7.41 (p, J=3.9 Hz, 1H), 7.31 (t, J=7.8 Hz, 1H), 7.16-7.10 (m, 3H), 7.07 (d, J=2.6 Hz, 1H), 7.02-6.95 (m, 3H), 6.86-6.76 (m, 2H), 5.55 (dd, J=11.7, 4.9 Hz, 1H), 4.63 (d, J=13.8 Hz, 1H), 4.54 (d, J=13.7 Hz, 1H), 4.46 (s, 2H), 3.75 (dd, J=17.9, 11.7 Hz, 1H), 3.67-3.48 (m, 12H), 3.14 (dd, J=17.9, 4.8 Hz, 1H) ppm. ¹³C NMR (201 MHz, CDCl₃): δ 169.5, 165.8, 164.4, 156.8, 155.0, 139.1, 135.2 (d, J=5.2 Hz), 134.4, 132.3, 132.1, 130.1, 129.6, 129.5, 129.1, 128.3, 127.3, 127.0, 125.5, 124.6, 121.8, 121.0, 120.4, 117.8, 116.4, 115.3, 106.2, 70.4, 70.2, 69.6 (d, J=2.9 Hz), 69.5, 67.4, 60.2, 42.2, 42.1, 40.0, 39.1 ppm. LCMS (ES): calcd. C₄₀H₃₉BrCl₂N₆O₈S ([M+H]+): m/z 913.1. found 913.4.

N-(1-(4-(3-(4-bromophenyl)-1-(2-chloroacetyl)-4,5-dihydro-1H-pyrazol-5-yl) phenoxy)-2-oxo-7,10,13-trioxa-3-azahexadecan-16-yl)-3-(N-(3-chloro-1H-indol-7-yl)sulfamoyl)benzamide (PROTAC 19): Yield: 23.2 mg (27%); ¹H NMR (400 MHz, CDCl₃): δ 9.75 (s, 1H), 8.53 (s, 1H), 8.26 (t, J=1.8 Hz, 1H), 7.99 (dt, J=7.9, 1.4 Hz, 1H), 7.64-7.54 (m, 5H), 7.44 (t, J=5.2 Hz, 1H), 7.39 (d, J=7.7 Hz, 1H), 7.34 (t, J=7.8 Hz, 1H), 7.16-7.13 (m, 2H), 7.12-7.07 (m, 1H), 7.05 (d, J=2.6 Hz, 1H), 6.96 (t, J=7.7 Hz, 1H), 6.91 (dd, J=7.6, 1.2 Hz, 1H), 6.84-6.80 (m, 2H), 5.56 (dd, J=11.7, 4.8 Hz, 1H), 4.61 (d, J=13.7 Hz, 1H), 4.53 (d, J=13.7 Hz, 1H), 4.46 (s, 2H), 3.75 (dd, J=17.9, 11.7 Hz, 1H), 3.66-3.57 (m, 8H), 3.54 (q, J=5.7 Hz, 2H), 3.51-3.48 (m, 2H), 3.44 (t, J=5.8 Hz, 2H), 3.39 (q, J=6.4 Hz, 2H), 3.15 (dd, J=17.9, 4.8 Hz, 1H), 1.85 (p, J=5.6 Hz, 2H), 1.74-1.65 (m, 2H) ppm. ¹³C NMR (101 MHz, CDCl₃): δ 168.7, 165.4, 164.3, 157.0, 154.9, 139.3, 135.6, 134.2, 132.1, 132.0, 130.0, 129.7, 129.5, 129.0, 128.3, 127.3, 127.0, 125.4, 124.8, 121.7, 121.1, 120.4, 117.6, 116.3, 115.3, 106.2, 70.6, 70.1, 69.9, 69.9, 69.7, 69.5, 67.4, 60.1, 42.2, 42.1, 39.2, 37.4, 28.8, 28.5 ppm. LCMS (ES): calcd. C₄₄H₄₇BrCl₂N₆O₈S ([M+H]+): m/z 985.2. found 984.8.

N-(6-(2-(4-(3-(4-bromophenyl)-1-(2-chloroacetyl)-4,5-dihydro-1H-pyrazol-5-yl) phenoxy) acetamido) hexyl)-3-(N-(3-chloro-1H-indol-7-yl)sulfamoyl)benzamide (PROTAC 20): Yield: 50.6 mg (65%); ¹H NMR (800 MHz, CDCl₃): δ 9.70 (d, J=2.7 Hz, 1H), 8.69 (s, 1H), 8.37 (t, J=1.7 Hz, 1H), 7.97 (dt, J=7.8, 1.4 Hz, 1H), 7.65-7.55 (m, 5H), 7.42 (d, J=7.7 Hz, 1H), 7.33 (t, J=7.8 Hz, 1H), 7.17-7.12 (m, 2H), 7.07 (d, J=2.4 Hz, 1H), 7.02-6.95 (m, 2H), 6.94 (t, J=5.8 Hz, 1H), 6.88-6.80 (m, 2H), 6.72 (t, J=6.1 Hz, 1H), 5.54 (dd, J=11.7, 4.7 Hz, 1H), 4.62 (d, J=13.7 Hz, 1H), 4.54-4.51 (m, 3H), 3.75 (dd, J=17.7, 11.7 Hz, 1H), 3.46-3.34 (m, 4H), 3.14 (dd, J=17.7, 4.8 Hz, 1H), 1.71 (s, 4H), 1.59-1.51 (m, 4H) ppm. ¹³C NMR (201 MHz, CDCl₃) δ 169.3, 166.3, 164.4, 157.0, 155.1, 139.1, 135.9, 134.5, 132.3, 132.1, 130.2, 130.0, 129.7, 129.2, 128.4, 127.4, 127.2, 125.6, 125.3, 121.9, 121.1, 120.6, 118.2, 116.7, 115.3, 67.5, 60.2, 42.4, 42.3, 39.9, 38.3, 29.0, 28.9 ppm. LCMS (ES): calcd. C₄₀H₃₉BrCl₂N₆O₆S ([M+H]+): m/z 881.1. found 880.5.

N-(10-(2-(4-(3-(4-bromophenyl)-1-(2-chloroacetyl)-4,5-dihydro-1H-pyrazol-5-yl) phenoxy) acetamido) decyl)-3-(N-(3-chloro-1H-indol-7-yl)sulfamoyl)benzamide (PROTAC 21): Yield: 17.0 mg (20%); ¹H NMR (400 MHz, CDCl₃): δ 9.67 (s, 1H), 8.43 (s, 1H), 8.04 (t, J=1.8 Hz, 1H), 7.92 (dt, J=7.9, 1.4 Hz, 1H), 7.74 (dt, J=8.1, 1.3 Hz, 1H), 7.65-7.57 (m, 4H), 7.46-7.35 (m, 3H), 7.18-7.10 (m, 2H), 7.06-6.99 (m, 2H), 6.82-6.74 (m, 2H), 6.66 (t, J=6.0 Hz, 1H), 6.23 (t, J=5.7 Hz, 1H), 5.58 (dd, J=11.7, 4.7 Hz, 1H), 4.66 (d, J=13.7 Hz, 1H), 4.56 (d, J=13.7 Hz, 1H), 4.42 (s, 2H), 3.78 (ddd, J=17.9, 11.7, 2.9 Hz, 1H), 3.36 (q, J=7.0, 6.4 Hz, 4H), 3.16 (dd, J=17.9, 4.8 Hz, 1H), 1.67 (s, 2H), 1.61-1.49 (m, 4H), 1.38-1.25 (m, 10H). ¹³C NMR (201 MHz, CDCl₃) δ 168.4, 166.1, 164.6, 157.0, 155.2, 139.1, 135.6, 134.2, 132.3 (d, J=4.5 Hz), 131.8, 130.1, 129.9, 129.6, 129.3, 128.4, 127.3, 127.1, 125.7, 125.4, 121.9, 121.1, 120.5, 118.5, 116.7, 115.3, 106.3, 67.5, 60.4, 42.3, 42.3, 40.5, 39.3, 29.4, 29.3, 29.1, 29.0, 29.0, 28.9, 26.8, 26.7 ppm. LCMS (ES): calcd. C₄₄H₄₇BrCl₂N₆O₆S ([M+H]+): m/z 937.2. found 938.0.

N-(6-(N-(2-(benzylamino)-1-(5-methylisoxazol-3-yl)-2-oxoethyl)-2-chloroacetamido) hexyl)-3-(N-(3-chloro-1H-indol-7-yl)sulfamoyl)benzamide (PROTAC 22): Yield: 35.0 mg (83%); ¹H NMR (400 MHz, CDCl₃): δ 9.55 (s, 1H), 8.34 (d, J=14.1 Hz, 2H), 8.00 (d, J=7.8 Hz, 2H), 7.59 (d, J=7.8 Hz, 1H), 7.42 (d, J=7.8 Hz, 1H), 7.34 (t, J=7.9 Hz, 1H), 7.28-7.14 (m, 5H), 7.10-7.04 (m, 1H), 7.04 (d, J=2.6 Hz, 1H), 6.97 (t, J=7.8 Hz, 1H), 6.90 (d, J=7.5 Hz, 1H), 6.14 (s, 1H), 5.40 (s, 1H), 4.35 (t, J=5.7 Hz, 2H), 4.20-4.13 (m, 2H), 3.64-3.52 (m, 1H), 3.52-3.34 (m, 2H), 3.36-3.22 (m, 1H), 2.40 (s, 3H), 1.62-1.51 (m, 4H), 1.42-1.30 (m, 4H) ppm. ¹³C NMR (101 MHz, CDCl₃): δ 171.0, 167.9, 166.5, 166.1, 160.0, 139.0, 137.1, 135.6, 133.7, 132.2, 130.0, 129.7, 129.1, 128.7, 127.5, 127.4, 127.1, 125.1, 121.8, 120.9, 120.4, 117.7, 116.5, 106.3, 102.1, 58.3, 49.9, 44.0, 41.0, 39.9, 28.8, 28.0, 26.2, 25.7, 12.3 ppm. LCMS (ES): calcd. C₃₆H₃₈Cl₂N₆O₆S ([M+H]+): m/z 753.2. found 753.0.

N-(10-(N-(2-(benzylamino)-1-(5-methylisoxazol-3-yl)-2-oxoethyl)-2-chloroacetamido) decyl)-3-(N-(3-chloro-1H-indol-7-yl)sulfamoyl)benzamide (PROTAC 23): Yield: 12.5 mg (22%); ¹H NMR (800 MHz, CDCl₃): δ 9.65 (s, 1H), 8.45 (s, 1H), 8.27 (s, 1H), 7.98 (d, J=7.8 Hz, 1H), 7.63 (dt, J=7.8, 1.5 Hz, 1H), 7.45 (d, J=7.9 Hz, 1H), 7.36 (t, J=7.8 Hz, 1H), 7.27 (d, J=7.4 Hz, 2H), 7.25-7.19 (m, 4H), 7.06 (d, J=2.6 Hz, 1H), 7.00 (t, J=7.7 Hz, 1H), 6.94 (d, J=7.6 Hz, 1H), 6.78 (t, J=6.4 Hz, 1H), 6.19 (s, 1H), 5.50 (s, 1H), 4.42 (d, J=4.1 Hz, 2H), 4.26 (d, J=12.7 Hz, 1H), 4.19 (d, J=12.7 Hz, 1H), 3.58-3.51 (m, 1H), 3.49-3.33 (m, 3H), 2.42 (s, 3H), 1.71-1.65 (m, 1H), 1.64-1.59 (m, 1H), 1.56 (p, J=7.3 Hz, 2H), 1.34-1.26 (m, 8H), 1.26-1.21 (m, 4H) ppm. ¹³C NMR (201 MHz, CDCl₃): δ 170.8, 167.8, 166.4, 165.8 (d, J=4.8 Hz), 160.2, 139.1, 137.2, 135.4, 131.9, 130.0, 129.8, 129.1, 128.7, 127.5 (d, J=6.6 Hz), 127.4, 127.1, 125.1, 121.8, 120.9, 120.4, 118.0, 116.6, 106.2, 102.3, 58.1, 50.2, 44.0, 41.2, 40.4, 29.1, 28.9, 28.6, 28.6, 26.5, 26.4, 12.3 ppm. LCMS (ES): calcd. C₄₀H₄₆Cl₂N₆O₆S ([M+H]+): m/z 809.3. found 809.1.

### General Procedure for Synthesis of HyT Molecule 24-27

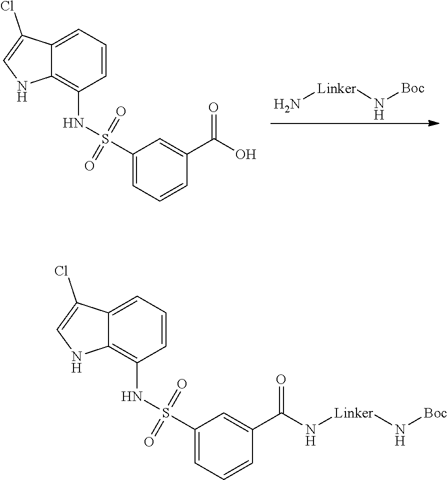

The indisulam carboxylic acid derivative (1.0 equiv.) was dissolved in DMF (2 mL) followed by the addition of 1-[Bis(dimethylamino)methylene]-1H-1,2,3-triazolo[4,5-b]pyridinium3-oxidhexafluoro-phosphate (HATU, 2.0 equiv.) and activation of the carboxylic acid for 30 min. Then, the reaction mixture was cooled to 0° C. and the respective mono-Boc protected diamine linker (0.5 equiv.) and then N, NV-Diisopropylethylamine (DIPEA, 2.0 equiv.) were sequentially charged into the reaction flask. The reaction was continued until full conversion was observed via TLC analysis (typically 1 h). Then, the solution was diluted with Dichloromethane (DCM, 10 mL), washed with water, and extracted with DCM (3×20 ml). The combined organic layers were washed with brine (2×50 mL), dried over Na₂SO₄, filtered, and concentrated in vacuo. The crude product was purified by column chromatography (DCM/MeOH 1 to 5%) to give an off-white solid.

Tert-butyl(2-(2-(2-(3-(N-(3-chloro-1H-indol-7-yl)sulfamoyl)benzamido)ethoxy)ethoxy)ethyl)carbamate (Hyt molecule 24): Yield: 130.00 mg, 79%; Rf=0.67 (DCM/MeOH, 9:1); 1H NMR (400 MHz, DMSO): δ 11.07 (s, 1H), 10.06 (s, 1H), 8.75 (t, J=5.5 Hz, 1H), 8.26 (t, J=1.8 Hz, 1H), 8.08-8.01 (m, 1H), 7.86-7.78 (m, 1H), 7.60 (t, J=7.8 Hz, 1H), 7.47 (d, J=2.7 Hz, 1H), 7.25 (d, J=8.0 Hz, 1H), 6.93 (t, J=7.8 Hz, 1H), 6.78-6.70 (m, 2H), 3.55-3.45 (m, 6H), 3.44-3.33 (m, 2H), 3.33-3.30 (m, 2H), 3.04 (q, J=6.0 Hz, 2H), 1.36 (s, 9H) ppm. 13C NMR (201 MHz, DMSO): δ 164.7, 155.6, 139.8, 135.1, 131.2, 129.4, 129.3, 126.2, 126.0, 123.0, 121.2, 120.0, 116.3, 114.9, 103.6, 77.6, 69.5, 69.5, 69.2, 68.7, 38.2, 30.8, 28.2 ppm. HRMS (ESI): m/z [M+H]+ calcd for C26H34ClN4O7S+ 581.1832. found 581.1837.

tert-butyl (1-(3-(N-(3-chloro-1H-indol-7-yl)sulfamoyl)phenyl)-1-oxo-6,9,12-trioxa-2-azapentadecan-15-yl)carbamate (Hyt molecule 25): Yield: 151.00 mg, 81%; Rf=0.64 (DCM/MeOH, 9:1); 1H NMR (400 MHz, DMSO) § 11.07 (s, 1H), 10.06 (s, 1H), 8.66 (t, J=5.6 Hz, 1H), 8.25 (t, J=1.8 Hz, 1H), 8.04 (dt, J=7.9, 1.3 Hz, 1H), 7.86-7.79 (m, 1H), 7.60 (t, J=7.8 Hz, 1H), 7.48 (d, J=2.7 Hz, 1H), 7.30-7.23 (m, 1H), 6.94 (t, J=7.8 Hz, 1H), 6.79-6.71 (m, 2H), 3.54-3.47 (m, 6H), 3.46-3.42 (m, 4H), 3.37 (t, J=6.3 Hz, 2H), 3.32-3.27 (m, 2H), 2.96 (q, J=6.6 Hz, 2H), 1.75 (h, J=6.7, 6.2 Hz, 2H), 1.59 (p, J=6.6 Hz, 2H), 1.37 (s, 9H) ppm. 13C NMR (101 MHz, DMSO): δ 164.6, 155.6, 139.7, 135.3, 131.2, 129.4, 129.2, 129.1, 126.2, 125.9, 123.0, 121.7, 120.0, 116.3, 114.9, 103.6, 77.4, 69.8, 69.7, 69.6, 69.5, 68.2, 68.1, 38.2, 37.2, 36.8, 29.7, 29.2, 28.2 ppm. HRMS (ESI): m/z [M+H]+ calcd for C30H42ClN4O8S+ 653.2407. found 653.2410.

tert-butyl (6-(3-(N-(3-chloro-1H-indol-7-yl)sulfamoyl)benzamido) hexyl)carbamate (Hyt molecule 26): Yield: 126.60 mg, 81%; Rf=0.34 (DCM/MeOH, 9:1); 1H NMR (400 MHz, DMSO): δ 11.06 (s, 1H), 10.06 (s, 1H), 8.65 (t, J=5.6 Hz, 1H), 8.27-8.22 (m, 1H), 8.07-8.00 (m, 1H), 7.86-7.78 (m, 1H), 7.60 (t, J=7.8 Hz, 1H), 7.48 (d, J=2.7 Hz, 1H), 7.26 (dt, J=8.0, 0.8 Hz, 1H), 6.94 (t, J=7.8 Hz, 1H), 6.80-6.72 (m, 2H), 3.23 (q, J=6.7 Hz, 2H), 2.90 (q, J=6.6 Hz, 2H), 1.49 (p, J=7.1, 6.4 Hz, 2H), 1.40-1.33 (m, 11H), 1.30-1.22 (m, 4H) ppm. 13C NMR (101 MHz, DMSO): δ 164.5, 155.6, 139.7, 135.4, 131.2, 129.4, 129.2, 129.1, 126.2, 125.9, 123.0, 121.7, 120.0, 116.3, 114.9, 103.6, 77.3, 40.4, 29.4, 28.9, 28.3, 26.2, 26.0 ppm. HRMS (ESI): m/z [M+H]+ calcd for C26H34ClN4O5S+ 549.1933. found 549.1937.

tert-butyl (10-(3-(N-(3-chloro-1H-indol-7-yl)sulfamoyl)benzamido) decyl)carbamate (Hyt molecule 27): Yield: 88.20 mg, 51%; Rf=0.45 (DCM/MeOH, 95:5); 1H NMR (400 MHz, MeOD): δ 8.13 (d, J=1.8 Hz, 1H), 8.00-7.93 (m, 1H), 7.81-7.74 (m, 2H), 7.53 (t, J=7.8 Hz, 1H), 7.34 (d, J=8.0 Hz, 1H), 7.24 (s, 1H), 6.89 (t, J=7.8 Hz, 1H), 6.61 (d, J=7.5 Hz, 1H), 3.34 (d, J=7.1 Hz, 2H), 3.01 (t, J=7.0 Hz, 2H), 1.58 (p, J=7.0 Hz, 2H), 1.47-1.40 (m, 11H), 1.39-1.24 (m, 12H) ppm. Note: NH protons missing due to H-D exchange. 13C NMR (101 MHz, MeOD): δ 168.3, 158.6, 141.3, 136.8, 132.4, 132.2, 131.0, 130.2, 128.4, 127.3, 123.4, 122.7, 120.9, 119.8, 117.3, 106.3, 79.8, 41.4, 41.1, 31.0, 30.6, 30.6, 30.4, 30.4, 30.3, 28.8, 28.0, 27.9 ppm. HRMS (ESI): m/z [M+H]+ calcd for C30H42ClN4O5S+ 605.2559. found 605.2556.

### Synthesis of HyT Molecule 28

1-(3-(N-(3-chloro-1H-indol-7-yl)sulfamoyl)phenyl)-1-oxo-6,9,12-trioxa-2-azapentadecan-15-aminium (intermediate for Hyt molecule 28): To a solution of Hyt molecule 25 (25.00 mg, 0.04 mmol, 1.0 equiv.) in DCM (2 mL) TFA (0.5 mL) was added dropwise. The reaction mixture was stirred until completion was indicated by TLC analysis (2 h). At this point, the solution was evaporated by co-evaporation with toluene (3×10 mL). The crude product was dissolved in a 1:1 mixture of MeCN and 50 mM HCl and lyophilized overnight. This process was repeated three times to give the title compounds as an off-white solid (22.00 mg, quant.). 1H NMR (400 MHz, DMSO): δ 11.29 (s, 1H), 10.21 (s, 1H), 8.71 (t, J=5.6 Hz, 1H), 8.29 (t, J=1.8 Hz, 1H), 8.08-8.01 (m, 1H), 7.89-7.78 (m, 4H), 7.59 (t, J=7.8 Hz, 1H), 7.47 (d, J=2.7 Hz, 1H), 7.24 (d, J=7.9 Hz, 1H), 6.94 (t, J=7.8 Hz, 1H), 6.85-6.78 (m, 1H), 3.55-3.38 (m, 12H), 3.30 (q, J=6.7 Hz, 2H), 2.86-2.78 (m, 2H), 1.83-1.69 (m, 4H) ppm. 13C NMR (101 MHz, DMSO): δ 164.8, 139.8, 135.0, 131.3, 129.3, 129.1, 126.2, 126.0, 122.9, 121.9, 120.0, 116.0, 114.8, 103.6, 69.7, 69.5, 69.5, 68.2, 67.3, 36.8, 36.7, 27.1 ppm. HRMS (ESI): m/z [M+H]+ calcd for C25H34ClN4O6S+ 553.1886. found 553.1883.

The indisulam carboxylic acid derivative (1.0 equiv., 0.09 mmol) was dissolved in DMF (2 mL) followed by the addition of 1-[Bis(dimethylamino)methylene]-1H-1,2,3-triazolo[4,5-b]pyridinium3-oxidhexafluorophosphate (HATU, 1.5 equiv.) and activation of the carboxylic acid for 30 min. Then, the reaction mixture was cooled to 0° C. and the deprotected HyT 25 (1.1 equiv.) and then N, NV-Diisopropylethylamine (DIPEA, 3.0 equiv.) were sequentially charged into the reaction flask. The reaction was continued until full conversion was observed via TLC analysis (typically 1 h). Then, the solution was diluted with Dichloromethane (DCM, 10 mL), washed with water, and extracted with DCM (3×20 ml). The combined organic layers were washed with brine (2×50 mL), dried over Na₂SO₄, filtered, and concentrated in vacuo. The crude product was purified by column chromatography (DCM/MeOH 1 to 5%).

N-(1-((3r,5r,7r)-adamantan-1-yl)-2-oxo-7,10,13-trioxa-3-azahexadecan-16-yl)-3-(N-(3-chloro-1H-indol-7-yl)sulfamoyl)benzamide (Hyt molecule 28): Yield: 15.5 mg, 37%; Rf=0.26 (DCM/MeOH 95:5); 1H NMR (400 MHz, DMSO): δ 11.06 (s, 1H), 10.06 (s, 1H), 8.66 (t, J=5.6 Hz, 1H), 8.24 (t, J=1.8 Hz, 1H), 8.07-8.00 (m, 1H), 7.85-7.78 (m, 1H), 7.67-7.55 (m, 2H), 7.47 (d, J=2.7 Hz, 1H), 7.29-7.22 (m, 1H), 6.93 (t, J=7.8 Hz, 1H), 6.78-6.71 (m, 1H), 3.53-3.47 (m, 6H), 3.46-3.41 (m, 4H), 3.38 (t, J=6.4 Hz, 2H), 3.29 (q, J=6.7 Hz, 2H), 3.05 (q, J=6.9 Hz, 2H), 1.91-1.86 (m, 3H), 1.79 (s, 2H), 1.74 (p, J=6.6 Hz, 2H), 1.66-1.59 (m, 4H), 1.58-1.50 (dd, J=13.2, 2.4 Hz, 10H) ppm. 13C NMR (101 MHz, DMSO): δ 169.8, 164.6, 139.7, 135.3, 131.2, 129.4, 129.3, 129.2, 126.2, 125.9, 123.0, 121.7, 120.0, 116.3, 114.9, 103.6, 69.8, 69.8, 68.2, 68.1, 50.1, 42.1, 38.3, 36.8, 36.5, 35.6, 32.1, 29.5, 29.2, 28.0 ppm. HRMS (ESI): m/z [M+H]+ calcd for C37H50ClN4O7S+ 729.3084. found 729.3088.

### General Procedure for Synthesis of HyT Molecule 29-41

The indisulam carboxylic acid derivative (1.0 equiv., 0.09 mmol) was dissolved in DMF (2 mL) followed by the addition of 1-[Bis(dimethylamino)methylene]-1H-1,2,3-triazolo[4,5-b]pyridinium3-oxidhexafluorophosphate (HATU, 1.5 equiv.) and activation of the carboxylic acid for 30 min. Then, the reaction mixture was cooled to 0° C. and the respective amine (1.1 equiv.) and then N,N-Diisopropylethylamine (DIPEA, 3.0 equiv.) were sequentially charged into the reaction flask. The reaction was continued until full conversion was observed via TLC analysis (typically 1 h). Then, the solution was diluted with Dichloromethane (DCM, 10 mL), washed with water, and extracted with DCM (3×20 ml). The combined organic layers were washed with brine (2×50 mL), dried over Na₂SO₄, filtered, and concentrated in vacuo. The crude product was purified by column chromatography (DCM/MeOH 1 to 5%) to give the indicated title compounds.

(E)-N-(3-chloro-1H-indol-7-yl)-3-(4-(4-(4-methoxyphenyl)-4-oxobut-2-enoyl) piperazine-1-carbonyl)benzenesulfonamide (Hyt molecule 29): Yield: 18.9 mg, 55%; Rf=0.42 (DCM/MeOH 95:5); 1H NMR (400 MHz, DMSO): δ 11.01 (s, 1H), 10.01 (s, 1H), 8.09-8.02 (m, 1H), 8.00-7.91 (m, 1H), 7.88-7.77 (m, 2H), 7.70-7.54 (m, 3H), 7.48-7.36 (m, 2H), 7.31-7.19 (m, 1H), 7.14-7.03 (m, 2H), 7.00-6.93 (m, 1H), 6.81-6.72 (m, 1H), 3.86 (d, J=5.4 Hz, 3H), 3.76-3.37 (m, 6H), 3.25-2.91 (m, 2H) ppm. 13C NMR (101 MHz, DMSO): δ 163.8, 163.5, 136.1, 131.5, 131.2, 130.9, 129.8, 129.7, 129.4, 129.4, 129.2, 126.2, 123.1, 121.6, 119.9, 117.0, 115.1, 114.3, 114.1, 103.6, 55.7, 55.6 ppm. CO cross peak identified at 188.0 ppm. CH2 cross peaks identified via 2D HSQC at 41.3, 45.6 and 47.4 ppm. HRMS (ESI): m/z [M+H]+ calcd for C30H28ClN4O6S+ 607.1413. found 607.1416.

### 3-(N-(3-chloro-1H-indol-7-yl)sulfamoyl)-N-(4-methoxybenzyl)benzamide (Hyt Molecule 30)

Yield: 32.9 mg, 82%; Rf=0.66 (DCM/MeOH 95:5); 1H NMR (400 MHz, DMSO): δ 11.07 (s, 1H), 10.05 (s, 1H), 9.18 (t, J=5.9 Hz, 1H), 8.28 (t, J=1.8 Hz, 1H), 8.11-8.05 (m, 1H), 7.86-7.79 (m, 1H), 7.60 (t, J=7.8 Hz, 1H), 7.46 (d, J=2.7 Hz, 1H), 7.28-7.20 (m, 3H), 6.98-6.84 (m, 3H), 6.78-6.70 (m, 1H), 4.39 (d, J=5.8 Hz, 2H), 3.73 (s, 3H) ppm. 13C NMR (101 MHz, DMSO): δ 164.5, 158.3, 139.9, 135.1, 131.2, 131.2, 129.4, 129.3, 129.3, 128.7, 126.2, 126.0, 123.0, 121.8, 120.0, 116.3, 114.9, 113.7, 103.6, 55.1, 42.2 ppm. LCMS (ESI): m/z [M+H]+ calcd for C23H21ClN3O4S+ 470.1. found 470.0.

3-(N-(3-chloro-1H-indol-7-yl)sulfamoyl)-N-(4-methoxyphenethyl)benzamide (Hyt molecule 31): Yield: 19.7 mg, 71%; Rf=0.65 (DCM/MeOH 9:1); 1H NMR (400 MHz, DMSO): δ 11.06 (s, 1H), 10.06 (s, 1H), 8.75 (t, J=5.6 Hz, 1H), 8.24 (t, J=1.8 Hz, 1H), 8.04-7.97 (m, 1H), 7.86-7.79 (m, 1H), 7.60 (t, J=7.9 Hz, 1H), 7.47 (d, J=2.7 Hz, 1H), 7.29-7.22 (m, 1H), 7.17-7.09 (m, 2H), 6.94 (t, J=7.8 Hz, 1H), 6.88-6.80 (m, 2H), 6.77-6.71 (m, 1H), 3.71 (s, 3H), 3.47-3.37 (m, 2H), 2.75 (t, J=7.4 Hz, 2H) ppm. 13C NMR (101 MHz, DMSO): δ 164.56, 157.69, 139.76, 135.33, 131.24, 131.18, 129.59, 129.40, 129.31, 129.17, 126.21, 125.93, 123.02, 121.75, 119.97, 116.25, 114.94, 113.77, 103.63, 54.96, 41.25, 34.05 ppm. HRMS (ESI): m/z [M+H]+ calcd for C24H23ClN3O4S+ 484.1093. found 484.1096.

3-(N-(3-chloro-1H-indol-7-yl)sulfamoyl)-N-(3-(4-methoxyphenyl)propyl)benzamide (Hyt molecule 32): Yield: 36.2 mg, 85%; Rf=0.56 (DCM/MeOH 95:5); 1H NMR (400 MHz, DMSO): δ 11.06 (s, 1H), 10.05 (s, 1H), 8.68 (t, J=5.5 Hz, 1H), 8.30-8.22 (m, 1H), 8.11-8.00 (m, 1H), 7.86-7.77 (m, 1H), 7.60 (t, J=7.8 Hz, 1H), 7.47 (d, J=2.7 Hz, 1H), 7.25 (d, J=8.0 Hz, 1H), 7.14-7.10 (m, 2H), 6.93 (t, J=7.8 Hz, 1H), 6.86-6.82 (m, 2H), 6.74 (d, J=7.6 Hz, 1H), 3.71 (s, 3H), 3.24 (q, J=6.6 Hz, 2H), 2.54 (t, J=7.7 Hz, 2H), 1.77 (p, J=7.4 Hz, 2H) ppm. 13C NMR (101 MHz, DMSO): δ 165.0, 157.8, 140.0, 135.8, 133.9, 131.6, 129.8, 129.7, 126.6, 126.4, 123.4, 121.8, 120.4, 116.2, 115.2, 114.1, 104.2, 55.4, 39.4, 32.1, 31.3 ppm. LCMS (ESI): m/z [M+H]+ calcd for C25H25ClN3O4S+ 498.1. found 498.0.

3-(N-(3-chloro-1H-indol-7-yl)sulfamoyl)-N-(4-sulfamoylphenethyl)benzamide (Hyt molecule 33): Yield: 24.7 mg, 54%; Rf=0.29 (DCM/MeOH 95:5); 1H NMR (400 MHz, DMSO): δ 10.91 (s, 1H), 8.79 (t, J=5.6 Hz, 1H), 8.14-8.07 (m, 1H), 7.97-7.87 (m, 1H), 7.86-7.77 (m, 1H), 7.74-7.67 (m, 2H), 7.58 (t, J=7.8 Hz, 1H), 7.44-7.33 (m, 3H), 7.26 (d, J=5.7 Hz, 1H), 6.92 (t, J=7.8 Hz, 1H), 6.69 (d, J=7.5 Hz, 1H), 3.48 (q, J=6.8 Hz, 2H), 2.88 (t, J=7.2 Hz, 2H) ppm. One NH missing due to D-H exchange. 13C NMR (101 MHz, DMSO): δ 165.9, 144.4, 142.3, 140.8, 135.6, 131.6, 130.3, 130.2, 130.1, 129.9, 126.9, 126.5, 126.3, 123.6, 121.8, 120.8, 117.2, 116.0, 104.5, 41.2, 35.1 ppm. LCMS (ESI): m/z [M+H]+ calcd for C23H22ClN4O5S2+ 533.1. found 532.9.

4-(N-(3-chloro-1H-indol-7-yl)sulfamoyl)-N-(4-methoxybenzyl)benzamide (Hyt molecule 34): Yield: 34.2 mg, 68%; Rf=0.54 (DCM/MeOH 95:5); 1H NMR (400 MHz, DMSO): δ 10.92 (s, 1H), 9.18 (t, J=6.0 Hz, 1H), 7.93-7.84 (m, 2H), 7.82-7.73 (m, 2H), 7.42 (s, 1H), 7.28 (d, J=8.0 Hz, 1H), 7.23-7.18 (m, 2H), 6.96-6.91 (m, 1H), 6.87-6.82 (m, 2H), 6.70 (d, J=7.4 Hz, 1H), 4.35 (d, J=6.0 Hz, 2H), 3.68 (s, 3H) ppm. One NH missing due to D-H exchange. 13C NMR (101 MHz, DMSO): δ 166.1, 158.9, 142.1, 138.7, 131.6, 130.4, 129.5, 128.6, 127.7, 126.9, 123.6, 121.9, 120.8, 117.7, 116.1, 114.4, 104.4, 55.7, 42.9 ppm. LCMS (ESI): m/z [M+H]+ calcd for C23H21ClN3O4S+ 470.1. found 470.0.

4-(N-(3-chloro-1H-indol-7-yl)sulfamoyl)-N-(4-methoxyphenethyl)benzamide (Hyt molecule 35): Yield: 27.4 mg, 66%; Rf=0.54 (DCM/MeOH 95:5); 1H NMR (400 MHz, DMSO): δ 11.06 (s, 1H), 10.09 (s, 1H), 8.70 (t, J=5.6 Hz, 1H), 7.92-7.84 (m, 2H), 7.84-7.76 (m, 2H), 7.48 (d, J=2.7 Hz, 1H), 7.27 (d, J=8.0 Hz, 1H), 7.15-7.10 (m, 2H), 6.95 (t, J=7.8 Hz, 1H), 6.86-6.82 (m, 2H), 6.79-6.73 (m, 1H), 3.71 (s, 3H), 3.46-3.36 (m, 2H), 2.80-2.71 (m, 2H) ppm. 13C NMR (101 MHz, DMSO): δ 164.9, 157.7, 141.4, 138.4, 131.2, 129.6, 129.4, 127.9, 126.9, 126.2, 123.0, 121.7, 120.0, 116.3, 115.0, 113.8, 103.6, 55.0, 41.2, 34.0 ppm. LCMS (ESI): m/z [M+H]+ calcd for C24H23ClN3O4S+ 484.1. found 484.0.

3-(N-(3-chloro-1H-indol-7-yl)sulfamoyl)-N-phenethylbenzamide (Hyt molecule 36): Yield: 38.3 mg, 89%; Rf=0.43 (DCM/MeOH 95:5); 1H NMR (400 MHz, DMSO): δ 11.07 (s, 1H), 10.07 (s, 1H), 8.79 (t, J=5.6 Hz, 1H), 8.25 (t, J=1.8 Hz, 1H), 8.05-7.98 (m, 1H), 7.87-7.80 (m, 1H), 7.61 (t, J=7.8 Hz, 1H), 7.48 (d, J=2.7 Hz, 1H), 7.31-7.24 (m, 3H), 7.26-7.18 (m, 3H), 6.94 (t, J=7.8 Hz, 1H), 6.78-6.72 (m, 1H), 3.53-3.43 (m, 2H), 2.90-2.79 (m, 2H) ppm. 13C NMR (101 MHz, DMSO): δ 165.0, 140.2, 139.8, 135.7, 131.6, 129.8, 129.6, 129.1, 128.8, 126.6, 126.6, 126.4, 123.5, 120.4, 116.7, 115.4, 104.1, 41.4, 35.4 ppm. LCMS (ESI): m/z [M+H]+ calcd for C23H21ClN3O3S+ 454.1. found 454.0.

3-(N-(3-chloro-1H-indol-7-yl)sulfamoyl)-N-(4-methylphenethyl)benzamide (Hyt molecule 37): Yield: 28.9 mg, 72%; Rf=0.39 (DCM/MeOH 95:5); 1H NMR (400 MHz, DMSO): δ 10.91 (s, 1H), 8.71 (t, J=5.6 Hz, 1H), 8.11-8.07 (m, 1H), 7.98-7.90 (m, 1H), 7.86-7.76 (m, 1H), 7.58 (t, J=7.8 Hz, 1H), 7.40 (s, 1H), 7.26 (d, J=7.7 Hz, 1H), 7.08-7.00 (m, 4H), 6.91 (t, J=7.8 Hz, 1H), 6.68 (d, J=7.5 Hz, 1H), 3.45-3.38 (m, 2H), 2.73 (t, J=7.3 Hz, 2H), 2.20 (s, 3H) ppm. One NH missing due to D-H exchange. 13C NMR (101 MHz, DMSO) δ 166.8, 141.9, 137.7, 136.9, 132.9, 131.1, 131.0, 130.8, 130.6, 130.2, 127.9, 127.5, 124.6, 122.9, 121.6, 120.8, 118.2, 117.0, 105.5, 42.7, 35.9, 22.2. LCMS (ESI): m/z [M+H]+ calcd for C24H23ClN3O3S+ 468.1. found 468.0.

N-(4-bromophenethyl)-3-(N-(3-chloro-1H-indol-7-yl)sulfamoyl)benzamide (Hyt molecule 38): Yield: 22.4 mg, 49%; Rf=0.5 (DCM/MeOH 95:5); 1H NMR (400 MHz, DMSO): δ 10.92 (s, 1H), 8.72 (t, J=5.6 Hz, 1H), 8.09 (s, 1H), 7.96-7.89 (m, 1H), 7.87-7.79 (m, 1H), 7.59 (t, J=7.8 Hz, 1H), 7.51-7.45 (m, 1H), 7.41 (d, J=4.6 Hz, 1H), 7.38 (s, 1H), 7.28 (d, J=7.9 Hz, 1H), 7.24-7.18 (m, 1H), 7.16-7.10 (m, 1H), 6.92 (t, J=7.8 Hz, 1H), 6.69 (d, J=7.6 Hz, 1H), 3.44 (q, J=6.8 Hz, 2H), 2.81-2.76 (m, 2H) ppm. One NH missing due to D-H exchange. 13C NMR (101 MHz, DMSO): δ 165.6, 140.0, 139.1, 138.0, 135.4, 131.8, 131.6, 131.5, 131.4, 130.0, 130.0, 129.8, 126.6, 126.2, 123.3, 121.8, 120.5, 119.6, 117.2, 115.7, 104.2, 41.0, 34.4 ppm. LCMS (ESI): m/z [M+H]+ calcd for C23H20ClN3O3S+ 534.0. found 533.8.

3-(N-(3-chloro-1H-indol-7-yl)sulfamoyl)-N-(4-(isopentyloxy) phenethyl)benzamide (Hyt molecule 39): Yield: 34.0 mg, 74%; Rf=0.51 (DCM/MeOH 95:5); 1H NMR (400 MHz, DMSO): δ 11.07 (s, 1H), 10.06 (s, 1H), 8.75 (t, J=5.6 Hz, 1H), 8.24 (t, J=1.8 Hz, 1H), 8.04-7.96 (m, 1H), 7.86-7.79 (m, 1H), 7.60 (t, J=7.8 Hz, 1H), 7.47 (d, J=2.7 Hz, 1H), 7.26 (d, J=8.0 Hz, 1H), 7.14-7.07 (m, 2H), 6.93 (t, J=7.8 Hz, 1H), 6.86-6.78 (m, 2H), 6.78-6.71 (m, 1H), 3.93 (t, J=6.6 Hz, 2H), 3.47-3.37 (m, 2H), 2.79-2.73 (m, 2H), 1.84-1.69 (m, 1H), 1.58 (q, J=6.7 Hz, 2H), 0.92 (s, 3H), 0.91 (s, 3H) ppm. 13C NMR (101 MHz, DMSO): δ 165.0, 157.6, 140.2, 135.8, 131.6, 131.6, 130.0, 129.8, 129.7, 129.6, 126.6, 126.4, 123.4, 120.4, 116.7, 115.2, 114.9, 114.7, 104.1, 66.2, 41.7, 37.9, 34.6, 25.0, 22.9 ppm. LCMS (ESI): m/z [M+H]+ calcd for C28H31ClN3O4S+ 540.2. found 540.1.

3-(N-(3-chloro-1H-indol-7-yl)sulfamoyl)-N-(4-phenoxyphenethyl)benzamide (Hyt molecule 40): Yield: 22.1 mg, 47%; Rf=0.61 (DCM/MeOH 95:5); 1H NMR (400 MHz, DMSO): δ 10.91 (s, 1H), 8.73 (t, J=5.7 Hz, 1H), 8.10 (t, J=1.8 Hz, 1H), 7.99-7.89 (m, 1H), 7.85-7.77 (m, 1H), 7.57 (t, J=7.8 Hz, 1H), 7.39 (s, 1H), 7.36-7.31 (m, 2H), 7.24 (d, J=8.0 Hz, 1H), 7.21-7.17 (m, 2H), 7.11-7.06 (m, 1H), 6.93-6.88 (m, 3H), 6.87-6.83 (m, 2H), 6.68 (d, J=7.5 Hz, 1H), 3.44 (q, J=7.1 Hz, 2H), 2.78 (t, J=7.2 Hz, 2H) ppm. 13C NMR (101 MHz, DMSO): δ 165.8, 157.5, 155.5, 140.2, 135.7, 135.1, 132.0, 130.9, 130.7, 130.3, 130.2, 130.0, 126.9, 126.4, 124.0, 123.6, 122.1, 120.8, 119.4, 118.9, 117.5, 115.9, 104.5, 41.6, 34.6 ppm. LCMS (ESI): m/z [M+H]+ calcd for C29H25ClN3O4S+ 546.1. found 546.1.

3-(N-(3-chloro-1H-indol-7-yl)sulfamoyl)-N-(2-(naphthalen-2-yl)ethyl)benzamide (Hyt molecule 41): Yield: 28.70 mg, 99%; Rf=0.70 (DCM/MeOH 9:1); 1H NMR (400 MHz, DMSO): δ 11.07 (s, 1H), 10.07 (s, 1H), 8.83 (t, J=5.6 Hz, 1H), 8.25 (t, J=1.8 Hz, 1H), 8.04-7.97 (m, 1H), 7.88-7.80 (m, 4H), 7.75-7.71 (m, 1H), 7.59 (t, J=7.8 Hz, 1H), 7.50-7.43 (m, 3zH), 7.40 (dd, J=8.4, 1.7 Hz, 1H), 7.26 (dt, J=8.0, 0.8 Hz, 1H), 6.92 (t, J=7.8 Hz, 1H), 6.77-6.68 (m, 1H), 3.62-3.53 (m, 2H), 3.00 (t, J=7.3 Hz, 2H) ppm. 13C NMR (101 MHz, DMSO) δ 164.6, 139.8, 137.1, 135.3, 133.1, 131.7, 131.2, 129.4, 129.3, 129.2, 127.8, 127.5, 127.5, 127.3, 126.7, 126.2, 126.0, 125.9, 125.4, 123.0, 121.7, 120.0, 116.3, 115.0, 103.6, 40.9, 35.1 ppm. HRMS (ESI): m/z [M+H]+ calcd for C27H23ClN3O3S+ 504.1144. found 504.1146.

### Synthesis of DCAF15 Ligand—(Ligand for Hyt 54-56) 3-(N-(3-cyano-4-methyl-1H-indol-7-yl)sulfamoyl)benzoic Acid

Methyl 3-(N-(3-cyano-4-methyl-1H-indol-7-yl)sulfamoyl)benzoate (850 mg, 2.3 mmol, 1. equiv.) was dissolved in THF/H₂O (1:1, 25 ml). LiOH (551 mg, 23.0 mmol, 10. equiv.) was added and the reaction mixture was stirred for 1-2 h until complete conversion of starting material. The mixture was acidified by addition of conc. HCl (until pH 3) and the product was extracted using DCM (3×50 ml). The combined organic phases were dried (Na₂SO₄) and put on rotary evaporator. An off-white solid was afforded (815 mg (quant.)); 1H NMR (400 MHz, DMSO): δ 13.44 (s, 1H), 12.40 (s, 1H), 10.45 (s, 1H), 8.24 (t, J=1.8 Hz, 1H), 8.15 (d, J=3.0 Hz, 1H), 8.10 (dt, J=7.8, 1.4 Hz, 1H), 7.96 (dt, J=8.0, 1.5 Hz, 1H), 7.63 (t, J=7.8 Hz, 1H), 6.83-6.76 (m, 1H), 6.71 (d, J=7.8 Hz, 1H), 2.55 (s, 3H) ppm. 13C NMR (101 MHz, DMSO): δ 165.9, 139.9, 135.1, 133.4, 131.6, 131.0, 130.2, 129.7, 128.9, 128.2, 127.6, 127.3, 126.5, 122.5, 120.5, 118.0, 117.4, 84.2, 17.7 ppm. LCMS (ESI): m/z [M+H]+ calcd for C17H14N3O4S+ 356.1. found 356.0.

N-(4-(benzyloxy) phenethyl)-3-(N-(3-chloro-1H-indol-7-yl)sulfamoyl)benzamide (HyT molecule 42): Yield: 12.0 mg, 25%; Rf=(DCM/MeOH 95:5); 1H NMR (400 MHz, DMSO): δ 11.08 (s, 1H), 10.07 (s, 1H), 8.76 (t, J=5.6 Hz, 1H), 8.24 (t, J=1.8 Hz, 1H), 8.04-7.97 (m, 1H), 7.86-7.79 (m, 1H), 7.60 (t, J=7.8 Hz, 1H), 7.47 (d, J=2.7 Hz, 1H), 7.45-7.35 (m, 4H), 7.36-7.27 (m, 1H), 7.26 (d, J=8.0 Hz, 1H), 7.17-7.09 (m, 2H), 6.98-6.88 (m, 3H), 6.78-6.69 (m, 1H), 5.05 (s, 2H), 3.47-3.38 (m, 2H), 2.76 (t, J=7.4 Hz, 2H) ppm. 13C NMR (101 MHz, DMSO) δ 164.5, 156.8, 139.9, 139.8, 137.2, 135.3, 131.5, 131.2, 129.6, 129.4, 129.3, 129.2, 128.4, 127.8, 127.6, 126.2, 125.9, 123.0, 121.8, 120.0, 116.2, 114.9, 114.7, 103.6, 69.1, 41.2, 34.1 ppm. HRMS (ESI): m/z [M+H]+ calcd for C30H27ClN3O4S+ 560.1406. found 560.1460.

N-(2-([1,1′-biphenyl]-4-yl)ethyl)-3-(N-(3-chloro-1H-indol-7-yl)sulfamoyl)benzamide (HyT molecule 43): Yield: (25 mg, 27%). Rf=0.47 (5:95 MeOH/DCM); 1H NMR (400 MHz, DMSO): δ 11.08 (s, 1H), 10.07 (s, 1H), 8.82 (t, J=5.6 Hz, 1H), 8.25 (t, J=1.8 Hz, 1H), 8.03 (dt, J=7.8, 1.3 Hz, 1H), 7.83-7.80 (m, 1H), 7.67-7.60 (m, 3H), 7.60-7.54 (m, 2H), 7.50-7.40 (m, 3H), 7.37-7.33 (m, 1H), 7.32-7.29 (m, 2H), 7.25 (d, J=8.0 Hz, 1H), 6.93 (t, J=7.8 Hz, 1H), 6.74 (dd, J=7.6, 1.0 Hz, 1H), 3.51 (q, J=6.9 Hz, 2H), 2.88 (t, J=7.3 Hz, 2H) ppm. 13C NMR (101 MHz, DMSO): δ 165.1, 140.5, 140.2, 139.2, 138.5, 135.7, 131.7, 129.9, 129.8, 129.7, 129.6, 129.4, 127.7, 127.1, 126.9, 126.6, 126.4, 123.5, 122.3, 120.4, 116.7, 115.4, 104.1, 41.4, 35.0 ppm. LCMS (ESI): m/z [M+H]+ calcd, for C29H25ClN3O3S+ 530.1. found 530.1.

4-(N-(3-chloro-1H-indol-7-yl)sulfamoyl)-N-(4-phenoxyphenethyl)benzamide (HyT molecule 44): Yield: 15 mg, 31%; Rf=0.49 (5:95 MeOH/DCM); 1H NMR (400 MHz, DMSO): δ 11.06 (s, 1H), 10.09 (s, 1H), 8.73 (t, J=5.6 Hz, 1H), 7.94-7.85 (m, 2H), 7.84-7.76 (m, 2H), 7.48 (d, J=2.7 Hz, 1H), 7.41-7.29 (m, 2H), 7.29-7.20 (m, 3H), 7.11-7.09 (m, 1H), 7.01-6.89 (m, 5H), 6.76 (dd, J=7.7, 1.0 Hz, 1H), 3.46 (q, J=7.8, 7.0 Hz, 2H), 2.81 (t, J=7.4 Hz, 2H) ppm. 13C NMR (101 MHz, DMSO): δ 165.4, 157.4, 155.3, 141.9, 138.9, 135.0, 130.6, 130.4, 129.7, 128.4, 127.4, 126.7, 123.6, 123.5, 122.3, 120.4, 119.2, 118.7, 116.8, 115.4, 104.1, 41.4, 34.6 ppm. LCMS (ESI): m/z [M+H]+ calcd, for C29H25ClN2O4S+ 546.1. found 546.1.

N-(4-(benzyloxy) phenethyl)-4-(N-(3-chloro-1H-indol-7-yl)sulfamoyl)benzamide (HyT molecule 45): Yield: 40.0 mg, 84%; Rf=(DCM/MeOH 95:5); 1H NMR (400 MHz, DMSO): δ 11.07 (s, 1H), 10.10 (s, 1H), 8.70 (t, J=5.6 Hz, 1H), 7.92-7.84 (m, 2H), 7.84-7.76 (m, 2H), 7.48 (d, J=2.7 Hz, 1H), 7.44-7.29 (m, 5H), 7.26 (d, J=8.0 Hz, 1H), 7.16-7.11 (m, 2H), 6.99-6.88 (m, 3H), 6.80-6.73 (m, 1H), 5.06 (s, 2H), 3.46-3.37 (m, 2H), 2.75 (t, J=7.4 Hz, 2H) ppm. 13C NMR (101 MHz, DMSO): δ 164.9, 156.8, 141.5, 138.4, 137.2, 131.5, 129.6, 129.4, 128.4, 127.9, 127.8, 127.6, 126.9, 126.2, 123.0, 121.8, 120.0, 116.3, 114.9, 114.7, 103.6, 69.1, 41.2, 34.0 ppm. HRMS (ESI): m/z [M+H]+ calcd for C30H27ClN3O4S+ 560.1406. found 560.1408.

tert-butyl (1-(4-(N-(3-chloro-1H-indol-7-yl)sulfamoyl)phenyl)-1-oxo-6,9,12-trioxa-2-azapentadecan-15-yl)carbamate (HyT molecule 46): Yield: 86.3 mg, 77%; Rf=0.38 (5:95 MeOH/DCM); 1H NMR (400 MHz, DMSO): δ 11.06 (s, 1H), 10.08 (s, 1H), 8.59 (t, J=5.5 Hz, 1H), 7.95-7.87 (m, 2H), 7.84-7.76 (m, 2H), 7.48 (d, J=2.8 Hz, 1H), 7.26 (d, J=7.9 Hz, 1H), 6.94 (t, J=7.7 Hz, 1H), 6.80-6.70 (m, 2H), 3.49 (dt, J=7.6, 3.2 Hz, 6H), 3.43 (t, J=5.5 Hz, 4H), 3.36 (d, J=6.2 Hz, 2H), 3.31-3.25 (m, 2H), 2.94 (q, J=6.5 Hz, 2H), 1.73 (p, J=6.5 Hz, 2H, 1.57 (p, J=6.6 Hz, 2H), 1.35 (s, 9H) ppm. 13C NMR (101 MHz, DMSO): δ 165.4, 155.4, 142.1, 139.1, 129.7, 128.4, 127.3, 126.5, 123.5, 122.0, 120.4, 116.7, 115.4, 104.1, 77.8, 70.2, 70.0, 68.6, 68.5, 37.7, 37.2, 30.2, 29.6, 28.7 ppm. LCMS (ESI): m/z [M−H]− calcd. for C30H40ClN4O8S− 651.2. found 651.1.

tert-butyl (6-(4-(N-(3-chloro-1H-indol-7-yl)sulfamoyl)benzamido) hexyl)carbamate (HyT molecule 47): Yield: (53 mg, 56%); Rf=0.37 (5:95 MeOH/DCM); 1H NMR (400 MHz, DMSO): δ 11.06 (s, 1H), 10.08 (s, 1H), 8.59 (t, J=5.6 Hz, 1H), 7.93-7.87 (m, 2H), 7.83-7.76 (m, 2H), 7.48 (d, J=2.7 Hz, 1H), 7.26 (d, J=7.9 Hz, 1H), 6.94 (t, J=7.8 Hz, 1H), 6.79-6.70 (m, 2H), 3.21 (q, J=6.6 Hz, 2H), 2.88 (q, J=6.6 Hz, 2H), 1.48 (p, J=7.2 Hz, 2H), 1.36 (s, 11H), 1.29-1.21 (m, 4H) ppm. 13C NMR (101 MHz, DMSO): δ 165.3, 156.0, 142.0, 139.0, 129.8, 128.4, 127.3, 126.6, 123.4, 121.9) 120.4, 116.6, 115.3, 104.1, 77.7, 40.2, 39.7, 29.9, 29.4, 28.7, 26.6, 26.4 ppm. LCMS (ESI): m/z [M−H]− calcd, for C26H32ClN4O5S− 547.2. found 547.0.

tert-butyl (10-(4-(N-(3-chloro-1H-indol-7-yl)sulfamoyl)benzamido) decyl)carbamate (HyT molecule 48): Yield: 55 mg, 53%; Rf=0.37 (5:95 MeOH/DCM); 1H NMR (400 MHz, DMSO): δ 11.06 (s, 1H), 10.08 (s, 1H), 8.59 (t, J=5.6 Hz, 1H), 7.93-7.87 (m, 2H), 7.83-7.76 (m, 2H), 7.48 (d, J=2.7 Hz, 1H), 7.26 (d, J=7.9 Hz, 1H), 6.94 (t, J=7.8 Hz, 1H), 6.79-6.70 (m, 2H), 3.21 (q, J=6.7 Hz, 2H), 2.87 (q, J=6.6 Hz, 2H), 1.47 (p, J=7.0 Hz, 2H), 1.39-1.30 (m, 11H), 1.28-1.18 (m, 12H) ppm. 13C NMR (101 MHz, DMSO): δ 165.3, 156.0, 141.8, 139.0, 129.8, 128.4, 127.3, 126.7, 123.5, 121.9, 120.4, 116.7, 115.4, 104.1, 77.7, 40.2, 39.8, 29.9, 29.4, 29.2, 28.7, 26.9, 26.7 ppm. LCMS (ESI): m/z [M−H]− calcd. for C30H40ClN4O5S− 603.2. found 603.2.

tert-butyl (10-(3-(N-(3-cyano-4-methyl-1H-indol-7-yl)sulfamoyl)benzamido) decyl)carbamate (HyT molecule 54): Yield: 49.4 mg, 96%; Rf=(DCM/MeOH 95:5); 1H NMR (400 MHz, DMSO): δ 11.98 (s, 1H), 10.00 (s, 1H), 8.65 (t, J=5.6 Hz, 1H), 8.23-8.14 (m, 2H), 8.09-8.01 (m, 1H), 7.82-7.74 (m, 1H), 7.61 (t, J=7.8 Hz, 1H), 6.79-6.70 (m, 2H), 6.51 (d, J=7.7 Hz, 1H), 3.23 (q, J=6.6 Hz, 2H), 2.92-2.83 (m, 2H), 2.56 (s, 3H), 1.54-1.44 (m, 2H), 1.36 (s, 9H), 1.35-1.31 (m, 2H), 1.29-1.20 (m, 12H) ppm. 13C NMR (101 MHz, DMSO): δ 164.5, 155.6, 139.6, 135.4, 135.3, 131.2, 131.0, 129.2, 127.8, 126.5, 126.0, 122.4, 120.2, 118.8, 117.4, 84.3, 77.3, 29.5, 29.0, 28.9, 28.9, 28.8, 28.7, 28.3, 26.5, 26.3 ppm. One CH2 peak missing due to superimposition with DMSO-d6. HRMS (ESI): m/z [M+H]+ calcd for C32H44N5O5S+ 610.3058. found 610.3063.

3-(N-(3-cyano-4-methyl-1H-indol-7-yl)sulfamoyl)-N-(4-phenoxyphenethyl)benzamide (HyT molecule 55): Yield: 39.5 mg, 85%; Rf=(DCM/MeOH 95:5); 1H NMR (400 MHz, DMSO): δ 11.98 (s, 1H), 10.02 (s, 1H), 8.79 (t, J=5.6 Hz, 1H), 8.21 (t, J=1.8 Hz, 1H), 8.17 (d, J=2.4 Hz, 1H), 8.07-8.00 (m, 1H), 7.83-7.76 (m, 1H), 7.62 (t, J=7.8 Hz, 1H), 7.41-7.32 (m, 2H), 7.28-7.20 (m, 2H), 7.15-7.07 (m, 1H), 7.00-6.89 (m, 4H), 6.78-6.71 (m, 1H), 6.51 (d, J=7.7 Hz, 1H), 3.52-3.43 (m, 2H), 2.82 (t, J=7.4 Hz, 2H), 2.55 (s, 3H) ppm. 13C NMR (101 MHz, DMSO): δ 164.6, 157.0, 154.9, 139.6, 135.3, 135.3, 134.6, 131.2, 131.0, 130.2, 130.0, 129.3, 129.3, 127.8, 127.0, 126.5, 126.0, 123.2, 122.4, 120.3, 118.8, 118.3, 117.4, 84.3, 41.0, 34.2, 17.7 ppm. HRMS (ESI): m/z [M+H]+ calcd for C31H27N4O4S+ 551.1748. found 551.1750.

3-(N-(3-cyano-4-methyl-1H-indol-7-yl)sulfamoyl)-N-(4-(isopentyloxy) phenethyl)benzamide (HyT molecule 56): 1H NMR (400 MHz, DMSO): § 11.98 (s, 1H), 10.01 (s, 1H), 8.75 (t, J=5.6 Hz, 1H), 8.23-8.15 (m, 2H), 8.06-7.99 (m, 1H), 7.82-7.75 (m, 1H), 7.61 (t, J=7.8 Hz, 1H), 7.15-7.07 (m, 2H), 6.86-6.80 (m, 2H), 6.79-6.73 (m, 1H), 6.51 (d, J=7.7 Hz, 1H), 3.93 (t, J=6.6 Hz, 2H), 3.47-3.38 (m, 2H), 2.75 (t, J=7.3 Hz, 2H), 2.56 (s, 3H), 1.83-1.69 (m, 1H), 1.58 (q, J=6.7 Hz, 2H), 0.92 (s, 3H), 0.91 (s, 3H) ppm. 13C NMR (101 MHz, DMSO): δ 165.0, 157.6, 140.1, 135.8, 131.6, 131.6, 131.4, 130.0, 129.7, 128.1, 127.0, 126.4, 122.8, 120.7, 119.2, 117.8, 114.7, 84.8, 66.2, 41.7, 37.9, 34.5, 25.0, 22.9, 18.1 ppm. HRMS (ESI): m/z [M+H]+ calcd for C30H32N4O4S+ 545.2218. found 545.2222.

### General Procedure for Preparation of Hyt-Linker Conjugate (Intermediates for Hyt 49-53)

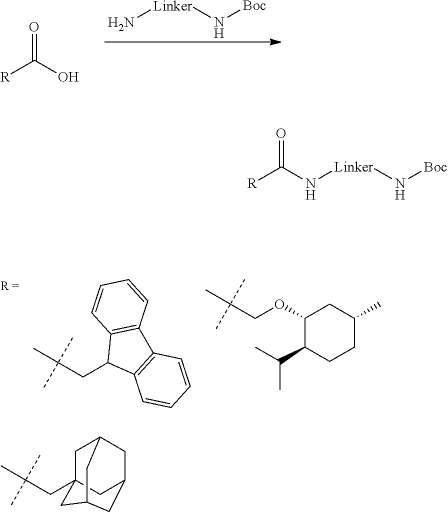

The corresponding carboxylic acid derivative (1.0 equiv.) was dissolved in DMF (2 mL) followed by the addition of 1-[Bis(dimethylamino)methylene]-1H-1,2,3-triazolo[4,5-b]pyridinium3-oxidhexafluoro-phosphate (HATU, 1.1 equiv.) and activation of the carboxylic acid for 30 min. Then, the reaction mixture was cooled to 0° C. and the respective mono-Boc protected diamine linker (0.5 equiv.) and then N,N-Diisopropylethylamine (DIPEA, 2.0 equiv.) were sequentially charged into the reaction flask. The reaction was continued until full conversion was observed via TLC analysis (typically 1 h). Then, the solution was diluted with Dichloromethane (DCM, 10 mL), washed with water and extracted with DCM (3×20 ml). The combined organic layers were washed with brine (2×50 mL), dried over Na₂SO₄, filtered, and concentrated in vacuo. The crude product was purified by column chromatography (DCM/MeOH 1 to 5%) to give an clear oil/white solid.

tert-butyl (1-(9H-fluoren-9-yl)-2-oxo-7,10,13-trioxa-3-azahexadecan-16-yl)carbamate (Intermediate for Hyt 49): Yield: 525 mg, 89%; Rf=0.40 (5:95 MeOH/DCM); 1H NMR (400 MHz, DMSO): δ 7.94 (t, J=5.5 Hz, 1H), 7.87 (dt, J=7.6, 0.9 Hz, 2H), 7.51 (dd, J=7.5, 1.0 Hz, 2H), 7.38 (tt, J=7.5, 0.9 Hz, 2H), 7.30 (td, J=7.4, 1.2 Hz, 2H), 6.74 (d, J=5.8 Hz, 1H), 4.35 (t, J=7.7 Hz, 1H), 3.55-3.50 (m, 4H), 3.50-3.45 (m, 4H), 3.43 (t, J=6.3 Hz, 2H), 3.37 (t, J=6.4 Hz, 2H), 3.22 (q, J=6.6 Hz, 2H), 2.95 (q, J=6.6 Hz, 2H), 2.49-2.47 (m, 2H), 1.69 (p, J=6.7 Hz, 2H), 1.59 (p, J=6.6 Hz, 2H), 1.36 (s, 9H) ppm. 13C NMR (101 MHz, DMSO): δ 171.0, 155.8, 147.2, 140.5, 127.7, 127.5, 125.0, 120.4, 77.9, 70.2, 70.0, 68.5, 44.0, 40.0, 37.7, 36.4, 30.2, 29.8, 28.7 ppm. LCMS (ESI): m/z [M−H]− calcd, for C30H41N2O6− 525.3. found 525.2.

tert-butyl (1-(((1R,2S,5R)-2-isopropyl-5-methylcyclohexyl)oxy)-2-oxo-7,10,13-trioxa-3-azahexadecan-16-yl)carbamate (Intermediate for Hyt 50): Yield: 446 mg, 74%; Rf=0.30 (5:95 MeOH/DCM); 1H NMR (400 MHz, DMSO): δ 7.48 (t, J=5.9 Hz, 1H), 6.74 (t, J=5.6 Hz, 1H), 3.90 (d, J=14.7 Hz, 1H), 3.77 (d, J=14.7 Hz, 1H), 3.54-3.49 (m, 4H), 3.49-3.44 (m, 4H), 3.40 (t, J=5.7 Hz, 2H), 3.37 (t, J=5.8 Hz, 2H), 3.19-3.08 (m, 3H), 2.95 (q, J=6.9 Hz, 2H), 2.17 (septd, J=7.0, 2.7 Hz, 1H), 2.03 (dtd, J=12.0, 3.8, 1.8 Hz, 1H), 1.69-1.61 (m, 3H), 1.60-1.52 (m, 3H), 1.41-1.27 (m, 10H), 1.22 (ddt, J=12.2, 10.2, 3.1 Hz, 1H), 0.99-0.89 (m, 1H), 0.88 (d, J=6.0 Hz, 3H), 0.86 (d, J=6.7 Hz, 3H), 0.84-0.81 (m, 1H), 0.79 (d, J=11.9 Hz, 1H), 0.74 (d, J=6.9 Hz, 3H) ppm. 13C NMR (101 MHz, DMSO): δ 169.8, 156.0, 79.8, 77.8, 70.2, 70.0, 68.9, 68.5, 68.2, 47.9, 40.2, 37.7, 36.2, 34.5, 31.3, 30.2, 29.7, 28.7, 25.6, 23.2, 22.6, 21.3, 16.5 ppm. LCMS (ESI): m/z [M+H]+ calcd, for C27H52N2O7+ 517.4. found 517.5.

tert-butyl (10-(2-(9H-fluoren-9-yl) acetamido) decyl)carbamate (Intermediate for Hyt 51): Yield: 521 mg, 97%; Rf=0.57 (5:95 MeOH/DCM) 1H NMR (400 MHz, DMSO): δ 7.91 (t, J=5.6 Hz, 1H), 7.87 (dt, J=7.6, 0.9 Hz, 2H), 7.51 (dd, J=7.5, 1.0 Hz, 2H), 7.38 (tt, J=7.5, 0.9 Hz, 2H), 7.28 (td, J=7.4, 1.2 Hz, 2H), 6.75 (t, J=5.7 Hz, 1H), 4.35 (t, J=7.7 Hz, 1H), 3.16 (q, J=6.5 Hz, 2H), 2.89 (q, J=6.8 Hz, 2H), 2.51-2.46 (m, 2H), 1.48-1.41 (m, 2H), 1.39-1.33 (m, 11H), 1.30-1.20 (m, 12H) ppm. 13C NMR (101 MHz, DMSO): δ 170.9, 155.9, 147.2, 140.5, 127.7, 127.4, 125.0, 120.4, 77.6, 44.0, 40.2, 40.0, 39.0, 29.9, 29.5, 29.4, 29.2, 28.7, 26.9, 26.7 ppm. LCMS (ESI): m/z [M−H]− calcd, for C30H41N2O3− 477.3. found 477.2.

tert-butyl (10-(2-(((1R,2S,5R)-2-isopropyl-5-methylcyclohexyl)oxy) acetamido) decyl)carbamate (Intermediate for Hyt 52): Yield: 274 mg, 50%; Rf=0.57 (5:95 MeOH/DCM); 1H NMR (400 MHz, DMSO): δ 7.42 (t, J=5.9 Hz, 1H), 6.74 (t, J=5.7 Hz, 1H), 3.89 (d, J=14.7 Hz, 1H), 3.77 (d, J=14.7 Hz, 1H), 3.17-3.03 (m, 3H), 2.88 (q, J=6.8 Hz, 2H), 2.17 (septd, J=7.0, 2.8 Hz, 1H), 2.03 (dtd, J=12.0, 3.7, 1.7 Hz, 1H), 1.66-1.56 (m, 1H), 1.60-1.51 (m, 1H), 1.43-1.29 (m, 14H), 1.28-1.17 (m, 13H), 1.00-0.89 (m, 1H), 0.88 (d, J=5.3 Hz, 3H), 0.86 (d, J=5.7 Hz, 3H), 0.84-0.80 (m, 1H), 0.80-0.76 (m, 1H), 0.74 (d, J=6.9 Hz, 3H) ppm. 13C NMR (101 MHz, DMSO): δ 169.7, 155.9, 79.8, 77.7, 68.2, 47.8, 40.2, 38.4, 34.5, 31.3, 29.9, 29.6, 29.4, 29.2, 28.7, 26.8, 26.7, 25.6, 23.2, 22.6, 21.3, 16.5 ppm. LCMS (ESI): m/z [M+H]+ calcd, for C27H53N2O4+ 469.4. found 469.3.

tert-butyl (10-(2-((3r,5r,7r)-adamantan-1-yl) acetamido) decyl)carbamate (Intermediate for Hyt 53): Yield: 436 mg, 76%; Rf=0.40 (5:95 MeOH/DCM); 1H NMR (400 MHz, DMSO): δ 7.61 (t, J=5.6 Hz, 1H), 6.74 (t, J=5.7 Hz, 1H), 2.99 (q, J=6.5 Hz, 2H), 2.87 (q, J=6.8 Hz, 2H), 1.93-1.86 (m, 3H), 1.79 (s, 2H), 1.68-1.54 (m, 6H), 1.56-1.51 (m, 6H), 1.40-1.31 (m, 13H), 1.25-1.20 (m, 12H) ppm. 13C NMR (101 MHz, DMSO): δ 170.1, 156.0, 77.7, 50.5, 42.6, 40.2, 38.6, 36.9, 32.6, 29.9, 29.6, 29.4, 29.1, 28.7, 28.5, 26.9, 26.7 ppm. LCMS (ESI): m/z [M+H]+ calcd, for C₂₇H₄₉N₂O₃+449.4. found 449.4.

### General Procedure for Synthesis of Hyt 49-53:

The Boc-protected linker-Hyt conjugate (1.1 equiv.) was dissolved in DCM (2 mL) and treated with TFA (0.75 mL). The reaction mixture was stirred at rt for 30 min to 1 h until complete deprotection and quenched by addition of sat. NaHCO₃ (15 mL). The aqueous solution was extracted with DCM (6×20 mL) until the organic phase is no longer fluorescent. The combined organic layers were dried using Na₂SO₄ and put on rotary evaporator. The residue was afterwards put on high vacuum. In a separate flask, 3-(N-(3-chloro-1H-indol-7-yl)sulfamoyl)benzoic acid (1.0 equiv.) is dissolved in DCM (2 mL), to which HATU (1.1 equiv.) was added. The deprotected amine and DIPEA (3.0 equiv.) were dissolved in DCM (4 mL) and added to the acid mixture, followed by stirring at rt for 1-18 h. The reaction is monitored by TLC and when reflecting zero change in spot intensities the reaction is quenched by diluting with water (10 mL) and extracting with DCM (3×10 mL). The combined organic phase was washed with brine (50 mL) dried using Na₂SO₄ and put on rotary evaporator. The crude product was purified by flash column chromatography yielding a white solid.

N-(1-(9H-fluoren-9-yl)-2-oxo-7,10,13-trioxa-3-azahexadecan-16-yl)-3-(N-(3-chloro-1H-indol-7-yl)sulfamoyl)benzamide (HyT molecule 49): Yield: 55 mg, 85%; Rf=0.44 (DCM/MeOH 95:5); 1H NMR (400 MHz, DMSO): δ 11.07 (s, 1H), 10.06 (s, 1H), 8.66 (t, J=5.6 Hz, 1H), 8.25 (s, 1H), 8.06-8.01 (m, 2H), 7.97-7.91 (m, 1H), 7.89-7.84 (m, 2H), 7.84-7.79 (m, 1H), 7.59 (t, J=7.8 Hz, 1H), 7.51 (d, J=7.5 Hz, 2H), 7.47 (d, J=2.7 Hz, 1H), 7.37 (t, J=7.4 Hz, 2H), 7.32-7.22 (m, 3H), 6.93 (t, J=7.8 Hz, 1H), 6.74 (d, J=7.6 Hz, 1H), 4.35 (t, J=7.7 Hz, 1H), 3.54-3.50 (m, 4H), 3.50-3.45 (m, 4H), 3.42 (q, J=6.2 Hz, 4H), 3.31-3.26 (m, 2H), 3.21 (q, J=6.5 Hz, 2H), 1.71 (dp, J=20.1, 6.8 Hz, 4H) ppm. 13C NMR (101 MHz, DMSO): δ 170.6, 164.6, 146.7, 140.1, 139.8, 135.3, 131.2, 129.4, 129.2, 129.1, 127.2, 127.0, 126.2, 125.9, 124.5, 123.0, 123.0, 121.6, 120.0, 120.0, 116.2, 114.9, 103.6, 69.8, 69.8, 69.6, 69.6, 68.2, 68.0, 43.6, 36.8, 35.9, 29.3, 29.2 ppm. LCMS (ESI): m/z [M+H]+ calcd for C40H43ClN4O7S+ 759.3. found 759.7.

3-(N-(3-chloro-1H-indol-7-yl)sulfamoyl)-N-(1-(((1R,2S,5R)-2-isopropyl-5-methylcyclohexyl)oxy)-2-oxo-7,10,13-trioxa-3-azahexadecan-16-yl)benzamide (HyT molecule 50): Yield: 36 mg, 56%; Rf=0.52 (DCM/MeOH 95:5); 1H NMR (400 MHz, DMSO): δ 11.06 (s, 1H), 10.05 (s, 1H), 8.65 (t, J=5.6 Hz, 1H), 8.25 (t, J=1.8 Hz, 1H), 8.09-8.00 (m, 1H), 7.85-7.78 (m, 1H), 7.59 (t, J=7.8 Hz, 1H), 7.51-7.44 (m, 2H), 7.25 (d, J=8.0 Hz, 1H), 6.93 (t, J=7.8 Hz, 1H), 6.74 (d, J=7.6 Hz, 1H), 3.93-3.73 (m, 2H), 3.54-3.41 (m, 10H), 3.39 (t, J=6.2 Hz, 2H), 3.32-3.26 (m, 2H), 3.18-3.06 (m, 3H), 2.16 (pd, J=6.9, 2.7 Hz, 1H), 2.06-1.96 (m, 1H), 1.74 (p, J=6.6 Hz, 2H), 1.68-1.50 (m, 4H), 1.36-1.27 (m, 1H), 1.27-1.15 (m, 1H), 0.99-0.89 (m, 1H), 0.88-0.83 (m, 6H), 0.83-0.74 (m, 2H), 0.72 (d, J=6.9 Hz, 3H) ppm. 13C NMR (101 MHz, DMSO): δ 169.4, 164.5, 139.7, 135.3, 131.2, 129.4, 129.2, 129.1, 126.2, 125.9, 123.0, 121.8, 119.9, 116.2, 114.9, 103.6, 79.3, 69.8, 69.7, 69.6, 69.6, 68.4, 68.2, 67.8, 47.4, 36.8, 35.8, 34.0, 30.8, 29.3, 29.2, 25.1, 22.7, 22.2, 20.9, 16.1 ppm. One CH2 group missing due to superimposition with DMSO. LCMS (ESI): m/z [M+H]+ calcd for C37H53ClN4O8S+ 749.3. found 749.6.

N-(10-(2-(9H-fluoren-9-yl) acetamido) decyl)-3-(N-(3-chloro-1H-indol-7-yl)sulfamoyl)benzamide (HyT molecule 51): Yield: 99 mg, 67%; Rf=0.41 (5:95 MeOH/DCM); 1H NMR (400 MHz, DMSO): δ 11.07 (s, 1H), 10.05 (s, 1H), 8.65 (t, J=5.6 Hz, 1H), 8.24 (t, J=1.9 Hz, 1H), 8.03-8.00 (m, 1H), 7.91 (t, J=5.6 Hz, 1H), 7.86 (d, J=7.5 Hz, 2H), 7.82-7.79 (m, 1H), 7.59 (t, J=7.8 Hz, 1H), 7.51 (d, J=7.5 Hz, 2H), 7.47 (d, J=2.7 Hz, 1H), 7.37 (t, J=7.4 Hz, 2H), 7.32-7.22 (m, 3H), 6.93 (t, J=7.8 Hz, 1H), 6.74 (d, J=7.5 Hz, 1H), 4.35 (t, J=7.7 Hz, 1H), 3.23 (q, J=6.7 Hz, 2H), 3.16 (q, J=6.5 Hz, 2H), 2.50-2.47 (m, 2H), 1.56-1.46 (m, 2H), 1.48-1.39 (m, 2H), 1.35-1.21 (m, 12H) ppm. 13C NMR (101 MHz, DMSO): δ 170.9, 164.9, 147.2, 140.5, 140.1, 135.8, 131.6, 129.8, 129.7, 129.5, 127.7, 127.4, 126.6, 126.4, 125.0, 123.5, 122.2, 120.4, 120.4, 116.7, 115.4, 104.1, 44.0, 39.8, 39.0, 29.5, 29.4, 29.2, 26.9 ppm. HRMS (ESI): m/z [M+H]+ calcd, for C40H44ClN4O4S+ 711.2767. found 711.2769.

3-(N-(3-chloro-1H-indol-7-yl)sulfamoyl)-N-(10-(2-(((1R,2S,5R)-2-isopropyl-5-methylcyclohexyl)oxy) acetamido) decyl)benzamide (HyT molecule 52): Yield: 95 mg, 73%; Rf=0.49 (5:95 MeOH/DCM); 1H NMR (400 MHz, DMSO): δ 11.07 (s, 1H), 10.05 (s, 1H), 8.64 (t, J=5.6 Hz, 1H), 8.24 (t, J=1.8 Hz, 1H), 8.03 (dt, J=7.8, 1.5 Hz, 1H), 7.85-7.78 (dt, J=8.0, 1.4 Hz, 1H), 7.59 (t, J=7.8 Hz, 1H), 7.47 (d, J=2.7 Hz, 1H), 7.42 (t, J=6.0 Hz, 1H), 7.25 (d, J=7.9 Hz, 1H), 6.93 (t, J=7.8 Hz, 1H), 6.74 (d, J=7.6 Hz, 1H), 3.88 (d, J=14.7 Hz, 1H), 3.77 (d, J=14.7 Hz, 1H), 3.22 (q, J=6.6 Hz, 2H), 3.14-3.04 (m, 3H), 2.16 (septd, J=7.0, 2.7 Hz, 1H), 2.02 (dtd, J=12.1, 5.2, 2.5 Hz, 1H), 1.63-1.54 (m, 1H), 1.59-1.52 (m, 1H), 1.48 (p, J=6.7 Hz, 2H), 1.39 (p, J=6.8 Hz, 2H), 1.32-1.17 (m, 14H), 0.98-0.88 (m, 1H), 0.87 (d, J=5.4 Hz, 3H), 0.85 (d, J=5.7 Hz, 3H), 0.83-0.79 (m, 1H), 0.79-0.75 (m, 1H), 0.73 (d, J=6.9 Hz, 3H) ppm. 13C NMR (101 MHz, DMSO): δ 169.7, 164.9, 140.1, 135.8, 131.6, 129.8, 129.7, 129.5, 126.6, 126.4, 123.4, 122.6, 120.4, 116.9, 115.4, 104.1, 79.8, 68.2, 47.8, 40.2, 39.7, 38.4, 34.5, 31.3, 29.6, 29.4, 29.4, 29.2, 26.9, 26.8, 25.6, 23.2, 22.6, 21.3, 16.5 ppm. LCMS (ESI): m/z [M+H]+ calcd, for C37H54ClN4O5S+ 701.3498. found 701.3502.

N-(10-(2-((3r,5r,7r)-adamantan-1-yl) acetamido) decyl)-3-(N-(3-chloro-1H-indol-7-yl)sulfamoyl)benzamide (HyT molecule 53): Yield: 96 mg, 61%; Rf=0.43 (5:95 MeOH/DCM); 1H NMR (400 MHz, DMSO): δ 11.07 (s, 1H), 10.05 (s, 1H), 8.64 (t, J=5.6 Hz, 1H), 8.24 (t, J=1.8 Hz, 1H), 8.03-8.00 (m, 1H), 7.82-7.79 (m, 1H), 7.63-7.56 (m, 2H), 7.47 (d, J=2.7 Hz, 1H), 7.25 (d, J=8.0 Hz, 1H), 6.93 (t, J=7.8 Hz, 1H), 6.74 (d, J=7.5 Hz, 1H), 3.22 (q, J=6.6 Hz, 2H), 2.99 (q, J=6.5 Hz, 2H), 1.92-1.85 (m, 3H), 1.79 (s, 2H), 1.67-1.53 (m, 6H), 1.55-1.52 (m, 6H), 1.52-1.44 (m, 2H), 1.40-1.31 (m, 2H), 1.30-1.20 (m, 12H) ppm. 13C NMR (101 MHz, DMSO): δ 170.1, 164.9, 140.1, 135.8, 131.6, 129.8, 129.7, 129.5, 126.6, 126.4, 123.4, 122.2, 120.4, 116.7, 115.4, 104.1, 50.6, 42.6, 39.8, 38.6, 36.9, 32.6, 29.6, 29.5, 29.4, 29.2, 28.5, 26.9 ppm. HRMS (ESI): m/z [M+H]+ calcd, for C37H50ClN4O4S+ 681.3236. found 681.3239.

### REFERENCES

Bricelj et al. E3 Ligase Ligands in Successful PROTACs: An Overview of Syntheses and Linker Attachment Points, Frontiers in Chemistry; July 2021; Volume 9; Article 707317.
Kleiger, G., and Mayor, T. Perilous Journey: A Tour of the Ubiquitin-Proteasome System. Trends Cel Biol. 2014, Vol 24, p. 352-359.
Han T., Goralski M., Gaskill N., Capota E., Kim J., C. Ting T. C., Xie Y., Williams N. S., and Nijhawan D. Anticancer sulfonamides target splicing by inducing RBM39 degradation via recruitment to DCAF15. SCIENCE, 16 Mar. 2017, Vol 356, Issue 6336

We claim:

1-18. (canceled)

19. A method for the treatment of amyloidosis by targeted protein degradation of DCAF15, said method comprising administering a therapeutic effective amount of a hydrophobic tag-based protein degradation (HyT-PD) molecule, a proteolysis targeting chimera (PROTAC), or a pharmaceutically acceptable salt of any thereof, said HyT-PD molecule or PROTAC comprising a ligand for DCAF15 (i.e. protein of interest) selected from a compound of Formula (DCAF15-I), (DCAF15-II), or (DCAF15-III) 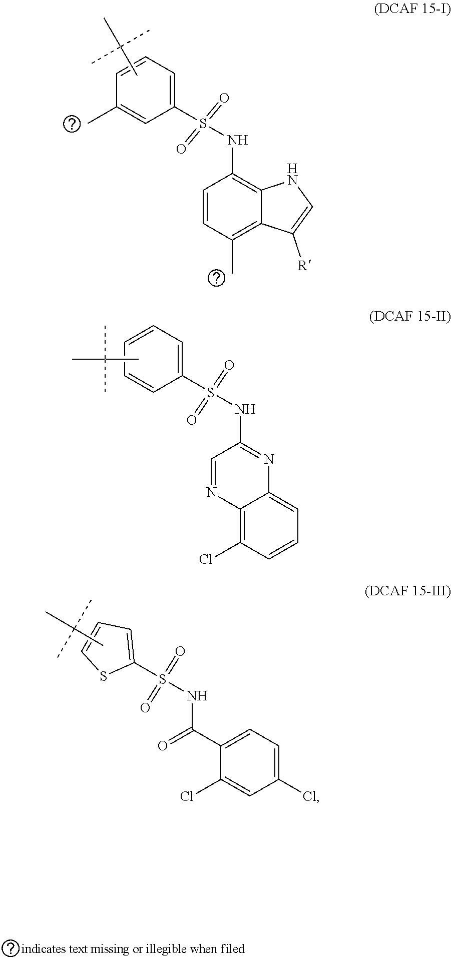
wherein R¹ is selected as F, Cl, Br, I, CN, Me, or CF₃; R² is selected as H, CN, CF₃, or Me; R³ is selected as CN or H; and wherein the dotted lines denote a covalent connection to a hydrophobic tag (HyT) via a linker, or to an E3 ubiquitin ligase ligand via a linker.

20. The method according to claim 19, said method comprising administering a therapeutic effective amount of a hydrophobic tag-based protein degradation (HyT-PD) molecule or a pharmaceutically acceptable salt thereof, said HyT-PD molecule comprising a ligand for DCAF15 (i.e. protein of interest) covalently connected to a hydrophobic tag (HyT) via a linker,
wherein
the ligand for DCAF15 is selected from a compound of Formula (DCAF15-I), (DCAF15-II), or (DCAF15-III) 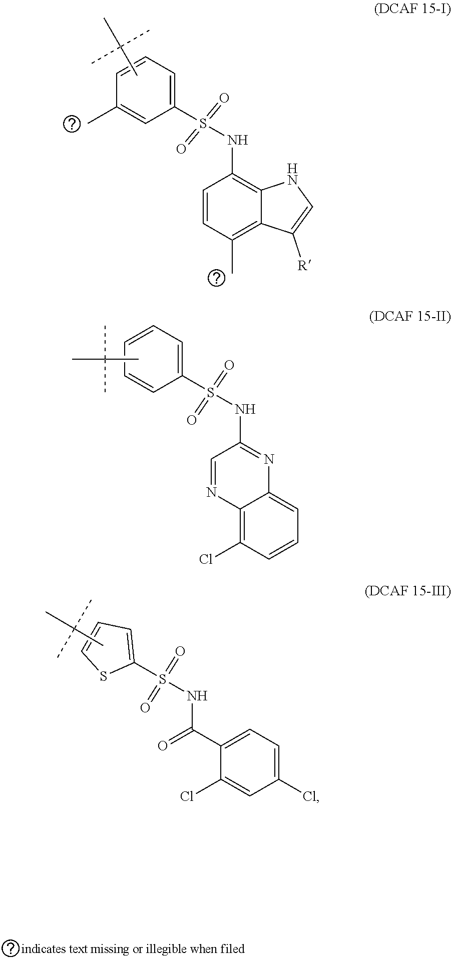
wherein R¹ is selected as F, Cl, Br, I, CN, Me, or CF₃; R² is selected as H, CN, CF₃, or Me; R³ is selected as CN or H; and wherein the dotted lines denote a covalent connection to the hydrophobic tag (HyT) via the linker;
the hydrophobic tag (HyT) is selected from a compound of Formula (I)-(VIII), 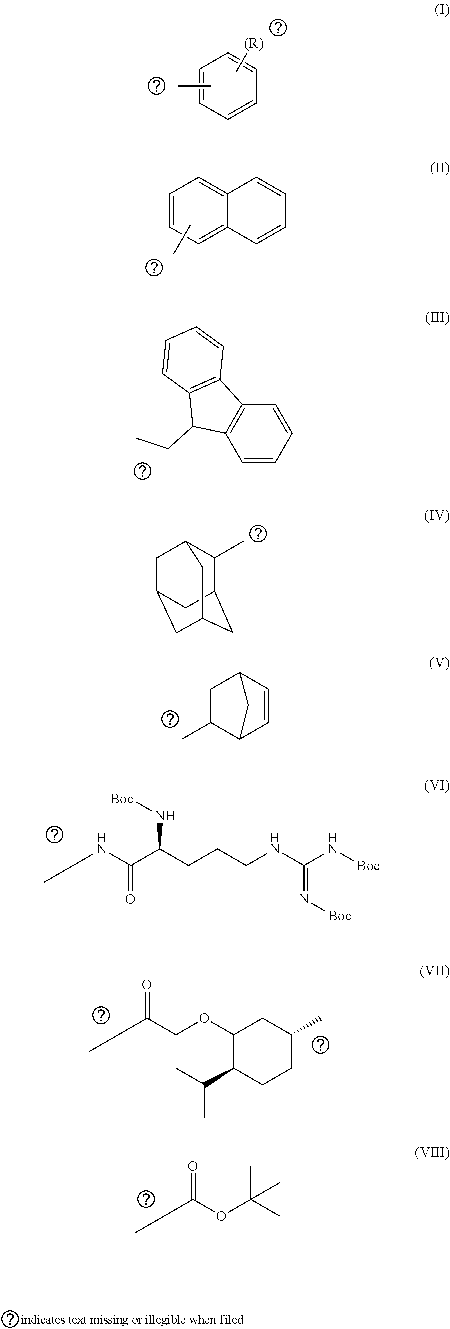
wherein the dotted lines denote a covalent connection to the DCAF15 ligand via the linker;
z is an integer of 0, 1, or 2;
R is/are independently selected from the list consisting of halogens, —O—(C₁-C₅-alkyl), —(C₁-C₅-alkyl), —O—(C₁-C₅-fluoroalkyl), —(C₁-C₅-fluoroalkyl), —O-phenyl, sulfonamide (—SO₂NH₂), and alkyl sulfonamide (—SO₂NH(C₁-C₅-alkyl) or —SO₂N (C₁-C₅-alkyl)₂).

21. The method according to claim 20, wherein the ligand for DCAF15 is selected from a compound of Formula (DCAF15-I).

22. The method according to claim 20, wherein R¹ is selected as Cl or CN; R² is selected as H or Me; and R³ is selected as H.

23. The method according to claim 20, wherein the hydrophobic tag (HyT) is selected from a compound for Formula (I)-(IV),

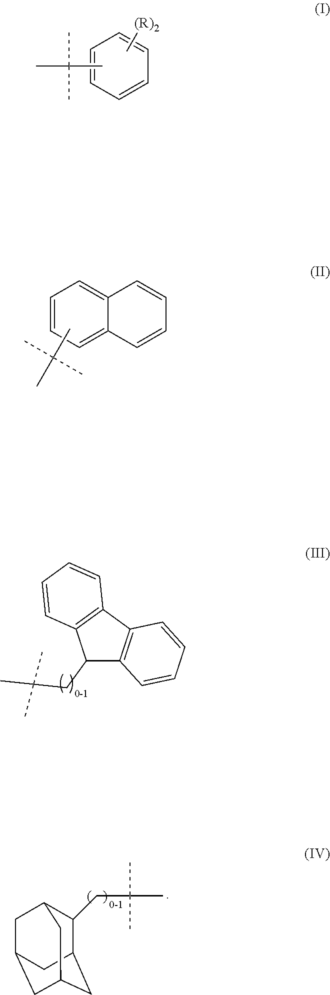

24. The method according to claim 20, wherein the HyT-PD molecule has the structure of compound nos. 24-56.

25. The method according to claim 19, said method comprising administering a therapeutic effective amount of a proteolysis targeting chimera (PROTAC) or a pharmaceutically acceptable salt thereof, said PROTAC comprising a ligand for DCAF15 (i.e. protein of interest) covalently connected to an E3 ubiquitin ligase ligand via a linker,
wherein
the ligand for DCAF15 is selected from a compound of Formula (DCAF15-I), (DCAF15-II), or (DCAF15-III) 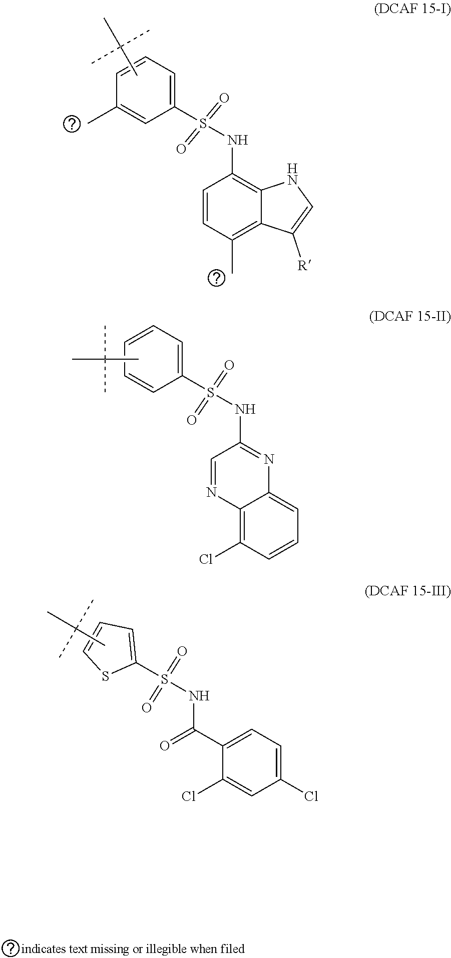
wherein R¹ is selected as F, Cl, Br, I, CN, Me, or CF₃; R² is selected as H, CN, CF₃ or Me; R³ is selected as CN or H; and wherein the dotted lines denote a covalent connection to the E3 ubiquitin ligase ligand via the linker;
the E3 ubiquitin ligase ligand is selected from a CRBN-ligand, a VHL-ligand, a RNF114-ligand, a DCAF11-ligand, a DCAF15-ligand, or a MDM2-ligand.

26. The method according to claim 25, wherein the E3 ligase ligand is selected from a CRBN-ligand or a VHL-ligand.

27. The method according to claim 25, wherein the ligand for DCAF15 is selected from a compound of Formula (DCAF15-I).

28. The method according to claim 25, wherein R¹ is selected as Cl or CN; R² is selected as H or Me; and R³ is selected as H.

29. The method according to claim 25, wherein the PROTAC has the structure of compound nos. 1-23.

30. A hydrophobic tag-based protein degradation (HyT-PD) molecule, a proteolysis targeting chimera (PROTAC), or a pharmaceutically acceptable salt of any thereof, for use in degrading DCAF15, said HyT-PD molecule or PROTAC comprising a ligand for DCAF15 (i.e. protein of interest) selected from a compound of Formula (DCAF15-I), (DCAF15-II), or (DCAF15-III) 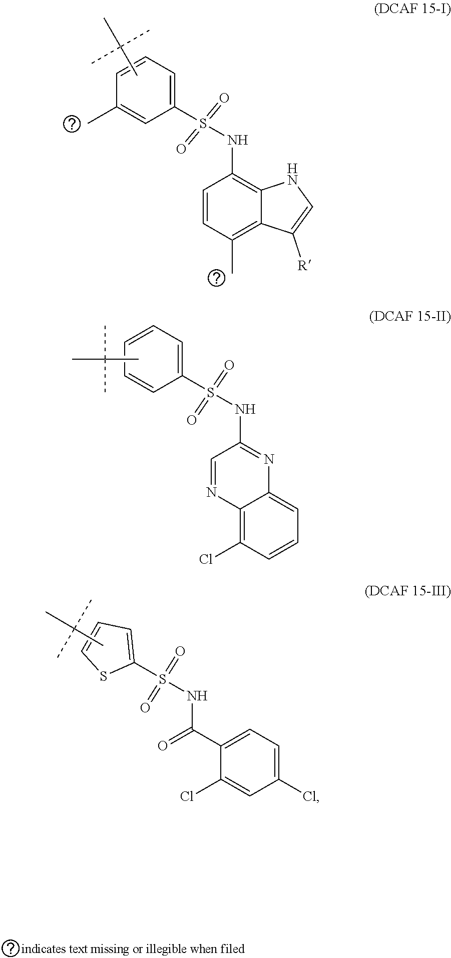
wherein R¹ is selected as F, Cl, Br, I, CN, Me, or CF₃; R² is selected as H, CN, CF₃, or Me; R³ is selected as CN or H; and wherein the dotted lines denote a covalent connection to a hydrophobic tag (HyT) via a linker, or to an E3 ubiquitin ligase ligand via a linker.

31. The HyT-PD molecule or a pharmaceutically acceptable salt thereof according to claim 30, said HyT-PD molecule comprising a ligand for DCAF15 (i.e. protein of interest) covalently connected to a hydrophobic tag (HyT) via a linker,
wherein
the ligand for DCAF15 is selected from a compound of Formula (DCAF15-I), (DCAF15-II), or (DCAF15-III) 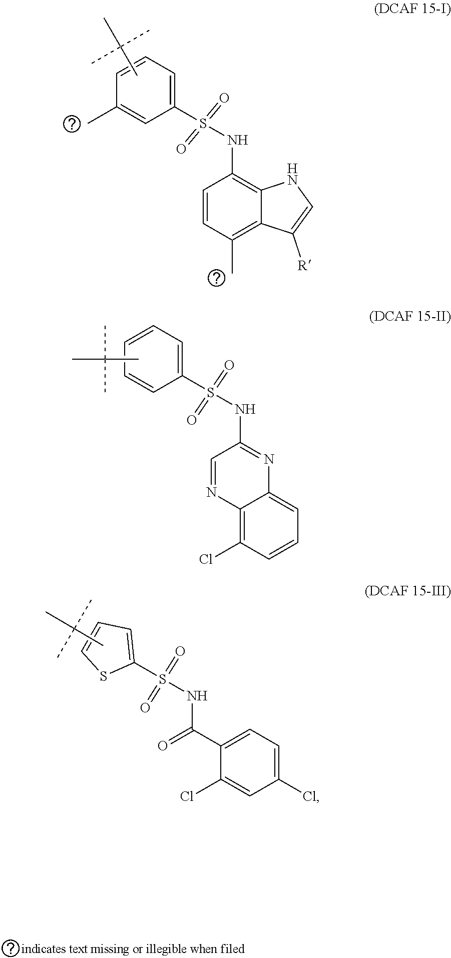
wherein R¹ is selected as F, Cl, Br, I, CN, Me, or CF₃; R² is selected as H, CN, CF₃ or Me; R³ is selected as CN or H; and wherein the dotted lines denote a covalent connection to the hydrophobic tag (HyT) via the linker;
the hydrophobic tag (HyT) is selected from a compound of Formula (I-VII) 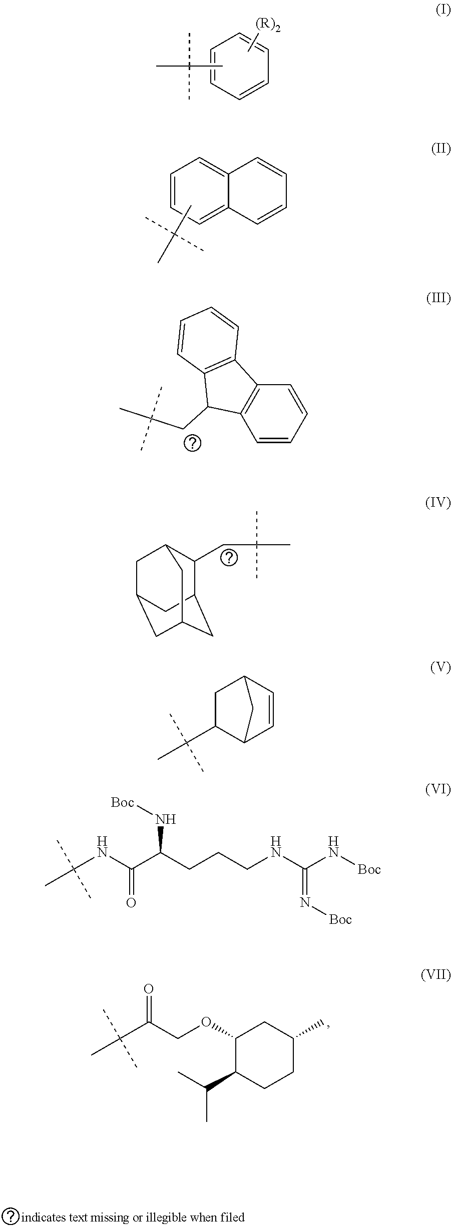
wherein the dotted lines denote a covalent connection to the DCAF15 ligand via the linker;
z is an integer of 0, 1, or 2;
R is/are independently selected from the list consisting of halogens, —O—(C₁-C₅-alkyl), —(C₁-C₅-alkyl), —O—(C₁-C₅-fluoroalkyl), —(C₁-C₅-fluoroalkyl), —O-phenyl, sulfonamide (—SO₂NH₂), and alkyl sulfonamide (—SO₂NH (C₁-C₅-alkyl) or —SO₂N (C₁-C₅-alkyl)₂).

32. The HyT-PD molecule according to claim 31, wherein the ligand for DCAF15 is selected from a compound of Formula (DCAF15-I).

33. The HyT-PD molecule according to claim 31, wherein R¹ is selected as Cl or CN; R² is selected as H or Me; and R³ is selected as H.

34. The HyT-PD molecule according to claim 31, wherein the hydrophobic tag (HyT) is selected from a compound for Formula (I)-(IV),

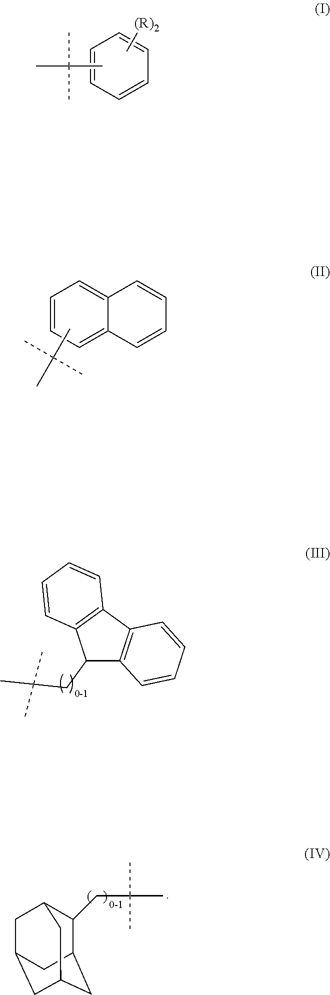

35. The HyT-PD molecule according to claim 31, wherein the HyT-PD molecule has the structure of compound nos. 24-56.

36. The proteolysis targeting chimera (PROTAC) or a pharmaceutically acceptable salt thereof according to claim 30, said PROTAC comprising a ligand for DCAF15 (i.e. protein of interest) covalently connected to an E3 ubiquitin ligase ligand via a linker,
wherein
the ligand for DCAF15 is selected from a compound of Formula (DCAF15-I), (DCAF15-II), or (DCAF15-III) 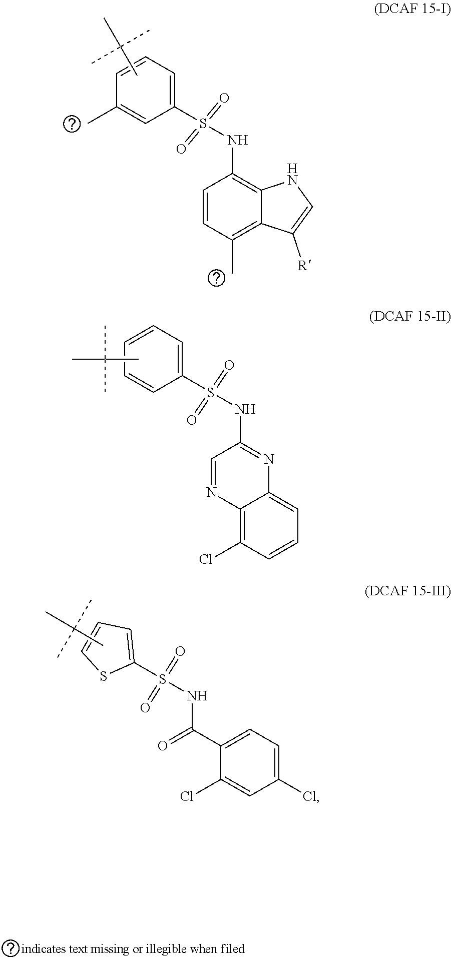
wherein R¹ is selected as F, Cl, Br, I, CN, Me, or CF₃; R² is selected as H, CN, CF₃, or Me; R³ is selected as CN or H; and wherein the dotted lines denote a covalent connection to the E3 ubiquitin ligase ligand via the linker;
the E3 ubiquitin ligase ligand is selected from a CRBN-ligand, a VHL-ligand, a RNF114-ligand, a DCAF11-ligand, or a MDM2-ligand.

37. The PROTAC according to claim 36, wherein the E3 ligase ligand is selected from a CRBN-ligand or a VHL-ligand.

38. The PROTAC according to claim 36, wherein the ligand for DCAF15 is selected from a compound of Formula (DCAF15-I).

39. The PROTAC according to claim 36, wherein R¹ is selected as Cl or CN; R² is selected as H or Me; and R³ is selected as H.

40. The PROTAC according to claim 36, wherein the PROTAC has the structure of compound nos. 1-23.
